(19) United States
(12) Patent Application Publication
(10) Pub. No.: US 2017/0002313 A1
(43) Pub. Date: Jan. 5, 2017

(54) COMPOSITIONS AND METHODS OF OBTAINING AND USING ENDODERM AND HEPATOCYTE CELLS

(71) Applicant: Genentech Inc., South San Francisco, CA (US)

(72) Inventors: Estelle Doudement, Paris (FR); Hirdesh Uppal, South San Francisco, CA (US)

(21) Appl. No.: 14/436,743

(22) PCT Filed: May 21, 2013
(86) PCT No.: PCT/EP2013/060372

§ 371 Date: Apr. 17, 2015

Related U.S. Application Data
(60) Provisional application No. 61/650,762, filed on May 23, 2012.

(51) Int. Cl. C12N 5/071; A61K 35/407; A61K 35/39; C12N 5/073

(52) U.S. Cl. CPC C12N 5/067; C12N 5/0603; C12N 5/0676; C12N 5/0678; A61K 35/407; A61K 35/39; C12N 2501/16; C12N 2501/415; C12N 2501/727; C12N 2501/999; C12N 2506/02; C12N 2506/45

(57) ABSTRACT

The invention provides for efficient methods for generating populations of endoderm cells and/or differentiated cells derived from endoderm cells (e.g., hepatic cells, pancreatic precursor cells, pancreatic cells, intestinal progenitor cells, intestinal cells, lung progenitor cells, lung cells, etc.). Also provided are compositions of endoderm cells and differentiated cells derived from endoderm cells (e.g., hepatic cells, pancreatic precursor cells, pancreatic cells, intestinal progenitor cells, intestinal cells, lung progenitor cells, lung cells, etc.) and methods of using such cells.

### CROSS REFERENCE TO RELATED APPLICATIONS

This application claims priority to U.S. Provisional Patent Application No. 61/650,762, filed May 23, 2012, the contents of which are incorporated herein by reference in its entirety.

### FIELD OF THE INVENTION

The invention is in the field of stem cells, endoderm cells, pancreatic progenitor cells, hepatocytes and other cells derived from endoderm cells. Generally, the invention relates to compositions and methods of making and using endoderm cells, pancreatic progenitor cells, hepatocytes, and/or other cells derived from endoderm cells.

### BACKGROUND OF THE INVENTION

Two properties that make stem cells uniquely suited to cell therapy applications are pluripotence and the ability to maintain these cells in culture for prolonged periods. Pluripotency is defined by the ability of stem cells to differentiate to derivatives of all three primary germ layers (endoderm, mesoderm, ectoderm) which, in turn, form all somatic cell types of the mature organism in addition to extraembryonic tissues (e.g. placenta) and germ cells. However, stem cells' pluripotency also poses unique challenges for the study and manipulation of these cells and their derivatives. Owing to the large variety of cell types that may arise in differentiating stem cell cultures, the vast majority of cell types are produced at very low efficiencies.

In order to use stem cells as a starting material to generate populations of cells that are useful in cell in research and therapy, it would be advantageous to overcome problems of production efficiency in terms of amount of conversion to the desired end population as well as the rate of conversion. For example, it would be useful to identify methods for generating populations of cell types, such as mesendoderm cells, endoderm cells and hepatocyte cells, which can be procured and/or maintained in cultures at increased proliferation rates, thereby providing a more plentiful and less costly supply of cells. Furthermore, for purposes of using various populations of cells (e.g., hepatocytes) for therapeutic purposes, it would be helpful to have populations of cells that have improved properties, such as maturation, to provide for better therapeutic potential. Thus, what is needed is robust populations of endoderm cells and populations of hepatocytes and methods for achieving the efficient, directed differentiation of stem cells into these cell types. The compositions and methods disclosed herein address these needs and provide additional benefits as well.

All references, publications, patents, and patent applications disclosed herein are hereby incorporated by reference in their entirety.

### BRIEF SUMMARY OF THE INVENTION

The invention provides, inter alia, compositions (e.g., populations) and methods for producing endoderm cells, pancreatic progenitor cells, hepatocytes, and other cells derived from endoderm cells that have unique properties. These populations of cells have utility for various screening and/or therapeutic uses.

Accordingly, in one aspect, the invention provides an isolated population of endoderm cells wherein at least 83% of the cells express SOX17, at least 77% of the cells express FoxA2, or at least 76% of the cells express CXCR4. In some embodiments according to (e.g., as applied to) the isolated population of endoderm cells above, at least 83% of the cells express SOX17 and at least 77% of the cells express FoxA2. In some embodiments according to (e.g., as applied to) the isolated population of endoderm cells above, at least 77% of the cells express FoxA2 and at least 76% of the cells express CXCR4. In some embodiments according to (e.g., as applied to) the isolated population of endoderm cells above, at least 83% of the cells express SOX17 and at least 76% of the cells express CXCR4. In some embodiments according to (e.g., as applied to) the isolated population of endoderm cells above, at least 83% of the cells express SOX17, at least 77% of the cells express FoxA2, and at least 76% of the cells express CXCR4. In some embodiments according to (e.g., as applied to) the isolated population of endoderm cells above, the endoderm cells have the capability to become hepatocytes, pancreatic cells, pancreatic progenitor cells, liver cells, or lung epithelial cells.

In another aspect, the invention provides bank of stable endoderm cells comprising one or more populations of endoderm cells wherein at least 83% of the cells express SOX17, at least 77% of the cells express FoxA2, and/or at least 76% of the cells express CXCR4, wherein the population maintains this phenotype for at least 10 passages. In some embodiments according to (e.g., as applied to) the bank of stable endoderm cells above, the endoderm cells have the capability to become hepatocytes, pancreatic cells, pancreatic progenitor cells, liver cells, or lung epithelial cells.

In another aspect, the invention provides methods obtaining a population of endoderm cells, the method comprising: contacting a population of stem cells with an effective amount of a selective inhibitor of PI3K alpha and an effective amount of an Activin A and culturing the cells under conditions sufficient to obtain the population of endoderm cells. In some embodiments according to (e.g., as applied to) any one of the methods above, at least 83% of the cells in the population of endoderm cells express SOX17, at least 77% of the cells in the population of endoderm cells express FoxA2, or at least 76% of the cells in the population of endoderm cells express CXCR4. In some embodiments according to (e.g., as applied to) any one of the methods above, at least 83% of the cells express SOX17 and at least 77% of the cells express FoxA2. In some embodiments according to (e.g., as applied to) any one of the methods above, at least 77% of the cells express FoxA2 and at least 76% of the cells express CXCR4. In some embodiments according to (e.g., as applied to) any one of the methods above, 83% of the cells express SOX17 and at least 76% of the cells express CXCR4. In some embodiments according to (e.g., as applied to) any one of the methods above, at least 83% of the cells express SOX17, at least 77% of the cells express FoxA2 and at least 76% of the cells express CXCR4. In some embodiments according to (e.g., as applied to) any one of the methods above, the endoderm cells have the capability to become hepatocytes, pancreatic cells, pancreatic progenitor cells, liver cells, or lung epithelial cells. In some embodiments according to (e.g., as applied to) any one of the methods above, the endoderm cells have greater viability and/or proliferation as compared to stem cells that have not been contacted with a selective inhibitor of PI3K alpha and Activin A.

In some embodiments according to (e.g., as applied to) any one of the methods above, the stem cells are adult stem cells, embryonic stem cells, or induced pluripotent stem cells. In some embodiments according to (e.g., as applied to) any one of the methods above, the stem cells are cultured in qualified matrigel. In some embodiments according to (e.g., as applied to) any one of the methods above, the stem cells are cultured in suspension.

In some embodiments according to (e.g., as applied to) any one of the methods above, the selective inhibitor of PI3K alpha is a compound which is a fused pyrimidine of formula (I):

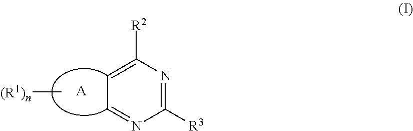

wherein A represents a thiophene or furan ring; n is 1 or 2; R¹ is a group of formula:

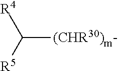

wherein m is 0 or 1; R³⁰ is H or C₁-C₆ alkyl; R⁴ and R⁵ form, together with the N atom to which they are attached, a 5- or 6-membered saturated N-containing heterocyclic group which includes 0 or 1 additional heteroatoms selected from N, S and O, which may be fused to a benzene ring and which is unsubstituted or substituted; or one of R⁴ and R⁵ is alkyl and the other is a 5- or 6-membered saturated N-containing heterocyclic group as defined above or an alkyl group which is substituted by a 5- or 6-membered saturated N-containing heterocyclic group as defined above; R² is selected from:

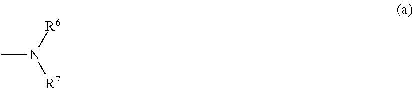

wherein R⁶ and R⁷ form, together with the nitrogen atom to which they are attached, a morpholine, thiomorpholine, piperidine, piperazine, oxazepane or thiazepane group which is unsubstituted or substituted; and

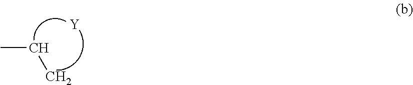

wherein Y is a C₂-C₄ alkylene chain which contains, between constituent carbon atoms of the chain and/or at one or both ends of the chain, 1 or 2 heteroatoms selected from O, N and S, and which is unsubstituted or substituted; and R³ is an indazole group which is unsubstituted or substituted; or a pharmaceutically acceptable salt thereof.

In some embodiments according to (e.g., as applied to) any one of the methods above, the fused pyrimidine is of formula (Ia):

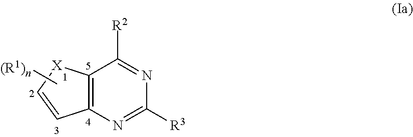

wherein X is S or O and R¹, R², R³ and n are as defined above.

In some embodiments according to (e.g., as applied to) any one of the methods above, the fused pyrimidine is of formula (Ib):

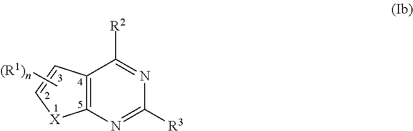

wherein X is S or O and R¹, R², R³ and n are as defined above.

In some embodiments according to (e.g., as applied to) any one of the methods above, the compound is selected from: 2-(1H-Indazol-4-yl)-6-(4-methyl-piperazin-1-ylmethyl)-4-morpholin-4-yl-thieno[3,2-d]pyrimidine; 4-[2-(1H-Indazol-4-yl)-4-morpholin-4-yl-thieno[3,2-d]pyrimidin-6-ylmethyl]-piperazine-1-sulfonic acid dimethylamide; {4-[2-(1H-Indazol-4-yl)-4-morpholin-4-yl-thieno[3,2-d]pyrimidin-6-ylmethyl]-piperazin-1-yl}-morpholin-4-yl-methanone; 4-[2-(1H-Indazol-4-yl)-4-morpholin-4-yl-thieno[3,2-d]pyrimidin-6-ylmethyl]-piperazine-1-carboxylic acid (2-methoxy-ethyl)-methyl-amide; {4-[2-(1H-Indazol-4-yl)-4-morpholin-4-yl-thieno[3,2-d]pyrimidin-6-ylmethyl]-piperazin-1-yl}-N,N-dimethyl-acetamide; 4-[2-(1H-Indazol-4-yl)-4-morpholin-4-yl-thieno[3,2-d]pyrimidin-6-ylmethyl]-piperazine-1-carboxylic acid dimethylamide; 2-(1H-Indazol-4-yl)-4-morpholin-4-yl-6-[4-(3-morpholin-4-yl-propane-1-sulfonyl)-piperazin-1-ylmethyl]-thieno[3,2-d]pyrimidine; {1-[2-(1H-Indazol-4-yl)-4-morpholin-4-yl-thieno[3,2-d]pyrimidin-6-ylmethyl]-piperidin-4-yl}-(2-methoxy-ethyl)-methyl-amine; (3-{4-[2-(1H-Indazol-4-yl)-4-morpholin-4-yl-thieno[3,2-d]pyrimidin-6-ylmethyl]-piperazine-1-sulfonyl}-propyl)-dimethyl-amine; 2-{4-[2-(1H-Indazol-4-yl)-4-morpholin-4-yl-thieno[3,2-d]pyrimidin-6-ylmethyl]-piperazin-1-yl}-2-methyl-propan-1-ol; 1′-[2-(1H-Indazol-4-yl)-4-morpholin-4-yl-thieno[3,2-d]pyrimidin-6-ylmethyl]-[1,4′]bipiperidinyl; 2-(1H-Indazol-4-yl)-4-morpholin-4-yl-6-(4-morpholin-4-yl-piperidin-1-ylmethyl)-thieno[3,2-d]pyrimidine; 2-(1H-Indazol-4-yl)-4-morpholin-4-yl-6-(4-pyrimidin-2-yl-piperazin-1-ylmethyl)-thieno[3,2-d]pyrimidine; 1-(2-Hydroxy-ethyl)-4-[2-(1H-indazol-4-yl)-4-morpholin-4-yl-thieno[3,2-d]pyrimidin-6-ylmethyl]-piperazin-2-one; 6-(4-Cyclopropylmethyl-piperazin-1-ylmethyl)-2-(1H-indazol-4-yl)-4-morpholin-4-yl-thieno[3,2-d]pyrimidine; 2-(1H-Indazol-4-yl)-4-morpholin-4-yl-6-(4-pyridin-2-yl-piperazin-1-ylmethyl)-thieno[3,2-d]pyrimidine; 2-(1H-Indazol-4-yl)-4-morpholin-4-yl-6-[4-(2,2,2-trifluoro-ethyl)-piperazin-1-ylmethyl]-thieno[3,2-d]pyrimidine; 2-(1H-Indazol-4-yl)-4-morpholin-4-yl-6-(4-thiazol-2-yl-piperazin-1-ylmethyl)-thieno[3,2-d]pyrimidine; 2-(6-Fluoro-1H-indazol-4-yl)-6-(4-methyl-piperazin-1-ylmethyl)-4-morpholin-4-yl-thieno[3,2-d]pyrimidine; 2-(1H-Indazol-4-yl)-4-morpholin-4-yl-6-(4-pyridin-2-ylmethyl-piperazin-1-ylmethyl)-thieno[3,2-d]pyrimidine; 2-(1H-Indazol-4-yl)-4-morpholin-4-yl-6-(4-thiazol-2-ylmethyl-piperazin-1-ylmethyl)-thieno[3,2-d]pyrimidine; 2-(1H-Indazol-4-yl)-6-[4-(5-methyl-furan-2-ylmethyl)-piperazin-1-ylmethyl]-4-morpholin-4-yl-thieno[3,2-d]pyrimidine; 1-[2-(1H-Indazol-4-yl)-4-morpholin-4-yl-thieno[3,2-d]pyrimidin-6-ylmethyl]-piperidine-4-carboxylic acid amide; 2-(1H-Indazol-4-yl)-6-[4-(2-methoxy-1,1-dimethyl-ethyl)-piperazin-1-ylmethyl]-4-morpholin-4-yl-thieno[3,2-d]pyrimidine; 2-(1H-Indazol-4-yl)-6-[(3R,5S)-4-(2-methoxy-ethyl)-3,5-dimethyl-piperazin-1-ylmethyl]-4-morpholin-4-yl-thieno[3,2-d]pyrimidine; 1-[2-(1H-Indazol-4-yl)-4-morpholin-4-yl-thieno[3,2-d]pyrimidin-6-ylmethyl]-piperidine-4-carboxylic acid (2-methoxy-ethyl)-methyl-amide; 1-[2-(1H-Indazol-4-yl)-4-morpholin-4-yl-thieno[3,2-d]pyrimidin-6-ylmethyl]-piperidine-4-carboxylic acid dimethylamide; 2-(1H-Indazol-4-yl)-4-morpholin-4-yl-6-(4-pyridin-3-ylmethyl-piperazin-1-ylmethyl)-thieno[3,2-d]pyrimidine; 1-[2-(1H-Indazol-4-yl)-4-morpholin-4-yl-thieno[3,2-d]pyrimidin-6-ylmethyl]-piperidine-4-carboxylic acid methylamide; 2-{4-[2-(1H-Indazol-4-yl)-4-morpholin-4-yl-thieno[3,2-d]pyrimidin-6-ylmethyl]-piperazin-1-yl}-N-methyl-isobutyramide; 2-{4-[2-(1H-Indazol-4-yl)-4-morpholin-4-yl-thieno[3,2-d]pyrimidin-6-ylmethyl]-piperazin-1-yl}-2-methyl-1-pyrrolidin-1-yl-propan-1-one; 2-(1H-Indazol-4-yl)-6-[4-(1-methyl-1H-imidazol-2-ylmethyl)-piperazin-1-ylmethyl]-4-morpholin-4-yl-thieno[3,2-d]pyrimidine; 2-(1H-Indazol-4-yl)-6-[4-(5-methyl-isoxazol-3-ylmethyl)-piperazin-1-ylmethyl]-4-morpholin-4-yl-thieno[3,2-d]pyrimidine; 1-{4-[2-(1H-Indazol-4-yl)-4-morpholin-4-yl-thieno[3,2-d]pyrimidin-6-ylmethyl]-piperazin-1-yl}-2-methyl-propan-2-ol; Cyclopropylmethyl-{1-[2-(1H-indazol-4-yl)-4-morpholin-4-yl-thieno[3,2-d]pyrimidin-6-ylmethyl]-piperidin-4-yl}-(2-methoxy-ethyl)-amine; 6-[4-(1-Ethyl-1-methoxymethyl-propyl)-piperazin-1-ylmethyl]-2-(1H-indazol-4-yl)-4-morpholin-4-yl-thieno[3,2-d]pyrimidine; 2-(1H-Indazol-4-yl)-6-[4-(1-methoxymethyl-cyclopropyl)-piperazin-1-ylmethyl]-4-morpholin-4-yl-thieno[3,2-d]pyrimidine; {1-[2-(1H-Indazol-4-yl)-4-morpholin-4-yl-thieno[3,2-d]pyrimidin-6-ylmethyl]-piperidin-4-yl}-(2-methoxy-ethyl)-(2,2,2-trifluoro-ethyl)-amine; 2-(1H-Indazol-4-yl)-6-[4-(2-methoxy-ethyl)-piperazin-1-ylmethyl]-4-morpholin-4-yl-thieno[3,2-d]pyrimidine; {1-[2-(1H-Indazol-4-yl)-4-morpholin-4-yl-thieno[3,2-d]pyrimidin-6-ylmethyl]-piperidin-4-yl}-methanol; 2-(1H-Indazol-4-yl)-4-morpholin-4-yl-6-(4-pyridin-4-ylmethyl-piperazin-1-ylmethyl)-thieno[3,2-d]pyrimidine; 2-(1H-Indazol-4-yl)-6-[4-(6-methyl-pyridin-2-ylmethyl)-piperazin-1-ylmethyl]-4-morpholin-4-yl-thieno[3,2-d]pyrimidine; 2-(1H-Indazol-4-yl)-6-[4-(4-methyl-thiazol-2-ylmethyl)-piperazin-1-ylmethyl]-4-morpholin-4-yl-thieno[3,2-d]pyrimidine; {1-[2-(1H-Indazol-4-yl)-4-morpholin-4-yl-thieno[3,2-d]pyrimidin-6-ylmethyl]-piperidin-4-yl}-pyridin-2-yl-amine; N-{1-[2-(1H-Indazol-4-yl)-4-morpholin-4-yl-thieno[3,2-d]pyrimidin-6-ylmethyl]-piperidin-4-yl}-2-methoxy-N-methyl-acetamide; N-{1-[2-(1H-Indazol-4-yl)-4-morpholin-4-yl-thieno[3,2-d]pyrimidin-6-ylmethyl]-piperidin-4-yl}-N-methyl-methanesulfonamide; {1-[2-(1H-Indazol-4-yl)-4-morpholin-4-yl-thieno[3,2-d]pyrimidin-6-ylmethyl]-piperidin-4-yl}-(3-methoxy-propyl)-methyl-amine; 6-((3S,5R)-3,5-Dimethyl-4-pyridin-2-ylmethyl-piperazin-1-ylmethyl)-2-(1H-indazol-4-yl)-4-morpholin-4-yl-thieno[3,2-d]pyrimidine; 2-(1H-Indazol-4-yl)-6-(4-methoxymethyl-piperidin-1-ylmethyl)-4-morpholin-4-yl-thieno[3,2-d]pyrimidine; {1-[2-(1H-Indazol-4-yl)-4-morpholin-4-yl-thieno[3,2-d]pyrimidin-6-ylmethyl]-piperidin-4-yl}-(2-methoxy-ethyl)-thiazol-2-ylmethyl-amine; 1-[2-(1H-Indazol-4-yl)-4-morpholin-4-yl-thieno[3,2-d]pyrimidin-6-ylmethyl]-4-pyridin-2-ylmethyl-piperidin-4-ol; {1-[2-(1H-Indazol-4-yl)-4-morpholin-4-yl-thieno[3,2-d]pyrimidin-6-ylmethyl]-piperidin-4-yl}-isopropyl-(2-methoxy-ethyl)-amine; 2-(1H-Indazol-4-yl)-4-morpholin-4-yl-6-[4-(pyridin-2-yloxy)-piperidin-1-ylmethyl]-thieno[3,2-d]pyrimidine; N-{1-[2-(1H-Indazol-4-yl)-4-morpholin-4-yl-thieno[3,2-d]pyrimidin-6-ylmethyl]-piperidin-4-yl}-N-(2-methoxy-ethyl)-methanesulfonamide; 2-{1-[2-(1H-Indazol-4-yl)-4-morpholin-4-yl-thieno[3,2-d]pyrimidin-6-ylmethyl]-piperidin-4-yl}-propan-2-ol; 2-(1H-Indazol-4-yl)-4-morpholin-4-yl-6-[4-(1-oxy-pyridin-3-ylmethyl)-piperazin-1-ylmethyl]-thieno[3,2-d]pyrimidine; 2-(1H-Indazol-4-yl)-4-morpholin-4-yl-6-(4-morpholin-4-ylmethyl-piperidin-1-ylmethyl)-thieno[3,2-d]pyrimidine; {1-[2-(1H-Indazol-4-yl)-4-morpholin-4-yl-thieno[3,2-d]pyrimidin-6-ylmethyl]-piperidin-4-ylmethyl}-(2-methoxy-ethyl)-methyl-amine; {1-[2-(1H-Indazol-4-yl)-4-morpholin-4-yl-thieno[3,2-d]pyrimidin-6-ylmethyl]-piperidin-4-ylmethyl}-dimethyl-amine; {1-[2-(1H-Indazol-4-yl)-4-morpholin-4-yl-thieno[3,2-d]pyrimidin-6-ylmethyl]-piperidin-3-yl}-(2-methoxy-ethyl)-methyl-amine; 1-[2-(1H-Indazol-4-yl)-4-morpholin-4-yl-thieno[3,2-d]pyrimidin-6-ylmethyl]-piperidine-3-carboxylic acid methylamide; 2-(1H-Indazol-4-yl)-6-(3-methoxymethyl-piperidin-1-ylmethyl)-4-morpholin-4-yl-thieno[3,2-d]pyrimidine; 2-(1H-Indazol-4-yl)-4-morpholin-4-yl-6-(4-pyridin-2-ylmethyl-piperidin-1-ylmethyl)-thieno[3,2-d]pyrimidine; 2-(1H-Indazol-4-yl)-6-[4-(2-methoxy-ethoxy)-piperidin-1-ylmethyl]-4-morpholin-4-yl-thieno[3,2-d]pyrimidine; 6-((3R,5S)-3,5-Dimethyl-4-thiazol-2-ylmethyl-piperazin-1-ylmethyl)-2-(1H-indazol-4-yl)-4-morpholin-4-yl-thieno[3,2-d]pyrimidine; 2-(1H-Indazol-4-yl)-4-morpholin-4-yl-6-[4-(1-oxy-pyridin-2-ylmethyl)-piperazin-1-ylmethyl]-thieno[3,2-d]pyrimidine; 2-(1H-Indazol-4-yl)-6-[4-(2-methoxy-ethyl)-piperidin-1-ylmethyl]-4-morpholin-4-yl-thieno[3,2-d]pyrimidine; 2-(1H-Indazol-4-yl)-6-(4-methanesulfonyl-piperidin-1-ylmethyl)-4-morpholin-4-yl-thieno[3,2-d]pyrimidine; {1-[2-(1H-Indazol-4-yl)-4-morpholin-4-yl-thieno[3,2-d]pyrimidin-6-ylmethyl]-piperidin-4-yl}-(3-methanesulfonyl-propyl)-methyl-amine; 2-(1H-Indazol-4-yl)-6-[4-(3-methoxy-propane-1-sulfonyl)-piperidin-1-ylmethyl]-4-morpholin-4-yl-thieno[3,2-d]pyrimidine; (R)-1-[2-(1H-Indazol-4-yl)-4-morpholin-4-yl-thieno[3,2-d]pyrimidin-6-ylmethyl]-piperidine-3-carboxylic acid methylamide; (S)-1-[2-(1H-Indazol-4-yl)-4-morpholin-4-yl-thieno[3,2-d]pyrimidin-6-ylmethyl]-piperidine-3-carboxylic acid methylamide; 6-(4-Imidazol-1-ylmethyl-piperidin-1-ylmethyl)-2-(1H-indazol-4-yl)-4-morpholin-4-yl-thieno[3,2-d]pyrimidine; 2-(1H-Indazol-4-yl)-4-morpholin-4-yl-6-morpholin-4-ylmethyl-thieno[3,2-d]pyrimidine; 2-(1H-Indazol-4-yl)-6-(3-methyl-piperidin-1-ylmethyl)-4-morpholin-4-yl-thieno[3,2-d]pyrimidine; {1-[2-(1H-Indazol-4-yl)-4-morpholin-4-yl-thieno[3,2-d]pyrimidin-6-ylmethyl]-piperidin-3-yl}-methanol; 2-{1-[2-(1H-Indazol-4-yl)-4-morpholin-4-yl-thieno[3,2-d]pyrimidin-6-ylmethyl]-piperidin-4-yl}-ethanol; 1-[2-(1H-Indazol-4-yl)-4-morpholin-4-yl-thieno[3,2-d]pyrimidin-6-ylmethyl]-4-thiazol-2-yl-piperidin-4-ol; 2-(1-Methyl-1H-indazol-4-yl)-6-(4-methyl-piperazin-1-ylmethyl)-4-morpholin-4-yl-thieno[3,2-d]pyrimidine; 2-(2-Methyl-2H-indazol-4-yl)-6-(4-methyl-piperazin-1-ylmethyl)-4-morpholin-4-yl-thieno[3,2-d]pyrimidine; 2-(1H-Indazol-4-yl)-4-morpholin-4-yl-6-(4-thiazol-4-ylmethyl-piperazin-1-ylmethyl)-thieno[3,2-d]pyrimidine; 1-{4-[2-(1H-Indazol-4-yl)-4-morpholin-4-yl-thieno[3,2-d]pyrimidin-6-ylmethyl]-piperazin-1-yl}-3-phenoxy-propan-2-ol; 6-[4-(1H-Imidazol-2-ylmethyl)-piperazin-1-ylmethyl]-2-(1H-indazol-4-yl)-4-morpholin-4-yl-thieno[3,2-d]pyrimidine; 6-[4-(3H-Imidazol-4-ylmethyl)-piperazin-1-ylmethyl]-2-(1H-indazol-4-yl)-4-morpholin-4-yl-thieno[3,2-d]pyrimidine; 2-(1H-Indazol-4-yl)-4-morpholin-4-yl-6-((2S,6R)-2,4,6-trimethyl-piperazin-1-ylmethyl)-thieno[3,2-d]pyrimidine; {4-[2-(1H-Indazol-4-yl)-4-morpholin-4-yl-thieno[3,2-d]pyrimidin-6-ylmethyl]-1-methanesulfonyl-piperazin-2-yl}-methanol; 2-(1H-Indazol-4-yl)-6-(4-methanesulfonyl-3-methoxymethyl-piperazin-1-ylmethyl)-4-morpholin-4-yl-thieno[3,2-d]pyrimidine; and the pharmaceutically acceptable salts of the above-mentioned free compounds.

In some embodiments according to (e.g., as applied to) any one of the methods above, the selective inhibitor of PI3K alpha is selected from the following compounds:

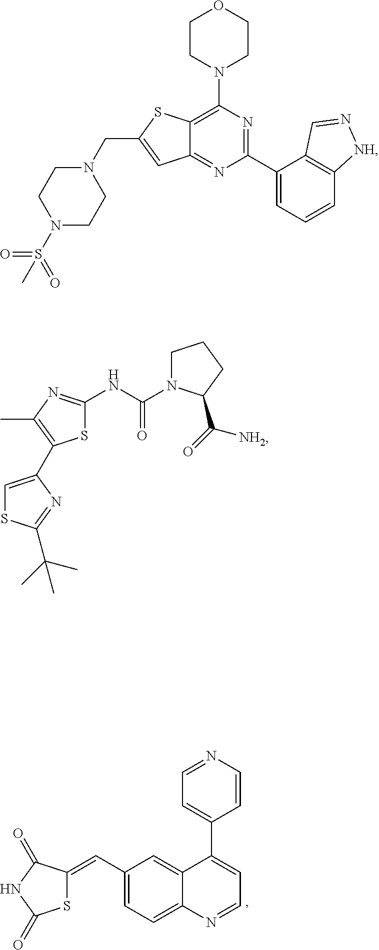 [SMILES: CS(=O)(=O)N1CCN(Cc2cc3nc(-c4cccc5[nH]ncc45)nc(N4CCOCC4)c3s2)CC1 ; Cc1nc(NC(=O)N2CCC[C@H]2C(N)=O)sc1-c1csc(C(C)(C)C)n1 ; O=C1NC(=O)/C(=C/c2ccc3nccc(-c4ccncc4)c3c2)S1]

### INK1117, and BYL719.

In some embodiments according to (e.g., as applied to) any one of the methods above, the selective inhibitor of PI3K alpha is selected from

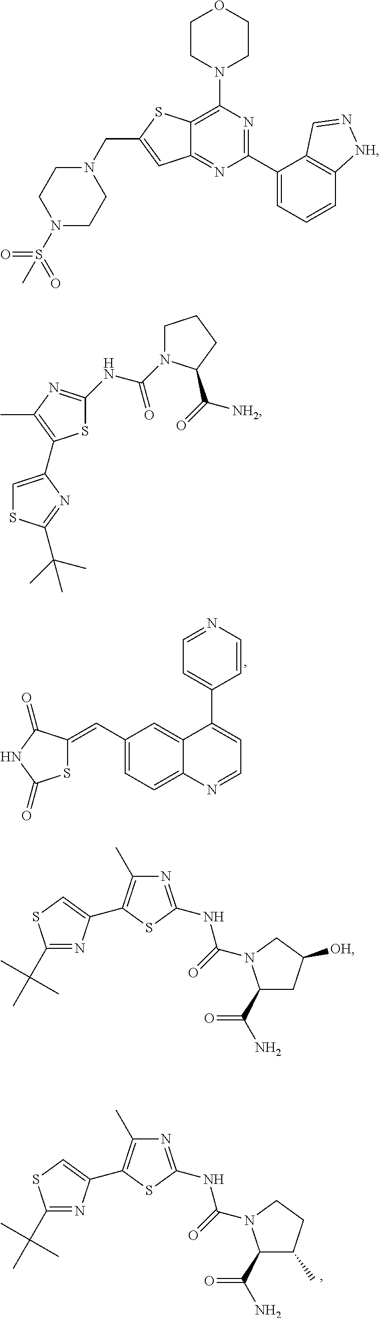 [SMILES: CS(=O)(=O)N1CCN(Cc2cc3nc(-c4cccc5[nH]ncc45)nc(N4CCOCC4)c3s2)CC1 ; Cc1nc(NC(=O)N2CCC[C@H]2C(N)=O)sc1-c1csc(C(C)(C)C)n1 ; O=C1NC(=O)/C(=C/c2ccc3nccc(-c4ccncc4)c3c2)S1 ; Cc1nc(NC(=O)N2C[C@@H](O)C[C@H]2C(N)=O)sc1-c1csc(C(C)(C)C)n1 ; Cc1nc(NC(=O)N2CC[C@H](C)[C@H]2C(N)=O)sc1-c1csc(C(C)(C)C)n1]

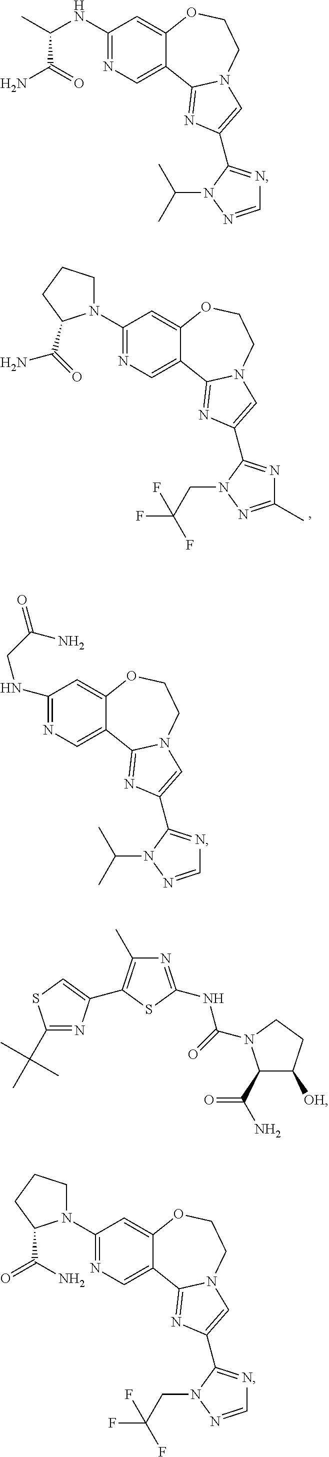

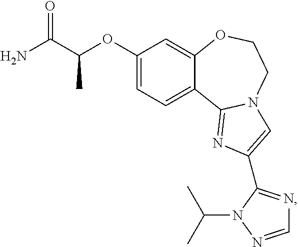 [SMILES: CC(C)n1ncnc1-c1cn2c(n1)-c1ccc(O[C@@H](C)C(N)=O)cc1OCC2]

### INK1117, and BYL719.

In some embodiments according to (e.g., as applied to) any one of the methods above, the selective inhibitor of PI3K alpha is 4-[2-(1H-indazol-4-yl)-6-[(4-methylsulfonylpiperazin-1-yl)methyl]thieno[3,2-d]pyrimidin-4-yl]morpholine. In some embodiments according to (e.g., as applied to) any one of the methods above, the selective inhibitor of P13K alpha is also an inhibitor of PI3K delta.

In some embodiments according to (e.g., as applied to) any one of the methods above, the effective amount of the selective inhibitor of PI3K alpha is 750 nM. In some embodiments according to (e.g., as applied to) any one of the methods above, the effective amount of Activin A is 100 ng/ml of medium. In some embodiments according to (e.g., as applied to) any one of the methods above, culturing the cells under conditions sufficient to obtain the population of endoderm cells comprises culturing the cells in the absence of Wnt3a.

In some embodiments according to (e.g., as applied to) any one of the methods above, the method further comprises contacting the population of stem cells with an effective amount of an mTOR inhibitor. In some embodiments according to (e.g., as applied to) any one of the methods above, the method further comprises contacting the population of stem cells with a selective inhibitor of PI3K delta.

In another aspect, the invention provides a population of endoderm cells obtained using any one of the methods above.

In another aspect, the invention provides methods of obtaining a population of endoderm cells, the method comprising: contacting a population of stem cells with an effective amount of an inhibitor of mTOR and an effective amount of an Activin A and culturing the cells under conditions sufficient to obtain the population of endoderm cells. In some embodiments according to (e.g., as applied to) any one of the methods above, wherein at least 61% of the cells in the population of endoderm cells express SOX17 or at least 40% of the cells in the population of endoderm cells express FoxA2. In some embodiments according to (e.g., as applied to) any one of the methods above, at least 61% of the cells in the population of endoderm cells express SOX17 and at least 40% of the cells in the population of endoderm cells express FoxA2. In some embodiments according to (e.g., as applied to) any one of the methods above, the endoderm cells have the capability to become hepatocytes, pancreatic cells, pancreatic progenitor cells, liver cells, or lung epithelial cells.

In some embodiments according to (e.g., as applied to) any one of the methods above, the inhibitor of mTOR is a siRNA or a small molecule. In some embodiments according to (e.g., as applied to) any one of the methods above, said small molecule is selected from the group consisting of:

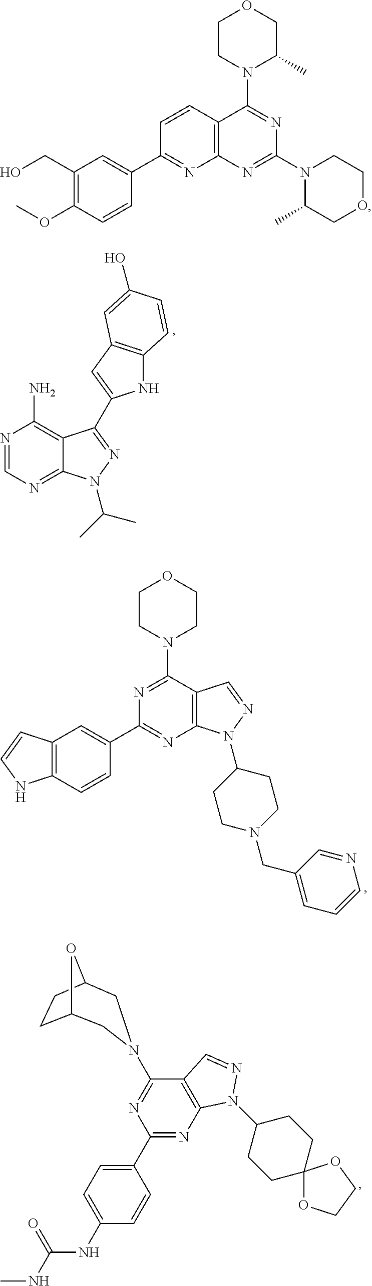

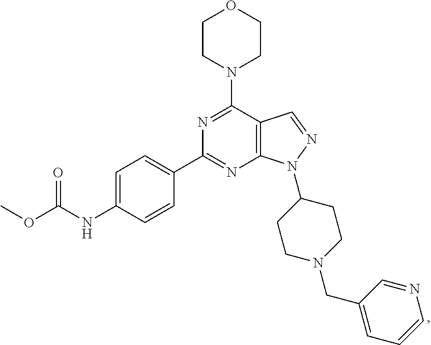 [SMILES: COC(=O)Nc1ccc(-c2nc(N3CCOCC3)c3cnn(C4CCN(Cc5cccnc5)CC4)c3n2)cc1]

AP23573, Torsel, INK128, AZD80555, AZD2012, CC-223, KU-0063794, OSI-027, sirolimus rapamycin, and everolimus.

In some embodiments according to (e.g., as applied to) any one of the methods above, said small molecule is selected from the group consisting of:

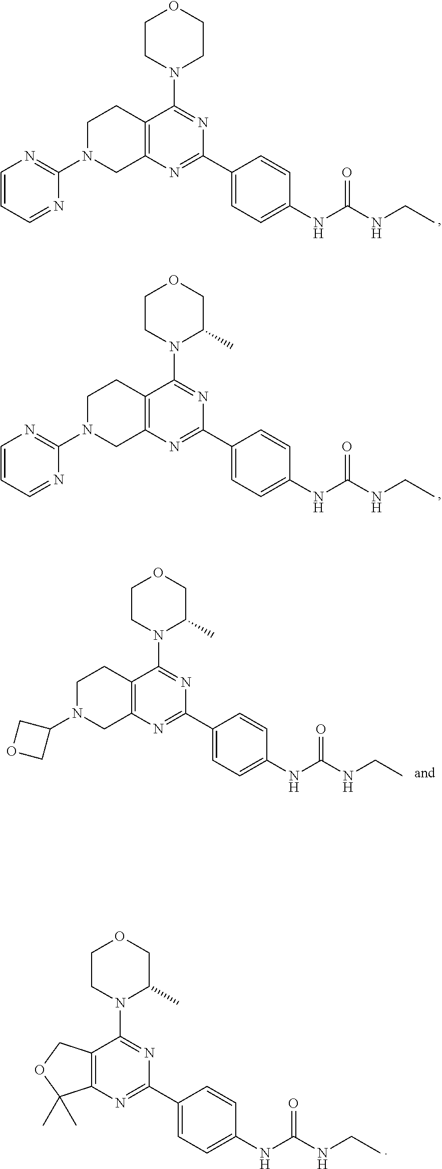 [SMILES: CCNC(=O)Nc1ccc(-c2nc3c(c(N4CCOCC4)n2)CCN(c2ncccn2)C3)cc1 ; CCNC(=O)Nc1ccc(-c2nc3c(c(N4CCOC[C@@H]4C)n2)CCN(c2ncccn2)C3)cc1 ; CCNC(=O)Nc1ccc(-c2nc3c(c(N4CCOC[C@@H]4C)n2)CCN(C2COC2)C3)cc1 ; CCNC(=O)Nc1ccc(-c2nc(N3CCOC[C@@H]3C)c3c(n2)C(C)(C)OC3)cc1]

Also provided by the invention is a population of endoderm cells obtained using any one of the methods above.

In another aspect, the invention provides methods for identifying a factor that promotes the differentiation of endoderm cells into a cell type of interest, the method comprising: contacting a population of endoderm cells, wherein at least 83% of the cells in the population express SOX17, at least 77% of the cells in the population express FoxA2, or at least 76% of the cells in the population express CXCR4, with the factor, monitoring the population of endoderm cells for differentiation into the cell type of interest, thereby identifying the factor that promotes the differentiation of endoderm cells into a cell type of interest.

In another aspect, the invention provides methods for identifying a factor that inhibits the differentiation of endoderm cells, the method comprising: contacting a population of endoderm cells, wherein at least 83% of the cells in the population express SOX17, at least 77% of the cells in the population express FoxA2, or at least 76% of the cells in the population express CXCR4, with the factor, monitoring the cells for differentiation, thereby identifying a factor that inhibits the differentiation of endoderm cells.

In another aspect, the invention provides methods for screening a drug candidate for toxicity, the method comprising: contacting a population of endoderm cells, wherein at least 83% of the cells in the population express SOX17, at least 77% of the cells in the population express FoxA2, or at least 76% of the cells in the population express CXCR4, with the drug and monitoring the cells for toxicity, thereby identifying whether the drug candidate is toxic.

In another aspect, the invention provides methods of providing a cell-based therapy to a patient in need thereof, comprising administering to the patient a population of endoderm cells, wherein at least 83% of the cells in the population express SOX17, at least 77% of the cells in the population express FoxA2, or at least 76% of the cells in the population express CXCR4. In some embodiments according to (e.g., as applied to) any one of the methods above, the patient is suffering from liver fibrosis, cirrhosis, liver failure, liver and pancreatic cancer, pancreatic failure, intestinal tissue replacement enzyme defects, Crohn's disease, inflammatory bowel syndrome, and intestinal cancer

In another aspect, the invention provides methods of obtaining a population of hepatocyte cells, the method comprising: culturing a population of endoderm cells, wherein at least 83% of the cells in the population express SOX17, at least 77% of the cells in the population express FoxA2, or at least 76% of the cells in the population express CXCR4, under conditions sufficient to obtain the population of hepatocyte cells. In some embodiments according to (e.g., as applied to) any one of the methods above, at least 56% of the hepatocyte cells in the population of hepatocyte cells express AFP. In some embodiments according to (e.g., as applied to) any one of the methods above, the endoderm cells are obtained by contacting a population of stem cells with an effective amount of a selective inhibitor of PI3K alpha and an effective amount of an Activin A and culturing the cells under conditions sufficient to obtain the population of hepatocyte cells.

In another aspect, the invention provides methods of obtaining a population of hepatocyte cells, the method comprising: culturing a population of stem cells with an effective amount of a selective inhibitor of PI3K alpha and an effective amount of Activin A and culturing the cells under conditions sufficient to obtain the population of hepatocyte cells. In some embodiments according to (e.g., as applied to) any one of the methods above, the conditions sufficient to obtain the population of hepatocyte cells comprise culturing the endoderm cells in medium containing an effective amount of Activin A and lacking other growth factors. In some embodiments according to (e.g., as applied to) any one of the methods above, the other growth factors are selected from the group consisting of: FGF2, FGF4, BMP2, and BMP4.

In another aspect, the invention provides a population of hepatocyte cells obtained using any one of the methods above.

In another aspect, the invention provides an isolated population of hepatocytes wherein one or more of the following: the hepatocytes secrete albumin, A1AT, or albumin and A1AT; CYP1A1/2 activity is inducible; and the hepatocytes express AFM, AFP, AGXT, ALB, CEBPA, CYP2C19, CYP2C9, CYP3A4, CYP3A7, CYP7A1, CABP1, FOXA1, FOXA2, GSTA1, HNF1A, HNF1B, HNF4a, IL6R, SERPINA1, SERPINA3, SERPINA7, SLCO2B1, TAT, VCAM1, or a combination thereof.

In another aspect, the invention provides a method of providing cell-based therapy to a patient in need thereof comprising administering to the patient an effective amount of a population of hepatocyte cells above.

In another aspect, the invention provides a method of screening for a drug candidate for toxicity comprising contacting a population of hepatocytes obtained by any one of the methods of described herein with a drug candidate, monitoring the hepatocytes for toxicity, thereby identifying whether the drug candidate is toxic.

In another aspect, the invention provides methods of obtaining pancreatic progenitor cells, said method comprising: culturing a population of stem cells with an effective amount of either (1) a mTOR inhibitor and an effective amount of Activin A or (2) a selective inhibitor of PI3K alpha and an effective amount of Activin A or (3) an mTOR inhibitor, a selective inhibitor of PI3K alpha, and effective amount of Activin A, and culturing the cells under conditions sufficient to obtain the population of endoderm cells; and culturing the endoderm cells under conditions sufficient to promote the differentiation of endoderm cells to pancreatic progenitor cells.

In another aspect, the invention provides methods of obtaining pancreatic progenitor cells, said method comprising: culturing a starting population of endoderm cells described above under conditions sufficient to promote the differentiation of endoderm cells to pancreatic progenitor cells.

In some embodiments according to (e.g., as applied to) any one of the methods above, the pancreatic progenitor cells can differentiate into pancreatic endocrine cells, pancreatic exocrine cells and pancreatic ductal cells. In some embodiments according to (e.g., as applied to) any one of the methods above, the pancreatic endocrine cells are selected from the group consisting of alpha cells, beta cells, delta cells and gamma cells. In some embodiments according to (e.g., as applied to) any one of the methods above, the pancreatic endocrine cells are capable of producing one or more of: glucagon, insulin, somatostatin, and pancreatic polypeptide.

In another aspect, the invention provides methods of obtaining differentiated pancreatic cells, said method comprising culturing pancreatic progenitor cells produced by any one of the methods above under conditions sufficient to promote the differentiation of pancreatic progenitor cells to differentiated pancreatic cells. In some embodiments according to (e.g., as applied to) any one of the methods above, the differentiated pancreatic cells is selected from the group consisting of pancreatic endocrine cells, pancreatic exocrine cells and pancreatic ductal cells. In some embodiments according to (e.g., as applied to) any one of the methods above, the differentiated pancreatic cells are capable of producing one or more of: glucagon, insulin, somatostatin, and pancreatic polypeptide.

In another aspect, the invention provides an isolated population of pancreatic progenitor cells produced by a method above. In another aspect, the invention provides an isolated population of pancreatic progenitor cells wherein the pancreatic progenitor cells express Pdx1, C-peptide, ARX, GLIS3, HNF1a, HNF1b, HNF4a, KRT19, MNX1, RFX6, SERPINA3, ONECUT1, NKX2-2, or any combination thereof. In another aspect, the invention provides an isolated population of differentiated pancreatic cells produced by a method above. In another aspect, the invention provides isolated population of differentiated pancreatic cells wherein the pancreatic cells form clusters in suspension and are viable in suspension.

In another aspect, the invention provides methods of providing cell-based therapy to a patient in need thereof comprising administering to the patient an effective amount of a population of pancreatic progenitor cells described above. In another aspect, the invention provides a method of providing cell-based therapy to a patient in need thereof comprising administering to the patient an effective amount of a population of differentiated pancreatic cells described above. In another aspect, the invention provides methods of screening for a drug candidate for toxicity comprising contacting a population of pancreatic cells obtained by any one of the methods above with a drug candidate, monitoring the pancreatic cells for toxicity, thereby identifying whether the drug candidate is toxic.

In another aspect, the invention provides for a population, including an isolated population, of endoderm cells wherein at least 75% of the cells express SOX17, at least 75% of the cells express FoxA2, or at least 75% of the cells express CXCR4. In some embodiments according to (e.g., as applied to) any one of the populations above, at least 83% of the cells express SOX17, at least 77% of the cells express FoxA2, or at least 76% of the cells express CXCR4. In some embodiments according to (e.g., as applied to) any one of the populations above, at least 83% of the cells express SOX17 and at least 77% of the cells express FoxA2. In some embodiments according to (e.g., as applied to) any one of the populations above, at least 77% of the cells express FoxA2 and at least 76% of the cells express CXCR4. In some embodiments according to (e.g., as applied to) any one of the populations above, at least 83% of the cells express SOX17 and at least 76% of the cells express CXCR4. In some embodiments according to (e.g., as applied to) any one of the populations above, at least 83% of the cells express SOX17, at least 77% of the cells express FoxA2, and at least 76% of the cells express CXCR4. In any of the embodiments of the isolated populations of endoderm cells described herein, the endoderm cells have the capability to become hepatocytes.

In another aspect, the invention provides for a bank of endoderm cells that comprise one or more populations of endoderm cells wherein at least 75% of the cells express SOX17, at least 75% of the cells express FoxA2, and/or at least 75% of the cells express CXCR4, wherein the population is cryogenically stored. In some embodiments according to (e.g., as applied to) any one of banks above, the bank of endoderm cells comprises one or more populations of endoderm cells wherein at least 83% of the cells express SOX17, at least 77% of the cells express FoxA2, and/or at least 76% of the cells express CXCR4, wherein the population is cryogenically stored. In some embodiments according to (e.g., as applied to) any one of banks above, the endoderm cells in the banks have the capability to become hepatocytes.

In another aspect, the invention provides methods of obtaining a population of endoderm cells by contacting a population of stem cells with an effective amount of a selective inhibitor of PI3K alpha and an effective amount of an Activin A and culturing the cells under conditions sufficient to obtain the population of endoderm cells. In some embodiments according to (e.g., as applied to) any one of the methods above, at least 75% of the cells in the population of endoderm cells express SOX17, at least 75% of the cells in the population of endoderm cells express FoxA2, or at least 75% of the cells in the population of endoderm cells express CXCR4. In some embodiments according to (e.g., as applied to) any one of the methods above, at least 83% of the cells in the population of endoderm cells express SOX17, at least 77% of the cells in the population of endoderm cells express FoxA2, or at least 76% of the cells in the population of endoderm cells express CXCR4. In some embodiments according to (e.g., as applied to) any one of the methods above, at least 83% of the cells express SOX17 and at least 77% of the cells express FoxA2. In some embodiments according to (e.g., as applied to) any one of the methods above, at least 77% of the cells express FoxA2 and at least 76% of the cells express CXCR4. In some embodiments according to (e.g., as applied to) any one of the methods above, at least 83% of the cells express SOX17 and at least 76% of the cells express CXCR4. In some embodiments according to (e.g., as applied to) any one of the methods above, at least 83% of the cells express SOX17, at least 77% of the cells express FoxA2 and at least 76% of the cells express CXCR4. In some embodiments according to (e.g., as applied to) any one of the methods above, the endoderm cells obtained by the methods described herein have the capability to become hepatocytes. In some embodiments according to (e.g., as applied to) any one of the methods above, the endoderm cells have greater viability and/or proliferation as compared to stem cells that have not been contacted with a selective inhibitor of PI3K alpha and Activin A. In some embodiments according to (e.g., as applied to) any one of the methods above, the endoderm cells have greater viability and/or proliferation as compared to a control that has not been contacted with a selective inhibitor of PI3K alpha.

In various embodiments, the stem cells used in the methods are adult stem cells, embryonic stem cells, or induced pluripotent stem cells. In some embodiments according to (e.g., as applied to) any one of the methods above, the stem cells are cultured in qualified matrigel, gelatin, or collagen. In some embodiments according to (e.g., as applied to) any one of the methods above, the stem cells are cultured in suspension.

In some embodiments according to (e.g., as applied to) any one of the methods above, a selective inhibitor of PI3K alpha is a compound which is a fused pyrimidine of formula (I):

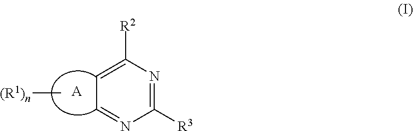

wherein A represents a thiophene or furan ring; n is 1 or 2; R¹ is a group of formula:

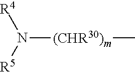

wherein m is 0 or 1; R³⁰ is H or C₁-C₆ alkyl; R⁴ and R⁵ form, together with the N atom to which they are attached, a 5- or 6-membered saturated N-containing heterocyclic group which includes 0 or 1 additional heteroatoms selected from N, S and O, which may be fused to a benzene ring and which is unsubstituted or substituted; or one of R⁴ and R⁵ is alkyl and the other is a 5- or 6-membered saturated N-containing heterocyclic group as defined above or an alkyl group which is substituted by a 5- or 6-membered saturated N-containing heterocyclic group as defined above; R² is selected from:

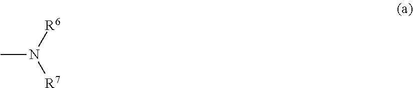

wherein R⁶ and R⁷ form, together with the nitrogen atom to which they are attached, a morpholine, thiomorpholine, piperidine, piperazine, oxazepane or thiazepane group which is unsubstituted or substituted; and

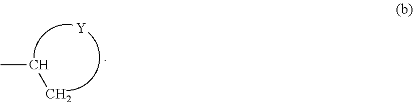

wherein Y is a C₂-C₄ alkylene chain which contains, between constituent carbon atoms of the chain and/or at one or both ends of the chain, 1 or 2 heteroatoms selected from O, N and S, and which is unsubstituted or substituted; and R³ is an indazole group which is unsubstituted or substituted; or a pharmaceutically acceptable salt thereof.

In some embodiments according to (e.g., as applied to) any one of the methods above, the fused pyrimidine of the selective inhibitor of PI3K alpha is of formula (Ia):

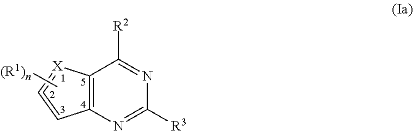

wherein X is S or O and R¹, R², R³ and n are as defined above.

In some embodiments according to (e.g., as applied to) any one of the methods above, the fused pyrimidine of the selective inhibitor of PI3K alpha is of formula (Ib):

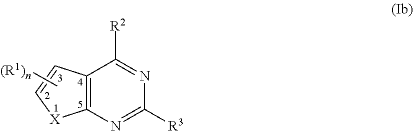

wherein X is S or O and R¹, R², R³ and n are as defined as above.

In some embodiments according to (e.g., as applied to) any one of the methods above, a selective inhibitor of PI3K alpha is a compound, or combination of compounds, selected from: 2-(1H-Indazol-4-yl)-6-(4-methyl-piperazin-1-ylmethyl)-4-morpholin-4-yl-thieno[3,2-d]pyrimidine; 4-[2-(1H-Indazol-4-yl)-4-morpholin-4-yl-thieno[3,2-d]pyrimidin-6-ylmethyl]-piperazine-1-sulfonic acid dimethylamide; {4-[2-(1H-Indazol-4-yl)-4-morpholin-4-yl-thieno[3,2-d]pyrimidin-6-ylmethyl]-piperazin-1-yl}-morpholin-4-yl-methanone; 4-[2-(1H-Indazol-4-yl)-4-morpholin-4-yl-thieno[3,2-d]pyrimidin-6-ylmethyl]-piperazine-1-carboxylic acid (2-methoxy-ethyl)-methyl-amide; {4-[2-(1H-Indazol-4-yl)-4-morpholin-4-yl-thieno[3,2-d]pyrimidin-6-ylmethyl]-piperazin-1-yl}-N,N-dimethyl-acetamide; 4-[2-(1H-Indazol-4-yl)-4-morpholin-4-yl-thieno[3,2-d]pyrimidin-6-ylmethyl]-piperazine-1-carboxylic acid dimethylamide; 2-(1H-Indazol-4-yl)-4-morpholin-4-yl-6-[4-(3-morpholin-4-yl-propane-1-sulfonyl)-piperazin-1-ylmethyl]-thieno[3,2-d]pyrimidine; {1-[2-(1H-Indazol-4-yl)-4-morpholin-4-yl-thieno[3,2-d]pyrimidin-6-ylmethyl]-piperidin-4-yl}-(2-methoxy-ethyl)-methyl-amine; (3-{4-[2-(1H-Indazol-4-yl)-4-morpholin-4-yl-thieno[3,2-d]pyrimidin-6-ylmethyl]-piperazine-1-sulfonyl}-propyl)-dimethyl-amine; 2-{4-[2-(1H-Indazol-4-yl)-4-morpholin-4-yl-thieno[3,2-d]pyrimidin-6-ylmethyl]-piperazin-1-yl}-2-methyl-propan-1-ol; 1′-[2-(1H-Indazol-4-yl)-4-morpholin-4-yl-thieno[3,2-d]pyrimidin-6-ylmethyl]-[1,4′]bipiperidinyl; 2-(1H-Indazol-4-yl)-4-morpholin-4-yl-6-(4-morpholin-4-yl-piperidin-1-ylmethyl)-thieno[3,2-d]pyrimidine; 2-(1H-Indazol-4-yl)-4-morpholin-4-yl-6-(4-pyrimidin-2-yl-piperazin-1-ylmethyl)-thieno[3,2-d]pyrimidine; 1-(2-Hydroxy-ethyl)-4-[2-(1H-indazol-4-yl)-4-morpholin-4-yl-thieno[3,2-d]pyrimidin-6-ylmethyl]-piperazin-2-one; 6-(4-Cyclopropylmethyl-piperazin-1-ylmethyl)-2-(1H-indazol-4-yl)-4-morpholin-4-yl-thieno[3,2-d]pyrimidine; 2-(1H-Indazol-4-yl)-4-morpholin-4-yl-6-(4-pyridin-2-yl-piperazin-1-ylmethyl)-thieno[3,2-d]pyrimidine; 2-(1H-Indazol-4-yl)-4-morpholin-4-yl-6-[4-(2,2,2-trifluoro-ethyl)-piperazin-1-ylmethyl]-thieno[3,2-d]pyrimidine; 2-(1H-Indazol-4-yl)-4-morpholin-4-yl-6-(4-thiazol-2-yl-piperazin-1-ylmethyl)-thieno[3,2-d]pyrimidine; 2-(6-Fluoro-1H-indazol-4-yl)-6-(4-methyl-piperazin-1-ylmethyl)-4-morpholin-4-yl-thieno[3,2-d]pyrimidine; 2-(1H-Indazol-4-yl)-4-morpholin-4-yl-6-(4-pyridin-2-ylmethyl-piperazin-1-ylmethyl)-thieno[3,2-d]pyrimidine; 2-(1H-Indazol-4-yl)-4-morpholin-4-yl-6-(4-thiazol-2-ylmethyl-piperazin-1-ylmethyl)-thieno[3,2-d]pyrimidine; 2-(1H-Indazol-4-yl)-6-[4-(5-methyl-furan-2-ylmethyl)-piperazin-1-ylmethyl]-4-morpholin-4-yl-thieno[3,2-d]pyrimidine; 1-[2-(1H-Indazol-4-yl)-4-morpholin-4-yl-thieno[3,2-d]pyrimidin-6-ylmethyl]-piperidine-4-carboxylic acid amide; 2-(1H-Indazol-4-yl)-6-[4-(2-methoxy-1,1-dimethyl-ethyl)-piperazin-1-ylmethyl]-4-morpholin-4-yl-thieno[3,2-d]pyrimidine; 2-(1H-Indazol-4-yl)-6-[(3R,5S)-4-(2-methoxy-ethyl)-3,5-dimethyl-piperazin-1-ylmethyl]-4-morpholin-4-yl-thieno[3,2-d]pyrimidine; 1-[2-(1H-Indazol-4-yl)-4-morpholin-4-yl-thieno[3,2-d]pyrimidin-6-ylmethyl]-piperidine-4-carboxylic acid (2-methoxy-ethyl)-methyl-amide; 1-[2-(1H-Indazol-4-yl)-4-morpholin-4-yl-thieno[3,2-d]pyrimidin-6-ylmethyl]-piperidine-4-carboxylic acid dimethylamide; 2-(1H-Indazol-4-yl)-4-morpholin-4-yl-6-(4-pyridin-3-ylmethyl-piperazin-1-ylmethyl)-thieno[3,2-d]pyrimidine; 1-[2-(1H-Indazol-4-yl)-4-morpholin-4-yl-thieno[3,2-d]pyrimidin-6-ylmethyl]-piperidine-4-carboxylic acid methylamide; 2-{4-[2-(1H-Indazol-4-yl)-4-morpholin-4-yl-thieno[3,2-d]pyrimidin-6-ylmethyl]-piperazin-1-yl}-N-methyl-isobutyramide; 2-{4-[2-(1H-Indazol-4-yl)-4-morpholin-4-yl-thieno[3,2-d]pyrimidin-6-ylmethyl]-piperazin-1-yl}-2-methyl-1-pyrrolidin-1-yl-propan-1-one; 2-(1H-Indazol-4-yl)-6-[4-(1-methyl-1H-imidazol-2-ylmethyl)-piperazin-1-ylmethyl]-4-morpholin-4-yl-thieno[3,2-d]pyrimidine; 2-(1H-Indazol-4-yl)-6-[4-(5-methyl-isoxazol-3-ylmethyl)-piperazin-1-ylmethyl]-4-morpholin-4-yl-thieno[3,2-d]pyrimidine; 1-{4-[2-(1H-Indazol-4-yl)-4-morpholin-4-yl-thieno[3,2-d]pyrimidin-6-ylmethyl]-piperazin-1-yl}-2-methyl-propan-2-ol; cyclopropylmethyl-{1-[2-(1H-indazol-4-yl)-4-morpholin-4-yl-thieno[3,2-d]pyrimidin-6-ylmethyl]-piperidin-4-yl}-(2-methoxy-ethyl)-amine; 6-[4-(1-Ethyl-1-methoxymethyl-propyl)-piperazin-1-ylmethyl]-2-(1H-indazol-4-yl)-4-morpholin-4-yl-thieno[3,2-d]pyrimidine; 2-(1H-Indazol-4-yl)-6-[4-(1-methoxymethyl-cyclopropyl)-piperazin-1-ylmethyl]-4-morpholin-4-yl-thieno[3,2-d]pyrimidine; {1-[2-(1H-Indazol-4-yl)-4-morpholin-4-yl-thieno[3,2-d]pyrimidin-6-ylmethyl]-piperidin-4-yl}-(2-methoxy-ethyl)-(2,2,2-trifluoro-ethyl)-amine; 2-(1H-Indazol-4-yl)-6-[4-(2-methoxy-ethyl)-piperazin-1-ylmethyl]-4-morpholin-4-yl-thieno[3,2-d]pyrimidine; {1-[2-(1H-Indazol-4-yl)-4-morpholin-4-yl-thieno[3,2-d]pyrimidin-6-ylmethyl]-piperidin-4-yl}-methanol; 2-(1H-Indazol-4-yl)-4-morpholin-4-yl-6-(4-pyridin-4-ylmethyl-piperazin-1-ylmethyl)-thieno[3,2-d]pyrimidine; 2-(1H-Indazol-4-yl)-6-[4-(6-methyl-pyridin-2-ylmethyl)-piperazin-1-ylmethyl]-4-morpholin-4-yl-thieno[3,2-d]pyrimidine; 2-(1H-Indazol-4-yl)-6-[4-(4-methyl-thiazol-2-ylmethyl)-piperazin-1-ylmethyl]-4-morpholin-4-yl-thieno[3,2-d]pyrimidine; {1-[2-(1H-Indazol-4-yl)-4-morpholin-4-yl-thieno[3,2-d]pyrimidin-6-ylmethyl]-piperidin-4-yl}-pyridin-2-yl-amine; N-{1-[2-(1H-Indazol-4-yl)-4-morpholin-4-yl-thieno[3,2-d]pyrimidin-6-ylmethyl]-piperidin-4-yl}-2-methoxy-N-methyl-acetamide; N-{1-[2-(1H-Indazol-4-yl)-4-morpholin-4-yl-thieno[3,2-d]pyrimidin-6-ylmethyl]-piperidin-4-yl}-N-methyl-methanesulfonamide; {1-[2-(1H-Indazol-4-yl)-4-morpholin-4-yl-thieno[3,2-d]pyrimidin-6-ylmethyl]-piperidin-4-yl}-(3-methoxy-propyl)-methyl-amine; 6-((3S,5R)-3,5-Dimethyl-4-pyridin-2-ylmethyl-piperazin-1-ylmethyl)-2-(1H-indazol-4-yl)-4-morpholin-4-yl-thieno[3,2-d]pyrimidine; 2-(1H-Indazol-4-yl)-6-(4-methoxymethyl-piperidin-1-ylmethyl)-4-morpholin-4-yl-thieno[3,2-d]pyrimidine; {1-[2-(1H-Indazol-4-yl)-4-morpholin-4-yl-thieno[3,2-d]pyrimidin-6-ylmethyl]-piperidin-4-yl}-(2-methoxy-ethyl)-thiazol-2-ylmethyl-amine; 1-[2-(1H-Indazol-4-yl)-4-morpholin-4-yl-thieno[3,2-d]pyrimidin-6-ylmethyl]-4-pyridin-2-ylmethyl-piperidin-4-ol; {1-[2-(1H-Indazol-4-yl)-4-morpholin-4-yl-thieno[3,2-d]pyrimidin-6-ylmethyl]-piperidin-4-yl}-isopropyl-(2-methoxy-ethyl)-amine; 2-(1H-Indazol-4-yl)-4-morpholin-4-yl-6-[4-(pyridin-2-yloxy)-piperidin-1-ylmethyl]-thieno[3,2-d]pyrimidine; N-{1-[2-(1H-Indazol-4-yl)-4-morpholin-4-yl-thieno[3,2-d]pyrimidin-6-ylmethyl]-piperidin-4-yl}-N-(2-methoxy-ethyl)-methanesulfonamide; 2-{1-[2-(1H-Indazol-4-yl)-4-morpholin-4-yl-thieno[3,2-d]pyrimidin-6-ylmethyl]-piperidin-4-yl}-propan-2-ol; 2-(1H-Indazol-4-yl)-4-morpholin-4-yl-6-[4-(1-oxy-pyridin-3-ylmethyl)-piperazin-1-ylmethyl]-thieno[3,2-d]pyrimidine; 2-(1H-Indazol-4-yl)-4-morpholin-4-yl-6-(4-morpholin-4-ylmethyl-piperidin-1-ylmethyl)-thieno[3,2-d]pyrimidine; {1-[2-(1H-Indazol-4-yl)-4-morpholin-4-yl-thieno[3,2-d]pyrimidin-6-ylmethyl]-piperidin-4-ylmethyl}-(2-methoxy-ethyl)-methyl-amine; {1-[2-(1H-Indazol-4-yl)-4-morpholin-4-yl-thieno[3,2-d]pyrimidin-6-ylmethyl]-piperidin-4-ylmethyl}-dimethyl-amine; {1-[2-(1H-Indazol-4-yl)-4-morpholin-4-yl-thieno[3,2-d]pyrimidin-6-ylmethyl]-piperidin-3-yl}-(2-methoxy-ethyl)-methyl-amine; 1-[2-(1H-Indazol-4-yl)-4-morpholin-4-yl-thieno[3,2-d]pyrimidin-6-ylmethyl]-piperidine-3-carboxylic acid methylamide; 2-(1H-Indazol-4-yl)-6-(3-methoxymethyl-piperidin-1-ylmethyl)-4-morpholin-4-yl-thieno[3,2-d]pyrimidine; 2-(1H-Indazol-4-yl)-4-morpholin-4-yl-6-(4-pyridin-2-ylmethyl-piperidin-1-ylmethyl)-thieno[3,2-d]pyrimidine; 2-(1H-Indazol-4-yl)-6-[4-(2-methoxy-ethoxy)-piperidin-1-ylmethyl]-4-morpholin-4-yl-thieno[3,2-d]pyrimidine; 6-((3R,5S)-3,5-Dimethyl-4-thiazol-2-ylmethyl-piperazin-1-ylmethyl)-2-(1H-indazol-4-yl)-4-morpholin-4-yl-thieno[3,2-d]pyrimidine; 2-(1H-Indazol-4-yl)-4-morpholin-4-yl-6-[4-(1-oxy-pyridin-2-ylmethyl)-piperazin-1-ylmethyl]-thieno[3,2-d]pyrimidine; 2-(1H-Indazol-4-yl)-6-[4-(2-methoxy-ethyl)-piperidin-1-ylmethyl]-4-morpholin-4-yl-thieno[3,2-d]pyrimidine; 2-(1H-Indazol-4-yl)-6-(4-methanesulfonyl-piperidin-1-ylmethyl)-4-morpholin-4-yl-thieno[3,2-d]pyrimidine; {1-[2-(1H-Indazol-4-yl)-4-morpholin-4-yl-thieno[3,2-d]pyrimidin-6-ylmethyl]-piperidin-4-yl}-(3-methanesulfonyl-propyl)-methyl-amine; 2-(1H-Indazol-4-yl)-6-[4-(3-methoxy-propane-1-sulfonyl)-piperidin-1-ylmethyl]-4-morpholin-4-yl-thieno[3,2-d]pyrimidine; (R)-1-[2-(1H-Indazol-4-yl)-4-morpholin-4-yl-thieno[3,2-d]pyrimidin-6-ylmethyl]-piperidine-3-carboxylic acid methylamide; (S)-1-[2-(1H-Indazol-4-yl)-4-morpholin-4-yl-thieno[3,2-d]pyrimidin-6-ylmethyl]-piperidine-3-carboxylic acid methylamide; 6-(4-Imidazol-1-ylmethyl-piperidin-1-ylmethyl)-2-(1H-indazol-4-yl)-4-morpholin-4-yl-thieno[3,2-d]pyrimidine; 2-(1H-Indazol-4-yl)-4-morpholin-4-yl-6-morpholin-4-ylmethyl-thieno[3,2-d]pyrimidine; 2-(1H-Indazol-4-yl)-6-(3-methyl-piperidin-1-ylmethyl)-4-morpholin-4-yl-thieno[3,2-d]pyrimidine; {1-[2-(1H-Indazol-4-yl)-4-morpholin-4-yl-thieno[3,2-d]pyrimidin-6-ylmethyl]-piperidin-3-yl}-methanol; 2-{1-[2-(1H-Indazol-4-yl)-4-morpholin-4-yl-thieno[3,2-d]pyrimidin-6-ylmethyl]-piperidin-4-yl}-ethanol; 1-[2-(1H-Indazol-4-yl)-4-morpholin-4-yl-thieno[3,2-d]pyrimidin-6-ylmethyl]-4-thiazol-2-yl-piperidin-4-ol; 2-(1-Methyl-1H-indazol-4-yl)-6-(4-methyl-piperazin-1-ylmethyl)-4-morpholin-4-yl-thieno[3,2-d]pyrimidine; 2-(2-Methyl-2H-indazol-4-yl)-6-(4-methyl-piperazin-1-ylmethyl)-4-morpholin-4-yl-thieno[3,2-d]pyrimidine; 2-(1H-Indazol-4-yl)-4-morpholin-4-yl-6-(4-thiazol-4-ylmethyl-piperazin-1-ylmethyl)-thieno[3,2-d]pyrimidine; 1-{4-[2-(1H-Indazol-4-yl)-4-morpholin-4-yl-thieno[3,2-d]pyrimidin-6-ylmethyl]-piperazin-1-yl}-3-phenoxy-propan-2-ol; 6-[4-(1H-Imidazol-2-ylmethyl)-piperazin-1-ylmethyl]-2-(1H-indazol-4-yl)-4-morpholin-4-yl-thieno[3,2-d]pyrimidine; 6-[4-(3H-Imidazol-4-ylmethyl)-piperazin-1-ylmethyl]-2-(1H-indazol-4-yl)-4-morpholin-4-yl-thieno[3,2-d]pyrimidine; 2-(1H-Indazol-4-yl)-4-morpholin-4-yl-6-((2S,6R)-2,4,6-trimethyl-piperazin-1-ylmethyl)-thieno[3,2-d]pyrimidine; {4-[2-(1H-Indazol-4-yl)-4-morpholin-4-yl-thieno[3,2-d]pyrimidin-6-ylmethyl]-1-methanesulfonyl-piperazin-2-yl}-methanol; and 2-(1H-Indazol-4-yl)-6-(4-methanesulfonyl-3-methoxymethyl-piperazin-1-ylmethyl)-4-morpholin-4-yl-thieno[3,2-d]pyrimidine; and the pharmaceutically acceptable salts of the above-mentioned free compounds.

In some embodiments according to (e.g., as applied to) any one of the methods above, a selective inhibitor of PI3K alpha is a compound, or combination of compounds, selected from the following:

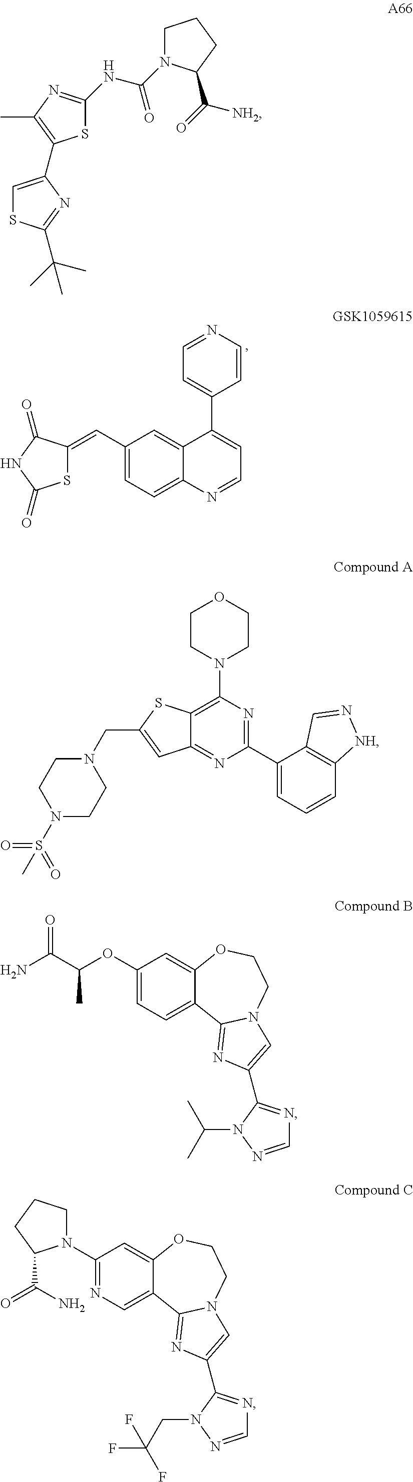

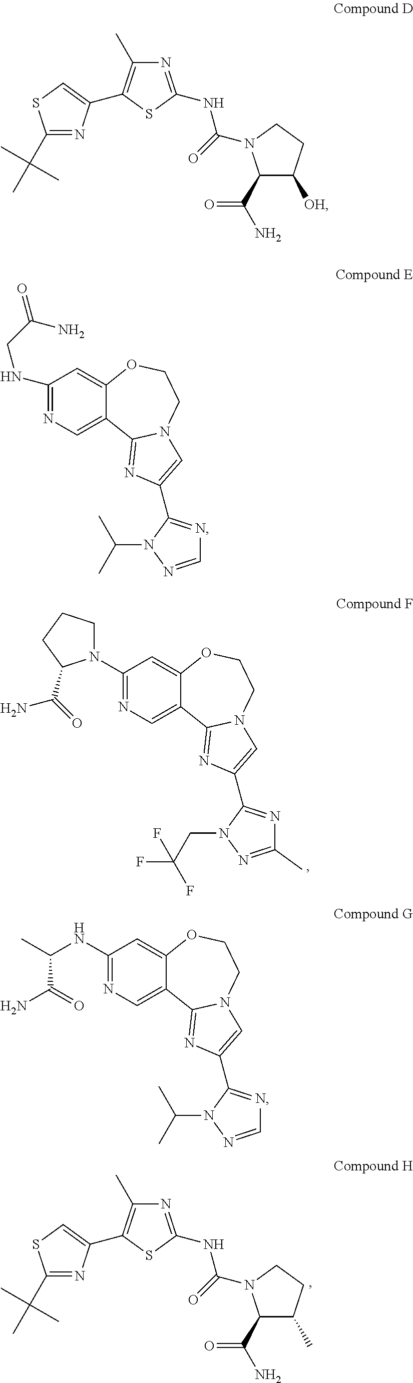

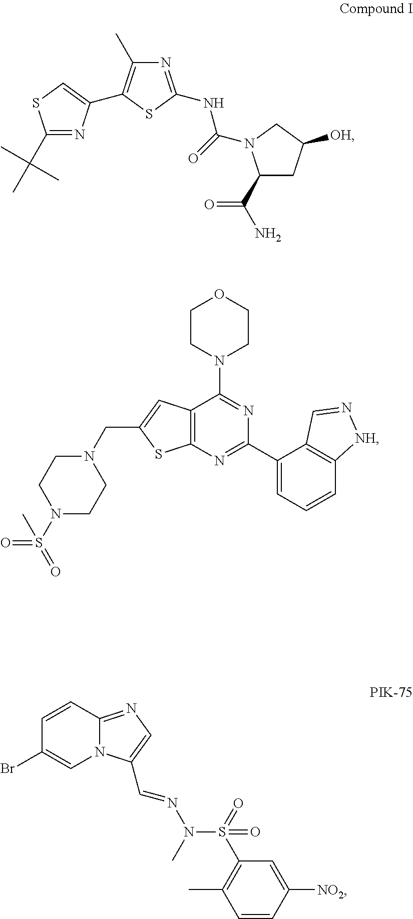 [SMILES: Cc1nc(NC(=O)N2C[C@@H](O)C[C@H]2C(N)=O)sc1-c1csc(C(C)(C)C)n1 ; CS(=O)(=O)N1CCN(Cc2cc3c(N4CCOCC4)nc(-c4cccc5[nH]ncc45)nc3s2)CC1 ; Cc1ccc([N+](=O)[O-])cc1S(=O)(=O)N(C)/N=C/c1cnc2ccc(Br)cn12]

### INK1117 and BYL7.

In some embodiments according to (e.g., as applied to) any one of the methods above, the selective inhibitor of PI3K alpha is a compound is 4-[2-(1H-indazol-4-yl)-6-[(4-methylsulfonylpiperazin-1-yl)methyl]thieno[3,2-d]pyrimidin-4-yl]morpholine.

In some embodiments according to (e.g., as applied to) any one of the methods above, the selective inhibitor of P13K alpha is also an inhibitor of PI3K delta.

In some embodiments according to (e.g., as applied to) any one of the methods above, the effective amount of the selective inhibitor of PI3K alpha is 750 nM. In some embodiments according to (e.g., as applied to) any one of the methods above, the effective amount of Activin A is 100 ng/ml of medium. In some embodiments according to (e.g., as applied to) any one of the methods above, culturing the cells under conditions sufficient to obtain the population of endoderm cells comprises culturing the cells in the absence of Wnt3a.

In some embodiments according to (e.g., as applied to) any one of the methods above, the method further comprises contacting the population of stem cells with an effective amount of an mTOR inhibitor. In some embodiments according to (e.g., as applied to) any one of the methods above, the selective inhibitor of PI3K alpha is also a selective inhibitor mTOR kinase. In some embodiments according to (e.g., as applied to) any one of the methods above, the method further comprises contacting the population of stem cells with a selective inhibitor of PI3K delta.

In some embodiments according to (e.g., as applied to) any one of the methods above, the invention provides a population of endoderm cells obtained by using any of the methods disclosed herein.

In another aspect, the invention provides methods of obtaining a population of endoderm cells that comprise contacting a population of stem cells with an effective amount of an inhibitor of mTOR and an effective amount of an Activin A and culturing the cells under conditions sufficient to obtain the population of endoderm cells. In some embodiments according to (e.g., as applied to) any one of the methods above, the population of endoderm cells obtained is a population wherein at least 61% of the cells in the population of endoderm cells express SOX17 or at least 40% of the cells in the population of endoderm cells express FoxA2. In some embodiments according to (e.g., as applied to) any one of the methods above, the population of endoderm cells obtained is a population wherein at least 61% of the cells in the population of endoderm cells express SOX17 and at least 40% of the cells in the population of endoderm cells express FoxA2. In some embodiments according to (e.g., as applied to) any one of the methods above, the population of endoderm cells obtained is a population wherein the endoderm cells have the capability to become hepatocytes.

In some embodiments according to (e.g., as applied to) any one of the methods above, the methods comprise contacting a population of stem cells with an effective amount of an inhibitor of mTOR and an effective amount of an Activin A wherein the inhibitor of mTOR is an siRNA or a small molecule. In some embodiments according to (e.g., as applied to) any one of the methods above, the inhibitor of mTOR is a small molecule is selected from the group consisting of:

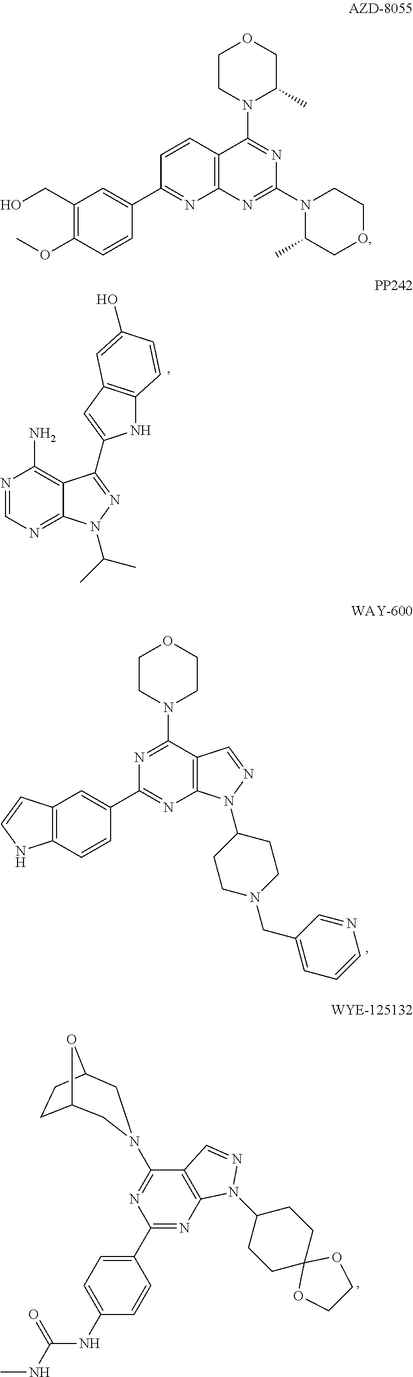

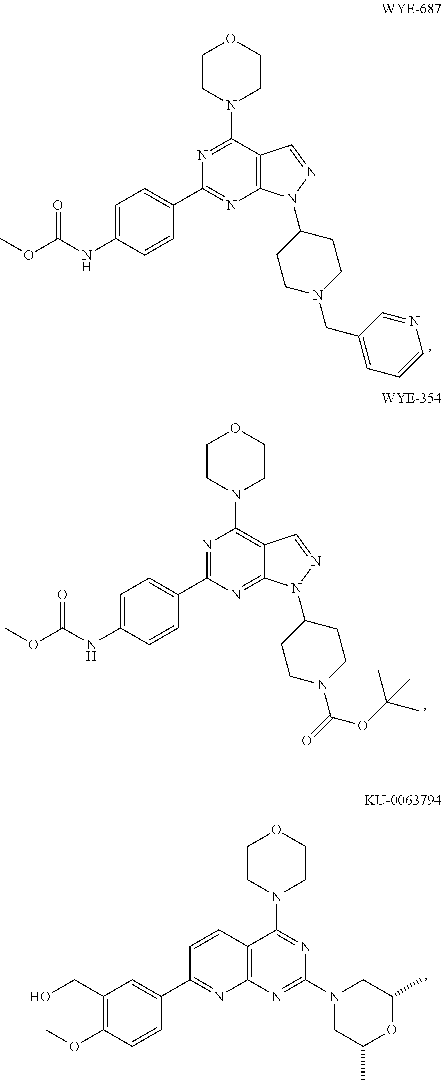

AP23573 (also known as ridaforolimus or deforolimus), Torsel (also known as Temsirolimus or CI-779), INK128, AZD2012, CC-223, OSI-027, sirolimus (rapamycin), and everolimus. In some embodiments according to (e.g., as applied to) any one of the methods above, the inhibitor of mTOR is a small molecule is selected from the group consisting of:

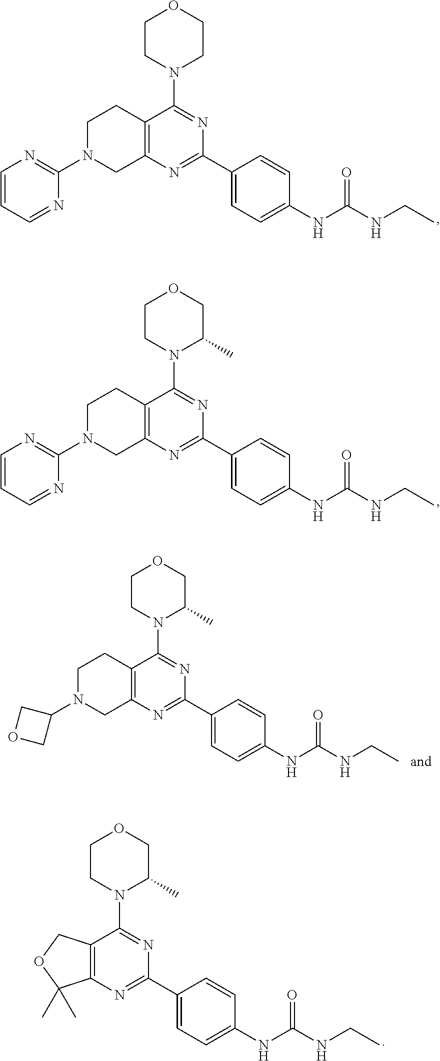 [SMILES: CCNC(=O)Nc1ccc(-c2nc3c(c(N4CCOCC4)n2)CCN(c2ncccn2)C3)cc1 ; CCNC(=O)Nc1ccc(-c2nc3c(c(N4CCOC[C@@H]4C)n2)CCN(c2ncccn2)C3)cc1 ; CCNC(=O)Nc1ccc(-c2nc3c(c(N4CCOC[C@@H]4C)n2)CCN(C2COC2)C3)cc1 ; "and" ; CCNC(=O)Nc1ccc(-c2nc(N3CCOC[C@@H]3C)c3c(n2)C(C)(C)OC3)cc1]

In another aspect, the invention provides methods for identifying a factor that promotes the differentiation of endoderm cells into a cell type of interest by contacting a population of endoderm cells, wherein at least 75% of the cells in the population express SOX17, at least 75% of the cells in the population express FoxA2, or at least 75% of the cells in the population express CXCR4, with the factor, monitoring the population of endoderm cells for differentiation into the cell type of interest, thereby identifying the factor that promotes the differentiation of endoderm cells into a cell type of interest. In some embodiments according to (e.g., as applied to) any one of the methods above, at least 83% of the cells in the population express SOX17, at least 77% of the cells in the population express FoxA2, or at least 76% of the cells in the population express CXCR4.

The invention also provides methods for identifying a factor that inhibits the differentiation of endoderm cells by contacting a population of endoderm cells, wherein at least 75% of the cells in the population express SOX17, at least 75% of the cells in the population express FoxA2, or at least 75% of the cells in the population express CXCR4, with the factor, monitoring the cells for differentiation, thereby identifying a factor that inhibits the differentiation of endoderm cells. In some embodiments according to (e.g., as applied to) any one of the methods above, at least 83% of the cells express SOX17, at least 77% of the cells express FoxA2, or at least 76% of the cells express CXCR4.

The invention also provides methods for screening a drug candidate for toxicity by contacting a population of endoderm cells with the drug and monitoring the cells for toxicity, wherein at least 83% of the cells express SOX17, at least 77% of the cells express FoxA2, or at least 76% of the cells i express CXCR4, thereby identifying whether the drug candidate is toxic.

The invention also provides a method of providing a cell-based therapy to a patient in need thereof, by administering to the patient a population of endoderm cells, at least 75% of the cells in the population express SOX17, at least 75% of the cells in the population express FoxA2, or at least 75% of the cells in the population express CXCR4. In some embodiments according to (e.g., as applied to) any one of the methods above, population of endoderm cells that is administered is a population wherein at least 83% of the cells in the population express SOX17, at least 77% of the cells in the population express FoxA2, or at least 76% of the cells in the population express CXCR4. In some embodiments according to (e.g., as applied to) any one of the methods above, the patient is suffering from liver fibrosis, cirrhosis, liver failure, diabetes, liver and pancreatic cancer, pancreatic failure, intestinal disorders including tissue replacement enzyme defects, Crohn's disease, inflammatory bowel syndrome, and intestinal cancer.

In another aspect, the invention provides methods of obtaining a population of hepatocytes by culturing a population of endoderm cells, wherein at least 75% of the cells in the population express SOX17, at least 75% of the cells in the population express FoxA2, or at least 75% of the cells in the population express CXCR4, under conditions sufficient to obtain the population of hepatocytes. In some embodiments according to (e.g., as applied to) any one of the methods above, the population of endoderm cells that is cultured under conditions sufficient to obtain the hepatocytes is a population wherein at least 83% of the cells in the population express SOX17, at least 77% of the cells in the population express FoxA2, or at least 76% of the cells in the population express CXCR4. In some embodiments according to (e.g., as applied to) any one of the methods above, the endoderm cells are obtained by contacting a population of stem cells with an effective amount of a selective inhibitor of PI3K alpha and an effective amount of an Activin A and culturing the cells under conditions sufficient to obtain the population of hepatocytes. In some embodiments according to (e.g., as applied to) any one of the methods above, the conditions sufficient to obtain the population of hepatocytes comprise culturing the endoderm cells in medium containing an effective amount of Activin A and lacking other growth factors. In some embodiments according to (e.g., as applied to) any one of the methods above, the other growth factors are selected from the group consisting of: HGF, retinoic acid, FGF8, FGF1, DMSO, FGF7, FGF10, OSM, Dexamethasone FGF2, FGF4, BMP2, and BMP4.

The invention also provides methods of obtaining a population of hepatocytes by culturing a population of stem cells with an effective amount of a selective inhibitor of PI3K alpha and an effective amount of an Activin A and culturing the cells under conditions sufficient to obtain the population of hepatocytes. In some embodiments according to (e.g., as applied to) any one of the methods above, the secretion of AFP by the population of hepatocytes obtained by a method described herein decreases over time.

The invention also provides populations of hepatocytes obtained using any one of the methods. In some embodiments according to (e.g., as applied to) any one of the populations above, the secretion of AFP by the hepatocytes in the population decreases over time.

The invention provides methods of providing cell-based therapy to a patient in need thereof by administering to the patient an effective amount of a population of hepatocytes obtained using any one of the methods described herein.

The invention also provides a method of screening for a drug candidate for toxicity by contacting a population of hepatocytes obtained by any one of the methods described herein with a drug candidate, monitoring the hepatocytes for toxicity, thereby identifying whether the drug candidate is toxic.

### BRIEF DESCRIPTION OF THE DRAWINGS

FIG. 1 shows that a population of endoderm cells was obtained by contacting a population of stem cells with a PI3K inhibitor.

FIG. 2 shows that a population of endoderm cells was obtained by contacting a population of stem cells with Compound A, a selective inhibitor of PI3K alpha that is also a selective inhibitor of PI3K delta.

FIG. 3 shows that the effect of Compound A on endoderm differentiation was independent of the culture medium.

FIG. 4 shows the effects of a variety of isoform-specific PI3K inhibitors on endoderm differentiation.

FIG. 5 shows that inhibition of PI3K alpha significantly increased endoderm differentiation as compared to the effects of the inhibition of PI3K beta, delta, or beta and delta.

FIG. 6 provides the results of a time course experiment.

FIG. 7 provides the results of a dose-response experiment.

FIG. 8 provides the results of a proliferation/viability assay in which the proliferation of endoderm cells obtained by methods of the invention was compared to endoderm cells obtained using other methods.

FIG. 9 provides the results of an ATP quantification in which the metabolic activities of endoderm cells obtained by contacting stem cells with Activin A and various doses of a PI3K alpha inhibitor were compared to stem cells and endoderm cells obtained by contacting stem cells with Activin A alone.

FIG. 10 shows the effects of a variety of mTOR inhibitors and Akt inhibitors on endoderm differentiation.

FIG. 11 shows the effects of a variety the mTOR inhibitors (everolimus, KU 0063794, and WYE-354) and one Akt inhibitor (GSK 690693) on endoderm differentiation.

FIG. 12 shows that the inhibition of mTOR, but not the inhibition of Akt, increased endoderm differentiation.

FIG. 13 shows that the simultaneous inhibition of mTOR and P13K alpha increased endoderm formation more efficiently than the inhibition of either mTOR or P13K alpha alone.

FIG. 14 shows that endoderm cells obtained by contacting a population of stem cells with a PI3K alpha inhibitor were able to convert to hepatocytes in the absence of BMP2 and FGF4.

FIG. 15 shows that hepatocytes obtained by methods of the invention show a gradual decrease in alpha fetal protein (AFP) production over time.

FIG. 16 shows the results of an experiment measuring AFP levels. Day 0-Day 3: Activin A or Activin A+PI3K inhibitor. Day 4-Day 10−DMEM/F12+Glutamax+B27. At day 10 of differentiation, medium is changed. Twenty-four hours later, the medium is diluted by 1/500 (to be in the range) and analyzed by AlphaLisa. When PI3K inhibitor is not used at the endoderm stage, AFP level is very low which indicates that hepatocytes levels are low. When PI3K inhibitor is used at the endoderm stage, expression fold of AFP is at almost 100 (for the 1/500 diluted sample) which indicates hepatocyte cell level is high. Expressing the data in fold allow for comparison of different samples/experiments. Fold=Signal medium contact with the cells/Signal raw medium without contact with cells.

FIG. 17 shows the results measuring albumin and HNF4a on stem cell derived hepatocytes at day 20. Stem cells derived hepatocytes population at Day 20: Day 0-Day 3: Activin A+PI3K inhibitors (Compound A). Day 3-Day 20: Basal medium (DMEM/F12+glutamax+B27).

FIG. 18 shows the results of dose response matrix experiments that were performed to determine the effects of varying degrees of mTOR inhibition and PI3K alpha inhibition on the expression of mesendoderm marker genes in cells after one day in culture.

FIG. 19 shows the results of dose response matrix experiments that were performed to determine the effects of varying degrees of mTOR inhibition and PI3K alpha inhibition on the expression of additional mesendoderm marker genes in cells after one day in culture.

FIG. 20 shows the results of dose response matrix experiments that were performed to determine the effects of varying degrees of mTOR inhibition and PI3K alpha inhibition on the expression of endoderm marker genes in cells after two days in culture.

FIG. 21 shows the results of dose response matrix experiments that were performed to determine the effects of varying degrees of mTOR inhibition and PI3K alpha inhibition on the expression of mesoderm marker genes in cells after two days in culture.

FIG. 22 shows the results of experiments that were performed to determine the concentrations of small molecule compounds that induce high levels of SOX17 expression with low cell toxicity.

FIG. 23 shows the kinase profiles of a variety of small molecule compounds that can be used in the methods of the invention.

FIG. 24 shows the results of experiments that were performed to determine the effects of a variety of small molecule compounds on endoderm differentiation.

FIG. 25 shows the results experiments that were performed to determine the effects of a variety of small molecule compounds on the expression of endoderm marker genes.

FIG. 26 shows the results of experiments that were performed to determine the effect of BMP on the maintenance and proliferation of endoderm cells obtained using methods of the invention.

FIG. 27 shows the results of experiments that were performed to determine the effects of various cell culture media on the expression of endoderm marker genes in endoderm cells obtained using methods of the invention.

FIG. 28 shows the results of experiments that were performed to assay the maintenance and proliferation of endoderm cells obtained by methods of the invention.

FIG. 29 shows the results of experiments performed to assess the degree of endoderm marker gene expression in endoderm cells obtained by methods of the invention that have been passaged 9 times.

FIG. 30A shows the results of experiments that were performed to compare the viability of pancreatic progenitor cells obtained by methods of the invention when grown in suspension to the viability of pancreatic progenitor cells obtained by other methods when grown in suspension. FIG. 30B shows a comparison of Pdx expression levels of pancreatic progenitor cells obtained by methods of the invention vs. pancreatic progenitor cells obtained by other methods at Day 13.

FIG. 31 shows the results of experiments that were conducted to compare the expression levels of pancreatic marker genes in AP pancreatic cells and AA pancreatic cells.

FIG. 32 shows the results of experiments that were conducted to compare the expression of endoderm marker genes in AP pancreatic cells and AA pancreatic cells.

FIG. 33 shows the results of experiments that were conducted to compare the secretion of AFP by AP hepatic cells and AA hepatic cells.

FIG. 34 shows the results of experiments that were conducted to compare the secretion of albumin by AP hepatic cells and AA hepatic cells.

FIG. 35 shows the results of experiments that were conducted to compare the secretion of A1AT by AP hepatic cells and AA hepatic cells.

FIG. 36 shows the results of experiments that were conducted to compare the expression of endoderm marker genes in AP hepatic cells and AA hepatic cells.

FIG. 37 shows the results of experiments that were conducted to compare the expression levels of hepatic marker genes in AP hepatic cells and AA hepatic cells.

FIG. 38 shows the results of experiments that were conducted to compare CYP activity in AP hepatic cells and AA hepatic cells.

FIG. 39 shows the results of experiments that were conducted to determine whether CYP activity can be induced in AP hepatic cells and AA hepatic cells.

### DETAILED DESCRIPTION OF THE INVENTION

The invention provides, inter alia, methods for efficient conversion of starting cell population (e.g., stem cells) to endoderm cells, pancreatic progenitor cells, hepatocytes, other differentiated cells derived from endoderm cells (e.g., intestinal progenitor cells, intestinal cells, lung progenitor cells, lung cells, etc.), populations of these cells and intermediate cell population(s), compositions comprising these cells as well as compositions comprising various cell populations and/or components as described herein, and uses thereof. Additionally, the invention provides for isolated populations of endoderm cells, isolated populations of pancreatic progenitor cells, isolated populations of hepatocyte progenitor cells, isolated populations of hepatocytes, isolated populations of multipotent cells derived from endoderm, and methods of their use. The methods described herein can convert a starting cell population to a highly homogenous population of endoderm cells, pancreatic cells, and/or hepatocytes with high efficiency. It is understood that pancreatic cells include, but are not limited to, pancreatic progenitor cells, and further differentiated cells, including, e.g., pancreatic ductal cells and pancreatic exocrine cells. It is also understood that hepatocyte cells include, but are not limited to, hepatocyte progenitor cells, and further differentiated cells, including, e.g., hepatic cells.

The population of endoderm cells of the invention is distinct from other populations of endoderm cells in that a significant percentage of the cells in the populations express endoderm markers, such as SOX17, FoxA2, and CXCR4. Thus, highly homogeneous populations of endoderm cells can be produced. Moreover, the populations of endoderm cells produced by the methods of the invention are more phenotypically stable and proliferative than populations of endoderm cells produced by other methods. Furthermore, the populations of endoderm cells described herein are observed to differentiate into hepatocytes with high efficiency in the absence of additional growth factors. The hepatocytes of this invention are distinct from other populations of hepatocytes in that a significant percentage of the hepatocytes have decreased alpha fetal protein (AFP), indicating the maturation of the hepatocytes. In addition, the populations of endoderm cells described herein are observed to differentiate into pancreatic progenitor cells or other differentiated cells derived from endoderm cells (e.g., intestinal progenitor cells, intestinal cells, lung progenitor cells, lung cells, etc.) The pancreatic progenitor cells of this invention are distinct from other populations of pancreatic progenitor cells in that a significant percentage of the pancreatic progenitor cells exhibit increased expression of pancreatic marker genes. Moreover, the pancreatic progenitor cells of this invention are morphologically distinct from other populations in that they are capable of forming three-dimensional cell clusters that express insulin and glucagon.

It is understood that reference to a population of cells described herein contemplates and includes isolated populations.

General Methods

The practice of the present invention will employ, unless otherwise indicated, conventional techniques of stem cell biology, cell culturing, molecular biology (including recombinant techniques), microbiology, cell biology, biochemistry and immunology, which are within the skill of the art. Such techniques are explained fully in the literature, such as, Molecular Cloning: A Laboratory Manual, third edition (Sambrook et al., 2001) Cold Spring Harbor Press; Oligonucleotide Synthesis (P. Herdewijn, ed., 2004); Animal Cell Culture (R. I. Freshney), ed., 1987); Methods in Enzymology (Academic Press, Inc.); Handbook of Experimental Immunology (D. M. Weir &C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991) Short Protocols in Molecular Biology (Wiley and Sons, 1999), Embryonic Stem Cells: A Practical Approach (Notaranni et al. eds., Oxford University Press 2006); Essentials of Stem Cell Biology (R. Lanza, ed., Elsevier Academic Press 2006); Stem Cell Assays (Methods in Molecular Biology) (Mohan C. Vemuri, Ed., Humana Press; first edition (Aug. 10, 2007); Mesenchymal Stem Cells: Methods and Protocols (Methods in Molecular Biology) (Darwin J. Prockop, Donald G. Phinney, Bruce A Bunnell, Eds., first edition (Mar. 7, 2008)); Handbook of Stem Cells (Robert Lanza, et al., Eds., Academic Press (Sep. 14, 2004); Stem Cell Culture Vol 86: Methods in Cell Biology (Jennie P. Mather, Ed., Academic Press, first edition (May 15, 2008)); Practical Hematopoietic Stem Cell Transplantation (Andrew J. Cant, et al. Eds., Wiley-Blackwell, first edition (Jan. 22, 2007)); Hematopoietic Stem Cell Protocols (Kevin D. Bunting, Ed., Humana Press, 2nd ed. edition (Jan. 31, 2008)); Bone Marrow and Stem Cell Transplantation (Methods in Molecular Medicine) (Meral Beksac, Ed., Humana Press; first edition (May 3, 2007)); Stem Cell Therapy and Tissue Engineering for Cardiovascular Repair: From Basic Research to Clinical Applications (Nabil Dib, et al., Eds., Springer, first edition (Nov. 16, 2005)); Blood And Marrow Stem Cell Transplantation: Principles, Practice, And Nursing Insights (Kim Schmit-Pokorny (Author) and Susan Ezzone (Editor), Jones & Bartlett Publishers; third edition (May 22, 2006)); Hematopoietic Stem Cell Protocols (Christopher A. Klug and Craig T. Jordan, Eds., Humana Press; first edition (Dec. 15, 2001)); and Clinical Bone Marrow and Blood Stem Cell Transplantation (Kerry Atkinson, et al., Eds., Cambridge University Press; third edition (Dec. 8, 2003)).

Unless defined otherwise, all technical and scientific terms used herein have the same meaning as is commonly understood by one of ordinary skill in the art to which this invention belongs.

### DEFINITIONS

As used herein, the term “selective inhibitor of a PI3K alpha” refers to any molecule or compound that selectively decreases the activity of a class I PI3K (PI3 kinase) where the PI3K has a p110 alpha catalytic subunit over (i.e., decreases the activity more than) at least one or more than one other class I PI3K isoform, e.g., PI3K with a p110 beta, p110 delta or p110 gamma catalytic subunit).

As used herein, the term “selective inhibitor of a PI3K delta” refers to any molecule or compound that selectively decreases the activity of a class I PI3K (PI3 kinase) where the PI3K has a p110 delta catalytic subunit over (i.e., decreases the activity more than) at least one or more than one other class I PI3K isoform, e.g., PI3K with a p110 alpha, p110 beta or p110 gamma catalytic subunit.

As used herein, an mTOR inhibitor refers to any molecule or compound that decreases the activity of protein complex comprising mTOR. In some embodiments, an mTOR inhibitor is a selective mTOR inhibitor meaning that it does not affect components in the PI3K signaling pathway upstream of mTOR, nor does it affect downstream substrates of mTOR.

As used herein, the term “isolated population” of endoderm cells (or hepatocytes, pancreatic progenitor cells, or other differentiated cells derived from endoderm cells including, but not limited to, intestinal progenitor cells, intestinal cells, lung progenitor cells, lung cells, etc.) refers to a population of one or more endoderm or hepatocyte cells that have been manipulated to provide a preparation of cells that is substantially free of additional components (e.g., cellular debris). Various aspects of isolated populations are described herein.

As used herein, the term “homogeneous population” of endoderm cells refers to a population of cells where a significant portion of the population are endoderm cells. Various embodiments reflecting homogeneity including degrees of homogeneity are described herein.

As used herein, the term “homogeneous population” of hepatocyte cells or hepatocytes refers to a population of cells where a significant portion of the population are hepatocytes.

As used herein, the term “homogeneous population” of pancreatic progenitor cells (and/or pancreatic cells) refers to a population of cells where a significant portion of the population are pancreatic progenitor cells (and/or pancreatic cells).

As used herein, “an effective amount” refers to an amount effective to achieve a goal (e.g., the desired result) of any of the methods described herein.

As used herein, the singular form “a”, “an”, and “the” includes plural references unless indicated otherwise.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X.”

It is understood that aspects and aspects of the invention described herein include “comprising,” “consisting,” and “consisting essentially of” aspects and aspects.

Mesendoderm Cells

Mesendoderm cells are the common progenitor of the mesoderm and endoderm lineages. Thus, the differentiation of stem cells into mesendoderm cells is a critical intermediate step in the efficient production of endoderm cells. Applicants have discovered that mTOR inhibition plays a distinct role from PI3K alpha inhibition during the differentiation of stem cells into mesendoderm and in the differentiation of mesendoderm into endoderm, as indicated by the expression levels of mesendoderm-specific marker genes, endoderm-specific marker genes, and mesoderm-specific marker genes, as described in further detail below. For mesendoderm differentiation, mTOR inhibition is important. The appropriate balance between mTOR inhibition and PI3K alpha inhibition is necessary for obtaining optimal endoderm cells populations, as described in further detail below.

Accordingly, the invention provides not only a population of mesendoderm cells but also methods of obtaining a population of mesendoderm cells by contacting a population of stem cells with an effective amount of an inhibitor of mTOR and an effective amount of Activin A and culturing the cells under conditions sufficient to obtain the population of mesendoderm cells. It is to be understood that these methods can be practiced using one or any combination of mTOR inhibitor(s) described herein. It is also to be understood that the mesendoderm cells obtained in this manner can differentiate into a population of endoderm cells, e.g., any population of endoderm cells described herein.

In some embodiments of the methods, the effective amount of an mTOR inhibitor upregulates the expression of mesendoderm marker genes in the cells after 6 hours in culture, after 8 hours in culture, after 10 hours in culture, after 12 hours in culture, after 14 hours in culture after 16 hours in culture after 18 hours in culture, after 20 hours in culture, after 22 hours in culture, after 24 hours in culture, or more than 24 hours in culture (such as after 26, 28, 30, 32, 34, 36, 38, 40, 42, 44, 46, 48, 50, 52, 56, 58, 60 or more than 60 hours in culture), including any range in between these values. In some embodiments, the mesendoderm marker genes that are upregulated are DKK1, EOMES, FGF17, FGF8, GATA6, MIXL1, T (Brachyury), WNT3A, GSC, LHX1, TBX6, or any combination thereof. In some embodiments, the mesendoderm marker genes that are upregulated are DKK1, FGF17, MIXL1, or any combination thereof. In some embodiments, the expression of DKK1, FGF17, MIXL1, or any combination thereof, is upregulated after 1 day in culture. In some embodiments, the mTOR inhibitor is an siRNA. In some embodiments, the effective amount of the mTOR siRNA is 0.2 nM, 2 nM, or 20 nM.

In some embodiments of the methods, the effective amount of an mTOR inhibitor upregulates the expression of endoderm marker genes in the cells after 6 hours in culture, after 8 hours in culture, after 10 hours in culture, after 12 hours in culture, after 14 hours in culture after 16 hours in culture after 18 hours in culture, after 20 hours in culture, after 22 hours in culture, after 24 hours in culture, or more than 24 hours in culture (such as after 26, 28, 30, 32, 34, 36, 38, 40, 42, 44, 46, 48, 50, 52, 56, 58, 60 or more than 60 hours in culture), including any range in between these values. In some embodiments, the endoderm marker genes that are upregulated in the cells after one day in culture are CDH2, CER1, CXCR4, FGF17, FoxA2, GATA4, GATA6, HHEx, HNF1B, KIT, SOX17, TDGF1, or any combination thereof. In some embodiments, the mTOR inhibitor is an siRNA. In some embodiments, the effective amount of the mTOR siRNA is 0.2 nM, 2 nM, or 20 nM.

In some embodiments of the methods, the effective amount of an mTOR inhibitor downregulates the expression of mesoderm marker genes in the cells after 6 hours in culture, after 8 hours in culture, after 10 hours in culture, after 12 hours in culture, after 14 hours in culture after 16 hours in culture after 18 hours in culture, after 20 hours in culture, after 22 hours in culture, after 24 hours in culture, or more than 24 hours in culture (such as after 26, 28, 30, 32, 34, 36, 38, 40, 42, 44, 46, 48, 50, 52, 56, 58, 60 or more than 60 hours in culture), including any range in between these values. In some embodiments, the mesoderm marker genes that are downregulated in the cells after two days in culture are PDGFRa, BMP4, GATA4, HAND1, ISL1, NCAM1, NKX2-5, TBX6, T (Brachyury) or any combination thereof. In some embodiments, the mTOR inhibitor is an siRNA. In some embodiments, the effective amount of the mTOR siRNA is 0.2 nM, 2 nM, or 20 nM.

As noted above, Applicants have also found that mTOR inhibition and P13K inhibition show synergy for mesendoderm formation. Accordingly, a population of mesendoderm cells can be obtained by contacting a starting source of cells (e.g. adult stem cells, embryonic stem cells, induced pluripotent stem cells) with one or more inhibitor(s) of mTOR and/or PI3K over a period of time to generate a population of mesendoderm cells. It is to be understood that a population of mesendoderm cells described herein can be obtained by using any combination of mTOR inhibitor(s) and/or PI3K alpha inhibitor(s) described herein. Alternatively, a dual mTOR/PI3K alpha inhibitor, e.g., any dual mTOR/PI3K alpha inhibitor described herein (e.g., NVPBKM120, GDC0941-PC), can be used to obtain a population of mesendoderm cells of the invention. It is also to be understood that the mesendoderm cells obtained in this manner can differentiate into a population of endoderm cells, e.g., any population of endoderm cells described herein.

In some embodiments of the methods, the effective amount of an mTOR inhibitor and/or an effective amount of a PI3K alpha inhibitor upregulates the expression of mesendoderm marker genes in the cells after 6 hours in culture, after 8 hours in culture, after 10 hours in culture, after 12 hours in culture, after 14 hours in culture after 16 hours in culture after 18 hours in culture, after 20 hours in culture, after 22 hours in culture, after 24 hours in culture, or more than 24 hours in culture (such as after 26, 28, 30, 32, 34, 36, 38, 40, 42, 44, 46, 48, 50, 52, 56, 58, 60 or more than 60 hours in culture), including any range in between these values. In some embodiments, the mesendoderm marker genes that are upregulated are DKK1, EOMES, FGF17, FGF8, GATA6, MIXL1, T (Brachyury), WNT3A, GSC, LHX1, TBX6, or any combination thereof. In some embodiments, the mesendoderm marker genes that are upregulated are LHX1, GATA6, EOMES, GSC and TBX6, or any combination thereof. In some embodiments, the expression of LHX1, GATA6, EOMES, GSC and TBX6, or any combination thereof, is upregulated after 1 day in culture. In some embodiments, the mTOR inhibitor is an siRNA. In some embodiments, the effective amount of the mTOR siRNA is 0.2 nM, 2 nM, or 20 nM. In some embodiments, the PI3K alpha inhibitor is an siRNA. In some embodiments, the effective amount of the PI3K alpha siRNA is 0.2 nM, 2 nM, or 20 nM. In some embodiments, the effective amount of mTOR siRNA is 20 nM and the effective amount of PI3K siRNA is 2 nM.

In some embodiments of the methods, the effective amount of an mTOR inhibitor and/or an effective amount of a PI3K alpha inhibitor upregulates the expression of endoderm marker genes in the cells after 6 hours in culture, after 8 hours in culture, after 10 hours in culture, after 12 hours in culture, after 14 hours in culture after 16 hours in culture after 18 hours in culture, after 20 hours in culture, after 22 hours in culture, after 24 hours in culture, or more than 24 hours in culture (such as after 26, 28, 30, 32, 34, 36, 38, 40, 42, 44, 46, 48, 50, 52, 56, 58, 60 or more than 60 hours in culture), including any range in between these values. In some embodiments, the endoderm marker genes that are upregulated are CDH2, CER1, CXCR4, FGF17, FoxA2, GATA4, GATA6, HHEx, HNF1B, KIT, SOX17, TDGF1, or any combination thereof. In some embodiments, the endoderm markers that are upregulated are CER1, Hhex and FGF17, and CXCR4. In some embodiments, the expression of CER1, Hhex and FGF17, and CXCR4, or any combination thereof, is upregulated after 2 days in culture. In some embodiments, the mTOR inhibitor is an siRNA. In some embodiments, the effective amount of the mTOR siRNA is 0.2 nM, 2 nM, or 20 nM. In some embodiments, the PI3K alpha inhibitor is an siRNA. In some embodiments, the effective amount of the PI3K alpha siRNA is 0.2 nM, 2 nM, or 20 nM. In some embodiments, the effective amount of mTOR siRNA is 20 nM and the effective amount of PI3K siRNA is 2 nM.

In some embodiments of the methods, the effective amount of an mTOR inhibitor and/or an effective amount of a PI3K alpha inhibitor downregulates the expression of mesoderm marker genes in the cells after 6 hours in culture, after 8 hours in culture, after 10 hours in culture, after 12 hours in culture, after 14 hours in culture after 16 hours in culture after 18 hours in culture, after 20 hours in culture, after 22 hours in culture, after 24 hours in culture, or more than 24 hours in culture (such as after 26, 28, 30, 32, 34, 36 or more than 36 hours in culture), including any range in between these values. In some embodiments, the mesoderm marker genes that are downregulated in the cells after two days in culture are PDGFRa, BMP4, GATA4, HAND1, ISL1, NCAM1, NKX2-5, TBX6, T (Brachyury) or any combination thereof. In some embodiments, the mesoderm marker genes that are downregulated are CER1, Hhex and FGF17, and CXCR4, or any combination thereof. In some embodiments, the expression of CER1, Hhex and FGF17, and CXCR4, or any combination thereof, is downregulated after 2 days in culture. In some embodiments, the mTOR inhibitor is an siRNA. In some embodiments, the effective amount of the mTOR siRNA is 0.2 nM, 2 nM, or 20 nM. In some embodiments, the PI3K alpha inhibitor is an siRNA. In some embodiments, the effective amount of the PI3K alpha siRNA is 0.2 nM, 2 nM, or 20 nM. In some embodiments, the effective amount of mTOR siRNA is 20 nM and the effective amount of PI3K siRNA is 20 nM. In some embodiments, the effective amount of mTOR siRNA is 20 nM and the effective amount of PI3K siRNA is 0.2-2 nM.

Applicants have confirmed distinct roles of mTOR and PI3K alpha inhibition in mesendoderm and endoderm formation. The inhibition of mTOR and the inhibition of PI3K alpha each can make a specific contribution of the expression of mesendoderm marker genes, endoderm marker genes, and mesoderm marker genes. For mesendoderm formation, mTOR inhibition is important. At this stage, the contribution of a high degree of PI3K alpha inhibition helps to enhance the effects of mTOR inhibition. A high degree of PI3K alpha inhibition can also be an important contributor for markers less affected by mTOR inhibition like LHX1. For further differentiation of mesendoderm into endoderm, both PI3K alpha and mTOR inhibition are important to get the highest expression of endoderm gene. PI3K alpha inhibition is important at this stage to prevent other lineages, especially mesoderm, from being formed.

Endoderm Cells

The differentiation of stem cells into mesendoderm cells, and further into endoderm cells is an important step in the efficient production of useful quantities of cells, e.g., hepatocytes, pancreatic progenitor cells, pancreatic cells, or other differentiated cells derived from endoderm cells, such as intestinal progenitor cells, intestinal cells, lung progenitor cells, lung cells, etc., for use in research and regenerative medicine. However, owing to the large variety of cell types that may arise in differentiating stem cell cultures, the vast majority of cell types are produced at very low efficiencies. Moreover, stem cell differentiation in vitro is rather asynchronous. As such, one group of cells may be expressing genes associated with gastrulation, while another group may be starting final differentiation. As an effective way to deal with the above-mentioned problems of mixed and asynchronous stem cell differentiation, the inventors have discovered novel methods for generating populations of endoderm cells that have unique properties. As further detailed below, the methods and/or protocols described herein can be used to efficiently produce a population of endoderm cells such that a significant portion of the population of cells is endoderm cells. These populations of endoderm cells can be efficiently converted to, e.g., hepatocytes, pancreatic progenitor cells, pancreatic cells, or other differentiated cells derived from endoderm cells, such as intestinal progenitor cells, intestinal cells, lung progenitor cells, lung cells, etc., rapidly and in a manner such that homogenous populations of hepatocytes, pancreatic progenitor cells, pancreatic cells, or other differentiated cells derived from endoderm cells, such as intestinal progenitor cells, intestinal cells, lung progenitor cells, lung cells, etc., are generated.

A population of endoderm cells can be made by culturing a starting source of cells with one or more selective inhibitor(s) of PI3K alpha and Activin A, over a period of days (e.g., 1-5 days) to generate a population of endoderm cells. A population of endoderm cells can be also be made by culturing a starting source of cell with one or more selective inhibitor(s) of PI3K delta and Activin A over a period of days (e.g., 1-5 days) to generate a population of endoderm cells. Alternatively, one or more selective inhibitor(s) of PI3K alpha and/or PI3K delta, in combination with Activin A can be used as well.

The methods of the present invention can be practiced using stem cells of various types, including embryonic stem cells (e.g., human embryonic stem cells), adult stem cells, and induced pluripotent stem cells. The methods of the present invention can be practiced on any known stem cell line. Stem cells are undifferentiated cells defined by their ability at the single cell level to both self-renew and differentiate to produce progeny cells, including self-renewing progenitors, non-renewing progenitors, and terminally differentiated cells. Sources of such stem cells include primary embryonic or fetal tissue, umbilical cord tissue, placental tissue, somatic cells, bone marrow, blood, and other cell types. Further details regarding the sources, preparation of, and culturing embryonic stem cells, adult stem cells, and/or induced pluripotent stem cells are described in, e.g., U.S. Pat. No. 7,326,572; U.S. Pat. No. 8,057,789; U.S. Pat. No. 7,259,011; U.S. Pat. No. 7,015,037; U.S. Pat. No. 7,659,118; U.S. Pat. No. 8,058,065; U.S. Pat. No. 8,048,675, and US Patent Application Publication No. US 2007/0281355, the contents of which are expressly incorporated herein by reference in their entirety. In all cases, no human embryos are destroyed in the process of obtaining stem cells for the methods and compositions described herein. The plurality of stem cells is not obtained by the prior destruction of human embryos.

In some methods of producing a population of endoderm cells described herein, stem cells are maintained on a feeder layer. In such methods, any feeder layer which allows the stem cells to be maintained in a pluripotent state can be used. One commonly used feeder layer for the cultivation of human embryonic stem cells is a layer of mouse fibroblasts. More recently, human fibroblast feeder layers have been developed for use in the cultivation of stem cells (see U.S. Patent Application Publication Nos. US 2002/0072117 and US 2010/0028307, the disclosures of which are incorporated herein by reference in their entirety). Alternative methods of the invention for producing a population endoderm cells permit the maintenance of pluripotent stem cells, e.g., human embryonic stem cells, without the use of a feeder layer. Methods of maintaining stem cells under feeder-free conditions have been described in U.S. Patent Application Publication No. US 2003/0175956, the disclosure of which is incorporated herein by reference in its entirety.

In certain embodiments of the methods, stem cells are maintained on a layer of qualified MATRIGEL® (Becton Dickenson). MATRIGEL® is a soluble preparation from Engelbreth-Holm-Swarm tumor cells that gels at room temperature to form a reconstituted basement membrane. Methods of the invention can also be performed on gelatin (Sigma). Additional culture substrates that are suitable for use in the methods described herein are detailed in U.S. Patent Application Publication No. US 2010/0028307. In certain embodiments of the methods, stem cells are maintained on a layer of collagen.

The stem cells used in the methods herein can be maintained in culture either with or without serum. In some embryonic stem cell maintenance procedures, serum replacement is used. In others, serum free culture techniques, such as those described in U.S. Patent Application Publication No. 2003/0190748, the disclosure of which is incorporated herein by reference in its entirety, are used.

In certain embodiments of the methods described herein, the isolated populations of endoderm cells described herein are obtained from stem cells cultured in suspension. Methods of culturing stem cells in this manner are known in the art and described in, e.g., Amit et al. (2011) Nature Protocols 6: 572-579; Zweigerdt et al. (2011) Nature Protocols 6: 689-700; Singh et al. (2010) Stem Cell Res 4: 165-170; Kehoe et al. (2010) Tissue Eng Part A. 16: 405-21; and Olmer et al. (2011) Stem Cell Research 5: 51-64. Additional methods of culturing stem cells in suspension are described in U.S. Pat. No. 8,008,075; U.S. Pat. No. 7,790,456; and U.S. Pat. No. 5,491,090, the contents of each of which are hereby incorporated herein by reference in their entirety. Another method of culturing stem cells in suspension is described in Example 1 below.

The invention contemplates any and all of the parameters, as described above and elsewhere herein, in any combination, to describe methods of obtaining a population of endoderm cells.

Methods of Producing Endoderm Cells

Endoderm cells can be obtained when a starting source of cells, such as stem cells (e.g. adult stem cells, embryonic stem cells, induced pluripotent stem cells) are contacted with any one of the following options (1) a selective inhibitor of PI3K alpha and Activin A; (2) a selective inhibitor of PI3k delta and Activin A, and (3) one or more selective inhibitors of PI3K alpha and/or PI3K delta and Activin A. As further detailed below, various types of compounds or classes of compounds can be used in conjunction with Activin A to produce a population of endoderm cells. Furthermore, inhibitors of mTOR can be used in conjunction with selective inhibitors of PI3K alpha and/or PI3K delta and Activin A for efficient production of endoderm cells.

Endoderm can also be obtained when a starting source of cells, such as stem cells (e.g., adult stem cells, embryonic stem cells, induced pluripotent stem cells) are contacted with an mTOR inhibitor

mTOR Kinase Inhibitors

mTOR kinase inhibitors can be used alone or in combination with other compounds (e.g., PI3K alpha inhibitors) to produce mesendoderm cells, endoderm cells, and differentiated cells derived from endoderm cells (e.g., intestinal progenitor cells, intestinal cells, lung progenitor cells, lung cells, hepatocytes, pancreatic cells, etc.). In certain embodiments, endoderm cells can be made by contacting a population of stem cells with an effective amount of a member of the TGF beta family (such as Activin A) and an effective amount of an inhibitor or mTOR kinase or a selective dual inhibitor of both PI3K and mTOR kinase, and in certain embodiment of a dual inhibitor of a PI3K alpha selective inhibitor and an mTOR kinase inhibitor. mTOR is a 289 kDa serine/threonine kinase that is considered a member of the phosphoinositide-3-kinase-like kinase (PIKK) family, because it contains a carboxyl terminal kinase domain that has significant sequence homology to the catalytic domain of phosphoinositide 3-kinase (PI3K) lipid kinases. In addition to the catalytic domain at the C-terminus, mTOR kinase also contains a FKBP12-Rapamycin binding (FRB) domain, a putative repressor domain near the C-terminus and up to 20 tandemly-repeated HEAT motifs at the N-terminus as well as a FRAP-ATM-TRRAP (FAT) and FAT C-terminus domain. See, Huang and Houghton, Current Opinion in Pharmacology, 2003, 3, 371-377.) In the literature, mTOR kinase is also referred to as FRAP (FKBP12 and rapamycin associated protein), RAFT1 (rapamycin and FKBP12 target 1), RAPT1 (rapamycin target 1)).

mTOR kinase can be activated by growth factors through the PI3K-Akt pathway or by cellular stresses, such as deprivation of nutrients or hypoxia. The activation of mTOR kinase is thought to play a central role in regulating cell growth and cell survival via a wide range of cellular functions including translation, transcription, mRNA turnover, protein stability, actin cytoskeleton reorganization and autophagy. For a detailed review of mTOR cell signaling biology and potential therapeutic effects of modulating the mTOR signaling interactions, see Sabatini, D. M. and Guertin, D. A. (2005) An Expanding Role for mTOR in Cancer TRENDS in Molecular Medicine, 11, 353-361; Chiang, G. C. and Abraham, R. T. (2007) Targeting the mTOR signaling network in cancer TRENDS 13, 433-442; Jacinto and Hall (2005) Tor signaling in bugs, brain and brawn Nature Reviews Molecular and Cell Biology, 4, 117-126; and Sabatini, D. M. and Guertin, D. A. (2007) Defining the Role of mTOR in Cancer Cell, 12, 9-22.

For example, there is evidence to show that PI3K-AKT signaling pathway, which lies upstream of mTOR kinase, is frequently over activated in cancer cells, which subsequently results in the hyperactivation of downstream targets like mTOR kinase. More specifically, the components of the PI3K-AKT pathway that are mutated in different human tumors include activation mutations of growth factor receptors and the amplification and overexpression of PI3K and AKT. In addition, there is evidence which shows that many tumor types, including glioblastoma, hepatocellular carcinoma, lung carcinoma, melanoma, endometrial carcinomas, and prostate cancer, contain loss-of-function mutations of negative regulators of the PI3K-AKT pathways, such as phosphatases and tensin homolog deleted on chromosome 10 (PTEN) and tuberous sclerosis complex (TSC1/TSC2), which also results in hyperactive signaling of mTOR kinase. The above suggests that inhibitors of mTOR kinase can be effective therapeutics for the treatment of diseases caused, at least in part, by the hyperactivity of the mTOR kinase signaling.

mTOR kinase exists as two physically and functionally distinct signaling complexes (i.e., mTORC1 and mTORC2). mTORC1, also known as the “mTOR-Raptor complex” or the “rapamycin-sensitive complex” because it binds to and is inhibited by the small molecule inhibitor rapamycin. mTORC1 is defined by the presence of the proteins mTOR, Raptor and mLST8. Rapamycin, itself, is a macrolide and was discovered as the first small molecule inhibitor of mTOR kinase. To be biologically active, rapamycin forms a ternary complex with mTOR and FKBP12, which is a cytosolic binding protein collectively called immunophilin. Rapamycin acts to induce the dimerization of mTOR and FKBP12. The formation of rapamycin-FKBP12 complex results in a gain-of-function, because the complex binds directly to mTOR and inhibits the function of mTOR.

A second, more recently discovered mTORC complex, mTORC2, is characterized by the presence of the proteins mTOR, Rictor, Protor-1, mLST8 and mSIN1. mTORC2 is also referred to as the “mTOR-Rictor complex” or the “rapamycin-insensitive” complex because it does not bind to rapamycin.

Both mTOR complexes play important roles in intracellular signaling pathways that affect a cell's growth, and proliferation, and survival. For example, the downstream target proteins of mTORC1 include Ribosomal S6 kinases (e.g., S6K1, S6K2) and eukaryotic initiation factor 4E binding protein (4E-BP1), which are key regulators of protein translation in cells. Also, mTORC2 is responsible for the phosphorylation of AKT (S473); and studies have shown that uncontrolled cell proliferation due to hyperactivation of AKT to be a hallmark of several cancer types.

In some embodiments, inhibiting the activity of mTOR in stem cells cultured in the presence of an effective amount of Activin A enhances endoderm differentiation. Accordingly, the invention provides methods of obtaining a population of endoderm cells by contacting a population of stem cells with an effective amount of an inhibitor of mTOR and an effective amount of Activin A and culturing the cells under conditions sufficient to obtain the population of endoderm cells. In some embodiments, endoderm is not obtained as efficiently by contacting stem cells with an effective amount of an Akt inhibitor, i.e., a component upstream of mTOR in the PI3K signaling pathway, and effective amount of Activin A. Exemplary AKT inhibitors include, e.g., Palomid 529, AT7867, and AKT inhibitors used in the Examples.

A population of endoderm cells obtained by contacting stem cells with an effective amount of an inhibitor of mTOR and an effective amount of Activin A can be a population in which, e.g., at least about 30%, at least about 35%, at least about 40%, or more than 40%, e.g., at least about 45%, at least about 50%, at least about 55%, at least about 60%, at least about 65%, at least about 70%, at least about 75%, or more than 75% of the cells express FoxA2. In certain aspects, a population of endoderm cells obtained by contacting stem cells with an inhibitor of mTOR and Activin A can be a population in which, e.g., at least about 30%, at least about 35%, at least about 40%, or more than 40%, e.g., at least about 45%, at least about 50%, at least about 55%, at least about 60%, at least about 65%, at least about 70%, at least about 75%, or more than 75%, e.g., at least about 80%, at least about 85%, at least about 90%, or more than 90% of the cells express FoxA2. A population of endoderm cells obtained by methods that include contacting stem cells with an inhibitor of mTOR can be a population in which, e.g., at least about 30%, at least about 35%, at least about 40%, or more than 40%, e.g., at least about 45%, at least about 50%, at least about 55%, at least about 60%, at least about 65%, at least about 70%, at least about 75%, or more than 75% of the cells express CXCR4. In some embodiments, these populations of endoderm cells are obtained after at least about 1, 2, or 3 days of culturing in media suitable for endoderm formation (see, e.g., Examples). In other embodiments, these populations of endoderm cells are obtained after at least about 4 or 5 days of culturing. In other embodiments, these populations of endoderm cells are obtained after more than 5 days of culturing.

A population of endoderm cells obtained by the methods can be a population in which, e.g., at least about 30%, at least about 35%, at least about 40%, or more than 40%, e.g., at least about 45%, at least about 50%, at least about 55%, at least about 60%, at least about 65%, at least about 70%, at least about 75%, or more than 75%, the cells express SOX 17 and Fox A2. For example, the methods can be used to obtain a population of stem cells in which at least about 40% of the cells express FoxA2 and at least 61% of the cells express SOX 17. In certain aspects, e.g., at least about 30%, at least about 35%, at least about 40%, at least about 50%, at least about 55%, at least about 60%, at least about 65%, at least about 70%, at least about 75% or more than about 75% of the cells in a population of endoderm cells produced by contacting stem cells with an effective amount of an mTOR inhibitor and an effective amount of Activin A express SOX17 and CXCR4. A population of endoderm cells obtained by contacting a population of stem cells with an mTOR inhibitor can be a population of cells in which, e.g., at least about 30%, at least about 35%, at least about 40%, or more than 40%, e.g., at least about 45%, at least about 50%, at least about 55%, at least about 60%, at least about 65%, at least about 70%, at least about 75%, or more than 75%, the cells express CXCR4 and Fox A2. Methods of the invention that include contacting a population of stem cells with an inhibitor of mTOR can be used to produce a population of cells in which, e.g., at least about 30%, at least about 35%, at least about 40%, or more than 40%, e.g., at least about 45%, at least about 50%, at least about 55%, at least about 60%, at least about 65%, at least about 70%, at least about 75%, or more than 75%, the cells express SOX 17, Fox A2, and CXCR4. In some embodiments, these populations of endoderm cells are obtained after at least about 1, 2, or 3 days of culturing in media suitable for endoderm formation (see, e.g., Examples). In other embodiments, these populations of endoderm cells are obtained after at least about 4 or 5 days of culturing. In other embodiments, these populations of endoderm cells are obtained after more than 5 days of culturing.

Methods of the invention that include contacting stem cells with an effective amount of an mTOR inhibitor and an effective amount of Activin A encompass contacting stem cells with an siRNA that specifically inactivates an mRNA transcribed from an mTOR gene. In these embodiments, the methods encompass contacting the stem cells with at least 5 nM, at least 6 nM, at least 7 nM, at least 8 nM, at least 9 nM, at least 10 nM, or greater than 10 nM of the siRNA.

An mTOR inhibitor used with the methods can be a small molecule. For example, any one or combination of small molecules depicted or listed below can be used in the methods:

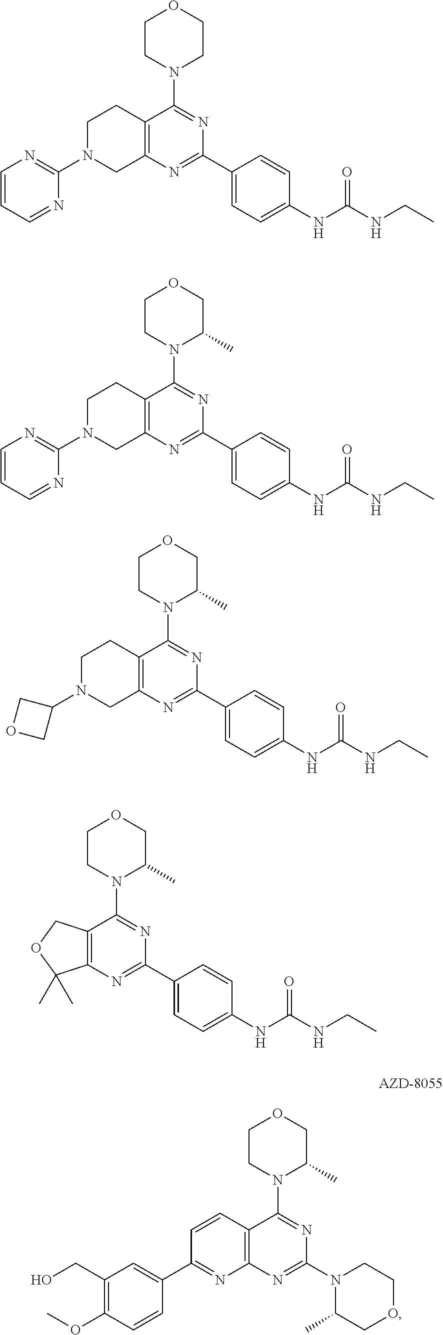

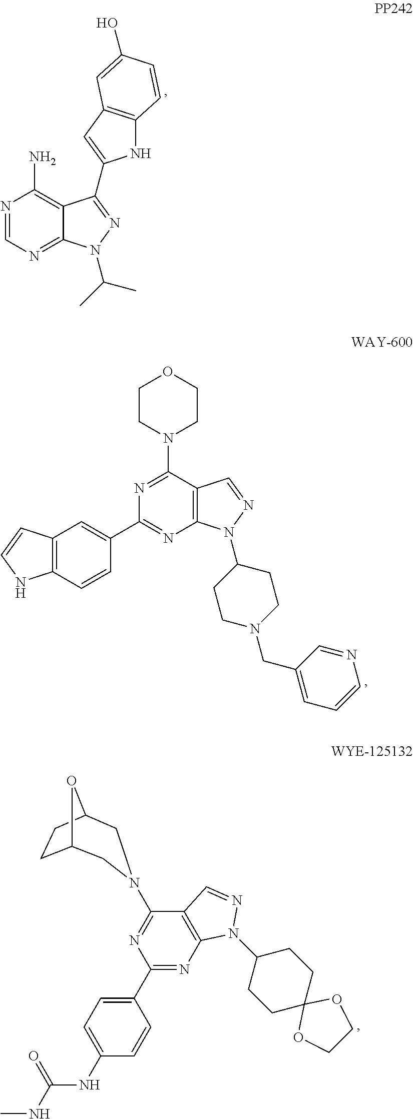 [SMILES: CC(C)n1nc(-c2cc3cc(O)ccc3[nH]2)c2c(N)ncnc21 ; c1cncc(CN2CCC(n3ncc4c(N5CCOCC5)nc(-c5ccc6[nH]ccc6c5)nc43)CC2)c1 ; CNC(=O)Nc1ccc(-c2nc(N3CC4CCC(C3)O4)c3cnn(C4CCC5(CC4)OCCO5)c3n2)cc1]

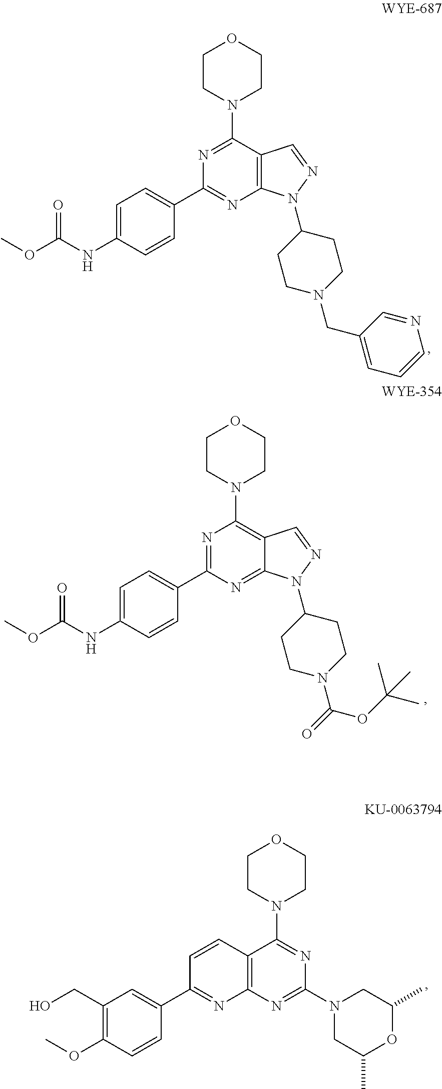

Merck's AP23573 (also known as ridaforolimus or deforolimus), Pfizer's Torsel (also known as Temsirolimus or CI-779), Intellikine's INK128, AstraZeneca's AZD2012, Celgene's CC-223, KU-0063794, OSI's OSI-027, sirolimus (rapamycin), and everolimus. Torin1 can also be used.

A dual inhibitor of PI3K and mTOR, and in certain embodiments of a dual inhibitor of a PI3K alpha and mTOR, used with the methods can be a small molecule. For example, any one or combination of small molecules depicted or listed below can be used in the methods:

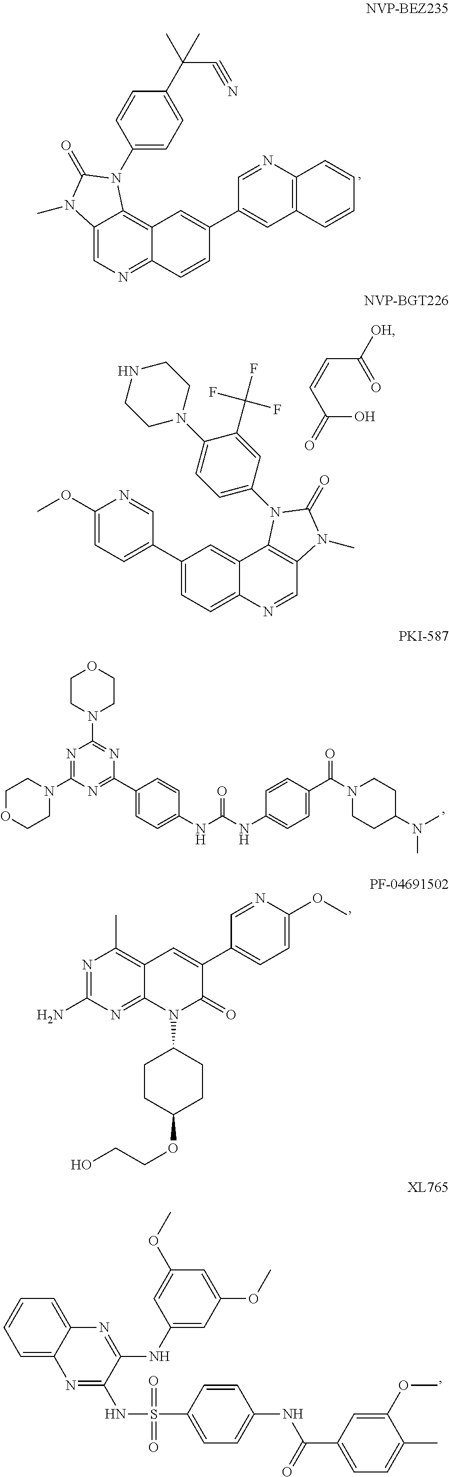

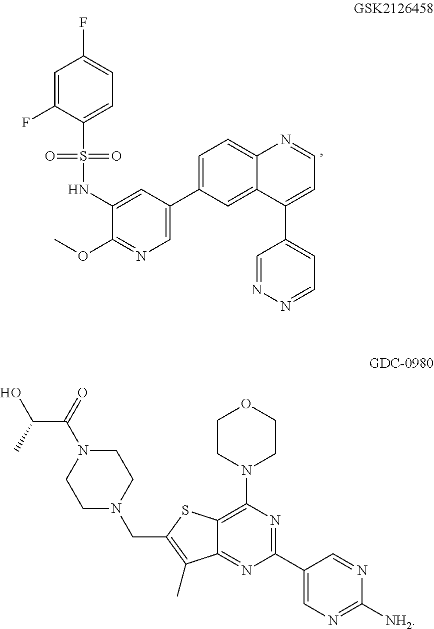 [SMILES: COc1ncc(-c2ccc3nccc(-c4ccnnc4)c3c2)cc1NS(=O)(=O)c1ccc(F)cc1F ; Cc1c(CN2CCN(C(=O)[C@H](C)O)CC2)sc2c(N3CCOCC3)nc(-c3cnc(N)nc3)nc12]

Methods of the invention that include contacting a population of stem cells with an effective amount of an mTOR inhibitor encompass contacting stem cells with an effective amount of an mTOR inhibitor or a dual inhibitor of PI3K (e.g., a PI3K alpha inhibitor) and mTOR kinase. In certain embodiments, the mTOR inhibitor is rapamycin or a rapamycin analog (e.g., everolimus, temsirolimus), KU0063794 or WYE-354. An effective amount of any one or combination of these mTOR inhibitors can be, e.g., about 1 nM to about 1 μM, between about 10 nM to about 950 nM, between about 25 nM to about 900 nM, between about 50 nM to about 800 nM, or approximately 750 nM.

An effective amount of any dual inhibitor of PI3K alpha and mTOR can be, e.g., about 1 nM to about 1 μM, between about 10 nM to about 950 nM, between about 25 nM to about 900 nM, between about 50 nM to about 800 nM, or approximately 750 nM.

Beneficially, the population of endoderm cells obtained by methods that include contacting stem cells with Activin A and an inhibitor of mTOR has the capability to differentiate into hepatocytes, pancreatic cells, and intestinal cells. The endoderm cells also have the capability to differentiate into lung cells, such as lung epithelial cells and airway progenitor cells.

The methods that include contacting a population of stem cells with an mTOR inhibitor encompass contacting stem cells with any one or combination of mTOR inhibitors described in US 2010/0069357, US 2010/0331305, US 2011/0086840, US 2011/0086841, U.S. Pat. No. 7,902,189B2, US 77/50003B2, US 2009/0270390A1, US 2009/0233926A1, US 2009/0018134A1, WO 2008/032077A1, WO 2008/032089A1, WO 2008/032091A1, WO 2008/032086A1, WO 2008/032072A1, WO 2008/032027A1, US 2010/0022534A1, WO 2008/032033A1, WO 2008/032036A1, WO 2008/032041A1, WO 2008/032060A1, WO 2006/051270A1, U.S. Pat. No. 5,536,729 U.S. Pat. No. 5,665,772, US 81/01602B2, US 75/04397B2, US 80/39469B2, US 81/29371B2, US 81/29371B2, US 2010/0068204A1, US 2010/0061982A1, US 2010/0041692A1, US 2010/0015141A1, US 2010/0003250A1, US 2009/0311217A1, US 2009/0298820A1, US 2009/0227575A1, US 2009/0192147A1, US 2009/0192176A1, US 2009/0181963A1, US 2009/0149458A1, US 2009/0149458A1, US 2008/0233127A1, US 2008/0234262A1, US 2010/0069357A1, US 2010/0331305A1, US 2011/0086840A1, US 2011/0086841A1, and Shuttleworth et al. (2011) Current Medicinal Chemistry 18: 2686-2714, the contents of which are expressly incorporated herein by reference in their entirety.

As described herein, certain embodiments of obtaining a population of endoderm cells entail contacting a population of stem cells with an effective amount of an mTOR inhibitor and an effective amount of a selective inhibitor of PI3K alpha. It will be appreciated that the methods encompass the use of any one or combination of PI3K alpha inhibitors noted herein with any one or combination of mTOR inhibitors described herein.

Phosphatidylinositol 3-Kinase

Phosphatidylinositol (PI) is one of a number of phospholipids found in cell membranes that participate in intracellular signal transduction. Cell signaling via 3′-phosphorylated phosphoinositides has been implicated in a variety of cellular processes, e.g., malignant transformation, growth factor signaling, inflammation, and immunity (Rameh et al. (1999) J. Biol. Chem. 274:8347-8350). The enzyme responsible for generating these phosphorylated signaling products, phosphatidylinositol 3-kinase (also referred to as PI 3-kinase or PI3K), was originally identified as an activity associated with viral oncoproteins and growth factor receptor tyrosine kinases that phosphorylate phosphatidylinositol (PI) and its phosphorylated derivatives at the 3′-hydroxyl of the inositol ring (Panayotou et al (1992) Trends Cell Biol 2:358-60). Phosphoinositide 3-kinases (PI3K) are lipid kinases that phosphorylate lipids at the 3-hydroxyl residue of an inositol ring (Whitman et al (1988) Nature, 332:664). The 3-phosphorylated phospholipids (PIP3s) generated by PI3-kinases act as second messengers recruiting kinases with lipid binding domains (including plekstrin homology (PH) regions), such as Akt and PDK1, phosphoinositide-dependent kinase-1 (Vivanco et al (2002) Nature Rev. Cancer 2: 489; Phillips et al (1998) Cancer 83:41).

The PI3 kinase family comprises at least 15 different enzymes sub-classified by structural homology and are divided into 3 classes based on sequence homology and the product formed by enzyme catalysis. The class I PI3 kinases are composed of 2 subunits: a 110 kd catalytic subunit and an 85 kd regulatory subunit (Otsu et al (1991) Cell 65:91-104; Hiles et al (1992) Cell 70:419-29). The regulatory subunits contain SH2 domains and bind to tyrosine residues phosphorylated by growth factor receptors with a tyrosine kinase activity or oncogene products, thereby inducing the PI3K activity of the p110 catalytic subunit which phosphorylates its lipid substrate. Class I PI3 kinases are involved in important signal transduction events downstream of cytokines, integrins, growth factors and immunoreceptors, which suggests that control of this pathway may lead to important therapeutic effects such as modulating cell proliferation and carcinogenesis. Class I PI3Ks can phosphorylate phosphatidylinositol (PI), phosphatidylinositol-4-phosphate, and phosphatidylinositol-4,5-biphosphate (PIP2) to produce phosphatidylinositol-3-phosphate (PIP), phosphatidylinositol-3,4-biphosphate, and phosphatidylinositol-3,4,5-triphosphate, respectively. Class II PI3Ks phosphorylate PI and phosphatidylinositol-4-phosphate. Class III PI3Ks can only phosphorylate PI. A key PI3-kinase isoform in cancer is the Class I PI3-kinase, p110 alpha as indicated by recurrent oncogenic mutations in p110 alpha (Samuels et al (2004) Science 304:554). (U.S. Pat. No. 5,824,492; U.S. Pat. No. 5,846,824; U.S. Pat. No. 6,274,327). Other isoforms may be important in cancer and are also implicated in cardiovascular and immune-inflammatory disease (Workman P (2004) Biochem Soc Trans 32:393-396; Patel et al (2004) Proc. Am. Assoc. of Cancer Res. (Abstract LB-247) 95th Annual Meeting, March 27-31, Orlando, Fla., USA; Ahmadi K and Waterfield M D (2004) “Phosphoinositide 3-Kinase: Function and Mechanisms” Encyclopedia of Biological Chemistry (Lennarz W J, Lane M D eds) Elsevier/Academic Press), Oncogenic mutations of p110 alpha have been found at a significant frequency in colon, breast, brain, liver, ovarian, gastric, lung, and head and neck solid tumors. PTEN abnormalities are found in glioblastoma, melanoma, prostate, endometrial, ovarian, breast, lung, head and neck, hepatocellular, and thyroid cancers.

Four distinct Class I PI3Ks have been identified, designated PI3K alpha, beta, delta, and gamma, each consisting of a distinct 110 kDa catalytic subunit and a regulatory subunit. Three of the catalytic subunits, i.e., p110 alpha, p110 beta, and p110 delta, each interact with the same regulatory subunit, p85; whereas p110 gamma interacts with a distinct regulatory subunit, p101. The patterns of expression of each of these PI3Ks in human cells and tissues are distinct. In each of the PI3K alpha, beta, and delta subtypes, the p85 subunit acts to localize PI3 kinase to the plasma membrane by the interaction of its SH2 domain with phosphorylated tyrosine residues (present in an appropriate sequence context) in target proteins (Rameh et al (1995) Cell, 83:821-30; Volinia et al (1992) Oncogene, 7:789-93).

It has previously been demonstrated that Activin A, a member of the TGF beta family, induces endoderm differentiation when PI3K signaling is suppressed (McLean et al. (2007) Stem Cells 25: 29; Ramasamy et al. (2010) Differentiation 80: S25). See also, e.g., US 2007/0281335, entitled, “Compositions and Methods For Self-Renewal and Differentiation in Human Embryonic Stem Cells”, which is hereby incorporated herein by reference in its entirety. A variety of PI3K inhibitors have been used to differentiate a population of endoderm cells from a population of stem cells (see, e.g., Knight (2010) Current Topics in Microbiology and Immunology 247: 263-277; McNamara et al. (2011) Future Med Chem 3: 549-565, however, these findings have not yielded an efficient conversion of a starting population of a cell source (such as stem cells) to endoderm cells, hepatocytes, or pancreatic progenitor cells.

Applicants have discovered that specifically inhibiting the activity of p110 alpha enhances endoderm differentiation more efficiently and more robustly than inhibiting the activity of p110 beta, p110 delta, or p110 gamma. Accordingly, the invention provides methods of obtaining a population of endoderm cells, e.g., a population of endoderm cells having any one or more of the characteristics of a population described herein. The methods include contacting a population of stem cells with an effective amount of Activin A and an effective amount of a selective inhibitor of the PI3K alpha isoform and culturing the stem cells under conditions sufficient to obtain the population of endoderm cells. In one embodiment, a selective inhibitor of a PI3K alpha specifically inhibits Class I PI3Ks where the PI3K has a p110 alpha catalytic subunit. In some embodiments, it does not affect the activities of Class I PI3Ks comprising p110 beta, delta, or gamma subunits; Class II PI3Ks; or Class III PI3Ks.

In such methods, in some embodiments, an effective amount of a selective inhibitor of PI3K alpha is one that inhibits PI3K alpha at potency (IC₅₀) of at least about 1 μM, at least about 750 nM, at least about 500 nM, at least about 250 nM, at least about 100 nM, at least about 50 nM, at least about 25 nM, at least about 10 nM, at least about 5 nM or at least about 1 nM.

In such methods, the selective inhibitor of PI3K alpha is one that inhibits PI3K alpha (IC₅₀) with at least 1000 fold selectivity over at least one other PI3K isoform, with at least 750 fold selectivity over other at least one other PI3K isoform, with at least 500 fold selectivity over at least one other PI3K isoform, with at least 250 fold selectivity over at least one other PI3K isoform, with at least 100 fold selectivity over at least one other PI3K isoform, with at least 50 fold selectivity against on other PI3K isoform, with at least 25 fold selectivity against one other PI3K isoform, with at least 10 fold selectivity against at least one other PI3K isoform, with at least 5 fold selectivity again at least one other PI3K isoform, or with at least 2 fold selectivity against at least one other PI3K isoform.

Accordingly methods of the invention can be used to obtain a population of endoderm cells in which at least about 50%, at least about 60%, at least about 65%, at least about 70%, at least about 75%, at least about 80%, at least about 81%, at least about 82%, or at least about 83% of the cells express SOX 17. The methods can also be used to obtain a population of endoderm cells in which greater than 83%, e.g., at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater than 99% of the cells in an isolated population of endoderm cells express SOX17. In one embodiment, 100% of the cells in an isolated population of endoderm cells express SOX17. In some embodiments, these populations of endoderm cells are obtained after at least about 1, 2, or 3 days of culturing in media suitable for endoderm formation (see, e.g., Examples). In other embodiments, these populations of endoderm cells are obtained after at least about 4 or 5 days of culturing. In other embodiments, these populations of endoderm cells are obtained after more than 5 days of culturing.

Additionally, the methods of the invention can be used to obtain a population of endoderm cells in which at least about 50%, at least about 60%, at least about 65%, at least about 70%, at least about 71%, at least about 72%, at least about 73%, at least about 74%, at least about 75%, at least about 76%, or at least about 77% of the cells express FoxA2. Additionally, the methods can produce a population of endoderm cells in which greater that about 77%, e.g., at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, greater than about 90%, greater than about 93%, greater than about 95%, greater than about 97%, or greater than about 99%, of the cells express FoxA2. In one embodiment, 100% of the cells in an isolated population of endoderm cells express FoxA2. In some embodiments, these populations of endoderm cells are obtained after at least about 1, 2, or 3 days of culturing in media suitable for endoderm formation (see, e.g., Examples). In other embodiments, these populations of endoderm cells are obtained after at least about 4 or 5 days of culturing. In other embodiments, these populations of endoderm cells are obtained after more than 5 days of culturing.

In some aspects, the invention provides methods for obtaining a population of endoderm cells in which at least about 50%, at least about 55%, at least about 60%, at least about 65%, at least about 70%, at least about 75%, or at least about 76% of the cell express CXCR4. A population of endoderm cells obtained by the methods provided herein can be a population in which greater than 76%, e.g., at least about 77%, at least about 78%, at least about 79%, at least about 80%, at least about 81%, at least about 82%, at least about 83%, at least about 84%, greater than 85%, greater than 86%, greater than 87%, greater than 88%, at least about 89%, at least about 90%, greater than about 90%, greater than about 93%, greater than about 95%, greater than about 97%, or greater than about 99%, of the cells express CXCR4. In one embodiment, 100% of the cells in an isolated population of endoderm cells express CXCR4. In some embodiments, these populations of endoderm cells are obtained after at least about 1, 2, or 3 days of culturing in media suitable for endoderm formation (see, e.g., Examples). In other embodiments, these populations of endoderm cells are obtained after at least about 4 or 5 days of culturing. In other embodiments, these populations of endoderm cells are obtained after more than 5 days of culturing.

Accordingly, the invention provides a method of obtaining a population of endoderm cells in which at least about 50%, at least about 65%, at least about 60%, at least about 70%, least about 75%, or greater than about 75% of the cells express both Sox17 and FoxA2. A population of endoderm cells of the invention can be, e.g., a population in which at least 83% of the cells express SOX17 and at least 77% of the cells express FoxA2. In some embodiments, these populations of endoderm cells are obtained after at least about 1, 2, or 3 days of culturing in media suitable for endoderm formation (see, e.g., Examples). In other embodiments, these populations of endoderm cells are obtained after at least about 4 or 5 days of culturing. In other embodiments, these populations of endoderm cells are obtained after more than 5 days of culturing.

The methods of the invention can be used to obtain a population of endoderm cells in which at least about 50%, at least about 55%, at least about 60%, at least about 65%, at least about 70%, or at least about 75% of the cells express both SOX17 and CXCR4. In certain aspects, the methods can be used to obtain a population of cells in which greater than 75%, e.g., at least about 76%, at least about 77%, at least about 78%, at least about 79%, at least about 80%, at least about 81%, at least about 82%, or at least about 83%, of the cells express both SOX17 and CXC4. For example, the invention provides a method to obtain a population of endoderm cells in which at least 83% of the cells express SOX17 and at least 76% of the cells express CXCR4. In some embodiments, these populations of endoderm cells are obtained after at least about 1, 2, or 3 days of culturing in media suitable for endoderm formation (see, e.g., Examples). In other embodiments, these populations of endoderm cells are obtained after at least about 4 or 5 days of culturing. In other embodiments, these populations of endoderm cells are obtained after more than 5 days of culturing.

Additionally, a population of endoderm cells produced by the methods of the invention can be a population in which at least about 50%, at least about 55%, at least about 60%, at least about 65%, at least about 70%, or at least about 75%, or greater than about 75% of the cells express both FoxA2 and CXCR4. For example, the invention provides a method of obtaining a population of endoderm cells in which at least about 77% of the cells express FoxA2 and at least about 76% of the cells express CXCR4. In some embodiments, these populations of endoderm cells are obtained after at least about 1, 2, or 3 days of culturing in media suitable for endoderm formation (see, e.g., Examples). In other embodiments, these populations of endoderm cells are obtained after at least about 4 or 5 days of culturing. In other embodiments, these populations of endoderm cells are obtained after more than 5 days of culturing.

A method provided by the invention can be used to obtain a population in which about 50%, about 55%, about 60%, about 65%, about 70%, about 75% or greater than 75%, e.g., at least 76%, at least 77%, at least 78%, at least 79%, at least 80%, at least 81%, at least 82%, at least 83%, or greater than 83% of the cells express SOX17, FoxA2, and CXCR4. For example, a method provided by the invention can be used to obtain a population in which at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or greater that 99% of the cells express SOX17, FoxA2, and CXCR4. In one embodiment, 100% of the cells in an isolated population of endoderm cells express SOX17, FoxA2, and CXCR4. In certain aspects a method provided herein can be used to obtain an isolated population of endoderm cells in which at least 83% of the cells express SOX17, at least 77% of the cells express FoxA2, and at least 76% of the cells express CXCR4. In certain aspects a method provided herein can be used to obtain an isolated population of endoderm cells in which at least 83% of the cells express SOX17, at least 77% of the cells express FoxA2, or at least 76% of the cells express CXCR4. In some embodiments, these populations of endoderm cells are obtained after at least about 1, 2, or 3 days of culturing in media suitable for endoderm formation (see, e.g., Examples). In other embodiments, these populations of endoderm cells are obtained after at least about 4 or 5 days of culturing. In other embodiments, these populations of endoderm cells are obtained after more than 5 days of culturing.

A method provided by the invention can be used to obtain a population of endoderm cells in which at least 62% of the cells express SOX17, at least 50% of the cells express FoxA2, and at least 35% of the cells express CXCR4 after 2 days in culture with an effective amount of Activin A and an effective amount of a PI3K inhibitor. In certain embodiments, a method of the invention can be used to obtain a population of endoderm cells in which at least 83% of the cells express SOX17, at least 77% of the cells express FoxA2, and at least 76% of the cells express CXCR4 after 3 days in culture with an effective amount of Activin A and an effective amount of a PI3K inhibitor. A method of the invention can be used to obtain a population of endoderm cells in which at least 88% of the cells express SOX17, at least 82% of the cells express FoxA2, and at least 75% of the cells express CXCR4 after 4 days in culture with an effective amount of Activin A and an effective amount of a PI3K inhibitor. In another embodiment, a method of the invention can be used to obtain a population of endoderm cells in which at least 91% of the cells express SOX17, at least 87% of the cells express FoxA2, and at least 82% of the cells express CXCR4 after 5 days in culture with an effective amount of Activin A and an effective amount of a PI3K inhibitor.

In another embodiment, a method of the invention can be used to obtain a population of endoderm cells in which greater than 91%, e.g., at least 92%, at least 93%, at least 94%, at least 95%, at least 96%, at least 97%, at least 98%, at least 99% or greater than 99% of the cells express SOX17, greater than 87%, e.g., at least 88%, at least 89%, at least 90%, at least 91%, at least 92%, at least 93%, at least 94%, at least 95%, at least 96%, at least 97%, at least 98%, at least 99% or greater than 99% of the cells express FoxA2, and greater than 82%, e.g., at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or greater than 99% of the cells express CXCR4 after 5 days in culture with an effective amount of Activin A and an effective amount of a PI3K inhibitor. In one embodiment, 100% of the cells in an isolated population of endoderm cells express SOX17, FoxA2 and CXCR4 when cultured with an effective amount of Activin A and an effective amount of a PI3K inhibitor. In some embodiments, these populations are obtained after 1, 2, 3, or 4 days in culture.

In some embodiments, the cell populations (e.g., population of endoderm cells) have the described lower limit of any one or more markers described herein (e.g., SOX17, FOXA2, CXCR4) coupled with an upper limit of any one or more markers described herein. The invention contemplates a range that encompasses both a lower limit and an upper percentage of any of the numerical values recited herein. For example, one embodiment contemplates a population of endoderm cells where about 50% to about 90% of the endoderm cells in the population express SOX17. As a further example, in some embodiments, an upper limit of percentages can be any of about the following: 75%, 80%, 85%, 90%, 95%, or 99%.

Accordingly, the methods of the invention can be used to obtain a population of endoderm cells with high efficiency. Advantageously, the population of endoderm cells is a homogenous population, which obviates the need to sort the cells (i.e., to enrich the population for endoderm cells) prior to their use in a downstream application. Beneficially, the methods of the invention can be used to obtain a population of endoderm cells that has the capability to differentiate into any one or more of the following: hepatocytes, pancreas cells, and intestinal cells.

In certain embodiments of the methods, stem cells are contacted with selective inhibitor of PI3K alpha and a member of the TGF beta family. The methods can include contacting the stem cells with a member of the TGF beta family selected from the group consisting of Nodal, Activin A, Activin B, Activin AB, TGF-beta, BMP2, BMP4, and mixtures of two or more of the foregoing. In the methods of the invention, the effective amount of a member of the TGF beta family can be between about 1 ng/ml to about 1 mg/ml, between about 5 ng/ml to about 600 ng/ml, between about 10 ng/ml to about 500 ng/ml, between about 25 ng/ml to about 250 ng/ml, between about 50 ng/ml to about 200 ng/ml, or approximately 100 ng/ml. In some embodiments of the invention, the stem cells are contacted with an effective amount of Activin A. In these methods, the effective amount of Activin A can be about 25 ng/ml, about 50 ng/ml, about 75 ng/ml, about 100 ng/ml, about 100 ng/ml, about 150 ng/ml, or about 200 ng/ml. In other embodiments of the invention, the stem cells are contacted with an effective amount of Activin A of about 1 ng/ml-600 ng/ml, 5 ng/ml-500 ng/ml, about 10 ng/ml-400 ng/ml, about 25 ng/ml-200 ng/ml, about 25 ng/ml-150 ng/ml, or about 25 ng/ml-100 ng/ml.

Certain methods of the invention encompass further contacting a population of stem cells with an effective amount of a member of the TGF beta family (such as Activin A), a selective inhibitor of a PI3K alpha, and an effective amount of an mTOR inhibitor. In some embodiments, the methods comprise contacting a population of stem cells with an effective amount of a selective inhibitor of a PI3K alpha and an effective amount of an mTOR inhibitor. In other aspects, methods of the invention can include contacting a population of stem cells with an effective amount of a dual inhibitor that is selective for PI3K alpha and an mTOR kinase. In other aspects, methods of the invention can include contacting a population of stem cells with an effective amount of a selective inhibitor of a PI3K alpha and an effective amount of a selective inhibitor of a PI3K delta.

In certain aspects, the methods of the invention encompass contacting a population of stem cells with an effective amount of Activin A and an effective amount of a selective inhibitor of PI3K alpha that has also been demonstrated to be a selective inhibitor of a PI3K delta, e.g., Compound A, which is 4-[2-(1H-indazol-4-yl)-6-[(4-methylsulfonylpiperazin-1-yl)methyl]thieno[3,2-d]pyrimidin-4-yl]morpholine, the structure of which is provided below:

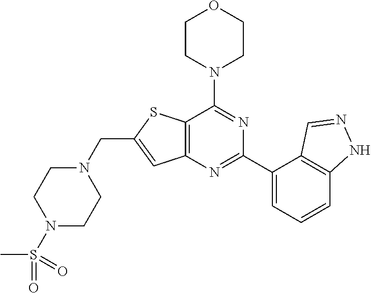 [SMILES: CS(=O)(=O)N1CCN(Cc2cc3nc(-c4cccc5[nH]ncc45)nc(N4CCOCC4)c3s2)CC1]

In such methods, the effective amount of a selective inhibitor of PI3K alpha that has also been demonstrated to be a selective inhibitor of a PI3K delta can be, e.g., at least 300 nM, at least 400 nM, at least 500 nM, or greater than 500 nM, e.g., 550 nM, at least 600 nM, at least 650 nM, at least 700 nM, or at least 750 nM. In certain aspects, the effective amount of a selective inhibitor of PI3K alpha that has also been demonstrated to be a selective inhibitor of a PI3K delta that can be used in the methods can be, e.g., greater than 750 nM, at least 800 nM, at least 850 nM, at least 900 nM, at least 950 nM, or greater than 950 nM.

In certain aspects of the methods, culturing stem cells under conditions sufficient to produce a population of endoderm cells, e.g., any population described herein, can include culturing the stem cells for at least 3 days, for at least 4 days, for at least 5 days, for at least 6 days, for at least 7 days, or for more than 7 days in medium containing an effective amount of Activin A and an effective amount of a selective inhibitor of a PI3K alpha.

Another aspect of the invention is that a population of endoderm cells can be obtained by contacting stem cells with an effective amount of Activin A and an effective amount of a selective inhibitor of a PI3K alpha and culturing the stem cells in the absence of Wnt3a. Thus, any of the methods described above and elsewhere herein can be performed in the absence of Wnt3a. Moreover, the methods are not limited by the medium in which the stem cells are cultured. In one aspect, the methods can be performed in, e.g., chemically defined medium or conditioned medium. For example, the stem cells can be cultured, in, e.g., DMEM/F12, RPMI, or any other stem cell culture medium known to those of skill in the art. In some embodiments, a pan-PI3K kinase is not used. A non-limiting example of a pan-PI3K that is not used is Ly294002.

Furthermore, any of the methods described above can be used to obtain a population of endoderm cells that exhibit greater viability and/or proliferation as compared to populations of endoderm cells obtained using other methods known in the art, i.e., populations of endoderm cells obtained from stem cells that have not been contacted with an effective amount of a selective inhibitor of PI3K alpha and an effective amount of Activin A. The endoderm cells obtained by the methods exhibit greater phenotypic stability and are more proliferative than endoderm cells obtained by other methods, e.g., from stem cells that have not been contacted with an effective amount of a selective inhibitor of PI3K alpha and an effective amount of Activin A. For example, the methods of the invention can be used to obtain a population of endoderm cells that are viable and proliferative after at least 3 days, at least 4 days, at least 5 days, at least 6 days, at least 7 days, at least 8 days, at least 9 days, at least 10 days, or more than 10 days in culture (e.g., greater than 11 days, greater than 12 days, greater than 13 days, greater than 14 days, or greater than 15 days in culture.) The methods of the invention can be used to obtain a population of endoderm cells that is phenotypically stable and proliferative after 2 passages, after 3 passages, after 4 passages, after 5 passages, after 6 passages, after 7 passages, after 8 passages, after 9 passages, for up to 10 passages, or after more than 10 passages (e.g., 11 passages or 12 passages.) In some embodiments of the methods, these populations of endoderm remain phenotypically stable and proliferative when grown in the absence of a feeder layer (e.g., a MATRIGEL layer or a collagen layer). In some embodiments of the methods, these populations of endoderm remain phenotypically stable and proliferative when grown in TesR2 medium+30% mouse embryonic fibroblast-conditioned medium (MEF). In some embodiments of the methods, these populations of endoderm remain phenotypically stable and proliferative when grown in in TesR2 medium+30% MEF and the presence of BMP4. In some embodiments of the methods, these populations of endoderm remain phenotypically stable and proliferative when grown in in TesR2 medium+30% MEF and the presence of BMP4. In some embodiments of the methods, these populations of endoderm remain phenotypically stable and proliferative when grown in in TesR2 medium+30% MEF and the presence of BMP4, and any combination of FGF2, VEGF and/or EGF. In addition, a population of endoderm cells obtained by any one of the methods described herein is contemplated within the scope of the invention. Beneficially, the population of endoderm cells obtained by methods that include contacting stem cells with an effective amount of Activin A and an effective amount of a selective inhibitor of PI3K alpha has the capability to differentiate into hepatocytes, pancreas cells, and intestinal cells.

Selective Inhibitors of PI3K Alpha

In certain embodiments of the methods described above, the selective inhibitor of PI3K alpha can be a compound which is a fused pyrimidine of formula (I) as disclosed in U.S. patent application number US 2008/0207611:

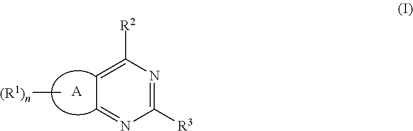

wherein A represents a thiophene or furan ring; n is 1 or 2; R¹ is a group of formula:

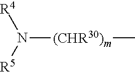

wherein m is 0 or 1; R³⁰ is H or C₁-C₆ alkyl; R⁴ and R⁵ form, together with the N atom to which they are attached, a 5- or 6-membered saturated N-containing heterocyclic group which includes 0 or 1 additional heteroatoms selected from N, S and O, which may be fused to a benzene

ring and which is unsubstituted or substituted; or one of R⁴ and R⁵ is alkyl and the other is a 5- or 6-membered saturated N-containing heterocyclic group as defined above or an alkyl group which is substituted by a 5- or 6-membered saturated N-containing heterocyclic group as defined above;

R² is selected from:

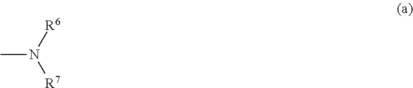

wherein R⁶ and R⁷ form, together with the nitrogen atom to which they are attached, a morpholine, thiomorpholine, piperidine, piperazine, oxazepane or thiazepane group which is unsubstituted or substituted; and

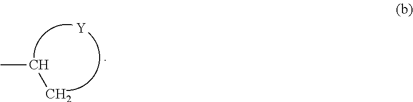

wherein Y is a C₂-C₄ alkylene chain which contains, between constituent carbon atoms of the chain and/or at one or both ends of the chain, 1 or 2 heteroatoms selected from O, N and S, and which is unsubstituted or substituted; and R³ is an indazole group which is unsubstituted or substituted; or a pharmaceutically acceptable salt thereof.

In certain embodiments to the methods, the PI3K alpha inhibitor can be a compound which is a fused pyrimidine ring of formula (Ia) as disclosed in U.S. patent application number US 2008/0207611:

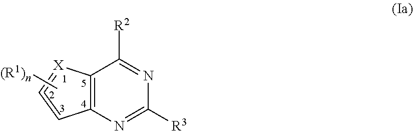

wherein X is S or O and R¹, R², R³ and n are defined as above.

Additionally, the PI3K alpha inhibitor used in the methods described herein can be a compound which is a fused pyrimidine ring of formula (Ib):

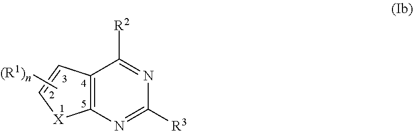

wherein X is S or O and R¹, R², R³ and n are defined as above.

In formula (I), formula (Ia) or formula Ib, the groups R¹, R², R³, R⁴, R⁵, R⁶, R⁷, R³⁰, A, Y, X, subscript m, where such groups appear in formula (I), formula (Ia) or formula Ib have the meaning as disclosed in US2008/0207611, which is incorporated herein by reference for all purposes.

In certain embodiments, the selective PI3K alpha inhibitor used in the methods of obtaining endoderm can be any one or combination of the following compounds: 2-(1H-Indazol-4-yl)-6-(4-methyl-piperazin-1-ylmethyl)-4-morpholin-4-yl-thieno[3,2-d]pyrimidine; 4-[2-(1H-Indazol-4-yl)-4-morpholin-4-yl-thieno[3,2-d]pyrimidin-6-ylmethyl]-piperazine-1-sulfonic acid dimethylamide; {4-[2-(1H-Indazol-4-yl)-4-morpholin-4-yl-thieno[3,2-d]pyrimidin-6-ylmethyl]-piperazin-1-yl}-morpholin-4-yl-methanone; 4-[2-(1H-Indazol-4-yl)-4-morpholin-4-yl-thieno[3,2-d]pyrimidin-6-ylmethyl]-piperazine-1-carboxylic acid (2-methoxy-ethyl)-methyl-amide; {4-[2-(1H-Indazol-4-yl)-4-morpholin-4-yl-thieno[3,2-d]pyrimidin-6-ylmethyl]-piperazin-1-yl}-N,N-dimethyl-acetamide; 4-[2-(1H-Indazol-4-yl)-4-morpholin-4-yl-thieno[3,2-d]pyrimidin-6-ylmethyl]-piperazine-1-carboxylic acid dimethylamide; 2-(1H-Indazol-4-yl)-4-morpholin-4-yl-6-[4-(3-morpholin-4-yl-propane-1-sulfonyl)-piperazin-1-ylmethyl]-thieno[3,2-d]pyrimidine; {1-[2-(1H-Indazol-4-yl)-4-morpholin-4-yl-thieno[3,2-d]pyrimidin-6-ylmethyl]-piperidin-4-yl}-(2-methoxy-ethyl)-methyl-amine; (3-{4-[2-(1H-Indazol-4-yl)-4-morpholin-4-yl-thieno[3,2-d]pyrimidin-6-ylmethyl]-piperazine-1-sulfonyl}-propyl)-dimethyl-amine; 2-{4-[2-(1H-Indazol-4-yl)-4-morpholin-4-yl-thieno[3,2-d]pyrimidin-6-ylmethyl]-piperazin-1-yl}-2-methyl-propan-1-ol; 1′-[2-(1H-Indazol-4-yl)-4-morpholin-4-yl-thieno[3,2-d]pyrimidin-6-ylmethyl]-[1,4′]bipiperidinyl; 2-(1H-Indazol-4-yl)-4-morpholin-4-yl-6-(4-morpholin-4-yl-piperidin-1-ylmethyl)-thieno[3,2-d]pyrimidine; 2-(1H-Indazol-4-yl)-4-morpholin-4-yl-6-(4-pyrimidin-2-yl-piperazin-1-ylmethyl)-thieno[3,2-d]pyrimidine; 1-(2-Hydroxy-ethyl)-4-[2-(1H-indazol-4-yl)-4-morpholin-4-yl-thieno[3,2-d]pyrimidin-6-ylmethyl]-piperazin-2-one; 6-(4-cyclopropylmethyl-piperazin-1-ylmethyl)-2-(1H-indazol-4-yl)-4-morpholin-4-yl-thieno[3,2-d]pyrimidine; 2-(1H-Indazol-4-yl)-4-morpholin-4-yl-6-(4-pyridin-2-yl-piperazin-1-ylmethyl)-thieno[3,2-d]pyrimidine; 2-(1H-Indazol-4-yl)-4-morpholin-4-yl-6-[4-(2,2,2-trifluoro-ethyl)-piperazin-1-ylmethyl]-thieno[3,2-d]pyrimidine; 2-(1H-Indazol-4-yl)-4-morpholin-4-yl-6-(4-thiazol-2-yl-piperazin-1-ylmethyl)-thieno[3,2-d]pyrimidine; 2-(6-Fluoro-1H-indazol-4-yl)-6-(4-methyl-piperazin-1-ylmethyl)-4-morpholin-4-yl-thieno[3,2-d]pyrimidine; 2-(1H-Indazol-4-yl)-4-morpholin-4-yl-6-(4-pyridin-2-ylmethyl-piperazin-1-ylmethyl)-thieno[3,2-d]pyrimidine; 2-(1H-Indazol-4-yl)-4-morpholin-4-yl-6-(4-thiazol-2-ylmethyl-piperazin-1-ylmethyl)-thieno[3,2-d]pyrimidine; 2-(1H-Indazol-4-yl)-6-[4-(5-methyl-furan-2-ylmethyl)-piperazin-1-ylmethyl]-4-morpholin-4-yl-thieno[3,2-d]pyrimidine; 1-[2-(1H-Indazol-4-yl)-4-morpholin-4-yl-thieno[3,2-d]pyrimidin-6-ylmethyl]-piperidine-4-carboxylic acid amide; 2-(1H-Indazol-4-yl)-6-[4-(2-methoxy-1,1-dimethyl-ethyl)-piperazin-1-ylmethyl]-4-morpholin-4-yl-thieno[3,2-d]pyrimidine; 2-(1H-Indazol-4-yl)-6-[(3R,5S)-4-(2-methoxy-ethyl)-3,5-dimethyl-piperazin-1-ylmethyl]-4-morpholin-4-yl-thieno[3,2-d]pyrimidine; 1-[2-(1H-Indazol-4-yl)-4-morpholin-4-yl-thieno[3,2-d]pyrimidin-6-ylmethyl]-piperidine-4-carboxylic acid (2-methoxy-ethyl)-methyl-amide; 1-[2-(1H-Indazol-4-yl)-4-morpholin-4-yl-thieno[3,2-d]pyrimidin-6-ylmethyl]-piperidine-4-carboxylic acid dimethylamide; 2-(1H-Indazol-4-yl)-4-morpholin-4-yl-6-(4-pyridin-3-ylmethyl-piperazin-1-ylmethyl)-thieno[3,2-d]pyrimidine; 1-[2-(1H-Indazol-4-yl)-4-morpholin-4-yl-thieno[3,2-d]pyrimidin-6-ylmethyl]-piperidine-4-carboxylic acid methylamide; 2-{4-[2-(1H-Indazol-4-yl)-4-morpholin-4-yl-thieno[3,2-d]pyrimidin-6-ylmethyl]-piperazin-1-yl}-N-methyl-isobutyramide; 2-{4-[2-(1H-Indazol-4-yl)-4-morpholin-4-yl-thieno[3,2-d]pyrimidin-6-ylmethyl]-piperazin-1-yl}-2-methyl-1-pyrrolidin-1-yl-propan-1-one; 2-(1H-Indazol-4-yl)-6-[4-(1-methyl-1H-imidazol-2-ylmethyl)-piperazin-1-ylmethyl]-4-morpholin-4-yl-thieno[3,2-d]pyrimidine; 2-(1H-Indazol-4-yl)-6-[4-(5-methyl-isoxazol-3-ylmethyl)-piperazin-1-ylmethyl]-4-morpholin-4-yl-thieno[3,2-d]pyrimidine; 1-{4-[2-(1H-Indazol-4-yl)-4-morpholin-4-yl-thieno[3,2-d]pyrimidin-6-ylmethyl]-piperazin-1-yl}-2-methyl-propan-2-ol; cyclopropylmethyl-{1-[2-(1H-indazol-4-yl)-4-morpholin-4-yl-thieno[3,2-d]pyrimidin-6-ylmethyl]-piperidin-4-yl}-(2-methoxy-ethyl)-amine; 6-[4-(1-Ethyl-1-methoxymethyl-propyl)-piperazin-1-ylmethyl]-2-(1H-indazol-4-yl)-4-morpholin-4-yl-thieno[3,2-d]pyrimidine; 2-(1H-Indazol-4-yl)-6-[4-(1-methoxymethyl-cyclopropyl)-piperazin-1-ylmethyl]-4-morpholin-4-yl-thieno[3,2-d]pyrimidine; {1-[2-(1H-Indazol-4-yl)-4-morpholin-4-yl-thieno[3,2-d]pyrimidin-6-ylmethyl]-piperidin-4-yl}-(2-methoxy-ethyl)-(2,2,2-trifluoro-ethyl)-amine; 2-(1H-Indazol-4-yl)-6-[4-(2-methoxy-ethyl)-piperazin-1-ylmethyl]-4-morpholin-4-yl-thieno[3,2-d]pyrimidine; {1-[2-(1H-Indazol-4-yl)-4-morpholin-4-yl-thieno[3,2-d]pyrimidin-6-ylmethyl]-piperidin-4-yl}-methanol; 2-(1H-Indazol-4-yl)-4-morpholin-4-yl-6-(4-pyridin-4-ylmethyl-piperazin-1-ylmethyl)-thieno[3,2-d]pyrimidine; 2-(1H-Indazol-4-yl)-6-[4-(6-methyl-pyridin-2-ylmethyl)-piperazin-1-ylmethyl]-4-morpholin-4-yl-thieno[3,2-d]pyrimidine; 2-(1H-Indazol-4-yl)-6-[4-(4-methyl-thiazol-2-ylmethyl)-piperazin-1-ylmethyl]-4-morpholin-4-yl-thieno[3,2-d]pyrimidine; {1-[2-(1H-Indazol-4-yl)-4-morpholin-4-yl-thieno[3,2-d]pyrimidin-6-ylmethyl]-piperidin-4-yl}-pyridin-2-yl-amine; N-{1-[2-(1H-Indazol-4-yl)-4-morpholin-4-yl-thieno[3,2-d]pyrimidin-6-ylmethyl]-piperidin-4-yl}-2-methoxy-N-methyl-acetamide; N-{1-[2-(1H-Indazol-4-yl)-4-morpholin-4-yl-thieno[3,2-d]pyrimidin-6-ylmethyl]-piperidin-4-yl}-N-methyl-methanesulfonamide; {1-[2-(1H-Indazol-4-yl)-4-morpholin-4-yl-thieno[3,2-d]pyrimidin-6-ylmethyl]-piperidin-4-yl}-(3-methoxy-propyl)-methyl-amine; 6-((3S,5R)-3,5-Dimethyl-4-pyridin-2-ylmethyl-piperazin-1-ylmethyl)-2-(1H-indazol-4-yl)-4-morpholin-4-yl-thieno[3,2-d]pyrimidine; 2-(1H-Indazol-4-yl)-6-(4-methoxymethyl-piperidin-1-ylmethyl)-4-morpholin-4-yl-thieno[3,2-d]pyrimidine; {1-[2-(1H-Indazol-4-yl)-4-morpholin-4-yl-thieno[3,2-d]pyrimidin-6-ylmethyl]-piperidin-4-yl}-(2-methoxy-ethyl)-thiazol-2-ylmethyl-amine; 1-[2-(1H-Indazol-4-yl)-4-morpholin-4-yl-thieno[3,2-d]pyrimidin-6-ylmethyl]-4-pyridin-2-ylmethyl-piperidin-4-ol; {1-[2-(1H-Indazol-4-yl)-4-morpholin-4-yl-thieno[3,2-d]pyrimidin-6-ylmethyl]-piperidin-4-yl}-isopropyl-(2-methoxy-ethyl)-amine; 2-(1H-Indazol-4-yl)-4-morpholin-4-yl-6-[4-(pyridin-2-yloxy)-piperidin-1-ylmethyl]-thieno[3,2-d]pyrimidine; N-{1-[2-(1H-Indazol-4-yl)-4-morpholin-4-yl-thieno[3,2-d]pyrimidin-6-ylmethyl]-piperidin-4-yl}-N-(2-methoxy-ethyl)-methanesulfonamide; 2-{1-[2-(1H-Indazol-4-yl)-4-morpholin-4-yl-thieno[3,2-d]pyrimidin-6-ylmethyl]-piperidin-4-yl}-propan-2-ol; 2-(1H-Indazol-4-yl)-4-morpholin-4-yl-6-[4-(1-oxy-pyridin-3-ylmethyl)-piperazin-1-ylmethyl]-thieno[3,2-d]pyrimidine; 2-(1H-Indazol-4-yl)-4-morpholin-4-yl-6-(4-morpholin-4-ylmethyl-piperidin-1-ylmethyl)-thieno[3,2-d]pyrimidine; {1-[2-(1H-Indazol-4-yl)-4-morpholin-4-yl-thieno[3,2-d]pyrimidin-6-ylmethyl]-piperidin-4-ylmethyl}-(2-methoxy-ethyl)-methyl-amine; {1-[2-(1H-Indazol-4-yl)-4-morpholin-4-yl-thieno[3,2-d]pyrimidin-6-ylmethyl]-piperidin-4-ylmethyl}-dimethyl-amine; {1-[2-(1H-Indazol-4-yl)-4-morpholin-4-yl-thieno[3,2-d]pyrimidin-6-ylmethyl]-piperidin-3-yl}-(2-methoxy-ethyl)-methyl-amine; 1-[2-(1H-Indazol-4-yl)-4-morpholin-4-yl-thieno[3,2-d]pyrimidin-6-ylmethyl]-piperidine-3-carboxylic acid methylamide; 2-(1H-Indazol-4-yl)-6-(3-methoxymethyl-piperidin-1-ylmethyl)-4-morpholin-4-yl-thieno[3,2-d]pyrimidine; 2-(1H-Indazol-4-yl)-4-morpholin-4-yl-6-(4-pyridin-2-ylmethyl-piperidin-1-ylmethyl)-thieno[3,2-d]pyrimidine; 2-(1H-Indazol-4-yl)-6-[4-(2-methoxy-ethoxy)-piperidin-1-ylmethyl]-4-morpholin-4-yl-thieno[3,2-d]pyrimidine; 6-((3R,5S)-3,5-Dimethyl-4-thiazol-2-ylmethyl-piperazin-1-ylmethyl)-2-(1H-indazol-4-yl)-4-morpholin-4-yl-thieno[3,2-d]pyrimidine; 2-(1H-Indazol-4-yl)-4-morpholin-4-yl-6-[4-(1-oxy-pyridin-2-ylmethyl)-piperazin-1-ylmethyl]-thieno[3,2-d]pyrimidine; 2-(1H-Indazol-4-yl)-6-[4-(2-methoxy-ethyl)-piperidin-1-ylmethyl]-4-morpholin-4-yl-thieno[3,2-d]pyrimidine; 2-(1H-Indazol-4-yl)-6-(4-methanesulfonyl-piperidin-1-ylmethyl)-4-morpholin-4-yl-thieno[3,2-d]pyrimidine; {1-[2-(1H-Indazol-4-yl)-4-morpholin-4-yl-thieno[3,2-d]pyrimidin-6-ylmethyl]-piperidin-4-yl}-(3-methanesulfonyl-propyl)-methyl-amine; 2-(1H-Indazol-4-yl)-6-[4-(3-methoxy-propane-1-sulfonyl)-piperidin-1-ylmethyl]-4-morpholin-4-yl-thieno[3,2-d]pyrimidine; (R)-1-[2-(1H-Indazol-4-yl)-4-morpholin-4-yl-thieno[3,2-d]pyrimidin-6-ylmethyl]-piperidine-3-carboxylic acid methylamide; (S)-1-[2-(1H-Indazol-4-yl)-4-morpholin-4-yl-thieno[3,2-d]pyrimidin-6-ylmethyl]-piperidine-3-carboxylic acid methylamide; 6-(4-Imidazol-1-ylmethyl-piperidin-1-ylmethyl)-2-(1H-indazol-4-yl)-4-morpholin-4-yl-thieno[3,2-d]pyrimidine; 2-(1H-Indazol-4-yl)-4-morpholin-4-yl-6-morpholin-4-ylmethyl-thieno[3,2-d]pyrimidine; 2-(1H-Indazol-4-yl)-6-(3-methyl-piperidin-1-ylmethyl)-4-morpholin-4-yl-thieno[3,2-d]pyrimidine; {1-[2-(1H-Indazol-4-yl)-4-morpholin-4-yl-thieno[3,2-d]pyrimidin-6-ylmethyl]-piperidin-3-yl}-methanol; 2-{1-[2-(1H-Indazol-4-yl)-4-morpholin-4-yl-thieno[3,2-d]pyrimidin-6-ylmethyl]-piperidin-4-yl}-ethanol; 1-[2-(1H-Indazol-4-yl)-4-morpholin-4-yl-thieno[3,2-d]pyrimidin-6-ylmethyl]-4-thiazol-2-yl-piperidin-4-ol; 2-(1-Methyl-1H-indazol-4-yl)-6-(4-methyl-piperazin-1-ylmethyl)-4-morpholin-4-yl-thieno[3,2-d]pyrimidine; 2-(2-Methyl-2H-indazol-4-yl)-6-(4-methyl-piperazin-1-ylmethyl)-4-morpholin-4-yl-thieno[3,2-d]pyrimidine; 2-(1H-Indazol-4-yl)-4-morpholin-4-yl-6-(4-thiazol-4-ylmethyl-piperazin-1-ylmethyl)-thieno[3,2-d]pyrimidine; 1-{4-[2-(1H-Indazol-4-yl)-4-morpholin-4-yl-thieno[3,2-d]pyrimidin-6-ylmethyl]-piperazin-1-yl}-3-phenoxy-propan-2-ol; 6-[4-(1H-Imidazol-2-ylmethyl)-piperazin-1-ylmethyl]-2-(1H-indazol-4-yl)-4-morpholin-4-yl-thieno[3,2-d]pyrimidine; 6-[4-(3H-Imidazol-4-ylmethyl)-piperazin-1-ylmethyl]-2-(1H-indazol-4-yl)-4-morpholin-4-yl-thieno[3,2-d]pyrimidine; 2-(1H-Indazol-4-yl)-4-morpholin-4-yl-6-((2S,6R)-2,4,6-trimethyl-piperazin-1-ylmethyl)-thieno[3,2-d]pyrimidine; {4-[2-(1H-Indazol-4-yl)-4-morpholin-4-yl-thieno[3,2-d]pyrimidin-6-ylmethyl]-1-methanesulfonyl-piperazin-2-yl}-methanol; and 2-(1H-Indazol-4-yl)-6-(4-methanesulfonyl-3-methoxymethyl-piperazin-1-ylmethyl)-4-morpholin-4-yl-thieno[3,2-d]pyrimidine; and the pharmaceutically acceptable salts of the above-mentioned free compounds. It is understood that the embodiments disclosed herein include salts thereof.

In some embodiments, the selective PI3K alpha inhibitor used in the methods can be any one or combination of the following selective PI3K alpha inhibitors or a combination of the following PI3K alpha inhibitors with another selective inhibitor of the PI3K pathway, e.g., another PI3K alpha inhibitor, PI3K delta inhibitor or an mTOR inhibitor:

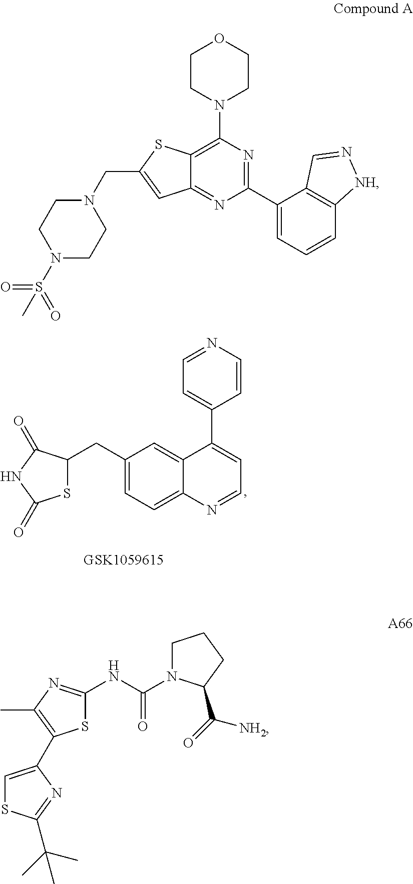 [SMILES: CS(=O)(=O)N1CCN(Cc2cc3nc(-c4cccc5[nH]ncc45)nc(N4CCOCC4)c3s2)CC1 ; O=C1NC(=O)C(Cc2ccc3nccc(-c4ccncc4)c3c2)S1 ; Cc1nc(NC(=O)N2CCC[C@H]2C(N)=O)sc1-c1csc(C(C)(C)C)n1]

Intellikine's INK1117, D106669, or Novartis's BYL719.

Methods of the invention can also make use of any one or combination the following PI3K alpha inhibitors or a combination of the following PI3K alpha inhibitors with another selective inhibitor of the PI3K pathway, e.g., another PI3K alpha inhibitor, a PI3K delta inhibitor, a mTOR inhibitor:

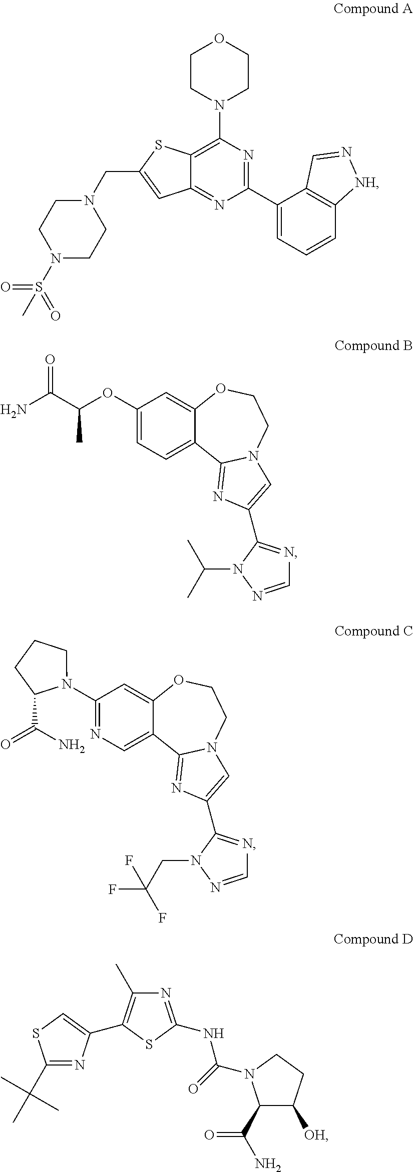

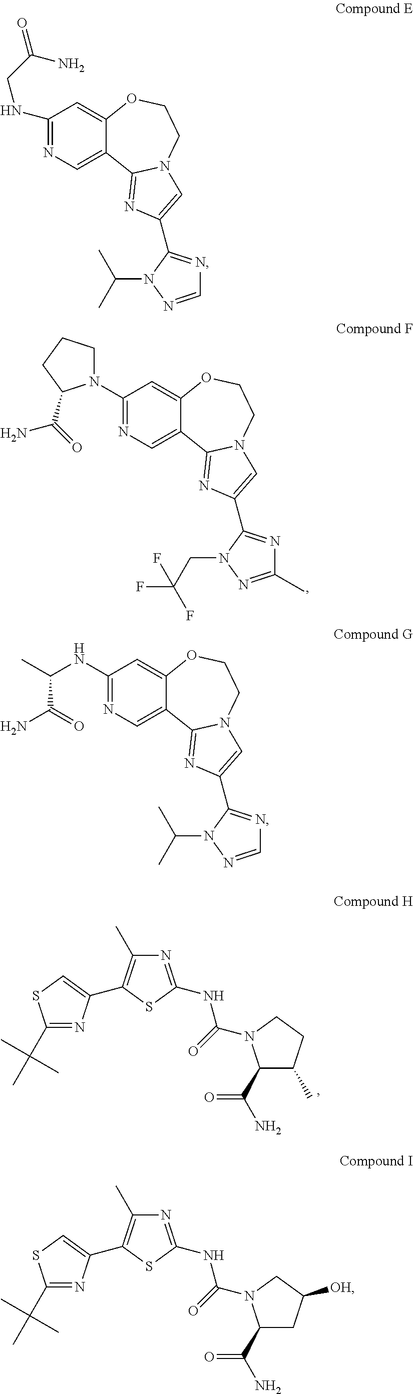

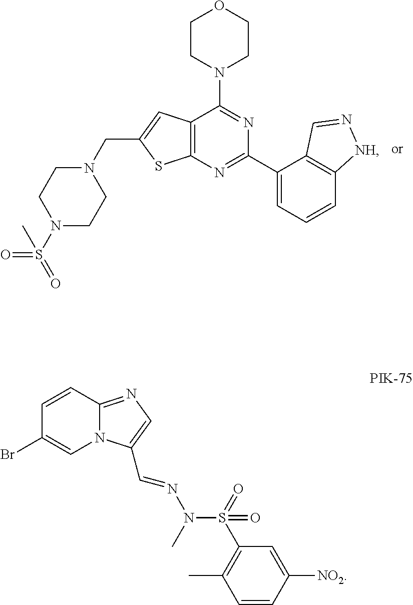 [SMILES: CS(=O)(=O)N1CCN(Cc2cc3c(N4CCOCC4)nc(-c4cccc5[nH]ncc45)nc3s2)CC1 ; "or" ; Cc1ccc([N+](=O)[O-])cc1S(=O)(=O)N(C)/N=C/c1cnc2ccc(Br)cn12]

In some embodiments the selective inhibitor of PI3K alpha is 4-[2-(1H-indazol-4-yl)-6-[(4-methylsulfonylpiperazin-1-yl)methyl]thieno[3,2-d]pyrimidin-4-yl]morpholine (also referred to herein as “Compound A”), the structure of which is provided below:

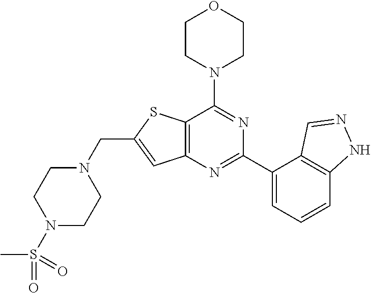 [SMILES: CS(=O)(=O)N1CCN(Cc2cc3nc(-c4cccc5[nH]ncc45)nc(N4CCOCC4)c3s2)CC1]

Additional PI3K alpha inhibitors that can be used in the methods provided by the invention are also described in US 2005/014771A1, US 2010/0137585A1, WO 2006/046040, US 2009/0156601, US 2008/0039459, US 2011/0105461, US 2008/0076768 (U.S. Pat. No. 7,781,433), WO 2007/132171, US 2008/0269210, US 2009/0118275, WO 2009/066084, US 2011/0172216, US 2009/0247567, US 2009/0318411, WO 2010/059788, US 2010/0233164, US 2011/007629, US 2011/0251202A1, US 2011/0003786A1, US 2011/0003818A1, US 2010/0298286A1, US 2010/0249126A1, US 2010/0105711A1, US 2010/0075965A1, US 2010/0311729A1, US 2010/0048547A1, US 2009/0163469A1, US 2009/0318410A1, US 2009/0286779A1, US 2009/0258882A1, US 2009/0318410A1, US 2009/0131457A1, US 2012/0059000A1, US 2011/0124641A1, US 2011/0172228A1, US 2011/0160232A1, US 2011/0281866A1, US 2011/0046165A1, US 2011/0077268A1, US 2011/0269779A1, US 2010/0184760A1, US 2010/0190749A1, US 2009/0312319A1, WO 2011/149937A1, WO 2011/022439A1, and WO 2010/129816A2, US 2008/0207611, U.S. Pat. No. 7,781,433, US 2008/0076758, US20080242665, and US 2011/0076291, the contents of each of which are incorporated by reference herein in their entirety.

Additional PI3K alpha inhibitors that are contemplated for use in the methods of the invention are described in US 2005/014771, US 2010/013758, US 2008/0207611, WO2006/046040, US 2009/0156601, US 2008/0039459, US 2011/0105461, US 2008/0076768, US 2008/0076758, WO 2007/132171, US 2008/0269210, US 2008/0242665, US 2009/0118275, WO 2009/066084, US 2011/0172216, US 2009/0247567, US 2009/0318411, WO 2010/059788, US 2010/0233164, US 2011/007629, US 2011/007629, and Shuttleworth et al. (2011) Current Medicinal Chemistry 18: 2686-2714, the contents of which are incorporated by reference herein in their entirety. Another PI3K alpha inhibitor that can be used be the methods of the invention is PI103.

It is understood that any combinations (two, three, four or more) of the selective inhibitors of PI3K alpha described in the references above may be used in the methods provided herein to produce a population of endoderm cells.

Inhibitors of PI3K Delta

In certain embodiments, endoderm cells can be made by contacting a population of stem cells with an effective amount of a selective inhibitor of PI3K alpha and a selective inhibitor of a PI3K delta. These encompass obtaining a population of endoderm cells by contacting stem cells with any one or combination of selective inhibitors of PI3K alpha described herein with any of the selective inhibitors of PI3K delta.

Additional PI3K delta inhibitors that can be used in the methods provided by the invention are described in US 2009/0131429, US 2009/0042884, US 2010/0016306, WO 2008/125839, WO 2008/125833, WO 2008/125835, US 2010/0190769, WO 2008/152387, WO 2008/152394, US 2011/0021496, WO 2009/053716, US 2010/0305096, US 2010/0305084, US 2011/0207713, US 2009/0131429, US 2009/0042884, US 2010/0016306, WO 2008/125839, WO2008/125833, WO 2008/125835, US 2010/0190769, WO 2008/152387, WO 2008/152394, US2011/0021496, WO 2009/053716, US 2010/0305096, US 2010/0305084, US 2011/0207713, the contents of which are expressly incorporated herein by reference in their entirety.

Population of Endoderm Cells

The invention provides populations of endoderm cells that result from the methods described herein. It is understood that the invention contemplates and encompasses the populations themselves as well as populations produced by the methods. Other related embodiments are further provided as described below and herein.

Phenotype of Endoderm Cells

A population or isolated population of endoderm cells provided by the invention can be described by various phenotypes related to the expression of one or more of the following biological markers: SOX 17, CXCR4, FoxA1, FoxA2, FoxA3, CD55 (or DAF1), Cer1 (Cerberus 1), HNF1a, HNF1b, HNF4a, Gata3, Gata4, Gata6, Hhex, and LHX1.

The presence and/or expression level of any one or more of these markers distinguishes a population of endoderm cells provided by the invention from populations of endoderm cells obtained using known methods of endoderm differentiation. These markers can be detected by standard methods known in the art including, but not limited to, immunohistochemistry, flow cytometry, and fluorescence imaging analysis. The details of such techniques can be found in Example 1. The markers described herein can be measured at different time points of culturing the endoderm cells, for example at 1 day, 2 day, 3 days, 4 days, 5 days, 6 days, 7 days, 8 days, 9 days, 10 days or more after the addition of Activin A and selective inhibitor of PI3K alpha and optionally, selective inhibitor of PI3K delta or mTOR kinase inhibitor.

The invention provides a population of endoderm cells in which at least about 50%, at least about 60%, at least about 65%, at least about 70%, at least about 75%, at least about 80%, at least about 81%, at least about 82%, or at least about 83% of the cells in the population express SOX 17. In some embodiments, the population of endoderm cells has these amounts of SOX17 after about 1 day, 2 days, 3 days, 4 days, 5 days or more in culture.

The invention also provides populations of endoderm cells in which greater than 83%, e.g., at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater than 99% of the cells in an isolated population of endoderm cells express SOX17. In some embodiments, the population of endoderm cells has these amounts of SOX17 after about 1 day, 2 days, 3 days, 4 days, 5 days or more in culture.

Additionally, the invention provides a population of endoderm cells in which least about 50%, at least about 60%, at least about 65%, at least about 70%, at least about 71%, at least about 72%, at least about 73%, at least about 74%, at least about 75%, at least about 76%, or at least about 77% of the cells express FoxA2. A population of endoderm cells of the invention can be a population in which greater that about 77%, e.g., at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, greater than about 90%, greater than about 93%, greater than about 95%, greater than about 97%, or greater than about 99%, of the cells express FoxA2.

In some embodiments, the population of endoderm cells has these amounts after about 1 day, 2 days, 3 days, 4 days, 5 days or more in culture.

In some aspects, the invention provides for a population of endoderm cells in which at least about 50%, at least about 55%, at least about 60%, at least about 65%, at least about 70%, at least about 75%, or at least about 76% of the cell express CXCR4. A population of endoderm cells of the invention can be a population in which greater than 76%, e.g., at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, greater than about 90%, greater than about 93%, greater than about 95%, greater than about 97%, or greater than about 99%, of the cells express CXCR4. In some embodiments, the population of endoderm cells has these amounts after about 1 day, 2 days, 3 days, 4 days, 5 days or more in culture.

Yet another way to characterize a population of endoderm cells of the invention is by the combinations of markers that they express. Accordingly, the invention provides a population of endoderm cells in which at least about 50%, at least about 65%, at least about 60%, at least about 70%, least about 75%, or greater than about 75% of the cells express both Sox17 and FoxA2. A population of endoderm cells of the invention can be, e.g., a population in which at least 83% of the cells express SOX17 and at least 77% of the cells express FoxA2.

A population of endoderm cells of the invention can be a population in which at least about 50%, at least about 55%, at least about 60%, at least about 65%, at least about 70%, or at least about 75% of the cells express both SOX17 and CXCR4. In certain aspects, a population of endoderm cells of the invention can be population of cells in which greater than 75%, e.g., at least about 76%, at least about 77%, at least about 78%, at least about 79%, at least about 80%, at least about 81%, at least about 82%, or at least about 83%, of the cells express both SOX17 and CXC4. For example, the invention provides a population of endoderm cells in which at least 83% of the cells express SOX17 and at least 76% of the cells express CXCR4. In some embodiments, the population of endoderm cells has these amounts after about 1 day, 2 days, 3 days, 4 days, 5 days or more in culture.

Additionally, a population of endoderm cells of the invention can be a population in which at least about 50%, at least about 55%, at least about 60%, at least about 65%, at least about 70%, or at least about 75%, or greater than about 75% of the cells express both FoxA2 and CXCR4. For example, the invention provides a population of endoderm cells in which at least about 77% of the cells express FoxA2 and at least about 76% of the cells express CXCR4. In some embodiments, the population of endoderm cells has these amounts after about 1 day, 2 days, 3 days, 4 days, 5 days or more in culture.

A population of endoderm cells of the invention can be a population in which at least about 50%, at least about 55%, at least about 60%, at least about 65%, at least about 70%, at least about 75% or greater than 75%, e.g., at least about 76%, at least about 77%, at least about 78%, at least about 79%, at least about 80%, at least about 81%, at least about 82%, at least about 83%, or greater than 83%, e.g., at least about 85%, at least about 87%, or greater than 87% of the cells express SOX17, FoxA2, and CXCR4. In further embodiments, an isolated population of endoderm cells can be a population in which at least 83% of the cells express SOX17, at least 77% of the cells express FoxA2, and at least 76% of the cells express CXCR4. In some embodiments, the population of endoderm cells has these amounts after about 1 day, 2 days, 3 days, 4 days, 5 days or more in culture.

Stable Endoderm

The endoderm cells provided by the invention can also be described by their ability to remain phenotypically stable as multipotent cells through multiple passages in culture, as well as their ability to proliferate (i.e., divide) while retaining this phenotype. Stable endoderm cells that can be maintained in a multipotent state can be used to study endoderm development and differentiation in vitro. Another way to characterize a stable population of endoderm cells of the invention is by its ability to remain proliferative (i.e., capable of cell division) through multiple passages in culture while retaining its phenotype. An expandable population of endoderm cells can provide large quantities of progenitor cells from which to obtain, e.g., hepatic cells, hepatocyte precursor cells, pancreatic precursor cells, pancreatic cells, hepatocytes, or other differentiated cells derived from endoderm cells (e.g., intestinal progenitor cells, intestinal cells, lung progenitor cells, lung cells, etc.), to meet clinical needs for cell therapy applications. Producing stable and expandable endoderm has been attempted in human (Seguin, et al. (2008) “Establishment of endoderm progenitors by SOX transcription factor expression in human embryonic stem cells.” Cell Stem Cell, 3(2): 182-19; Cheng, et al. (2012). “Self-renewing endodermal progenitor lines generated from human pluripotent stem cells.” Cell Stem Cell, 10(4): 371-384) and mouse cells (Morrison, et al. (2008). “Anterior definitive endoderm from ESCs reveals a role for FGF signaling.” Cell Stem Cell, 3(4): 402-415). However, these methods still include a sorting step to obtain CXCR4+ cells. A phenotypically stable, proliferative population of endoderm cells of the invention can be obtained using methods that do not include any sorting steps.

Thus, a population of endoderm cells of the invention can be a population that is characterized by its ability to maintain any of the phenotypes described above related to the expression of one or more of SOX 17, CXCR4, FoxA1, FoxA2, FoxA3, CD55 (or DAF1), Cer1 (Cerberus 1), HNF1a, HNF1b, HNF4a, Gata3, Gata4, Gata6, Hhex, and LHX1 over a period of days, e.g., at least 3 days, at least 4 days, at least 5 days, at least 6 days, at least 7 days, at least 8 days, at least 9 days, at least 10 days, or more than 10 days in culture, such as greater than 11 days, greater than 12 days, greater than 13 days, greater than 14 days, greater than 15 days in culture, greater than 16 days in culture, greater than 17 days in culture, greater than 18 days in culture, greater than 19 days in culture, greater than 20 days in culture, greater than 21 days in culture, greater than 22 days in culture, greater than 23 days in culture, or greater than 24 days in culture, including any range in between these values. In some embodiments, a population of endoderm cells of the invention can be a population that is phenotypically stable as characterized by its ability to maintain any of the phenotypes described above related to the expression of one or more of SOX 17, CXCR4, FoxA1, FoxA2, FoxA3, CD55 (or DAF1), Cer1 (Cerberus 1), HNF1a, HNF1b, HNF4a, Gata3, Gata4, Gata6, Hhex, and LHX1 for at least about 2 passages, for at least about 3 passages, for at least about 4 passages, for at least about 5 passages, for at least about 6 passages, for at least about 7 passages, for at least about 8 passages, for at least about 9 passages, for up to 10 passages, or for at least about more than 10 passages (e.g., 11 passages or 12 passages), including any range in between these values.

In some embodiments, a population of endoderm cells is a population that can remain phenotypically stable as multipotent cells, as characterized by its ability to maintain any of the phenotypes described above related to the expression of one or more of SOX 17, CXCR4, FoxA1, FoxA2, FoxA3, CD55 (or DAF1), Cer1 (Cerberus 1), HNF1a, HNF1b, HNF4a, Gata3, Gata4, Gata6, Hhex, and LHX1, when grown in the absence of a feeder layer (e.g., a MATRIGEL layer or a collagen layer). In some embodiments a population of endoderm cells is a population that can remain phenotypically stable as multipotent cells, as characterized by its ability to maintain any of the phenotypes described above related to the expression of one or more of SOX 17, CXCR4, FoxA1, FoxA2, FoxA3, CD55 (or DAF1), Cer1 (Cerberus 1), HNF1a, HNF1b, HNF4a, Gata3, Gata4, Gata6, Hhex, and LHX1, when grown in TesR2 medium+30% mouse embryonic fibroblast-conditioned medium (MEF). In some embodiments, a population of endoderm cells is a population that can remain phenotypically stable as multipotent cells, as characterized by its ability to maintain any of the phenotypes described above related to the expression of one or more of SOX 17, CXCR4, FoxA1, FoxA2, FoxA3, CD55 (or DAF1), Cer1 (Cerberus 1), HNF1a, HNF1b, HNF4a, Gata3, Gata4, Gata6, Hhex, and LHX1, when grown in in TesR2 medium+30% MEF and the presence of BMP4. In some embodiments of the methods, a population of endoderm cells is a population that can remain phenotypically stable as multipotent cells, as characterized by its ability to maintain any of the phenotypes described above related to the expression of one or more of SOX 17, CXCR4, FoxA1, FoxA2, FoxA3, CD55 (or DAF1), Cer1 (Cerberus 1), HNF1a, HNF1b, HNF4a, Gata3, Gata4, Gata6, Hhex, and LHX1, when grown in in TesR2 medium+30% MEF and the presence of BMP4, FGF2, VEGF, and EGF. In some embodiments, a population of endoderm cells is a population that can remain phenotypically stable as multipotent cells, as characterized by its ability to maintain any of the phenotypes described above related to the expression of one or more of SOX 17, CXCR4, FoxA1, FoxA2, FoxA3, CD55 (or DAF1), Cer1 (Cerberus 1), HNF1a, HNF1b, HNF4a, Gata3, Gata4, Gata6, Hhex, and LHX1, when obtained using a method that does not include a sorting step.

A population of endoderm cells of the invention can be a population that remains proliferative over a period of days, e.g., at least 3 days, at least 4 days, at least 5 days, at least 6 days, at least 7 days, at least 8 days, at least 9 days, at least 10 days, or more than 10 days in culture, such as greater than 11 days, greater than 12 days, greater than 13 days, greater than 14 days, greater than 15 days in culture, greater than 16 days in culture, greater than 17 days in culture, greater than 18 days in culture, greater than 19 days in culture, greater than 20 days in culture, greater than 21 days in culture, greater than 22 days in culture, greater than 23 days in culture, or greater than 24 days in culture, including any range in between these values. In some embodiments, a population of endoderm cells of the invention can be a population that remains proliferative for at least about 2 passages, for at least about 3 passages, for at least about 4 passages, for at least about 5 passages, for at least about 6 passages, for at least about 7 passages, for at least about 8 passages, for at least about 9 passages, for up to 10 passages, or for at least about more than 10 passages (e.g., 11 passages or 12 passages).

In some embodiments, a population of endoderm cells is a population that can remain proliferative when grown in the absence of a feeder layer (e.g., a MATRIGEL layer or a collagen layer). In some embodiments a population of endoderm cells is a population that can remain proliferative when grown in TesR2 medium+30% mouse embryonic fibroblast-conditioned medium (MEF). In some embodiments, a population of endoderm cells is a population that can remain proliferative when grown in in TesR2 medium+30% MEF and the presence of BMP4. In some embodiments of the methods, a population of endoderm cells is a population that can remain proliferative when grown in in TesR2 medium+30% MEF and the presence of BMP4, FGF2, VEGF, and EGF. In some embodiments, a population of endoderm cells is a population that can remain proliferative when obtained using a method that does not include a sorting step.

One aspect of the invention is that a population of endoderm cells, e.g., a population that is phenotypically stable and/or proliferative, can be cryogenically preserved in the form of a bank of endoderm cells. Such banks can be thawed for future therapeutic or experimental use. The banks of phenotypically stable and proliferative endoderm cells can be cryogenically stored using methods known to those of skill in the art.

In some embodiments, an isolated population of endoderm cells (e.g., any of the populations of endoderm cells described herein) is manipulated to provide a preparation of cells that is substantially free of additional components (e.g., cellular debris). In some embodiments, the cell preparation is at least about 60%, by weight, volume, or number, free from other components that are present when the cell is produced or cultured. In various aspects, the cell is at least about 75%, or at least about 85%, or at least about 90%, 91%, 92%, 93%, 94%, 95%, 96%, 97%, or at least about 98%, or at least about 99%, by weight, volume, or number, pure. In some aspects, the percentage refers to a percentage of endoderm or hepatocyte cells in a cell culture or population. A population or isolated population of endoderm or hepatocyte cells can be obtained, for example, by purification from a natural source, e.g., by mechanical or physical or chemical extraction, fluorescence-activated cell-sorting, or other techniques known to the skilled artisan. The purity can be assayed by any appropriate method, such as fluorescence-activated cell-sorting (FACS) or by visual examination.

In some embodiments, a homogenous population of endoderm cells can be made as described herein. A homogeneous population of endoderm cells is a population of cells where a significant portion of the population are endoderm cells. A significant portion is more than about 50%, 55%, 60%, 65%, 70%, 75%, 80%, 85%, 90%, 91%, 92%, 93%, 94%, 95%, 96%, 97%, 98%, or 99% of the cells in the population are endoderm cells.

The population of endoderm cells of the invention has the capability to differentiate into any one or more of the following: hepatocytes, pancreatic cells, and intestinal cells. Accordingly, a population of endoderm cells having any of the characteristics described above can be beneficially used in the development of pure tissue or cell types.

One aspect of the invention is that a population of endoderm cells, e.g., a population having any of the characteristics of a population described above, can be cryogenically preserved in the form of a bank of endoderm cells. Such banks can be thawed for future therapeutic or experimental use. The banks of endoderm cells can be cryogenically stored using methods known to those of skill in the art.

Another aspect of the invention is an in vitro cell culture in medium comprising an effective amount of Activin A and an effective amount of a PI3K alpha inhibitor, wherein the cells comprise stem cells, endoderm cells, and/or cells differentiated from stem cells, i.e., any of a variety of endoderm precursor cells. The invention contemplates and encompasses any intermediate cell types in the pathway that leads to the formation of any of the populations of endoderm cells described herein from a population of stem cells.

The invention also contemplates articles of manufacture (e.g., devices, medical devices, implantation devices, instruments, cell culture vessels, cell culture plates, scaffolding) that comprise the endoderm cells, hepatocytes cells, and any intermediates.

The invention contemplates any and all of the parameters, as described above and elsewhere herein, in any combination, to describe and characterize a population of endoderm cells.

Methods of Using Endoderm Cells

The invention provides endoderm cells that can be used in a variety of research and therapeutic applications. For example, endoderm cells from the populations described herein can be used to further studies in cell and tissue differentiation. The endoderm cells can also be used in toxicity assays for testing new drug candidates. Moreover, endoderm cell derivatives, including hepatocytes, pancreas cells, and intestinal cells, can be used for regenerative medicine and therapeutic use.

Methods for Identifying Factors that Promote Differentiation of Endoderm Cells into a Cell Type of Interest

The subject invention provides a ready source of endoderm cells for research applications, such as studying developmental signaling pathways. Accordingly, the invention provides methods for screening factors for potentiators that promote the differentiation of a population of endoderm cells into a cell type of interest, e.g., a hepatocyte, a pancreas cell, or an intestinal cell. The methods include contacting a population of endoderm cells, e.g., a population provided by the invention or obtained using any one of the methods provided by the invention, with the factor and monitoring the population of endoderm cells for differentiation into the cell.

The effects of a contacting the endoderm cells with such a factor can be identified by monitoring, e.g., the ratios of expressed phenotypes, cell viability, and alterations in gene expression of a test population of endoderm cells as compared to a population of endoderm cells that have not been contacted with the factor. Methods of monitoring and comparing phenotypes between these populations of cells are well known to those of skill in the art. For example, physical characteristics of the cells can be analyzed by observing cell morphology and growth via microscopy. Increased or decreased levels of proteins, such as cell-type specific enzymes, receptors, and other cell surface molecules can be analyzed with any technique known in the art which can identify the alteration of the level of such molecules. These techniques include immunohistochemistry, using antibodies against such molecules, or biochemical analyses. Such biochemical analyses include protein assays, enzymatic assays, receptor binding assays, enzyme-linked immunosorbent assays (ELISA), electrophoretic analysis, analysis with high performance liquid chromatography (HPLC), Western blots, and radioimmune assays (RIA). Nucleic acid analysis, such as Northern blots, S1 mapping, primer extension, and polymerase chain reaction (PCR) can be used to examine the levels of mRNA coding for these molecules, or for enzymes which synthesize these molecules.

Methods for Identifying Factors that Inhibit Differentiation of Endoderm Cells

In studying developmental signaling pathways, it can be equally important to identify factors that inhibit a population of endoderm cells from differentiating. Methods provided by the invention for identifying such factors include contacting a population of endoderm cells, e.g., a population provided by the invention or obtained using any one of the methods provided by the invention, with the factor and monitoring the population of endoderm cells for differentiation into the cell. The effects of contacting the endoderm cells with such a factor can be identified by monitoring phenotypes, cell viability, and alterations in gene expression of a test population of endoderm cells as compared to a population of endoderm cells that have not been contacted with the factor. Phenotypes of the test population can be monitored as described above.

Cell-Based Therapies

In another aspect, the invention provides methods for treating a variety of disorders by administering endoderm cells, e.g., from populations described herein, from populations obtained from methods described herein, or from banks of one or more population(s) of endoderm cells, to a patient in need thereof. A highly homogenous population of endoderm cells can be administered directly to a subject at a site, such as the liver, and the endoderm cells can differentiate into hepatocytes. For cell therapy, the cells may be administered directly to the subject to treat an adverse medical condition. Such medical conditions can include, for example, liver fibrosis, cirrhosis, liver failure, liver and pancreatic cancer, pancreatic failure, intestinal disorders including tissue replacement enzyme defects, Crohn's disease, inflammatory bowel syndrome, and intestinal cancer.

The endoderm cells of the invention can be administered as autografts, syngeneic grafts, allografts, and xenografts, for example. If rejection or other issues related to transfer of non-autologous cells in a recipient arise, then compositions and methods of addressing such rejection known to one of skill in the area of transplant rejection can be used. Additionally, the endoderm cells of the invention can be administered to a patient, e.g., intravascularly, intracranially, intracerebrally, intramuscularly, intradermally, intravenously, intraocularly, orally, nasally, topically, or by open surgical procedure, depending upon the anatomical site or sites to which the cells are to be delivered.

The cells of the subject invention can be administered to a patient in isolation or within a pharmaceutical composition comprising the cells and a pharmaceutically acceptable carrier. As used herein, a pharmaceutically acceptable carrier includes solvents, dispersion media, coatings, antibacterial and antifungal agents, isotonic agents, and the like. Pharmaceutical compositions can be formulated according to known methods for preparing pharmaceutically useful compositions. Formulations are described in a number of sources that are well known and readily available to those of ordinary skill in the art. For example, Remington's Pharmaceutical Science (Martin E. W., Easton Pa., Mack Publishing Company, 19th ed.) describes formulations that can be used in connection with the subject invention. Formulations suitable for parenteral administration, for example, include aqueous sterile injection solutions, which may contain antioxidants, buffers, bacteriostats, and solutes that render the formulation isotonic with the blood of the intended recipient; and aqueous and nonaqueous sterile suspensions that may include suspending agents and thickening agents. It should be understood that in addition to the ingredients particularly mentioned above, the formulations of the subject invention can include other agents conventional in the art having regard to the type of formulation and route of administration in question.

Methods for Producing Hepatocyte Cells

A unique population of hepatocyte cells, e.g., any one of the populations described herein, can be produced in an efficient and rapid manner by using methods of the invention described herein. Notably, the hepatocyte population can be produced without using any growth factors. The population of hepatocytes is different from other hepatocyte populations by the phenotype of the hepatocytes (e.g., lower AFP levels, an increase in albumin levels, and/or an increase in A1AT levels), indicating greater maturity of the hepatocytes.

Hepatocytes can be obtained by contacting a starting source of cells (e.g., stem cells) with Activin A and an effective amount of an inhibitor of PI3K alpha (e.g., Compound A) and culturing the cells under conditions sufficient to obtain a population of endoderm cells that will efficiently differentiate into hepatocytes. Methods of culturing populations of endoderm cells are described infra. Such a population of endoderm cells, or a population of endoderm cells obtained by using any methods of the invention, can be plated in one or more of any type of culture vessel, such as a plastic culture dish or multi-well plate, and/or maintained on a feeder layer in proliferation medium or hepatocyte medium. The hepatocyte medium can be DMEM/F12, GlutaMAX™ (Life Technologies) or L glutamine, and B-27® Supplement (Life Technologies); William's E (Life Technologies, CM6000) and Primary Hepatocyte Maintenance Supplements (Life Technologies, CM4000), with or without dexamethasone; RPMI, GlutaMAX™ or L glutamine, and B-27® Supplement; DMEM, GlutaMAX™ or L glutamine, and B-27® Supplement; DMEM/F12 and serum (lacking B-27®); DMEM and serum (lacking B-27®); RPMI and serum (lacking B-27®); William's E and serum (lacking B-27®); DMEM/F12 and KOSR, DMEM and KOSR, RPMI and KOSR, or William's E and KOSR.

In some embodiments, a significant portion of the cells in the population of endoderm cells differentiates into hepatocytes. In some aspects, at least about 50%, 55%, 60%, 65%, 70%, 75%, 80%, 85%, 90%, 91%, 92%, 93%, 94%, 95%, 96%, 97%, 98%, 99% or more of the cells in the endoderm cell population differentiate into hepatocytes. In some aspects, the differentiation occurs in at least 1 day, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days or more after the endoderm cells have been cultured in hepatocyte media. Without being bound by theory, the longer the population of endoderm cells is cultured in hepatocyte media, the greater the amount of the cells in the endoderm population will differentiate into hepatocytes. Using endoderm cells that have been cultured in endoderm media (exemplary endoderm media described in the Examples section) for at least 5 days allows for the production of highly homogeneous populations of hepatocytes. The differentiation can occur in the absence of growth factors, such as FGF.

Thus, in one embodiment, the methods of obtaining a population of hepatocyte cells includes contacting a population of stem cells (e.g., embryonic stem cells, adult stem cells, induced pluripotent stem cells) with an effective amount of Activin A and an effective amount of an inhibitor of PI3K alpha (e.g., Compound A) and culturing the cells under conditions sufficient to obtain the population of hepatocyte cells. In some embodiments, a population of endoderm cells as described herein are obtained after about 1, 2, 3, 4 or 5 days of culture. In other embodiments, the population of endoderm cells are removed from endoderm media and then cultured in hepatocyte media (as described in the Examples) without growth factors or PI3K inhibitors to produce a population of hepatocytes that are mature, as indicated by lower AFP levels and increased albumin levels.

As indicated in the Examples, when PI3K inhibitors (e.g., PI3K alpha or PI3K delta inhibitors) are not used at the endoderm stage, the AFP levels are low. In contrast, when PI3K inhibitors (e.g., PI3K alpha or PI3K delta inhibitors) are used at the endoderm stage, then the AFP levels can be higher (e.g., nearly 100-fold) higher. Thus, one of skill in the art can adjust the level of maturity for the hepatocytes by the use of PI3K inhibitors.

In certain embodiments, culturing the endoderm cells under conditions sufficient to obtain the population of hepatocytes can comprise culturing the endoderm cells in the absence of one or more of any of the following: HGF, retinoic acid, FGF8, FGF1, DMSO, FGF7, FGF10, OSM, Dexamethasone, FGF2, FGF4, BMP2, and BMP4.

Accordingly, a population of hepatocyte cells obtained by any one of the methods described above is also a feature of the invention. Beneficially, the once the population of hepatocytes is obtained, it can be maintained in medium in the absence of growth factors. This makes the hepatocytes obtained according to the methods herein particularly advantageous for use in downstream applications.

Compositions of Hepatocyte Cells

Hepatocyte cells of the invention are unique from other hepatocytes in their phenotype. A population of hepatocyte cells provided by the invention can be described by various phenotypes related to the expression of biological markers. These markers can be detected by standard methods known in the art including, but not limited to, immunohistochemistry, flow cytometry, and fluorescence imaging analysis.

The details of such techniques can be found in Example 13. Non-limiting examples of markers that can be used include one or more of the following:

| | |
|---|---|
| CYP2E1 | cytochrome P450, family 2, subfamily E, polypeptide 1 |
| CYP1A2 | cytochrome P450, family 1, subfamily A, polypeptide 2 |
| CYP3A7 | cytochrome P450, family 3, subfamily A, polypeptide 7 |
| CYP2C8 | cytochrome P450, family 2, subfamily C, polypeptide 8 |
| CYP2D6 | cytochrome P450, family 2, subfamily D, polypeptide 6 |
| CYP2B6 | cytochrome P450, family 2, subfamily B, polypeptide 6 |
| CYP2C9 | cytochrome P450, family 2, subfamily C, polypeptide 9 |
| CYP3A4 | cytochrome P450, family 3, subfamily A, polypeptide 4 |
| CYP3A5 | cytochrome P450, family 3, subfamily A, polypeptide 5 |
| CYP2C19 | cytochrome P450, family 2, subfamily C, polypeptide 19 |
| CYP7A1 | cytochrome P450, family 7, subfamily A, polypeptide 1 |
| TAT | Hs00910225_m1 |
| SERPINA1 | serpin peptidase inhibitor, clade A (alpha-1 antiproteinase, |
| | antitrypsin), member 1 |
| ADH1A | alcohol dehydrogenase 1A (class I), alpha polypeptide |
| CEL | carboxyl ester lipase (bile salt-stimulated lipase) |
| SERPINA3 | serpin peptidase inhibitor, clade A (alpha-1 antiproteinase, |
| | antitrypsin), member 3 |
| SERPINA7 | serpin peptidase inhibitor, clade A (alpha-1 antiproteinase, |
| | antitrypsin), member 7 |
| SDS | serine dehydratase |
| AGXT | alanine-glyoxylate aminotransferase |
| NNMT | nicotinamide N-methyltransferase |
| G6P | glucose-6-phosphatase |
| KRT18 | keratin 18 |
| KRT19 | keratin 19 |
| UGT1A1 | UDP glucuronosyltransferase 1 family, polypeptide A1 |
| UGT1A4 | UDP glucuronosyltransferase 1 family, polypeptide A4 |
| UGT1A3 | UDP glucuronosyltransferase 1 family, polypeptide A3 |
| UGT2B4 | UDP glucuronosyltransferase 2 family, polypeptide B4 |
| GSTM4 | glutathione S-transferase mu 4 |
| SULT2A1 | sulfotransferase family, cytosolic, 2A, |
| | dehydroepiandrosterone (DHEA)-preferring, member 1 |
| SULT1A1; | sulfotransferase family, cytosolic, 1A, phenol-preferring, |
| SULT1A2 | member 1; sulfotransferase family, cytosolic, 1A, phenol- |
| | preferring, member 2 |
| FGFR4 | fibroblast growth factor receptor 4 |
| EGFR | epidermal growth factor receptor |
| ASGPR | asialoglycoprotein receptor |
| AFP | alpha-fetoprotein |
| ALB | albumin |
| AFM | afamin |
| TAT | tyrosine aminotransferase |
| SERPINA1 | thyroxine binding globulin |
| FOXA1 | forkhead box A1 |
| FOXA2 | forkhead box A2 |
| FOXA3 | forkhead box A3 |
| HNF4A | hepatocyte nuclear factor 4, alpha |
| HNF1A | HNF1 homeobox A |
| HNF1B | HNF1 homeobox B |
| ONECUT1 | one cut homeobox 1 |
| HHEX | hematopoietically expressed homeobox |
| GATA4 | GATA binding protein 4 |
| GATA6 | GATA binding protein 6 |
| TBX3 | T-box 3 |
| CEBPA | CCAAT/enhancer binding protein (C/EBP), alpha |
| CEBPB | CCAAT/enhancer binding protein (C/EBP), beta |
| SLCO1B1 | solute carrier organic anion transporter family, |
| | member 1B1 |
| SLCO1B3 | solute carrier organic anion transporter family, |
| | member 1B3 |
| SLCO2B1 | solute carrier organic anion transporter family, |
| | member 2B1 |
| ABCB1 | ATP-binding cassette, sub-family B (MDR/TAP), |
| | member 1 |
| ABCG2 | ATP-binding cassette, sub-family G (WHITE), |
| | member 2 |
| ABCB11 | ATP-binding cassette, sub-family B (MDR/TAP), |
| | member 11 |
| ABCC2 | ATP-binding cassette, sub-family C (CFTR/MRP), |
| | member 2 |
| ABCC3 | ATP-binding cassette, sub-family C (CFTR/MRP), |
| | member 3 |
| ABCC4 | ATP-binding cassette, sub-family C (CFTR/MRP), |
| | member 4 |
| ABCB4 | ATP-binding cassette, sub-family B (MDR/TAP), |
| | member 4 |
| A1AT | Alpha1-antitrypsin |
| CK18 | Cytokeratin 18 |
| GSTA1 | Glutothione-S-Transferase 1 |
| FABP1 | Fatty acid-binding protein 1 |
| IL6R | Interleukin-6 receptor |
| VCAM1 | Vascular cell adhesion molecule 1 |

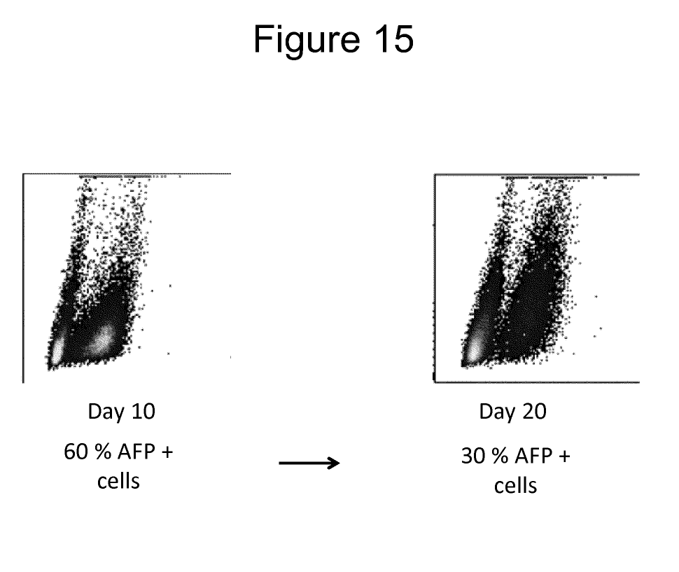

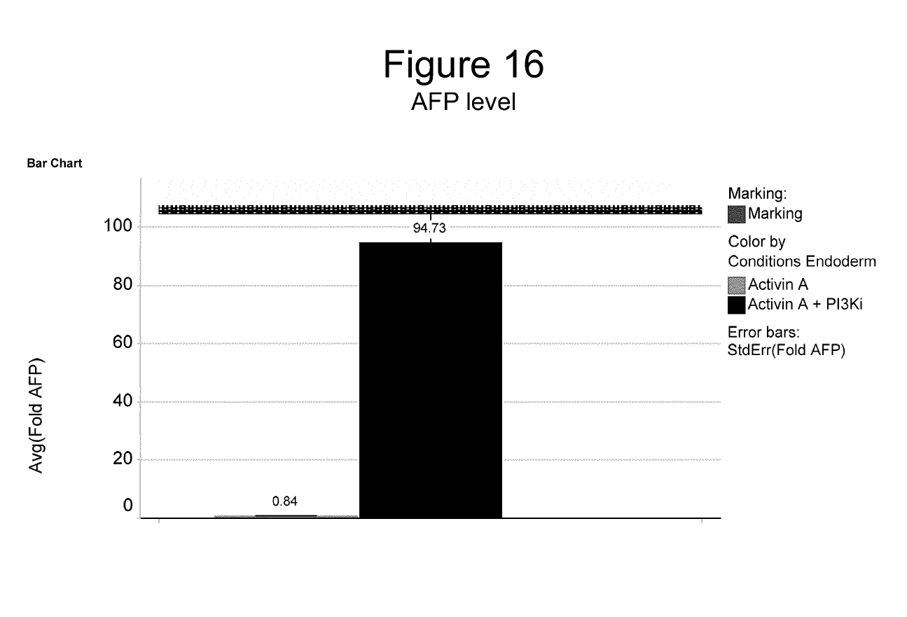

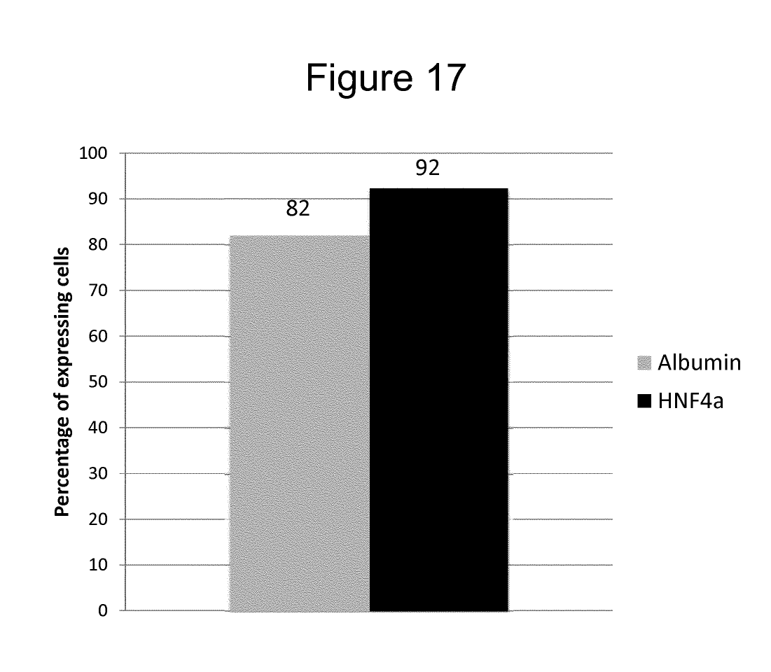

In one embodiment, hepatocytes made by the methods of the invention have decreased AFP levels as compared to HepG2 cells. In another embodiment and shown in the examples, the hepatocytes initially show an increase in AFP production comparable to AFP expression levels detected in HepG2 cells. This is followed by a decrease in AFP production levels (see FIG. 15 and Example 14 below). The decrease in AFP production levels is indicative of the maturation of hepatocyte cells. One embodiment exemplified by FIG. 16 shows that when PI3K inhibitor is used at the endoderm stage, the AFP level is almost 100 fold as compared to a control where PI3K alpha selective inhibitor is not added. Another embodiment exemplified by FIG. 17 is that stem cell derived hepatocytes at day 20 show expression of markers of albumin and HNF4a which is indicative of their transformation to hepatocytes. Accordingly, in some embodiments, the invention encompasses a population (e.g. homogeneous population) of hepatocytes or hepatocyte cells in which at least about 50%, at least about 55%, at least about 60%, at least about 65%, at least about 70%, at least about 75%, at least about 80%, at least about 81%, at least about 82%, or at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, greater than 99% or 100% of the cells in the population have decreased AFP levels. The decreased AFP levels can be 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, or more fold higher as compared to a control where PI3K alpha selective inhibitor is not added. The decreased AFP levels can also be measured by percentage decrease as shown in the Examples and figures.

Another aspect of the invention is an in vitro cell culture comprising cells in medium without growth factors, wherein the cells comprise endoderm cells, hepatocyte cells, and cells differentiated from endoderm cells, i.e., any of a variety of hepatocyte precursor cells. For example, the medium can be DMEM/F12, GlutaMAX™ (Life Technologies) or L glutamine, and B-27® Supplement (Life Technologies); William's E (Life Technologies, CM6000) and Primary Hepatocyte Maintenance Supplements (Life Technologies, CM4000), with or without dexamethasone; RPMI, GlutaMAX™ or L glutamine, and B-27® Supplement; DMEM, GlutaMAX™ or L glutamine, and B-27® Supplement; DMEM/F12 and serum (lacking B-27®); DMEM and serum (lacking B-27®); RPMI and serum (lacking B-27®); William's E and serum (lacking B-27®); DMEM/F12 and KOSR, DMEM and KOSR, RPMI and KOSR, or William's E and KOSR.

The invention provides a population (e.g. homogeneous population) of hepatocytes or hepatocyte cells in which at least about 50%, at least about 55%, at least about 60%, at least about 65%, at least about 70%, at least about 75%, at least about 80%, at least about 81%, at least about 82%, or at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, greater than 99% or 100% of the cells in the population express any one or more (e.g., 2, 3, 4, 5, 6, 7, or more) of the hepatocytes markers as described herein. In some embodiments, the appearance of these hepatocytes markers is measured after about 1 day, 2 days, 3 days, 4 days, 5 days or more in culture (e.g., 6 days, 7 days, 8 days, 9 days, 10 days, 11 days, 12 days, 13 days, 14 days, 15 days, 16 days, 17 days, 18 days, 19 days, 20 days or more in culture).

The invention provides a population (e.g. homogeneous population) of hepatocytes or hepatocyte cells in which in which at least about 50%, at least about 55%, at least about 60%, at least about 65%, at least about 70%, at least about 75%, at least about 80%, at least about 81%, at least about 82%, or at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, greater than 99% or 100% of the cells in the population secrete albumin. The secreted albumin levels can be 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, or more fold higher as compared to a control where PI3K alpha selective inhibitor is not added.

The invention provides a population (e.g. homogeneous population) of hepatocytes or hepatocyte cells in which in which at least about 50%, at least about 55%, at least about 60%, at least about 65%, at least about 70%, at least about 75%, at least about 80%, at least about 81%, at least about 82%, or at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, greater than 99% or 100% of the cells in the population secrete A1AT. The secreted A1AT levels can be 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, or more fold higher as compared to a control where PI3K alpha selective inhibitor is not added.

The invention encompasses a homogeneous population of hepatocyte cells (or hepatocytes). In some embodiments, a homogeneous population of hepatocyte cells (or hepatocytes) can be a population of cells where a significant portion of the population are hepatocytes. A significant portion is more than about 50%, 55%, 60%, 65%, 70%, 75%, 80%, 85%, 90%, 91%, 92%, 93%, 94%, 95%, 96%, 97%, 98%, or 99% of the cells in the population are hepatocytes.

In some embodiments, the cell populations (e.g., population of hepatocytes) have the described lower limit of any one or more markers described herein (e.g., AFP and markers in the table above) coupled with an upper limit of any one or more markers described herein. The invention contemplates a range that encompasses both a lower limit and an upper percentage of any of the numerical values recited herein. For example, one embodiment contemplates a population of endoderm cells where about 50% to about 90% of the hepatocytes in the population have decreased AFP. As a further example, in some embodiments, an upper limit of percentages can be any of about the following: 75%, 80%, 85%, 90%, 95%, or 99%. In some embodiments, the marker is AFP.

In some embodiments, CYP enzyme activity can be induced in the hepatocyte cell populations described herein. In some embodiments, the CYP activity is detected via mass spectrometry. In some embodiments, the activities of any one or more of CYP2B6, CYP3A4/5, CYP1A1/2, and aldehyde oxidase (AO) can be induced. In some embodiments, CYP enzyme activity and/or aldehyde oxidase (AO) activity is induced by a 10 μM rifampicin, 1 mM phenobarbital, and 1 μM 3-methylcholanthrene (3MC).

The invention contemplates and encompasses a culture comprising any intermediate cell types in the pathway that leads to the formation of any of the populations of hepatocyte cells described herein from a population of endoderm cells. Isolated population of hepatocytes (including those produced by the methods disclosed herein) and any intermediate cell types in the pathway that leads to the formation of any of the populations of hepatocyte cells are also encompassed by the invention.

Methods of Using Hepatocyte Cells

Hepatocyte cells derived from a population of endoderm cells provided by the invention can find advantageous use in a variety of research and clinical applications, including, e.g., absorption, distribution, metabolism, excretion, and toxicity studies and therapeutic liver regeneration. The invention provides populations of hepatocyte cells that can be used to treat degenerative liver diseases or inherited deficiencies of liver function. Because the liver controls the clearance and metabolism of drugs (e.g. small-molecule drugs), the hepatocyte cells provided by the invention can also be used to evaluate and/or model the effect of candidate drugs on liver cells in vivo.

Cell-Based Therapies

Liver diseases, such as hepatitis and cirrhosis, are becoming one of the most common causes of mortality in developing countries, and liver transplant is often the only available treatment. However, there is a shortage of suitable donor livers. The use of hepatocyte cells for therapeutic liver regeneration would offer a vast improvement over current cell therapy procedures that utilize cells from donor livers for the treatment of liver disease. The invention provides a source of hepatocytes that can be developed for such treatments.

Thus, in certain aspects, the invention provides methods of providing cell-based therapy to a patient in need thereof by administering to the patient hepatocyte cells obtained from any of the populations or obtain by using any of the methods described herein.

The hepatocyte cells can be administered at any site that has adequate access to the circulation, typically within the abdominal cavity. For some metabolic and detoxification functions, it is advantageous for the cells to have access to the biliary tract. Accordingly, the cells can be administered near the liver (e.g., in the treatment of chronic liver disease) or the spleen (e.g., in the treatment of fulminant hepatic failure). In one method, the cells administered into the hepatic circulation either through the hepatic artery, or through the portal vein, by infusion through an in-dwelling catheter. A catheter in the portal vein can be manipulated so that the cells flow principally into the spleen, or the liver, or a combination of both.

In another method, the cells can be administered by placing a bolus in a cavity near the target organ, typically in an excipient or matrix that will keep the bolus in place. In another method, the cells can be injected directly into a lobe of the liver or the spleen.

Human conditions that may be appropriate for such therapy include hepatic failure due to any cause, viral hepatitis, drug-induced liver injury, cirrhosis, inherited hepatic insufficiency (such as Wilson's disease, Gilbert's syndrome, or a₁-antitrypsin deficiency), hepatobiliary carcinoma, autoimmune liver disease (such as autoimmune chronic hepatitis or primary biliary cirrhosis), and any other condition that results in impaired hepatic function. For human therapy, the dose should take into account any adjustments for the body weight of the subject, nature and severity of the affliction, and the replicative capacity of the administered cells. A physician or managing clinician can determine the mode of treatment and the appropriate dose.

Methods for Screening Drug Candidates for Toxicity

Studying the metabolism of a drug and its toxicity are necessary steps in the development of new pharmaceutical compounds. Cost-effective development of new pharmaceutical agents can depend on the ability to prescreen drug candidates in cell-based assays. Hepatocytes are considered a cellular model of reference, as they express the majority of drug-metabolizing enzymes, respond to enzyme inducers, and are capable of generating an in vitro metabolic profile that is similar to a metabolic profile that can be obtained in vivo. The compositions and methods of the present invention provide a source of hepatocyte cells that can be used as reagents for testing a drug candidate's toxicity. Accordingly, the invention provides methods for screening candidate drugs for toxicity that include contacting a population of hepatocyte cells, e.g., a population provided by the invention or obtained using any one of the methods provided by the invention, with the drug candidate and monitoring the population of hepatocyte cells for toxicity.

Assessment of the activity of candidate pharmaceutical compounds generally involves combining the hepatocyte cells of this invention with the candidate compound, determining any change in the morphology, marker phenotype, or metabolic activity of the cells that is attributable to the compound (compared with untreated cells or cells treated with an inert compound), and then correlating the effect of the compound with the observed change. The screening may be done either because the compound is designed to have a pharmacological effect on liver cells, or because a compound designed to have effects elsewhere may have unintended hepatic side effects. Two or more drugs can be tested in combination (by combining with the cells either simultaneously or sequentially), to detect possible drug-drug interaction effects.

Cytotoxicity can be determined in the first instance by the effect on cell viability, survival, morphology, and leakage of enzymes into the culture medium. More detailed analysis is conducted to determine whether compounds affect cell function (such as gluconeogenesis, ureogenesis, and plasma protein synthesis) without causing toxicity. Lactate dehydrogenase (LDH) is a good marker because the hepatic isoenzyme (type V) is stable in culture conditions, allowing reproducible measurements in culture supernatants after 12-24 h incubation. Leakage of enzymes such as mitochondrial glutamate oxaloacetate transaminase and glutamate pyruvate transaminase can also be used.

Other current methods to evaluate hepatotoxicity include determination of the synthesis and secretion of albumin, cholesterol, and lipoproteins; transport of conjugated bile acids and bilirubin; ureagenesis; cytochrome p450 levels and activities; glutathione levels; release of alpha-glutathione s-transferase; ATP, ADP, and AMP metabolism; intracellular K⁺ and Ca²⁺ concentrations; the release of nuclear matrix proteins or oligonucleosomes; and induction of apoptosis (indicated by cell rounding, condensation of chromatin, and nuclear fragmentation). DNA synthesis can be measured as [³H]-thymidine or BrdU incorporation. Effects of a drug on DNA synthesis or structure can be determined by measuring DNA synthesis or repair. [³H]-thymidine or BrdU incorporation, especially at unscheduled times in the cell cycle, or above the level required for cell replication, is consistent with a drug effect. Unwanted effects can also include unusual rates of sister chromatid exchange, determined by metaphase spread (see, e.g., A. Vickers (pp 375-410 in In vitro Methods in Pharmaceutical Research, Academic Press, 1997) for further elaboration. Further methods for screening drug candidates for potential hepatotoxicity are described in Castell et al., In vitro Methods in Pharmaceutical Research, Academic Press, 1997).

Methods for Producing Pancreatic Progenitor Cells

A unique population of pancreatic progenitor cells, e.g., any one of the populations described herein, can be produced in an efficient and rapid manner by using methods of the invention described herein. The population of pancreatic progenitor cells is different from other pancreatic progenitor cell populations by the phenotype of the pancreatic progenitor cells (e.g., increased expression of pancreatic lineage marker genes, enhanced capability to form cell clusters, ability to grow in suspension), indicating greater maturity of the pancreatic progenitor cells.

Pancreatic progenitor cells can be obtained by contacting a starting source of cells (e.g., stem cells) with Activin A and an effective amount of an inhibitor of PI3K alpha, e.g., Compound A, and culturing the cells under conditions sufficient to obtain a population of endoderm cells that will efficiently differentiate into pancreatic progenitor cells. Methods of culturing populations of endoderm cells are described infra.

Such a population of endoderm cells, or a population of endoderm cells obtained by using any methods of the invention, can be plated in one or more of any type of culture vessel, such as a plastic culture dish or multi-well plate, and/or maintained on a feeder layer in proliferation medium.

Pancreatic progenitor cells can be obtained by culturing a population of endoderm cells described herein for at least 1 day, for at least two days, for at least 3 days, or for more than three days in medium supplemented with 50 ng/ml FGF10, 20 ng/ml FGF7, 100 ng/ml Noggin and a hedgehog inhibitor and then culturing the cells for at least one day, for at least 2 days, for at least three days, for at least four days, or for more than four days in in the same cocktail additionally supplemented with of 2 uM retinoic acid (Sigma). Following at least 4 days, at least 5 days, at least 6 days, at least 7 days, at least 8 days, at least 9 days, at least 10 days, or more than 10 days in culture in the presence of 50 ng/ml FGF10, 20 ng/ml FGF7, 100 ng/ml Noggin, a hedgehog inhibitor, and 2 uM retinoic acid, the cells can then be cultured for at least 1 day, for at least 2 days, for at least 3 days, or for more than 3 with 1 uM Notch inhibitor DAPT, 10 mM Nicotinamide, and 50 ng/ml Exendin 4. For maturation, cells can be cultured for at least 4 additional days at least 5 additional days, at least six additional days, at least seven additional days, or for more than 7 additional days in 50 ng/ml Exendin 4, 50 ng/ml EGF and 50 ng/ml IGF1. It is to be understood that the differentiation of pluripotent stem cells to pancreatic cells can be carried out in various basal media.

In certain embodiments, methods of obtaining pancreatic progenitor cells can include culturing endoderm cells with (−)− indolactam V, KAAD cyclopamine, betacellulin, HGF, Follistatin, SU5402 (FGFR specific tyrosine kinase inhibitor), FGF4, FGF2, BMP4, or any combination thereof.

In some embodiments, a significant portion of the cells in the population of endoderm cells differentiates into pancreatic progenitor cells. In some aspects, at least about 50%, 55%, 60%, 65%, 70%, 75%, 80%, 85%, 90%, 91%, 92%, 93%, 94%, 95%, 96%, 97%, 98%, 99% or more of the cells in the endoderm cell population differentiate into pancreatic progenitor cells. In some aspects, the differentiation occurs in at least 1 day, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days or more after the endoderm cells have been cultured according to a method described above. Using endoderm cells that have been cultured in according to a method described herein allows for the production of highly homogeneous populations of pancreatic progenitor cells.

In other embodiments, the population of endoderm cells cultured according to a method described herein can produce a population of pancreatic progenitor cells that are mature. As shown in the Examples, and described in further detail below, when PI3K inhibitors (e.g., PI3K alpha or PI3K delta inhibitors such as Compound A) are used at the endoderm stage, the expression of pancreatic lineage marker genes in the resulting pancreatic progenitor cells can be higher, clusters formed by the cells can be larger and more numerous, and the cells can be more viable in suspension as compared to a control where PI3K alpha selective inhibitor is not added. Thus, one of skill in the art can adjust the level of maturity for the pancreatic progenitor cells by the use of PI3K inhibitors.

The pancreatic progenitor cells obtained by a method described herein can be further differentiated into pancreatic exocrine cells. In certain embodiments, the pancreatic progenitor cells can be cultured in the presence of glucagon-like-peptide 1 (GLP1), dexamethasone, dorsomorphin, or any combination thereof to form pancreatic exocrine cells. In certain embodiments, the pancreatic progenitor cells can be cultured as described in Delaspre, et al. (2013). “Directed pancreatic acinar differentiation of mouse embryonic stem cells via embryonic signaling molecules and exocrine transcription factors.” PLoS One, 8(1), e54243, to form pancreatic exocrine cells. In certain embodiments, the pancreatic progenitor cells can be cultured as described in Shirasawa, et al. (2011). “A novel stepwise differentiation of functional pancreatic exocrine cells from embryonic stem cells.” Stem Cells Dev, 20(6), 1071-1078, to form a population of pancreatic exocrine cells.

The pancreatic progenitor cells obtained by a method described herein can be further differentiated into pancreatic ductal cells. In certain embodiments, the pancreatic progenitor cells can be cultured in the presence of EGF, FGF10, PDGF-AA, or any combination thereof to form a population of pancreatic ductal cells. In certain embodiments, the pancreatic progenitor cells can be cultured as described in Rhodes, et al. (2012). “Induction of mouse pancreatic ductal differentiation, an in vitro assay.” In Vitro Cell Dev Biol Anim, 48(10), 641-649 to form a population of pancreatic ductal cells.

Accordingly, a population of pancreatic progenitor cells, pancreatic exocrine cells, and/or a population of pancreatic ductal cells obtained by any one of the methods described above is also a feature of the invention.

Compositions of Pancreatic Progenitor Cells

Pancreatic progenitor cells of the invention are different from other pancreatic progenitor cells in their phenotype. A population of pancreatic progenitor cells provided by the invention can be described by various phenotypes related to the expression of biological markers. These markers can be detected by standard methods known in the art including, but not limited to, immunohistochemistry, flow cytometry, and fluorescence imaging analysis. Non-limiting examples of markers that can be used include Pdx1, ARX, GCG, GLIS3, HNF1A, HNF1B, HNF4a, INS, KRT19, MNX1, NEUROD1, NKX202, ONECUT1, RFX6, SERPINA3, SST, or any combination thereof.

Accordingly, in some embodiments, the invention encompasses a population (e.g. homogeneous population) of pancreatic progenitor cells in which at least about 50%, at least about 55%, at least about 60%, at least about 65%, at least about 70%, at least about 75%, at least about 80%, at least about 81%, at least about 82%, or at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, greater than 99% or 100% of the cells in the population express Pdx1, ARX, GCG, GLIS3, HNF1A, HNF1B, HNF4a, INS, KRT19, MNX1, NEUROD1, NKX202, ONECUT1, RFX6, SERPINA3, SST, C-peptide, or any combination thereof. The expression levels of Pdx1, ARX, GCG, GLIS3, HNF1A, HNF1B, HNF4a, INS, KRT19, MNX1, NEUROD1, NKX202, ONECUT1, RFX6, SERPINA3, SST, C-peptide, or any combination thereof can be 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, or more fold higher as compared to a control where PI3K alpha selective inhibitor is not added. The expression levels of Pdx1, ARX, GCG, GLIS3, HNF1A, HNF1B, HNF4a, INS, KRT19, MNX1, NEUROD1, NKX202, ONECUT1, RFX6, SERPINA3, SST, C-peptide, or any combination thereof can be detected after 7 days, after 8 days, after 9 days, after 10 days, after 11 days, after 12 days, or more than 12 days (e.g., more than 13, 14, 15, 16, 17, 18, or days) of differentiation.

A population of pancreatic progenitor cells provided by the invention can be described by various phenotypes related to cell morphology, e.g., the formation of three dimensional cell clusters. Notably, when PI3K inhibitors (e.g., PI3K alpha or PI3K delta inhibitors such as Compound A) are used at the endoderm stage, the three-dimensional clustered formed by the resulting pancreatic progenitor can be larger and more numerous compared to a control where PI3K alpha selective inhibitor (e.g., Compound A) is not added. The formation of such clusters can be monitored visually (e.g., using a microscope). In certain embodiments, pancreatic progenitor cells of provided can form larger and more numerous cell clusters after day 3, day 4, day 5, day 6, day 7, day 8, day 9, day 10, day 11, day 12, day 13, day 14, day 15, day 16, day 17, day 18, day 19, day 20, or after day 21 compared to a control where PI3K alpha selective inhibitor is not added.

Moreover, the pancreatic progenitor cells of the invention are capable of expressing insulin, glucagon, and C peptide, are capable growing in suspension and are viable in suspension longer compared to a where PI3K alpha selective inhibitor (e.g., Compound A) is not added. In some embodiments, the pancreatic progenitor cells provided herein remain viable after 1 day, after 2 days, after 3 days, after 4 days, after 5 days, after 6 days, after 7 days, after 8 days, after 9 days, after 10 days, after 11 days, after 12 days, after 13 days, after 14 days, after 15 days, after 16 days, after 17 days, after 18 days, after 19 days, after 20 day or after more than 20 days in suspension.

The invention contemplates and encompasses a culture comprising any intermediate cell types in the pathway that leads to the formation of any of the populations of pancreatic progenitor cells described herein from a population of endoderm cells. Isolated population of pancreatic progenitor cells (including those produced by the methods disclosed herein) and any intermediate cell types in the pathway that leads to the formation of any of the populations of pancreatic progenitor cells are also encompassed by the invention.

Methods of Using Pancreatic Progenitor Cells

Pancreatic progenitor cells, pancreatic ductal cells, pancreatic endocrine cells, and pancreatic exocrine cells derived from a population of endoderm cells provided by the invention can find advantageous use in a variety of research and clinical applications, including, e.g., cell-based therapies. The invention provides populations of pancreatic progenitor cells that can be used to treat pancreatic diseases, e.g., diabetes mellitus, or inherited deficiencies of pancreas function, e.g., exocrine pancreatic insufficiency associated with cystic fibrosis or Schwachman-Diamond syndrome.

Cell-Based Therapies

Chronic pancreatic diseases and disorders, such as pancreatitis and diabetes mellitus, are becoming increasingly prevalent in developing countries. While pancreas transplantation can significantly improve both quality and quantity of life, the donor organ shortage remains a major challenge. The use of pancreatic progenitor cells for therapeutic pancreas regeneration would offer a vast improvement over current cell therapy procedures that utilize cells from donor pancreases for the treatment of pancreatic disease. The invention provides a source of pancreatic progenitor cells that can be developed for such treatments.

Thus, in certain aspects, the invention provides methods of providing cell-based therapy to a patient in need thereof by administering to the patient a population comprising pancreatic progenitor cells or a population obtained by using any of the methods described herein

The pancreatic progenitor cells can be administered at any site that has adequate access to the circulation, typically within the abdominal cavity. Accordingly, the cells can be administered near the pancreas (e.g., in the treatment of chronic pancreatic disease). In one method, the cells can be administered through the portal vein of the liver, by infusion through an in-dwelling catheter, or through a small incision in the abdomen. A catheter in the portal vein can be manipulated so that the cells flow principally into the liver.

In another method, the cells can be administered by placing a bolus in a cavity near the target organ, typically in an excipient or matrix that will keep the bolus in place. In another method, the cells can be injected directly into the pancreas.

Human conditions that may be appropriate for such therapy include any cause, including injury to the pancreas, exocrine pancreatic insufficiency (resulting from, e.g., cystic fibrosis, Schwachman-Diamond syndrome, chronic pancreatitis, or obstruction of the pancreatic duct), pancreatic adenocarcinoma, islet cell neuroendocrine tumors, autoimmune pancreatic disease (such as autoimmune pancreatitis or type I diabetes), type II diabetes mellitus, and any other condition that results in impaired pancreatic function. For human therapy, the dose should take into account any adjustments for the body weight of the subject, nature and severity of the affliction, and the replicative capacity of the administered cells. A physician or managing clinician can determine the mode of treatment and the appropriate dose.

Methods for Screening Drug Candidates for Toxicity

As noted above, studying the metabolism of a drug and its toxicity are necessary steps in the development of new pharmaceutical compounds. Cost-effective development of new pharmaceutical agents can depend on the ability to prescreen drug candidates in cell-based assays. The compositions and methods of the present invention provide a source of pancreatic progenitor cells and/or pancreatic cells that can be used as reagents for testing a drug candidate's toxicity. Accordingly, the invention provides methods for screening candidate drugs for toxicity that include contacting a population of pancreatic progenitor cells and/or pancreatic cells, e.g., a population provided by the invention or obtained using any one of the methods provided by the invention, with the drug candidate and monitoring the population of pancreatic progenitor cells and/or pancreatic cells for toxicity.

Assessment of the activity of candidate pharmaceutical compounds generally involves combining the pancreatic progenitor cells and/or pancreatic cells of this invention with the candidate compound, determining any change in the morphology, marker phenotype, or metabolic activity of the cells that is attributable to the compound (compared with untreated cells or cells treated with an inert compound), and then correlating the effect of the compound with the observed change. The screening may be done either because the compound is designed to have a pharmacological effect on pancreatic progenitor cells and/or pancreatic cells, or because a compound designed to have effects elsewhere may have unintended hepatic side effects. Two or more drugs can be tested in combination (by combining with the cells either simultaneously or sequentially), to detect possible drug-drug interaction effects.

Cytotoxicity can be determined in the first instance by the effect on cell viability, survival, morphology, and leakage of enzymes into the culture medium. More detailed analysis is conducted to determine whether compounds affect cell function (such as gluconeogenesis, ureogenesis, and plasma protein synthesis) without causing toxicity.

Other current methods to evaluate toxicity include ATP, ADP, and AMP metabolism; intracellular K⁺ and Ca²⁺ concentrations; the release of nuclear matrix proteins or oligonucleosomes; and induction of apoptosis (indicated by cell rounding, condensation of chromatin, and nuclear fragmentation). DNA synthesis can be measured as [³H]-thymidine or BrdU incorporation. Effects of a drug on DNA synthesis or structure can be determined by measuring DNA synthesis or repair. [³H]-thymidine or BrdU incorporation, especially at unscheduled times in the cell cycle, or above the level required for cell replication, is consistent with a drug effect. Unwanted effects can also include unusual rates of sister chromatid exchange, determined by metaphase spread (see, e.g., A. Vickers (pp 375-410 in In vitro Methods in Pharmaceutical Research, Academic Press, 1997) for further elaboration. Further methods for screening drug candidates for potential toxicity are described in Castell et al., In vitro Methods in Pharmaceutical Research, Academic Press, 1997).

Methods of Making Lung Cells, Thyroid Cells, and Airway Progenitor Cells

Populations of lung cells, thyroid cells, and/or airway progenitor cells can be produced in an efficient and rapid manner by using methods of the invention described herein. Lung cells, thyroid cells, and/or airway progenitor cells can be obtained by contacting a starting source of cells (e.g., stem cells) with Activin A and an effective amount of an inhibitor of PI3K alpha, e.g., Compound A, and culturing the cells under conditions sufficient to obtain a population of endoderm cells that will efficiently differentiate into lung cells, thyroid cells, or airway progenitor cells. Methods of culturing populations of endoderm cells are described infra. Such a population of endoderm cells, or a population of endoderm cells obtained by using any methods of the invention, can be plated in one or more of any type of culture vessel, such as a plastic culture dish or multi-well plate, and/or maintained on a feeder layer in proliferation medium.

Lung cells and/or thyroid cells can be obtained by culturing a population of endoderm cells described herein in basal medium basal medium supplemented with 100 ng/ml Noggin and 10 mM SB431542 (TGFbeta inhibitor). After 24 hours, the media can be replaced with Nkx2-1 induction media: cSFDM supplemented with 100 ng/ml mWnt3a, 10 ng/ml mKGF, 10 ng/ml hFGF10, 10 ng/ml mBMP4, 20 ng/ml hEGF, 500 ng/ml mFGF2 and 100 ng/ml Heparin Sodium Salt (Sigma). The cells can then be cultured for 7 days in cSFDM supplemented with mFGF2 (500 ng/ml), hFGF10 (100 ng/ml), and 100 ng/ml Heparin Sodium Salt (Sigma). On day 22, cells can be cultured in lung maturation media: Ham's F12 media+15 mM HEPES (pH 7.4)+0.8 mM CaCl2+0.25% BSA+5 mg/ml insulin+5 mg/ml transferrin+5 ng/ml Na selenite+50 nM Dexamethasone+0.1 mM 8-Br-cAMP+0.1 mM IBMX+10 ng/ml KGF. In some embodiments, the endoderm cells can be cultured as described in Longmire, et al. (2012). “Efficient derivation of purified lung and thyroid progenitors from embryonic stem cells.” Cell Stem Cell, 10(4), 398-411.

Alternatively, to produce lung cells and/or airway progenitor cells, at day 3 of differentiation, a population endoderm cells described herein can be exposed to 500 nM A-83-01 (TGF beta inhibitor) with or without 4 uM Dorsomorphin (BMP inhibitor) or 20 ng/ml BMP4 for up to 2 days, 3 days, 4 days, or more than 4 days. The cells can then be exposed for at least 2 days, at least 3 days, or for more than 3 days to 10 ng/ml BMP4, 20 ng/ml FGF2+10 nM GSK3iXV. To obtain airway progenitor cells, the cells can then be exposed to 20 ng/ml BMP7, 20 ng/ml FGF7, 100 nM IWR-1 (WNT antagonist), and 1 mM PD98059 for at least one day, at least 2 days, or for more than 2 days. In some embodiments, a population endoderm cells described herein can be cultured as described in Mou, et al. (2012). “Generation of multipotent lung and airway progenitors from mouse ESCs and patient-specific cystic fibrosis iPSCs. Cell Stem Cell, 10(4), 385-397.

In some embodiments, a significant portion of the cells in the population of endoderm cells differentiates into lung, thyroid, and/or airway progenitor cells. In some aspects, at least about 50%, 55%, 60%, 65%, 70%, 75%, 80%, 85%, 90%, 91%, 92%, 93%, 94%, 95%, 96%, 97%, 98%, 99% or more of the cells in the endoderm cell population differentiate into lung, thyroid, and/or airway progenitor cells. In some aspects, the differentiation occurs in at least 1 day, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days or more after the endoderm cells have been cultured according to a method described herein.

Uses of Lung Cells, Thyroid Cells, and Airway Progenitor Cells

Lung cells, thyroid cells, and airway progenitor cells derived from a population of endoderm cells provided by the invention can find advantageous use in a variety of research and clinical applications, including, e.g., cell-based therapies. The invention provides populations of lung cells, thyroid cells, and airway progenitor cells that can be used to treat lung injury, respiratory diseases, e.g., acute respiratory distress syndrome, emphysema, mesothelioma, etc., and thyroid disease, e.g., thyroid cancer, Hashimoto's chronic lymphocytic thyroiditis, etc.

Cell-Based Therapies

While lung transplantation can significantly improve both quality and quantity of life, for patients with lung injuries or lung disease, the donor organ shortage remains a major challenge. The use of lung cells, thyroid cells, or airway progenitor cells for therapeutic lung or thyroid regeneration would offer a vast improvement over current therapies for the treatment of lung or thyroid disease. The invention provides a source of lung cells, thyroid cells, or airway progenitor cells that can be developed for such treatments.

Thus, in certain aspects, the invention provides methods of providing cell-based therapy to a patient in need thereof by administering to the patient a population comprising lung cells, thyroid cells, or airway progenitor cells obtained from any of the populations or a population obtained by using any of the methods described herein.

The lung cells, thyroid cells, or airway progenitor cells can be administered at any site that has adequate access to the circulation. Accordingly, the cells can be administered at an artery at or near the lungs (e.g., in the treatment of lung disease) or at or near the neck (e.g., in the treatment of thyroid disease). In one method, the cells can be administered via inhalation, by infusion through an in-dwelling catheter, or through a small incision in the lung or thyroid.

In another method, the cells can be administered by placing a bolus in a cavity near the target organ, typically in an excipient or matrix that will keep the bolus in place. In another method, the cells can be injected directly into the lung or thyroid.

Human conditions that may be appropriate for such therapy include any cause, including injury to the lung (such as fibrosing injuries), lung cancers (such as mesothelioma and others), emphysema, asthma, cystic fibrosis, chronic obstructive pulmonary disease (COPD), interstitial lung disease, thyroid injury, thyroid cancer, Crohn's disease, Grave's disease, Hashimoto's chronic lymphocytic thyroiditis, and others. For human therapy, the dose should take into account any adjustments for the body weight of the subject, nature and severity of the affliction, and the replicative capacity of the administered cells. A physician or managing clinician can determine the mode of treatment and the appropriate dose.

Uses of Intestinal Cells

Intestinal cells derived from a population of endoderm cells provided by the invention can find advantageous use in a variety of research and clinical applications, including, e.g., cell-based therapies. The invention provides populations of intestinal cells that can be used to inflammatory bowel disease (IBD), celiac disease, Crohn's disease, ulcers, ulcerative colitis, intestinal cancer, etc.

Cell-Based Therapies

The use of intestinal cells for therapeutic regeneration would offer a vast improvement over current therapies for the treatment intestinal disease. The invention provides a source of intestinal cells that can be developed for such treatments.

Thus, in certain aspects, the invention provides methods of providing cell-based therapy to a patient in need thereof by administering to the intestinal cells obtained from any of the populations or obtained by using any of the methods described herein

The intestinal cells can be administered at any site that has adequate access to the circulation. Accordingly, the cells can be administered at an artery at or near the abdomen. In one method, the cells can be administered by infusion through an in-dwelling catheter, or through a small incision in the abdomen. In another method, the cells can be administered by placing a bolus in a cavity near the target organ, typically in an excipient or matrix that will keep the bolus in place. In another method, the cells are injected directly into the abdomen.

Human conditions that may be appropriate for such therapy include any cause, including injury intestine, intestinal cancers, inflammatory bowel syndrome, celiac disease, Crohn's disease, bowel injury, ulcers, angiodysplasia, disorders of intestinal absorption or secretion, and others. For human therapy, the dose should take into account any adjustments for the body weight of the subject, nature and severity of the affliction, and the replicative capacity of the administered cells. A physician or managing clinician can determine the mode of treatment and the appropriate dose.

The following examples are provided for illustrative purposes and are not intended to limit the scope of the invention in any manner.

### EXAMPLES

### Example 1

### Methods and Materials for Endoderm Differentiation

Endoderm Markers

A variety of cell-type specific markers were used to monitor the differentiation of stem cells into endoderm cells in the flow cytometry, fluorescence imaging, and immunoassay experiments described below. To detect endoderm conversion, hESC-derived cell samples were stained for the expression of SOX17, FoxA2, and CXCR4 proteins. SOX17, FoxA2, and CXCR4 are proteins that are expressed by endoderm cells but not by stem cells. To detect stem cells, cell samples were stained for the expression of OCT4, a protein expressed by stem cells but not by endoderm cells.

Endoderm Differentiation Protocol Using hESCs and Matrigel

Undifferentiated human embryonic stem cells (hES) were maintained at a density of 40,000 cells/cm² on qualified matrigel (BD, #354277) in TesR™2 medium (STEMCELL™ Technologies #05860). Cultures were manually passaged twice a week. To prepare for endoderm differentiation, hESC cells were passaged into TesR™2 medium overnight. The following day, the TesR™2 medium was replaced with basal medium (DMEM/F12+Glutamax (Invitrogen, #10565) supplemented with B27 (Invitrogen, #17504-044)). No human embryos were destroyed in the process of obtaining stem cells for these methods. Furthermore, the plurality of stem cells is not obtained by the prior destruction of human embryos.

Unless otherwise noted, basal medium was supplemented with 100 μg/ml human Activin A (Peprotech, #120-14). When indicated, basal medium was also supplemented with an effective amount of, e.g., a growth factor such as 50 μg/ml human Wnt3a (R&D, #5036-WN-010), an isoform-specific P13K inhibitor or an mTOR inhibitor. After the three day treatment, the hES-derived cells were harvested, labeled, and analyzed via flow cytometry, imaging, or AlphaLISA.

Endoderm Differentiation Protocol Using hESCs and Suspension

The endoderm differentiation protocol is performed using hESC cultured in suspension. Confluent undifferentiated hESC grown on qualified matrigel are dissociated via incubation with TrypLE (Life Technologies, #12563-029) until the cells dissociate from the plate. The cells are then diluted with DMEM:F12 (50:50), collected in a conical tube, and centrifuged at 300×g for 8 minutes. After the supernatant is aspirated, the pelleted cells are all resuspended into a single cell suspension and counted using a hemocytometer. 20 mls of 4×10⁴ cells/mL are plated in a T75 Corning Low Attachment Flask in TeSR2 Media supplemented with 10 μM ROCK inhibitor Y-26732 and Pen/Strep solution. The medium is changed every other day by collecting the suspended cells, allowing them to settle in conical tubes, and gently pipetting off the old media. The cells begin to form clusters which expand outward from a spherical center. Tight clusters with defined spherical edges denote a retention in pluripotency while single cells or a cluster with an ill-defined border region typically denotes spontaneous differentiation and/or cell death. The cells are passaged at 3-4 day intervals by collecting the media in each flask and allowing the cell clusters to settle. The old media is gently pipetted off, and the clusters are dissociated into single cells using TrypLE, as described above. The dissociated cells are then plated as described above, i.e., 20 mls of 4×10⁴ cells/mL are plated per T75 Corning Low Attachment Flask in TeSR2 Media supplemented with 10 μM ROCK inhibitor Y-26732 and Pen/Strep solution.

To prepare for endoderm differentiation, hESC cells cultured in suspension are passaged onto plates in TesR™2 medium overnight. The following day, the TesR™2 medium is replaced with basal medium (DMEM/F12+Glutamax (Invitrogen, #10565) supplemented with B27 (Invitrogen, #17504-044)). The cells are differentiated into endoderm as described above.

Unless otherwise noted, basal medium was supplemented with 100 mg/ml human Activin A (Peprotech, #120-14). When indicated, basal medium was also supplemented with an effective amount of, e.g., a growth factor such as 50 μg/ml human Wnt3a (R&D, #5036-WN-010), an isoform-specific P13K inhibitor or an mTOR inhibitor. After the three day treatment, the hES-derived cells were harvested, labeled, and analyzed via flow cytometry, imaging, or AlphaLISA.

Endoderm Differentiation Protocol Using Non-Embryonic Stem Cells and Matrigel

Non-embryonic stem cells (adult stem cells or induced pluripotent stem (iPS) cells) are maintained at a density of 40,000 cells/cm² on qualified matrigel (BD, #354277) in TesR™2 medium (STEMCELL™ Technologies #05860). Cultures are manually passaged twice a week. To prepare for endoderm differentiation, adult stem cells or iPS cells are passaged into TesR™2 medium overnight. The following day, the TesR™2 medium is replaced with basal medium (DMEM/F12+Glutamax (Invitrogen, #10565) supplemented with B27 (Invitrogen, #17504-044)). Another option for culturing iPS cells is to use a mix of TesR2 and mouse embryonic fibroblast (MEF) conditioned medium (R&D Systems, #AR005). The cells are then differentiated into endoderm as described above.

Endoderm Differentiation Using Non-Embryonic Stem Cells and Suspension

This examples describes endoderm differentiation protocol using non-embryonic stem cells (adult stem cells or induced pluripotent stem (iPS) cells) cultured in suspension. Confluent undifferentiated adult stem cells or induced pluripotent stem (iPS) cells grown on qualified matrigel are dissociated via incubation with TrypLE (Life Technologies, #12563-029) until the cells dissociate from the plate. The cells are then diluted with DMEM:F12 (50:50), collected in a conical tube, and centrifuged at 300×g for 8 minutes. After the supernatant is aspirated, the pelleted cells are all resuspended into a single cell suspension and counted using a hemocytometer. 20 mls of 4×10⁴ cells/mL are plated in a T75 Corning Low Attachment Flask in TeSR2 Media supplemented with 10 μM ROCK inhibitor Y-26732 and Pen/Strep solution. The medium is changed every other day by collecting the suspended cells, allowing them to settle in conical tubes, and gently pipetting off the old media. The cells begin to form clusters which expand outward from a spherical center. Tight clusters with defined spherical edges denote a retention in pluripotency while single cells or a cluster with an ill-defined border region typically denotes spontaneous differentiation and/or cell death. The cells are passaged at 3-4 day intervals by collecting the media in each flask and allowing the cell clusters to settle. The old media is gently pipetted off, and the clusters are dissociated into single cells using TrypLE, as described above. The dissociated cells are then plated as described above, i.e., 20 mls of 4×10⁴ cells/mL are plated per T75 Corning Low Attachment Flask in TeSR2 Media supplemented with 10 μM ROCK inhibitor Y-26732 and Pen/Strep solution.

To prepare for endoderm differentiation, adult stem cells or iPS cells cultured in suspension are passaged onto plates in TesR™2 medium overnight. The following day, the TesR™2 medium is replaced with basal medium (DMEM/F12+Glutamax (Invitrogen, #10565) supplemented with B27 (Invitrogen, #17504-044)). Another option for culturing iPS cells is to use a mix of TesR2 and mouse embryonic fibroblast (MEF) conditioned medium (R&D Systems, #AR005). The cells are then differentiated into endoderm as described above.

Flow Cytometry Protocol

To prepare the cells for flow cytometry, hESC-derived cells grown under endoderm differentiation conditions were dissociated using Accutase (Innovative Cell technologies, #AT-104). Briefly, the cells were washed once in PBS, incubated with Accutase for 10 minutes at room temperature, pelleted, and washed in cold PBS. The Accutase-dissociated hESC-derived cell samples were stained with anti-CXC4 antibody, anti-SOX17 antibody, or anti-FoxA2 antibody. Additional cell samples were stained with isotype control antibodies (e.g., IgG1 or IgG2).

The cells that were to be stained with anti-CXCR4 antibody were washed once with cold DPBS and then directly stained with mouse anti-human CD184 (CXCR4)-IgG2-PE (BD, #555974) for one hour at 4° C. The cells that were to be stained with anti-SOX17 antibody or anti-FoxA2 antibody were first fixed for 25 minutes at 4° C. in fixation buffer (BD, #554655) and permeabilized for 30 minutes on ice in Perm Buffer III (BD, #554656). Anti-SOX17 staining was performed using a mouse anti-SOX17 IgG1-PE antibody (BD, #561591) at room temperature for 30 minutes. Anti-FoxA2 staining was performed using a mouse anti-human FoxA2 IgG1 (BD, #561589) under the same conditions. A control sample of cells were fixed as described above and stained with either an anti-IgG1-PE antibody (BD, #554680) at room temperature for 30 minutes or an anti-IgG2-PE antibody (BD, #55574) for one hour at 4° C.

The antibody-stained hESC-derived cells were then analyzed via flow cytometry using a BD LSRFortessa™ cell analyzer. The threshold parameter was set to 15,000; the SSC and FSC parameters were set and SSC were set to allow the entire population of cells to fit within the range of recorded data; and the voltage was set so that unstained cells or cells stained with isotype control antibodies had a fluorescence less than 10³. Approximately 1×10⁶ cells were analyzed per sample.

Imaging Protocol

Prior to immunofluorescence imaging, the hESC-derived cells washed three times at room temperature with PBS and fixed for 20 minutes in 4% methanol-free formaldehyde that had been diluted in PBS. The cell samples were then rinsed three times at room temperature in PBS and blocked for one hour at room temperature in blocking buffer (0.3% Triton X-100 and 5% goat serum in 1×PBS). The cell samples were again rinsed three times in PBS following the blocking step. Cell samples in which SOX17 expression was detected were incubated for 2 hours at room temperature with 2 μg/ml mouse anti-SOX17 Clone P7969 primary antibody (BD, #561590) in blocking buffer. Cell samples in which FoxA2 expression was detected were incubated for 2 hours at room temperature in a 1:500 dilution of rabbit anti-FoxA2 primary antibody (CS, #3143) in blocking buffer. Alternatively, these incubations can be performed overnight at 4° C.

Prior to staining with secondary antibody, the cell samples were rinsed three times in PBS. Cells stained with anti-SOX17 antibody were then incubated with 2 μg/ml goat anti-mouse-Alexa488 secondary antibody (Invitrogen, #A11029) for 1 hour at room temperature. Cells stained with anti-FoxA2 antibody were then incubated with 2 μg/ml goat anti-rabbit-Alexa594 secondary antibody (Invitrogen, #A11037) under the same incubation conditions.

Nuclear staining was performed following staining with secondary antibody. Briefly, cell samples were washed three times with PBS and stained for ten minutes at room temperature with Hoechst 33258 (Invitrogen, #H3569) diluted 1/10000 in PBS. Following the incubation, the cells were washed again with PBS. The Hoechst-stained cells were then imaged using a Zeiss microscope or the Perkin Elmer Operetta system using standard fluorescent microscopy techniques.

AlphaLISA Protocol

To detect OCT4, the cells were washed three times in PBS and lysed with 50 ul AlphaLISA Lysis Buffer (Perkin Elmer, #AL003C). The lysis buffer was added to each cell sample and mixed with the cells five times. The cell samples were then incubated in a plate shaker at room temperature for 15 minutes. 5 μl of the lysate from each sample was then transferred to a 384 well OptiPlate (Perkin Elmer, #6005629). 5 μl of 10 ug/ml anti-rabbit acceptor bead (Perkin Elmer, #AL104M) and 5 ml of 0.2 nM of rabbit anti-OCT4 antibody (Cell Signaling, #2890) was added to each well and incubated for 2 hours at room temperature. Following the incubation, 5 μl of 0.5 nM mouse anti-OCT4 antibody (BD, #611203) and 5 ml of 0.5 nM biotinylated goat anti-mouse antibody (Invitrogen, #B2763) were added to each well and incubated at room temperature for 2 hours. Following the incubation, 10 ml of streptavidin donor beads (Perkin Elmer, #6760002B) were added to each well and incubated for 30 minutes. The OptiPlates were then analyzed using an Envision Multilabel Plate Reader (Perkin Elmer, #2104-0010). All beads and antibodies were diluted as necessary in IAB Buffer (Perkin Elmer, #AL000C)+50 mM NaCl. Four replicate assays were performed.

To detect SOX17, the cells were washed three times in PBS and lysed with 50 ul Roche Complete Lysis Buffer (Roche, #04719956001). The lysis buffer was added to each cell sample and mixed with the cells five times. The cell samples were then incubated in a plate shaker at room temperature for 15 minutes. 5 μl of the lysate from each sample was then transferred to a 384 well OptiPlate (Perkin Elmer, #6005629). 5 μl of 10 ug/ml anti-rabbit acceptor bead (Perkin Elmer, #AL104M) and 5 ml of 1 nM of rabbit anti-SOX17 antibody (Sigma, #AV33271) was added to each well and incubated for 2 hours at room temperature. Following the incubation, 5 μl of 0.5 nM mouse anti-SOX antibody (Sigma, #SAB3300093) and 5 ml of 0.5 nM biotinylated goat anti-mouse antibody (Invitrogen, #B2763) were added to each well and incubated at room temperature for 2 hours. Following the incubation, 10 ml of streptavidin donor beads (Perkin Elmer, #6760002B) were added to each well and incubated for 30 minutes. The OptiPlates were then analyzed using an Envision Multilabel Plate Reader (Perkin Elmer, #2104-0010). All beads and antibodies were diluted as necessary in IAB Buffer (Perkin Elmer, #AL000C) Four replicate assays were performed.

siRNA Knockdown Protocol

hESC cell samples were prepared as described above and differentiated in basal medium supplemented with Activin A alone. During passage into basal medium, the cells were transfected with an appropriate siRNA listed in Table 3 or Table 4 below using a lipid-based transfection system (Lipofeactamine RNAimax, Invitrogen, #133778-150). The cells were incubated with the siRNAs for 20 hours. Following the incubation, the medium was changed and replaced with medium supplemented with Activin A alone. The results of PI3K knockdown experiments are shown in Example 2 below. The results of Akt and mTOR knockdown experiments are shown in Example 8 below.

### Example 2

### Endoderm Differentiation Using hESCs

The effects of a variety of commercially available PI3K inhibitors on endoderm differentiation were compared. hESC cell samples were prepared as described above and differentiated in basal medium or basal medium supplemented with Activin A; Activin A and 50 μg/ml human Wnt3a (R&D, #5036-WN-010); or Activin A, Wnt3A and one of the P13K inhibitors listed in Table 1 below.

TABLE 1

| PI3K Inhibitor | Concentration Used in Example 2 |
|---|---|
| Compound A | 500 nM |
| Wortmannin | 250 nM |
| PIK90 | 500 nM |
| LY294002 | 5 μM |

With the exception of Compound A, the compounds shown in Table 1 are not are isoform-selective PI3K inhibitors.

The structure of Compound A is provided below:

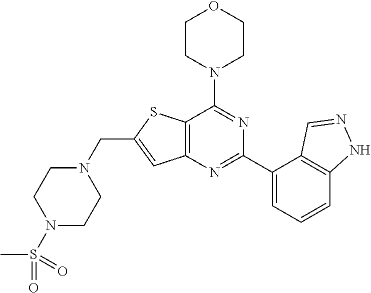 [SMILES: CS(=O)(=O)N1CCN(Cc2cc3nc(-c4cccc5[nH]ncc45)nc(N4CCOCC4)c3s2)CC1]

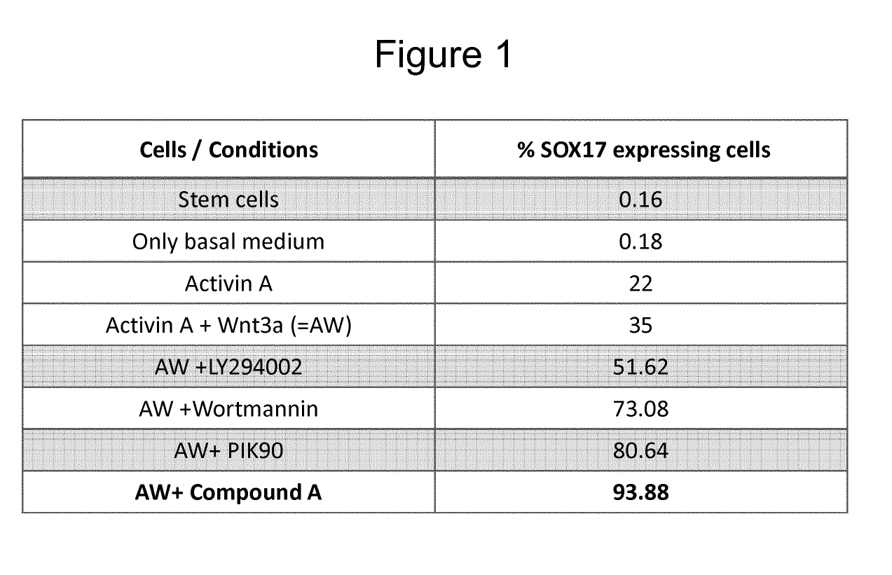

After a 3-day treatment, cells were harvested and stained with anti-SOX17 antibody as described above in preparation for flow cytometry analysis. The results of the flow cytometry analysis are shown in FIG. 1. Cells cultured with Activin A, Wnt3A, and a P13K inhibitor listed in Table 1 exhibited enhanced conversion into endoderm. hESC cells cultured with the selective P13K alpha inhibitor Compound A exhibited the most enhanced conversion into endoderm, with about 93.5-93.9% (e.g., 93.88%) of the hESC-derived cells expressing the SOX17 marker, which is superior over the other non-isoform selective PI3K inhibitors tested, including LY294002.

### Example 3

### Wnt3a was not Necessary for Differentiation to Endoderm

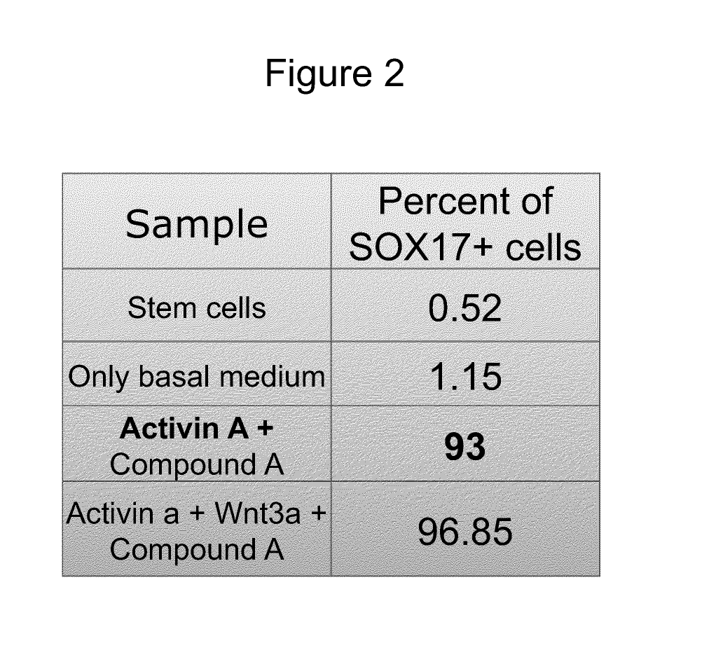

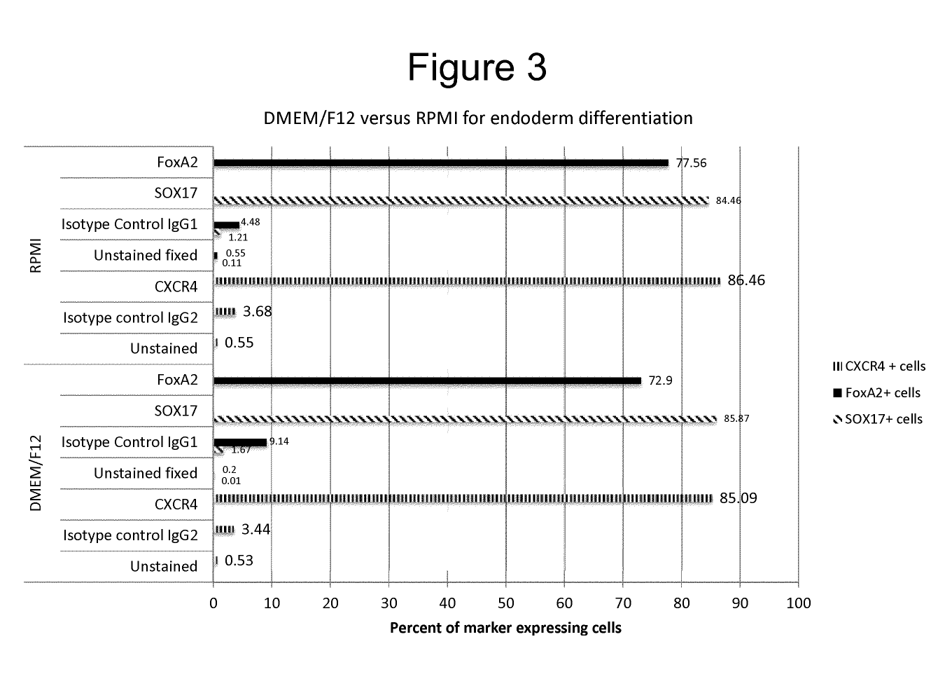

Experiments were performed to determine whether the growth factor Wnt3a was necessary for endoderm differentiation. hESC cell samples were prepared as described above and differentiated in basal medium; basal medium supplemented with Activin A, 50 μg/ml Wnt3a and Compound A, or basal medium with Activin A and 750 nM Compound A alone. After a 3-day treatment, cells were harvested and stained with anti-SOX17 antibody as described above in preparation for flow cytometry analysis. The results of the flow cytometry analysis are shown in FIG. 2. These results indicate that hESC-derived cells cultured with Compound A and Activin A in the absence of Wnt3a converted to endoderm with an efficiency that is slightly lower than, but comparable to that of cells cultured with Compound A, Activin A and Wnt3a. As shown in FIG. 3, this effect was independent of the basal medium used.

### Example 4

### Isoform-Specific PI3K Inhibitors

The effects of isoform-specific (e.g., isoform-selective) PI3K inhibitors on endoderm differentiation were compared. hESC cell samples were prepared as described above and differentiated in basal medium supplemented with the isoform-specific PI3K inhibitor and growth factors indicated below in Table 2.

TABLE 2

| PI3K Inhibitor | Tested with | Isotype |
|---|---|---|
| LY294002 | AW | General |
| Compound L | AW | delta |
| Compound M | AW | delta |
| Compound N | AW | delta |
| Compound R | AW | delta |
| Compound B | A | alpha |
| Compound C | A | alpha |
| Compound D | A | alpha |
| Compound E | A | alpha |
| Compound F | A | alpha |
| Compound G | A | alpha |
| Compound H | A | alpha |
| Compound I | A | alpha |
| Compound J | A | alpha/delta |
| Compound O | A | delta |
| Compound P | AW | delta |
| Compound Q | AW | delta |
| Compound S | A | gamma |
| Compound A | A | alpha/delta |
| Compound K | A | beta/delta |

AW=Activin A+Wnt3a
A=Activin A alone

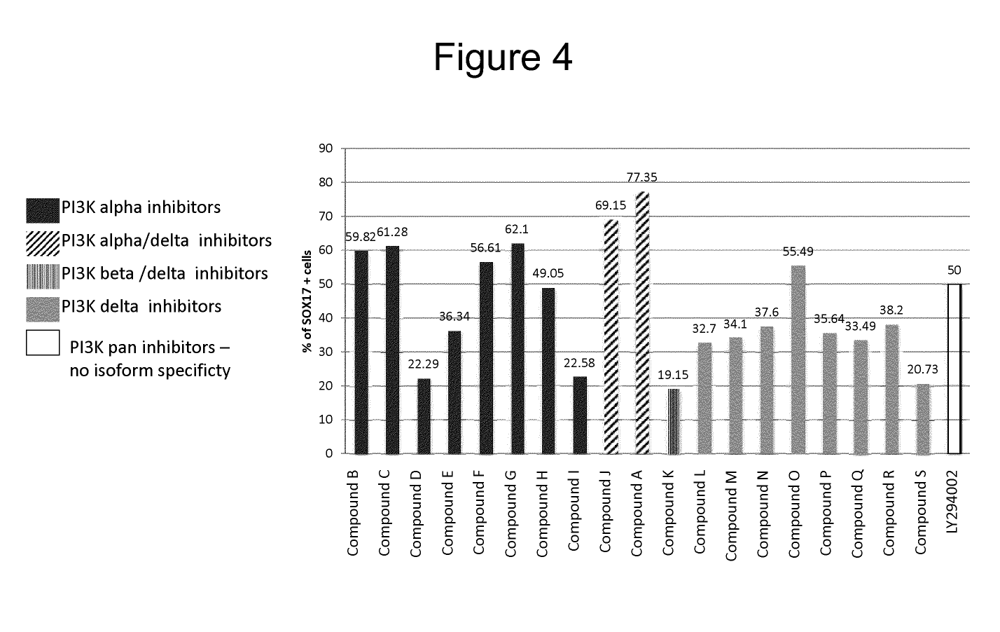

After a 3-day treatment, cells were harvested and stained with anti-SOX17 antibody as described above in preparation for flow cytometry analysis. The results of the analysis are shown in FIG. 4. These results indicate PI3K inhibitors that specifically affect the P13K alpha isoform or both the PI3K alpha and PI3K delta isoforms enhance endoderm differentiation more effectively than inhibitors of other PI3K isoforms. The inhibitors that exhibit the most pronounced effect on endoderm differentiation were Compound A and Compound J, which inhibit both the PI3K alpha and delta isoforms. About 69.15% of the hESC-derived cells cultured with Compound J and about 77.35% of the hESC-derived cells cultured with Compound A expressed the endoderm-specific marker SOX17.

These results were confirmed in knockdown experiments in which the expression of a specific PI3K isoform was inhibited using an siRNA. Briefly, during passage into basal medium, hESC were transfected with either 20 nM of a negative control siRNA, 20 nM of PI3K alpha specific siRNA (i.e., 10 nM s10520 and 10 nM s10521), 20 nM of PI3K beta specific siRNA (i.e., 10 nM s10524 and 10 nM s10525), 20 nM of a PI3K delta specific siRNA (i.e., 10 nM s10529 and 10 nM s10530), or 20 nM each of PI3K alpha, beta, and delta specific siRNAs (i.e., 10 nM of each of s10520, s10521, s10524, s10525, s10529, and s10530). The preceding siRNAs are commercially available from Life Technologies and noted in Table 3 below. The cells were incubated with the siRNAs for 20 hours. Following the incubation, the medium was changed and replaced with medium supplemented with 100 ng/ml Activin A. A control sample was prepared in which hESC cells were differentiated in basal medium with Activin A supplemented with 750 nM of the PI3K inhibitor Compound A.

TABLE 3

| Conditions | siRNA ID | Concentration |
|---|---|---|
| PI3K alpha | s10521 | 10 nM |
| | s10520 | 10 nM |
| PI3KD | S10530 | 10 nM |
| | S10529 | 10 nM |
| PI3KB | S10524 | 10 nM |
| | S10525 | 10 nM |
| PI3K ALPHA + PI3KB + PI3KD | s10521 | 10 nM |
| | s10520 | 10 nM |
| | S10530 | 10 nM |
| | S10529 | 10 nM |
| | S10524 | 10 nM |
| | S10525 | 10 nM |
| Negative control | Neg control #1 | 20 nM |

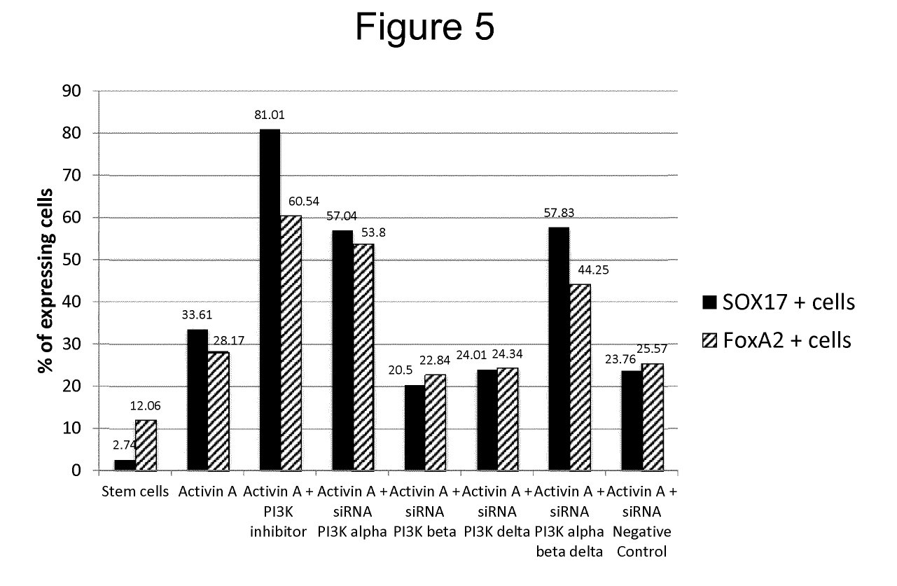

After a 3-day treatment, cells were harvested and stained with an anti-SOX17 or an anti-FoxA2 antibody as described above in preparation for flow cytometry analysis. The results of this analysis are shown in FIG. 5. hESC-derived cells cultured with a PI3K alpha-specific siRNA exhibited a high endoderm conversion rate, with 68% of the cells expressing SOX17 and 62% of the cells expressing FoxA2. In contrast hESC-derived cells cultured with a PI3K beta-specific siRNA, a PI3K delta-specific siRNA, or with both a PI3K beta-specific and PI3K delta-specific siRNAs exhibited a low endoderm conversion rate, with about 25% of the cells expressing SOX17 and ˜10% of the cells expressing FoxA2. siRNAs specific for the PI3K alpha isoform, but not for the PI3K beta isoform or the PI3K delta isoform, were found to increase endoderm conversion.

### Example 5

### Time Course for Endoderm Differentiation

A time course experiment was performed to determine the conversion efficiency and differentiation efficiency of hESC-derived cells cultured in basal medium supplemented with Activin A and Compound A. hESC cells were cultured as described above and differentiated in basal medium lacking Wnt3a and supplemented with Activin A and 750 nM Compound A. Six samples of cells were prepared. One sample of cells was harvested per day for six days starting 24 hours after treatment with Compound A+Activin A. The hESC-derived cell samples were stained with anti-SOX17, anti-FoxA2 or anti-CXCR4 antibody in preparation for flow cytometry analysis.

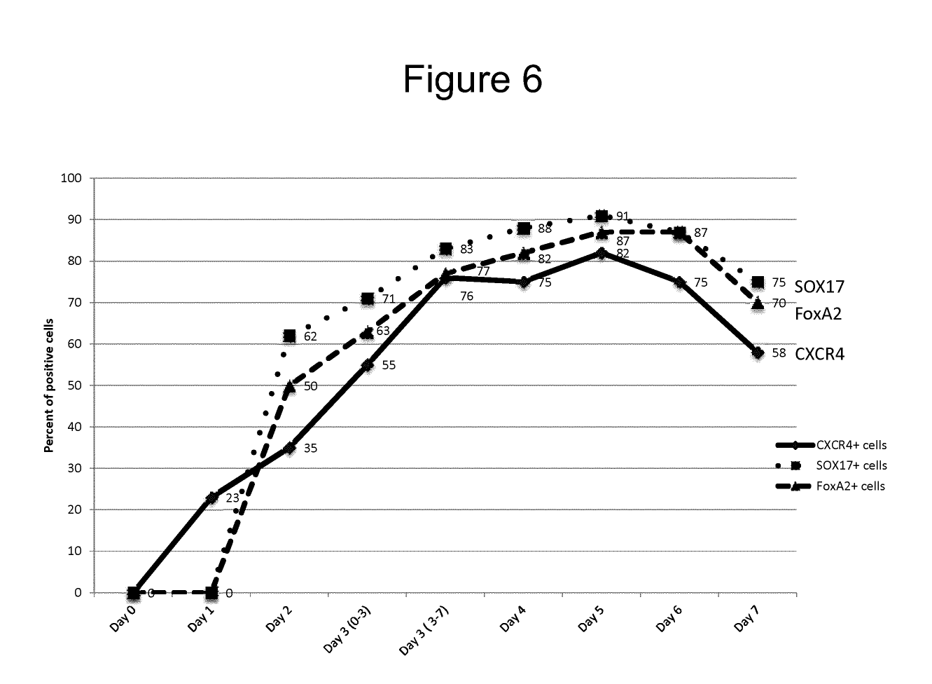

As shown in FIG. 6, the conversion efficiency was high on day 3 and begins to plateau. Differentiation efficiency was highest on day 5, when 91% of the hESC-derived cells express SOX17, 87% express FoxA2, and 82% express CXCR4. These results indicate that endoderm differentiation of hESC cells treated with Activin A+Compound A was time dependent.

### Example 6

### Dose Response

A dose response experiment was performed to determine the concentration of Compound A that most effectively enhances endoderm differentiation. hESC cells were cultured as described above and differentiated in basal medium lacking Wnt3a and supplemented with Activin A and either 0, 100 nM, 250 nM, 500 nM, 750 nM or 1000 nM Compound A. Undifferentiated human embryonic stem cells were maintained as described above. After a 3-day treatment, cells were harvested and stained with anti-SOX17 antibody as described above in preparation for flow cytometry analysis.

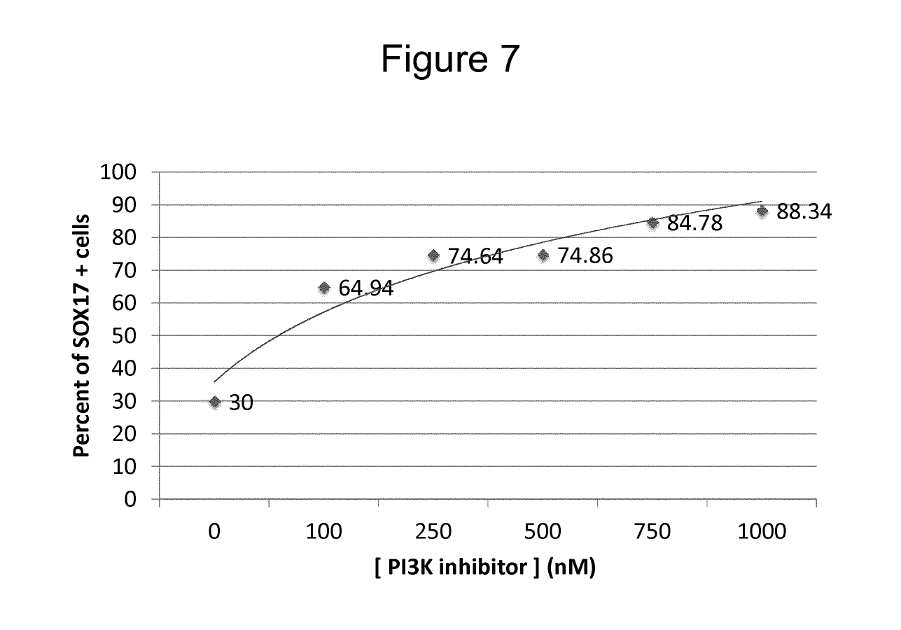

The results of the dose response experiment are depicted in FIG. 7. SOX17 expression increases with increasing concentrations of Compound A. hESC cells cultured in basal medium supplemented with Activin A and 750 nM Compound A exhibited the most enhanced endoderm differentiation, with 84% of the hESC-derived cells expressing SOX17. These results were confirmed in an imaging experiment in which the expression of SOX17 and FoxA2 were monitored.

An immunoassay (AlphaLISA®, Perkin Elmer) performed as described above using anti-SOX17 and anti-OCT4 antibodies confirmed that SOX17 expression on hESC-derived cells increases with increasing concentrations of Compound A up to 750 nM, while the expression of OCT4, a stem cell marker, decreases. This indicates that the endoderm differentiation coincides with a decrease in stem cell pluripotency.

### Example 7

### Viability and Proliferation

A time course was performed to monitor the viability and proliferation of endoderm cells obtained by treatment with Activin A and Compound A. A variety of culture conditions were tested. hESC cells were cultured as described above and differentiated in basal medium lacking Wnt3a and supplemented with Activin A and 750 nM Compound A; in TesR™2; in basal medium alone; or in basal medium supplemented with Activin A, Wnt3a, and 5 μM LY294002. The hESC-derived cells grown under each condition were assayed for their proliferation and viability once a day for 12 days with no medium change using Roche's xCELLigence System according to standard protocol.

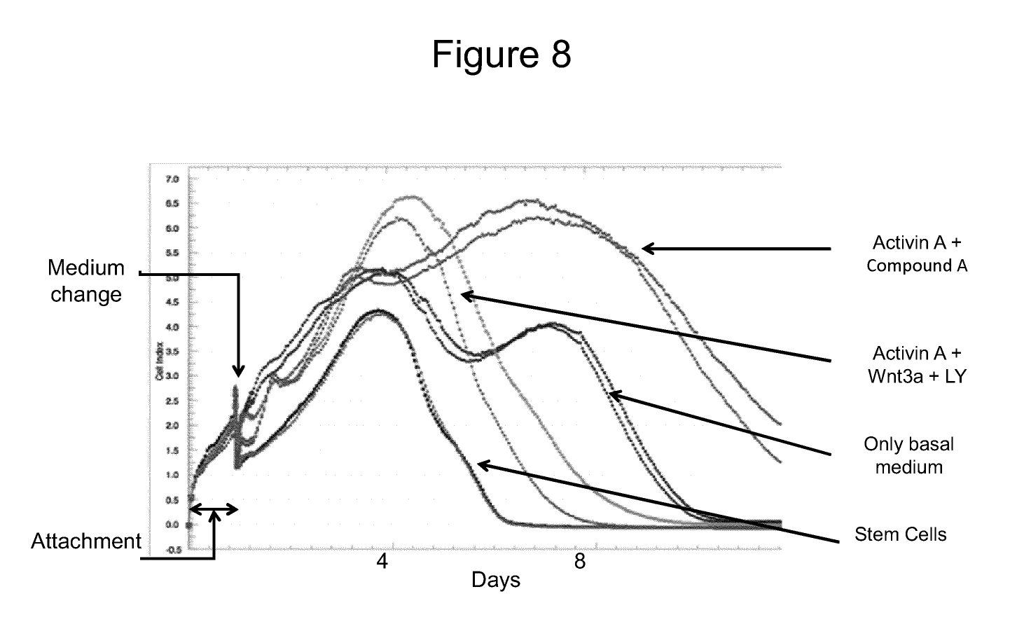

xCELLigence measured proliferation and viability as a function of impedance signal. High impedance signals indicated cell attachment to the surface of the culture dish, which is associated with increased proliferation. In contrast low impedance signals indicated cells' detachment from the surface of the culture dish, which is associated with cell death. As indicated in FIG. 8, endoderm cells obtained by treatment with Activin A and Compound A remain viable and proliferative past day 4. In contrast, stem cells and endoderm cells obtained by treatment with Activin A, Wnt3a, and LY294002 begin to exhibit cell death on Day 4. The experiments whose results are depicted in FIG. 8 were performed in duplicate. Accordingly, there are two curves for each condition.

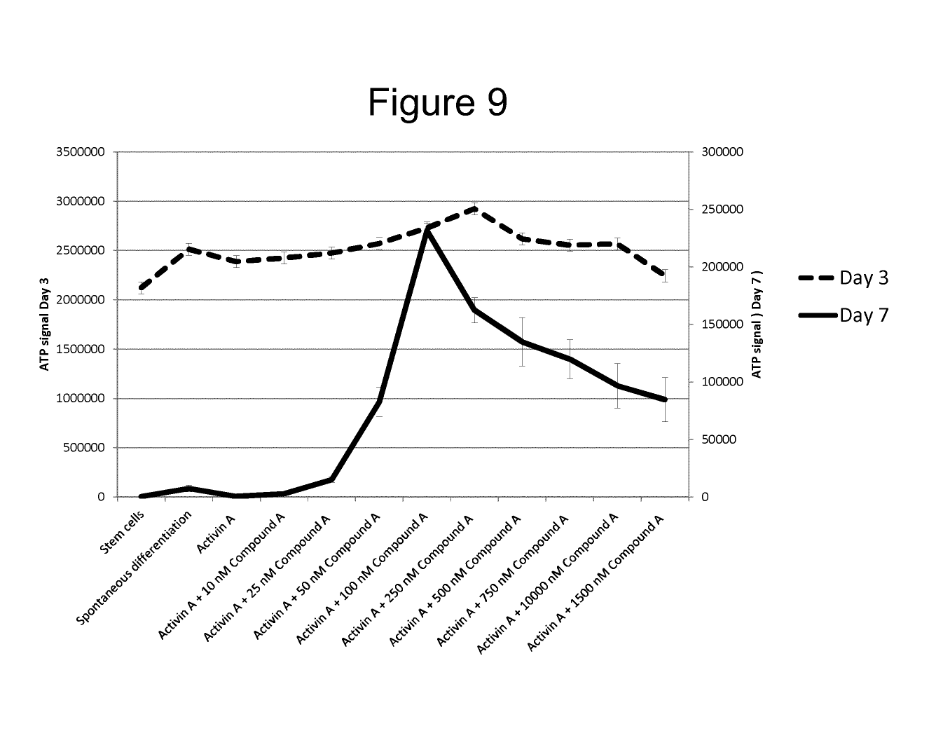

These results were confirmed using the CellTiter-Glo Luminescent Cell Viability Assay (Promega, #G7571) according to standard protocol and analyzed using an EnVision® Multilabel Reader from Perkin Elmer. In this assay, metabolically active cells in each sample tested were quantified as a function of ATP levels produced by the sample. Briefly, stem cells, endoderm cells obtained by spontaneous differentiation, endoderm cells obtained by Activin A treatment, and endoderm cells obtained by treatment with Activin and either 10 nM, 25 nM, 50 nM, 100 nM, 250 nM, 500 nM, 750 nM, 1 μM, or 1.5 μM Compound A were tested using the CellTiter-Glo Luminescent Cell Viability Assay 3 days and 7 days following the beginning of treatment. As indicated in FIG. 9, endoderm cells obtained by treatment with Activin A and 100 nM, 250, 500, 750, 1 μM, or 1.5 μM Compound A exhibited greater viability at 7 days than cells grown under the other conditions.

### Example 8

### Stable Endoderm

The production of phenotypically stable and expandable (i.e., proliferative) endoderm has been previously attempted with human cells (Seguin, et al. (2008) “Establishment of endoderm progenitors by SOX transcription factor expression in human embryonic stem cells.” Cell Stem Cell, 3(2): 182-19; Cheng, et al. (2012). “Self-renewing endodermal progenitor lines generated from human pluripotent stem cells.” Cell Stem Cell, 10(4): 371-384) and mouse cells (Morrison, et al. (2008). “Anterior definitive endoderm from ESCs reveals a role for FGF signaling.” Cell Stem Cell, 3(4): 402-415). Certain endoderm differentiation protocols include a costly and labor-intensive sorting step in order to obtain CXCR4⁺ cells. Different strategies (e.g., using different reporter lines and different growth factors) have been used to develop stable endoderm, but these strategies have not led to reproducible results.

The time course experiments in Example 5 above show that the expression of endodermal markers was maintained over six days when hESC-derived cells were cultured in basal medium supplemented with Activin A and Compound A (FIG. 6). Based on these data, further experiments were performed to determine whether the stability of this endoderm population could be extended further than six days notably over passages.

The proliferation and maintenance of AA and AP cells were compared:

AA cells: Stem cells→(Activin A)→Endoderm

AP cells: Stem cells→(Activin A+Compound A)→Endoderm

As depicted in the flowcharts above, hESC cells were cultured as described in Example 5 and differentiated in basal medium lacking Wnt3a and supplemented with Activin A alone (AA cells) or Activin A and 750 nM Compound A (AP cells).

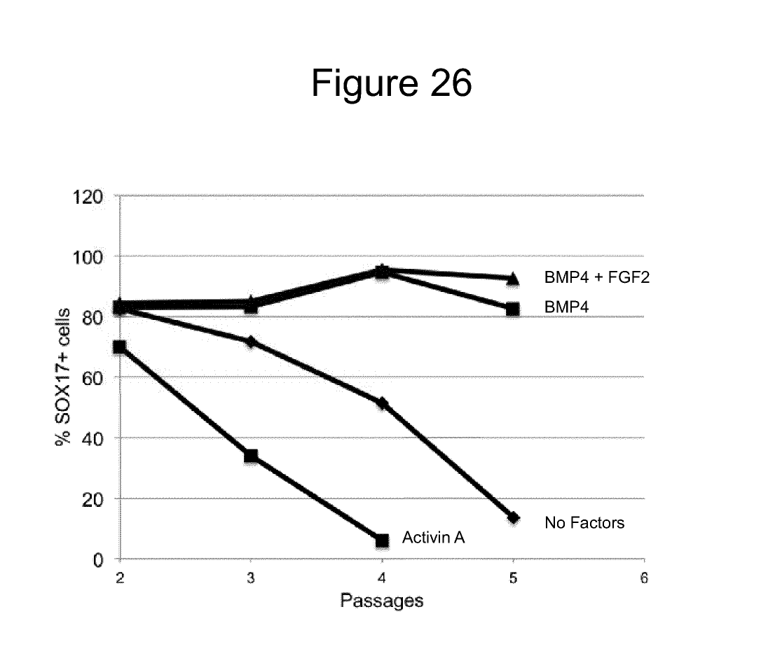

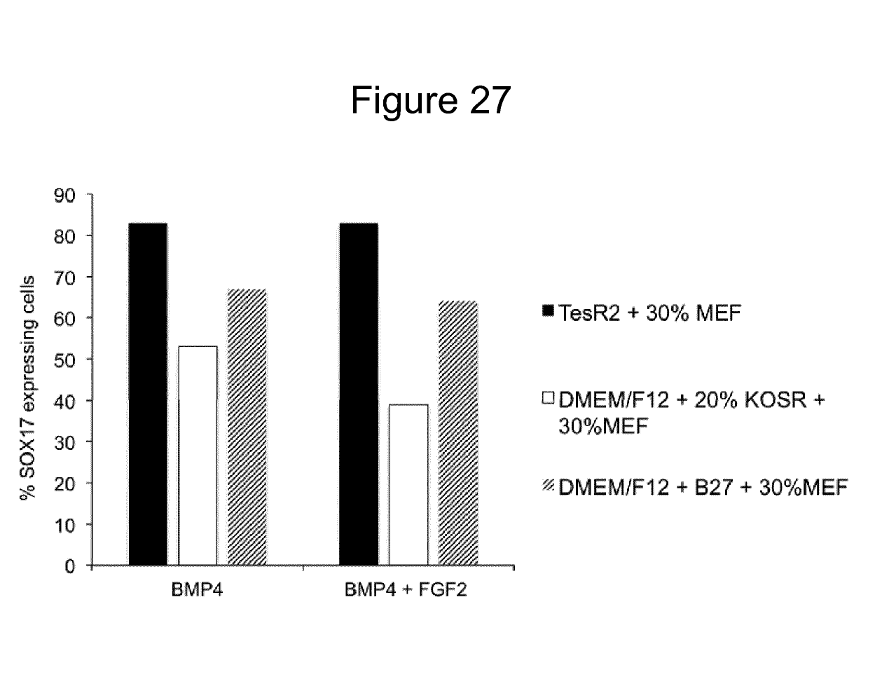

On Day 3, AP cells were directly passaged into matrigel- or collagen-coated flasks without being sorted. AP cells were maintained in a cocktail of four growth factors, BMP4, FGF2, VEGF and EGF, based on previous work described in Cheng et al. (2012). “Self-renewing endodermal progenitor lines generated from human pluripotent stem cells.” Cell Stem Cell, 10(4), 371-384. BMP4 was necessary to maintain SOX17 expression. Without BMP4, SOX17 expression quickly decreased and was lost at passage 4 or 5 (see FIG. 26). FGF2, VEGF and EGF were also found to be important for endoderm proliferation. Without these factors, AP cells stopped proliferating at passage 4. The choice of basal medium was also critical. Since no feeder cell layers were used in our system, 30% MEF conditioned medium was added to improve proliferation. As shown in FIG. 27, maintaining the AP endoderm population with these factors in TesR2 medium supplemented with 30% mouse embryonic fibroblast (MEF) conditioned medium produced the highest level of SOX17-expressing cells at Day 3. The data in FIG. 27 was obtained from endoderm cells that were passaged twice.

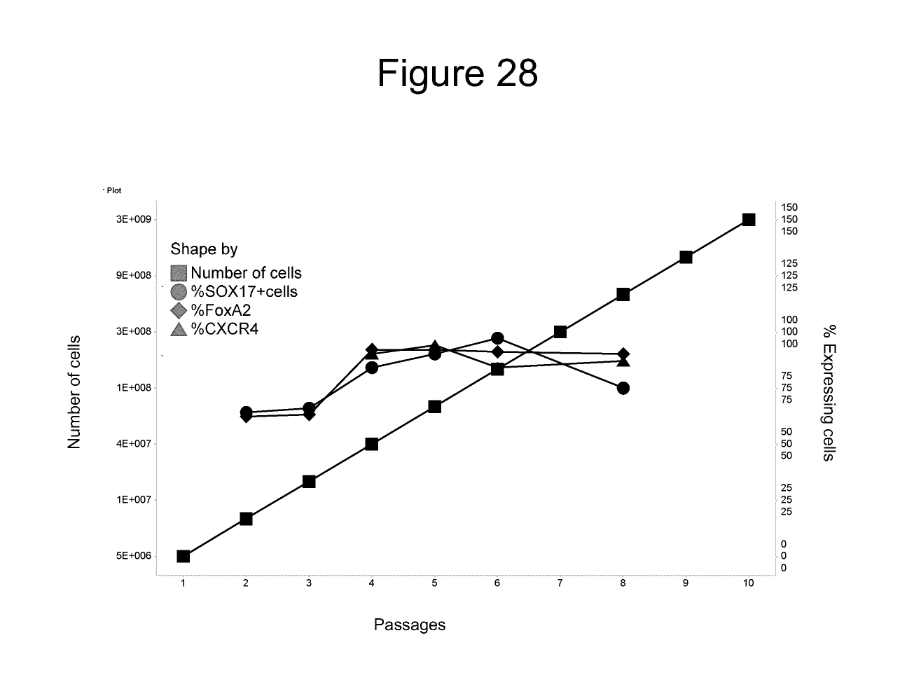

AP cells were highly proliferative over 10 passages with 3.5 days doubling time under these optimized conditions (see FIG. 28). AA cells (i.e., hESC-derived stem cells differentiated with Activin A only) could also be maintained with the same protocol. However, only a small portion of the AA population (around 20%) was positive for CXCR4 and FoxA2, and the AA cells stopped proliferating after 4 passages. Addition of Compound A to basal medium+Activin A during the first 3 days of hESC-derived stem cell differentiation permitted the maintenance (i.e., phenotypic maintenance) of an almost pure population of endodermal cells that remained proliferative for over 10 passages without any sorting steps. When Compound A was added to the basal medium, the population of AP cells presented over 70% cells positive for Sox17 and FoxA2 the first 3 passages. After passage 4, the AP population remained almost pure with 80-90% of the cells expressing SOX17, CXCR4 and FoxA2. The choice of basal medium was also critical in this context, as well. Cells grown in DMEM/F12+20% KOSR+30% MEF did not proliferate after being passaged two times.

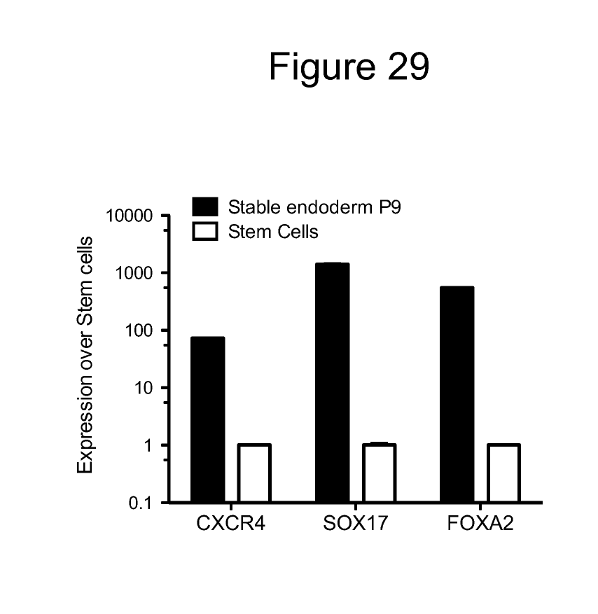

Expression of SOX17, CXCR4 and FoxA2 was monitored by flow cytometry and confirmed via immunofluorescence and gene expression (see FIG. 29). Immunofluorescence experiments confirmed that SOX17 was expressed by AP cells at passage 12. Additional immunofluorescence experiments performed to monitor AFP expression indicated that AP endoderm cells showed no signs of differentiation into hepatocyte-like cells at passage 12. These data show that the AP population of cells, derived from stem cells cultured in basal medium, Activin A and Compound A, was stable as a homogeneous and proliferative endoderm population over ten passages without any sorting steps and without using feeder cell layers.

### Example 9

### Production of Endoderm by Akt Inhibition or mTOR Inhibition

PI3K inhibitors generally inhibit signaling mediated by Akt kinase and mTOR. hESC cells were cultured in basal medium supplemented with one of a variety of commercially available Akt or mTOR inhibitors listed in Table 4 below to investigate whether direct inhibition of the Akt or mTOR pathway would result in effective endoderm production. hESC cells were cultured as described above and differentiated in basal medium lacking Wnt3a and supplemented with Activin A and 750 nM of one of the inhibitors listed in Table 4. After a 3-day treatment, cells were harvested and stained with anti-SOX17 antibody as described above in preparation for AlphaLISA analysis.

TABLE 4

| Target | Name |
|---|---|
| mTOR | everolimus |
| mTOR | Pimecrolimus |
| mTOR | Rapamycin |
| mTOR | Temsirolimus |
| AKT | Enzastaurin, Free Base |
| AKT | PKC412 |
| mTOR | AZD 8055 |
| PI3K beta | TGX 221 |
| AKT | GSK 690693 |
| mTOR | PP242 |

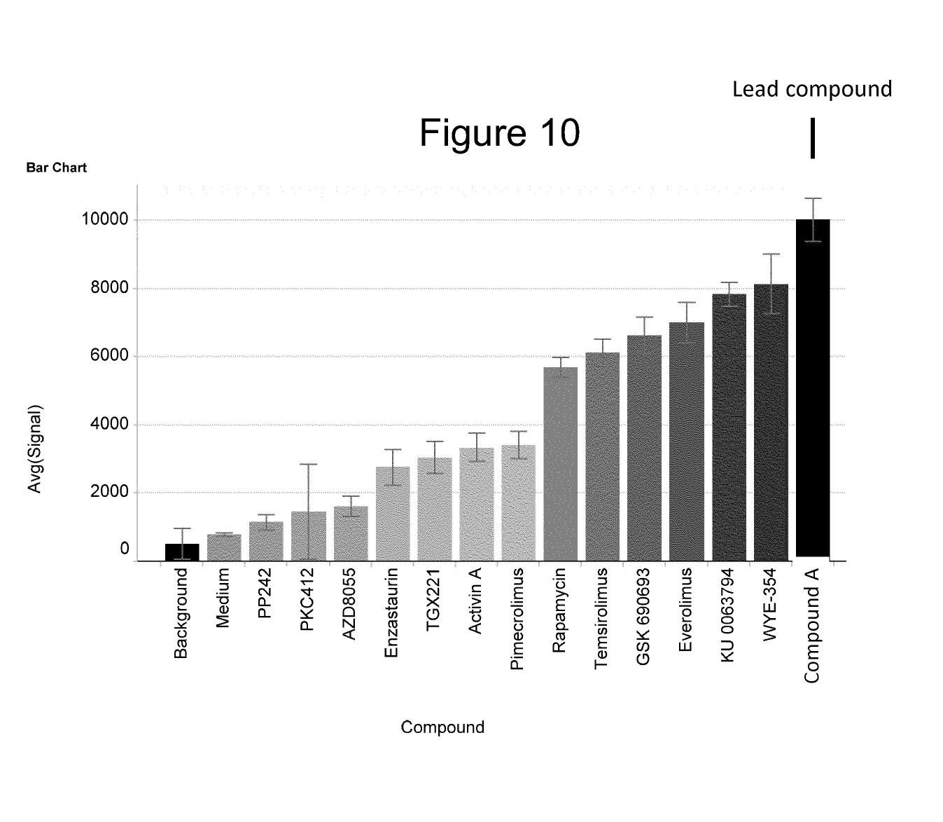

The results of the analysis are shown in FIG. 10. hESC cells treated with the mTOR inhibitors everolimus, KU0063794, or WYE-354 showed better endoderm conversion than cells cultured in Activin A alone or cells cultured with an Akt inhibitor. For example, endoderm conversion in cells treated with everolimus, KU0063794, or WYE-354 was more efficient than endoderm conversion in cells treated with the Akt inhibitor GSK690693.

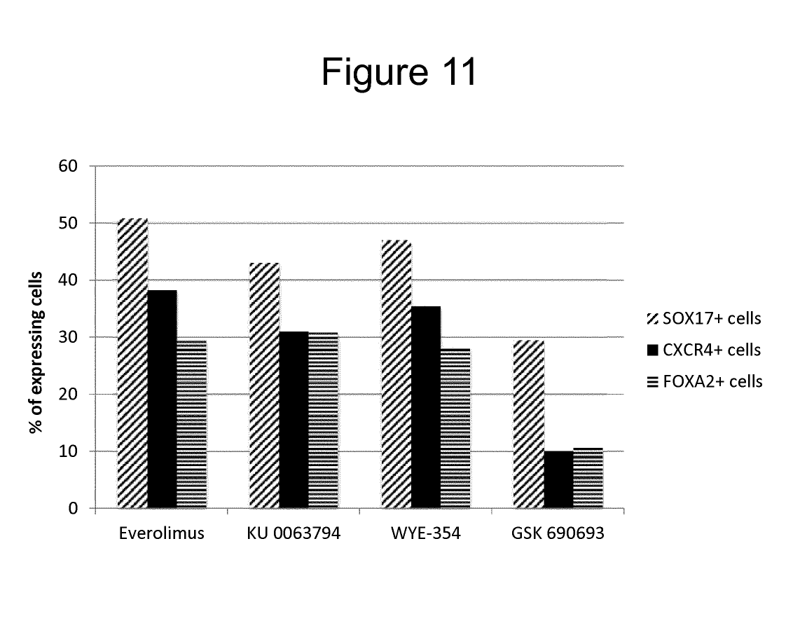

These results were repeated in flow cytometry experiments. hESC cells were cultured as described above and differentiated in basal medium lacking Wnt3a and supplemented with Activin A and 750 nM of everolimus, KU00633794, WTE-354, or GSK690639. After a 3-day treatment, cells were harvested and stained with anti-SOX17 antibody, anti-FoxA2 antibody, or anti-CXCR4 antibody in preparation for flow cytometry analysis. The results of this analysis are shown in FIG. 11. hESC cells treated with everolimus, KU00633794, WTE-354, or GSK69063 exhibit a higher degree of endoderm conversion. In contrast, only 20% of hESC-derived cells cultured in Activin A alone express SOX17.

These results were confirmed in knockdown experiments using siRNAs specific for Ak1, Akt2, Akt3, or mTOR. The knockdown experiments were performed as described above using AKT- or mTOR-specific siRNAs. These siRNAs are commercially available from Life Technologies and noted in Table 5 below. The cells were incubated with the siRNAs for 20 hours. Following the incubation, the medium was changed and replaced with medium supplemented with Activin A alone. A control sample was prepared in which hESC cells were differentiated in basal medium with Activin A supplemented with 750 nM of PI3K inhibitor Compound A.

TABLE 5

| siRNA ID | Gene Symbol |
|---|---|
| s659 | AKT1 |
| s660 | AKT1 |
| s1216 | AKT2 |
| s1217 | AKT2 |
| s19429 | AKT3 |
| s19427 | AKT3 |
| s603 | MTOR |
| s604 | MTOR |

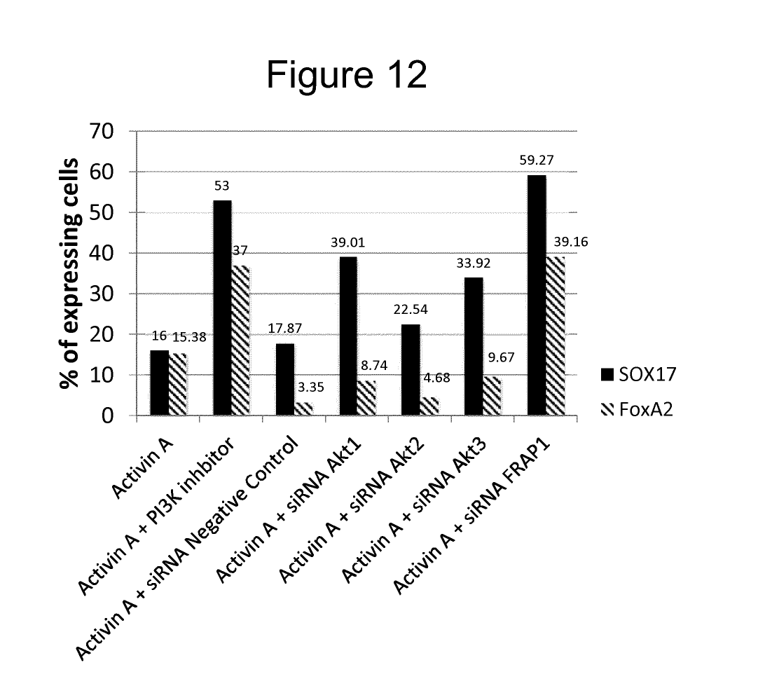

As shown in FIG. 12, inhibition of mTOR expression increases endoderm conversion (with about 61% of the hESC-derived cells expressing SOX17 and about 40% of the cells expressing FoxA2) as well as the inhibition of PI3K alpha expression (with about 57% of the hESC-derived cells expressing SOX17 and about 38% of the cells expressing FoxA2). The inhibition of Akt 1, Akt2, or Akt3 expression does not increase the endoderm conversion as significantly as the inhibition of mTOR.

### Example 10

### Additive or Synergistic Effects of PI3K Alpha and mTOR Inhibition

A knockdown experiment was performed to determine whether the simultaneous knockdown of PI3K alpha and mTOR expression had an additive or synergistic effect on endoderm differentiation. During passage into basal medium, the cells were transfected with either 20 nM of a negative control siRNA, 20 nM of a PI3K alpha specific siRNA, 20 nM of an mTOR specific siRNA, or 20 nM each of a PI3K alpha specific siRNA and an mTOR specific siRNA. The cells were incubated with the siRNAs for 20 hours. Following the incubation, the medium was changed and replaced with basal medium supplemented with Activin A and 750 nM of PI3K inhibitor Compound A or with basal medium supplemented with Activin A alone. After three days, the cell samples were harvested and stained with anti-SOX17 antibody or anti-FoxA2 antibody in preparation for flow cytometry analysis.

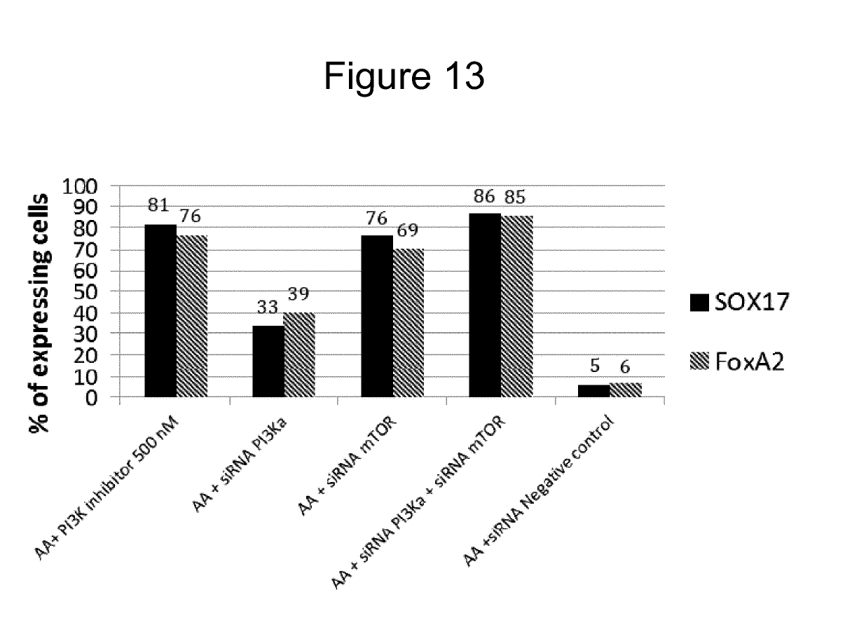

The results of the analysis are depicted in FIG. 13. The simultaneous knockdown of PI3K alpha expression and mTOR expression promotes higher levels of endoderm conversion (with 86% of the hESC-derived cells expression SOX17 and 85% of the cell expressing FoxA2) than the knockdown of either PIK alpha expression alone (with 33% of the hESC-derived cells expression SOX17 and 39% of the cell expressing FoxA2) or mTOR expression alone (with 76% of the hESC-derived cells expression SOX17 and 69% of the cell expressing FoxA2). The endoderm conversion rate in the absence of PI3K alpha and mTOR expression was comparable to that of the PI3K alpha inhibitor.

### Example 11

### Monitoring the Effects of Combinations of Varying Concentrations of mTOR Inhibitor and PI3K Alpha Inhibitor on the Expression of Mesendoderm, Endoderm, and Mesoderm Marker Genes

As described above, the combined effects of mTOR inhibition and PI3K inhibition promoted higher levels of endoderm conversion relative to either mTOR inhibition alone or PI3K alpha inhibition alone. 4×4 dose response matrix experiments were then performed using varying concentrations of mTOR siRNA (0, 0.2 nM, 2 nM, and 20 nM) and a varying concentrations of PI3K alpha siRNA (0, 0.2 nM, 2 nM, and 20 nM) to evaluate the combined effects of mTOR inhibition and PI3K inhibition on the expression of individual endoderm marker genes. As described above, stem cell differentiation was carried out in presence of Activin A, and the expression of mesendoderm marker genes DKK1, EOMOES, FGF17, FGF8, GATA6, MIXL1, Brachyury (T), WNT3a, GSC, LHX1, and TBX6 and endoderm marker genes CDH2, CER1, CXCR4, FGF17, FoxA2, GATA4, GATA6, HHEx, HNF1B, KIT, SOX17, and TDGF1 were analyzed on Day 1 and Day 2.

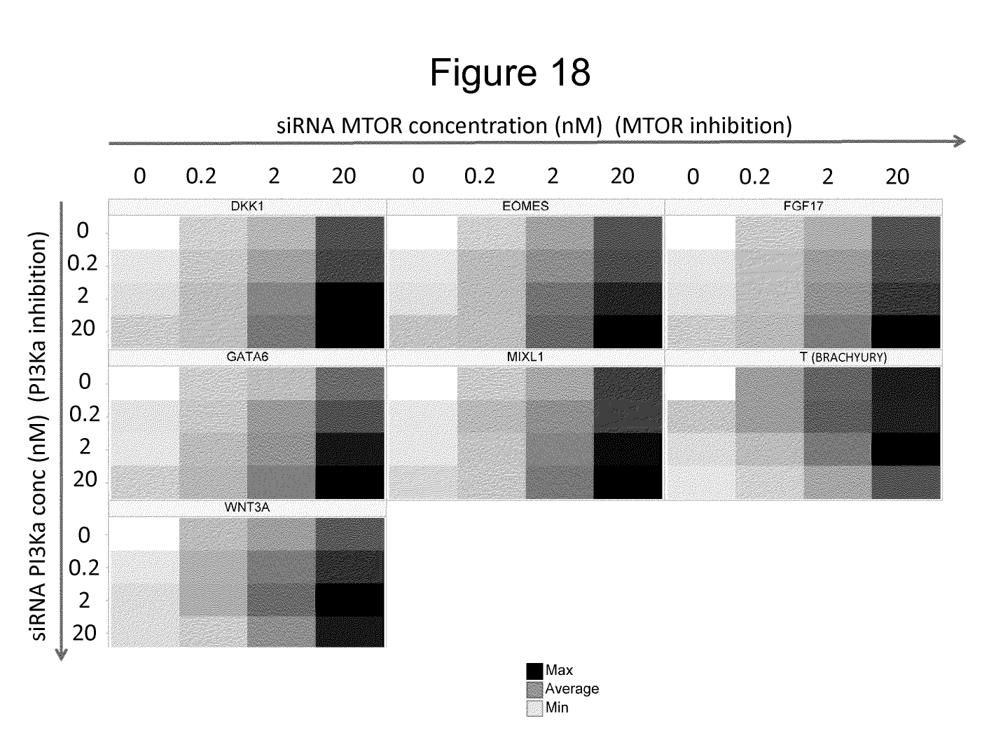

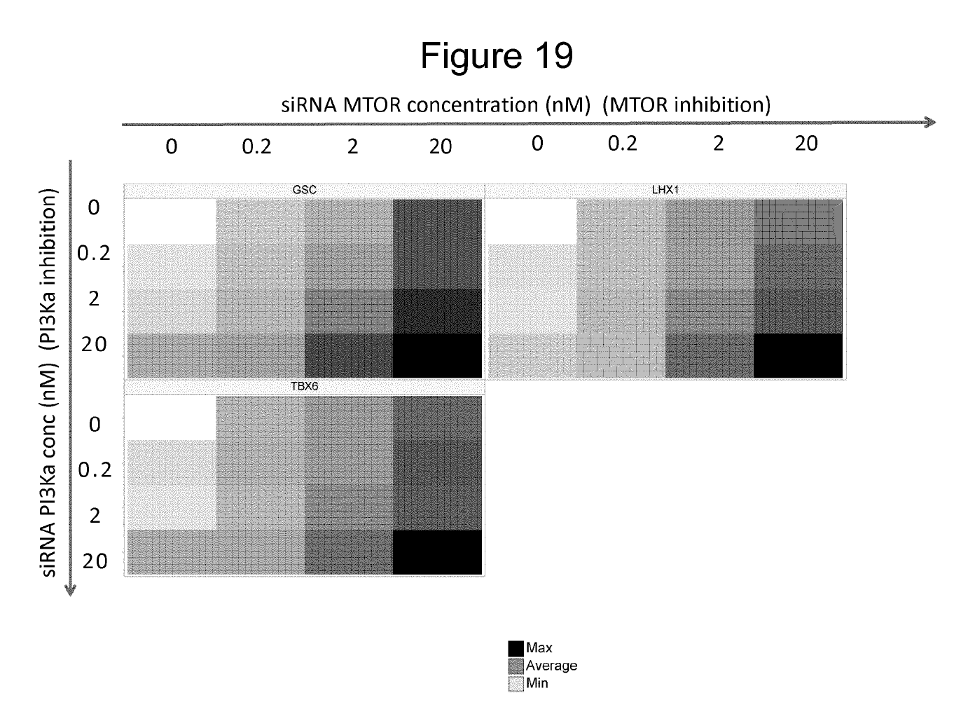

At Day 1, most mesendoderm genes were clearly upregulated when higher concentrations of mTOR siRNA were used, confirming the predominant role of mTOR in mesendoderm formation (FIGS. 18 and 19). The effects of PI3K alpha inhibition on marker gene expression varied depending on the marker gene analyzed. For some markers like DKK1, FGF17, MIXL1, mTOR inhibition had a strong effect on expression even without PI3K alpha inhibition. For other markers like LHX1, GATA6, EOMES, GSC and TBX6, PI3K alpha inhibition was necessary to reach the maximal expression (FIGS. 18 and 19).

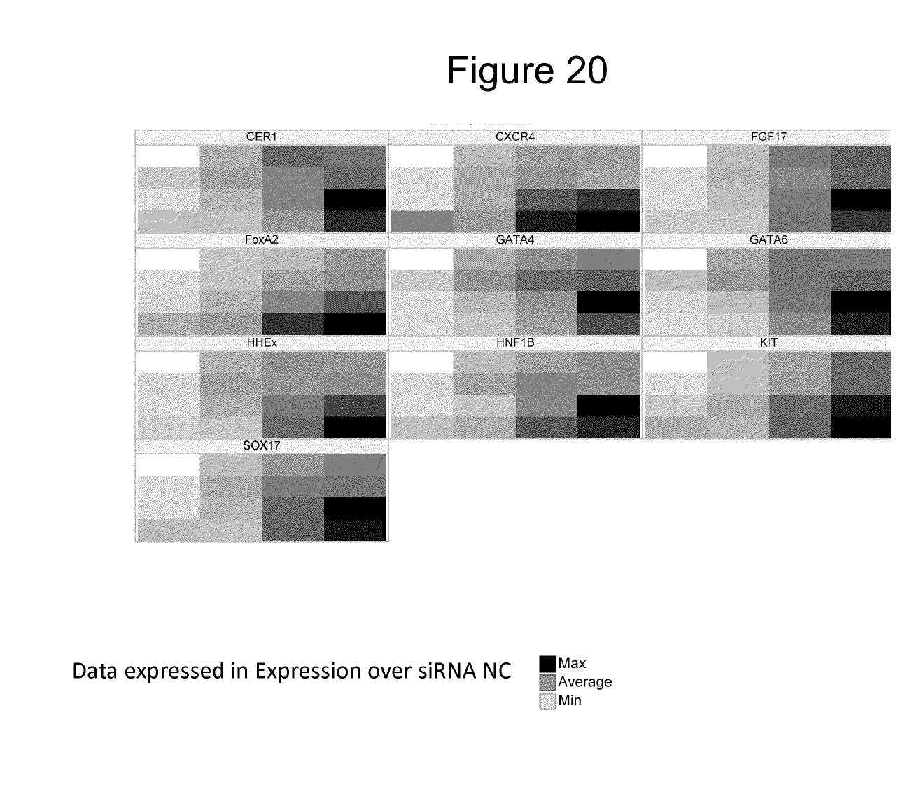

At Day 2, most of endodermal markers required both PI3K alpha and mTOR inhibition to reach their highest expression levels (FIG. 20). For some markers, e.g., FoxA2, there was an equivalent contribution of mTOR and PI3K alpha inhibition to expression level. For other markers, e.g., CER1, Hhex and FGF17, PI3K alpha inhibition strongly up-regulated endoderm gene expression, but only when mTOR inhibition had already elevated gene expression to a certain level. For the marker CXCR4, mTOR alone was not sufficient to up regulate its expression, and PI3K alpha inhibition was required.

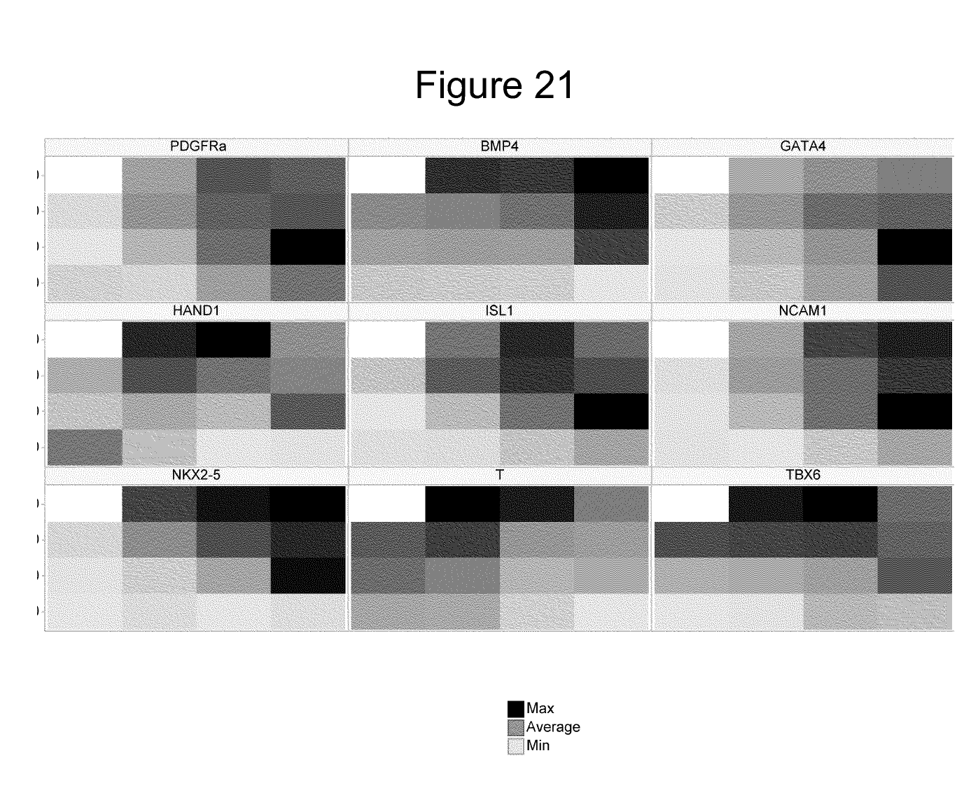

4×4 dose response matrix experiments were performed as described above to analyze the effects of different degrees of mTOR inhibition and PI3K alpha inhibition on the expression of mesoderm marker genes PDGFRa, BMP4, GATA4, HAND1, ISL1, NCAM1, NKX2-5, TBX6, and T (Brachyury) were analyzed (FIG. 21). PI3K alpha inhibition had a unique effect on mesoderm markers (FIG. 21). Even low concentrations of PI3K alpha siRNA prevented high expression of mesoderm markers ISL1, NKX2-5 and ectoderm marker NCAM1 that are typically caused by mTOR inhibition. Increased concentrations of mTOR siRNA correlated with increased expression of mesoderm marker genes. Moreover, increased concentrations of PI3K alpha siRNA were necessary to counteract this effect of mTOR inhibition. For the key mesoderm marker BMP4, only high PI3K alpha inhibition prevented its up regulation. Interestingly, to down regulate the mesoderm marker Brachyury, both mTOR and PI3K alpha inhibition were needed.

The dose matrix experiments confirmed distinct roles of MTOR inhibition and PI3K alpha inhibition in the expression of mesendoderm, endoderm, and mesoderm marker genes. Moreover, different levels of mTOR inhibition and PI3K alpha inhibition had specific effects on the expression of marker genes. For mesendoderm formation, mTOR inhibition is crucial. At this stage, high PI3K alpha inhibition contribution lies in enhancing mTOR inhibition effect, but PI3K alpha inhibition can also be an important contributor for markers less affected by mTOR inhibition (e.g., LHX1). For further differentiation of mesendoderm into endoderm, both PI3K alpha and mTOR inhibition are required to get the highest expression of endoderm marker genes. PI3K alpha inhibition is essential at this stage to prevent other lineages, especially mesoderm, from being formed.

### Example 12

### Characterizing Small Molecule Inhibitors that Promote Endoderm Differentiation

The siRNA dose response matrix experiments described above were performed to identify the important targets, PI3K alpha and mTOR, whose inhibition is necessary for endoderm formation and to investigate the distinct roles of mTOR inhibition and PI3K alpha inhibition in the expression of mesendoderm, endoderm, and mesoderm marker genes. Accordingly, small molecule inhibitors were screened for their abilities to promote endoderm formation. However, different small molecules for a specific target may still have different potencies and isoform specificities. Moreover, such compounds also often have off target effects that may affect the differentiation, and the compounds may be toxic to the cells at high concentrations. Experiments were performed to identify compounds that provide the optimal balance of PI3K alpha and mTOR inhibition (e.g., as identified in the 4×4 dose response matrix experiments) for promoting endoderm differentiation.

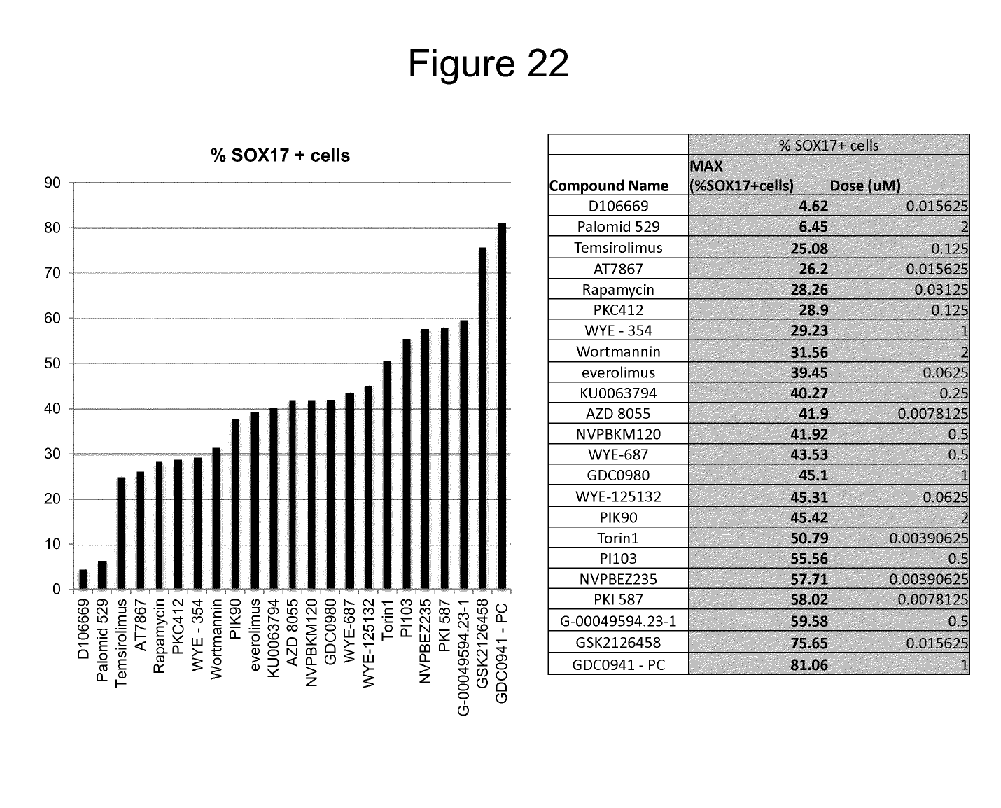

To facilitate extended characterization, it was necessary to determine the optimal concentration of each of the compounds for use subsequent experiments. The optimal concentration of each compound was determined based on two parameters: highest efficiency of endoderm differentiation and low toxicity. Each compound was tested with Activin A in a dose response manner. The concentration that gave the highest % SOX17 expressing cells at Day 3 without causing more than 30% cell death compared to control was determined for each compound. The results of this analysis are shown in FIG. 22. The yield of differentiation went from 4% to 81% SOX17+ cells at day 3.

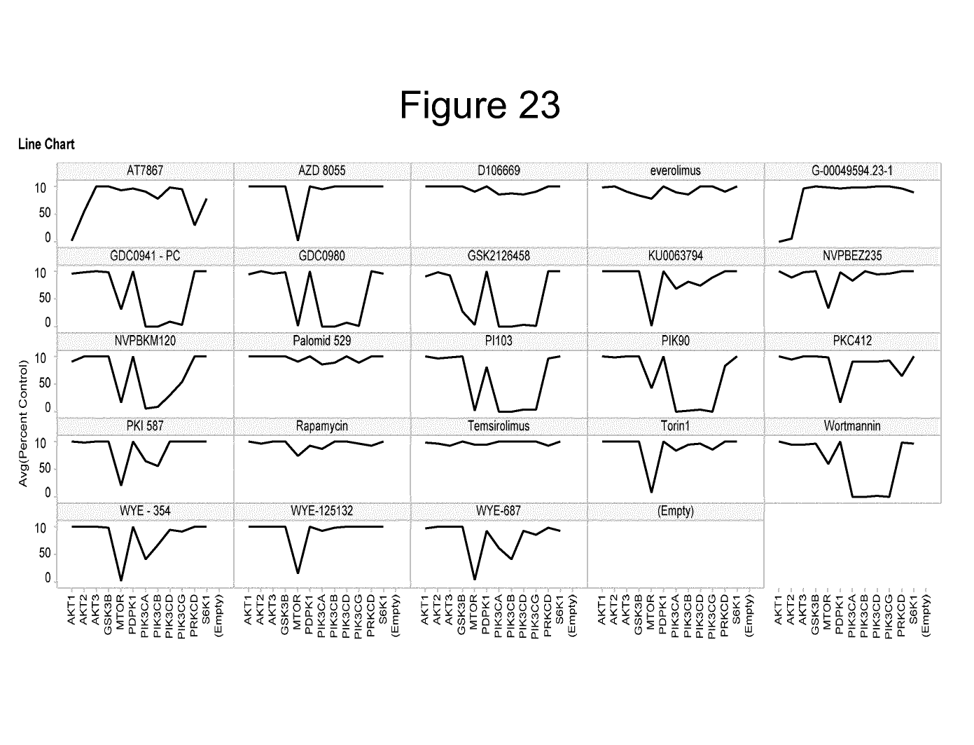

These compounds were further characterized regarding their effects on endoderm formation and on PI3K/AKT/MTOR pathway, and these results were compared to the findings from the dose matrix experiments described above. The effect of each compound on the PI3K/AKT/MTOR pathway was evaluated in two ways: via a kinase profile (FIG. 23) and via phospho imaging assays (i.e., immunofluorescence assays using antibodies specific for the phosphorylated form of mTOR or AKT). In vitro kinase profiling provided percent inhibition for numerous targets within the PI3K cell signaling pathway, and cell-based imaging assays were performed to directly visualize the effect of each compound on phosphorylated Akt and phosphorylated mTOR in the cell system used for differentiation. As shown in FIG. 23, D1066, PKC, and Palomid 529 did not show reduction of phosphorylation of mTOR or Akt. In addition, these compounds did not exhibit any effects on Akt or mTOR phosphorylation in the cell-based imaging assays. PKC412 exhibited potent reduction of phosphorylation, but may be toxic. Based on these assays, the compounds were grouped into 4 categories: AKT inhibitors, MTORC1 inhibitors, MTORC1/2 inhibitors, and dual PI3K/MTOR inhibitors (Table 6 below).

TABLE 6

| | | Avg(Avg (Ph | Max(Avg (Ph | |
|---|---|---|---|---|
| | PI3K alpha_MTOR | MTOR_60m_%C | AKT_60min_In- | |
| Compound ID | score | # Spots)) | tensity)) | GROUP |
| AT7867 | PI3K alpha−_MTOR− | 73.59 | 122.15 | AKT |
| AZD 8055 | PI3K alpha−_MTOR+++ | 15.8 | 89.81 | MTORC1/2 |
| everolimus | PI3K alpha+_MTOR+ | 36.46 | 99.76 | MTORC1 |
| G-00049594.23-1 | PI3K alpha−_MTOR− | 62.47 | 114.75 | AKT |
| GDC0941 - PC | PI3K alpha+++_MTOR++ | 17.71 | 85.7 | Dual PI3K/MTOR |
| GDC0980 | PI3K alpha+++_MTOR+++ | 9.99 | 87.61 | Dual PI3K/MTOR |
| GSK2126458 | PI3K alpha+++_MTOR+++ | 15.55 | 86.8 | Dual PI3K/MTOR |
| KU0063794 | PI3K alpha+_MTOR+++ | 25.83 | 91.25 | MTORC1/2 |
| NVPBEZ235 | PI3K alpha+_MTOR++ | 43.44 | 88.3 | Dual PI3K/MTOR |
| NVPBKM120 | PI3K alpha+++_MTOR+++ | 60.28 | 91.48 | Dual PI3K/MTOR |
| PI103 | PI3K alpha+++_MTOR+++ | 5.59 | 83.58 | Dual PI3K/MTOR |
| PIK90 | PI3K alpha+++_MTOR++ | 42.17 | 86.72 | Dual PI3K/MTOR |
| PKI 587 | PI3K alpha+_MTOR+++ | 77.99 | 91.07 | Dual PI3K/MTOR |
| Rapamycin | PI3K alpha+_MTOR+ | 57.77 | 99.75 | MTORC1 |
| Temsirolimus | PI3K alpha−_MTOR− | 42.12 | 95.21 | MTORC1 |
| Torin1 | PI3K alpha+_MTOR+++ | 66.6 | 89.48 | MTORC1/2 |
| Wortmannin | PI3K alpha+++_MTOR+ | 10.47 | 89.77 | PI3K |
| WYE - 354 | PI3K alpha++_MTOR+++ | 10.49 | 89.35 | Dual PI3K/MTOR |
| WYE-125132 | PI3K alpha−_MTOR+++ | 17.55 | 86.7 | MTORC1/2 |
| WYE-687 | PI3K alpha+_MTOR+++ | 12.81 | 86.65 | MTORC1/2 |

In column 2 of Table 6, the PI3K alpha_MTOR score reflects the ability of a compound to inhibit the phosphorylation of PI3K alpha and mTOR, where + indicates minimal inhibition, and +++ indicates maximal inhibition. In column 3 of Table 6, the score indicates the ratio of phosphorylated mTOR (as measured by fluorescence intensity) in cells treated with a compound and the phosphorylated mTOR (as measured by fluorescence intensity) in cells that were not treated with the compound. In column 4 of Table 6, the score indicates the ratio of phosphorylated AKT (as measured by fluorescence intensity) in cells treated with a compound and the phosphorylated AKT (as measured by fluorescence intensity) in cells that were not treated with the compound. A reported effect of AKT inhibitors is an increase in phosphorylated AKT.

The effect of each compound on endoderm differentiation was evaluated by ranking the expression of numerous relevant lineage markers. Each compound was given a score for its effect on single marker expression relative to other compounds tested giving rise to an overall score for mesendoderm, endoderm and mesoderm formation. Higher scores indicated that the marker genes from a specific lineage (e.g., mesendoderm, endoderm, or mesoderm) were highly expressed. The score for each compound was determined as follows: marker gene expression was compared between cells grown in the presence of a particular compound+Activin A and cells grown in Activin A alone. If the ratio of expression levels was <1, then that marker gene was given a score of 0. If the ratio of expression levels was between 1 and the median expression level of all compounds, then that marker gene was given a score of 1. If the ratio of expression levels was between the median expression level and 70% of the maximum expression level of all of the compounds, then the marker gene was given a score of 2. If the ratio of expression levels was between 70% of the maximum expression level of all of the compounds and the maximum expression level of all of the compounds, then the marker gene was given a score of 3. The endoderm marker genes monitored were CER1 CXCR4 FGF17 FoxA2 HNF1B SOX17; the mesoderm marker genes monitored were BMP4, ISL1, KDR, HAND1; and the mesendoderm marker genes monitored were DKK1, EOMES, MIXL1, GATA4, GATA6, LHX1, WNT3a, T, GSC, TBX6.

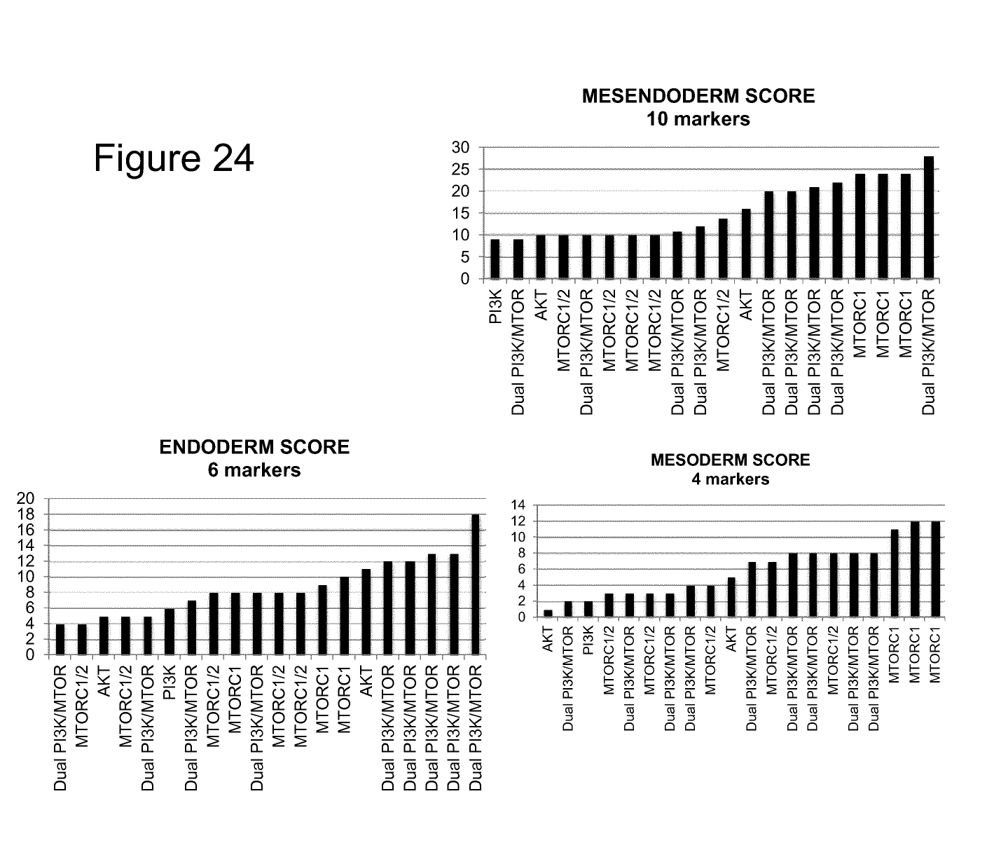

The results of this analysis are displayed in FIG. 24. MTORC1 and dual PI3K/MTOR inhibitors induced the highest expression of mesendoderm markers. MTORC1/2 and AKT inhibitors did not show a strong effect on mesendoderm formation compared to dual PI3K/MTOR inhibitors. Dual PI3K/MTOR inhibitors induced the highest expression of endoderm markers. However, as previously shown in Example 10, different PI3K/MTOR inhibitors had different effects on the expression level of each endoderm marker gene, and the differences between dual PI3K/MTOR inhibitors and mTORC1 were more or less significant depending on the marker.

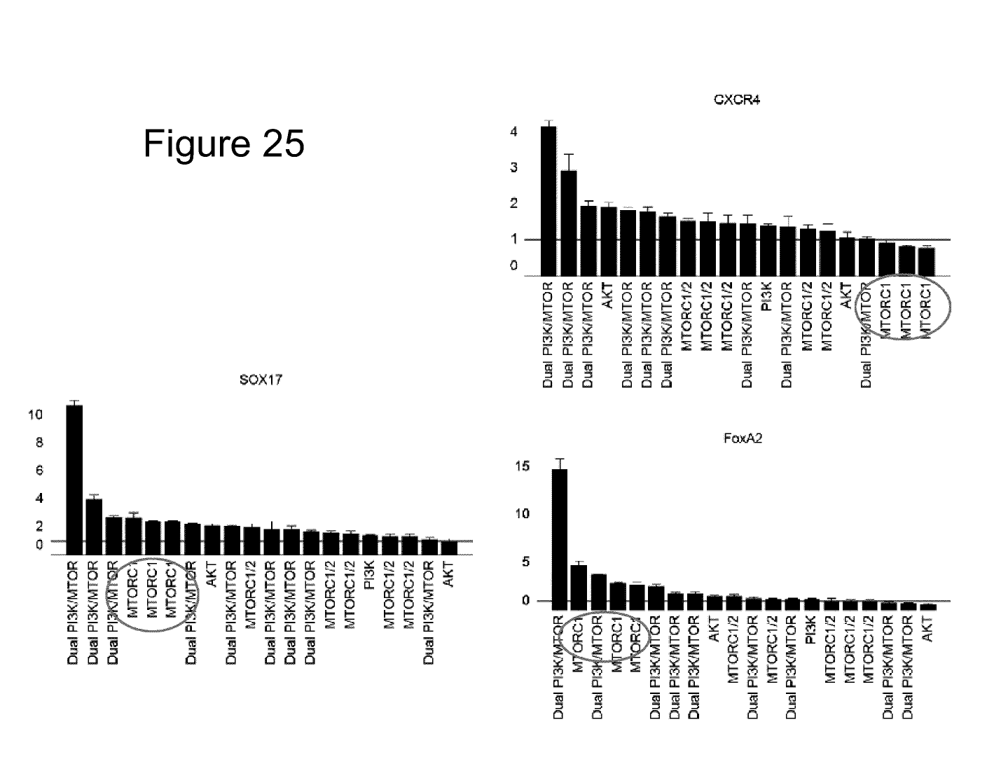

As shown in FIG. 25, the expression level of each endoderm marker is affected differently by each compound tested. Interestingly, MTORC1 inhibitors were able to increase the expression important endoderm genes like SOX17 and FOXA2 but CXCR4 was among other important endodermal marker genes whose expression was not increased by MTORC1 inhibitors. MTORC1 inhibitors increased SOX17 and FoxA2 expression compared to baseline at a level comparable to some dual PI3K/MTOR inhibitors. However, MTORC1 inhibitors did not increase CXCR4 expression, which confirmed the importance of PI3K inhibition for CXCR4 expression shown in Example 10. For mesoderm marker gene expression, MTORC1 inhibitors showed much higher score than dual PI3K/MTOR inhibitors. Interestingly, MTORC1 inhibitors were able to upregulate the expression of endoderm marker genes SOX17 and FOXA2, yet they did not upregulate the expression of endoderm marker gene CXCR4.

These results correlate with the observations from Example 10: mTOR inhibition had an important role at day 1 for mesendoderm formation. At Day 2, both PI3K and mTOR inhibition are important for endoderm formation and PI3K alpha inhibition is particularly important to prevent mesoderm from forming.

### Example 13

### Hepatocyte Differentiation

Hepatocyte Markers

A variety of cell-type specific markers were used to monitor the differentiation of endoderm cells into hepatocyte cells the flow cytometry and fluorescence imaging experiments described below. To detect endoderm conversion, endoderm-derived cell samples were stained for the expression of AFP or HNF4a proteins, which are expressed by hepatocyte cells but not by endoderm cells.

Hepatocyte Differentiation Protocol

Undifferentiated human embryonic stem cells (hESC were maintained at a density of 40,000 cells/cm² on qualified matrigel feeder layers (BD, #354277) in TesR™2 medium (STEMCELL™ Technologies #05860). Cultures were manually passaged twice a week. To prepare for endoderm differentiation, hESC cells were passaged into TesR™2 medium overnight. The following day, the TesR™2 medium was replaced with basal medium (DMEM/F12+Glutamax (Invitrogen, #10565) supplemented with B27 (Invitrogen, #17504-044)). The basal medium was supplemented with 100 μg/ml human Activin A (Peprotech, #120-14) and 750 nM Compound A. After the three day treatment, the hES-derived endoderm cells were differentiated in hepatoblast medium (DMEM/F12+Glutamax (Invitrogen, #10565) supplemented with B27 (Invitrogen, #17504-044)). When indicated, the hepatoblast medium was supplemented with 10, 20, or 40 ng/ml of recombinant human FGF2 (Peprotech, #AF-100-18B); 10, 20, or 40 ng/ml of recombinant human FGF4 (Peprotech, #AF-100-31); 20, 40 or 60 ng/ml recombinant human BMP2 (Peprotech, #AF-120-02); 20, 40 or 60 ng/ml recombinant human BMP4 (Peprotech, #AF-120-05); or 0.25% or 0.5% DMSO. After 10-day treatment, the endoderm-derived cells were harvested using TrypLE. Briefly, the cells were washed once in PBS, incubated with TrypLE for 5 minutes at 37° C. The incubated cells were then diluted 10-fold with PBS, pelleted, and prepared for further analysis.

Flow Cytometry Protocol

Prior to flow cytometry analysis, the Accutase-dissociated endoderm-derived cell samples were stained with an anti-AFP primary antibody followed by a secondary antibody.

The harvested endoderm-derived cells were washed with cold DPBS. The cells were then fixed for 25 minutes at 4° C. in fixation buffer (BD, #554655), permeabilized for 15 minutes with a saponin-based Perm/Wash Buffer I (BD, #557885), and stained with a 1:500 dilution of a mouse monoclonal anti-AFP Clone C3 IgG2a (Sigma, #A8452) in the permeabilization buffer. After a 30 minutes incubation at room temperature, the cells were washed two times in the permeabilization/wash buffer and then stained for 25 minutes at room temperature with 20 ml of a rat-anti-mouse IgG2a-PE secondary antibody (BD, #340269). The cells were washed three more times in permeabilization/wash buffer prior to flow cytometry analysis.

In addition, endoderm cells cultured in basal medium supplemented with Activin A and Compound A were stained as a negative control. HepG2 cells, which were derived from a well-differentiated hepatocellular carcinoma, were also stained as a positive control. The cells were analyzed via flow cytometry as described above. Approximately 1.5×10⁶ cells were analyzed per sample.

Imaging Protocol

Prior to immunofluorescence imaging, the endoderm-derived cell samples were stained to detect AFP expression. First, the cell samples were prepared for antibody staining as described above in the Methods and Materials for endoderm differentiation. Cell samples in which AFP expression was to be detected were incubated for overnight at 4° C. in a 1:500 dilution of mouse anti-AFP Clone C3 primary antibody (Sigma, #A8452) in blocking buffer. Cell samples in which HNF4a expression was to be detected were incubated overnight at 4° C. in a 1:100 dilution of rabbit monoclonal anti-HNF4a clone C11F12 (Cell Signaling, #3113) in blocking buffer. Cells stained with anti-AFP antibody were then incubated with 2 μg/ml goat anti-mouse-Alexa488 secondary antibody (Invitrogen, #A11029) for 1 hour at room temperature. Cells stained with anti-HNF4a antibody were then incubated with 2 μg/ml goat anti-rabbit-Alexa594 secondary antibody (Invitrogen, #A11037) under the same incubation conditions.

The stained cells were then imaged as described above for endoderm cells.

### Example 14

### Hepatocyte Differentiation in the Absence of Growth Factors

Endoderm cells were treated with different combinations of growth factors and tested for their capability to differentiate into hepatocytes. hESC were differentiated into endoderm cells as described above. After three days in basal medium supplemented with Activin A and Compound A, the endoderm cells were cultured on matrigel in hepatoblast medium. The medium was supplemented with 10, 20, or 40 ng/ml of recombinant human FGF2; 10, 20, or 40 ng/ml of recombinant human FGF4; 20, 40 or 60 ng/ml recombinant human BMP2; 20, 40 or 60 ng/ml recombinant human BMP4; or 0.25% or 0.5% DMSO. A control sample of endoderm cells obtained by culture with Activin A and Compound A for three days was further cultured in hepatoblast medium in the absence of any additional growth factors. After 10 days of treatment, the endoderm-derived cells were prepared for flow cytometry and imaging analysis as described above. With the exception of stem cells, endoderm cells cultured under all the conditions described above differentiated into hepatocytes.

To confirm that endoderm cells obtained by treatment with Activin A and Compound A could differentiate into hepatocytes, the above experiment was repeated. An additional control sample was prepared in which endoderm cells obtained by culture with Activin A alone were cultured in hepatoblast medium in the absence of any additional growth factors. The medium in each culture was changed every two days and the cells were harvested and stained in preparation for flow cytometry analysis.

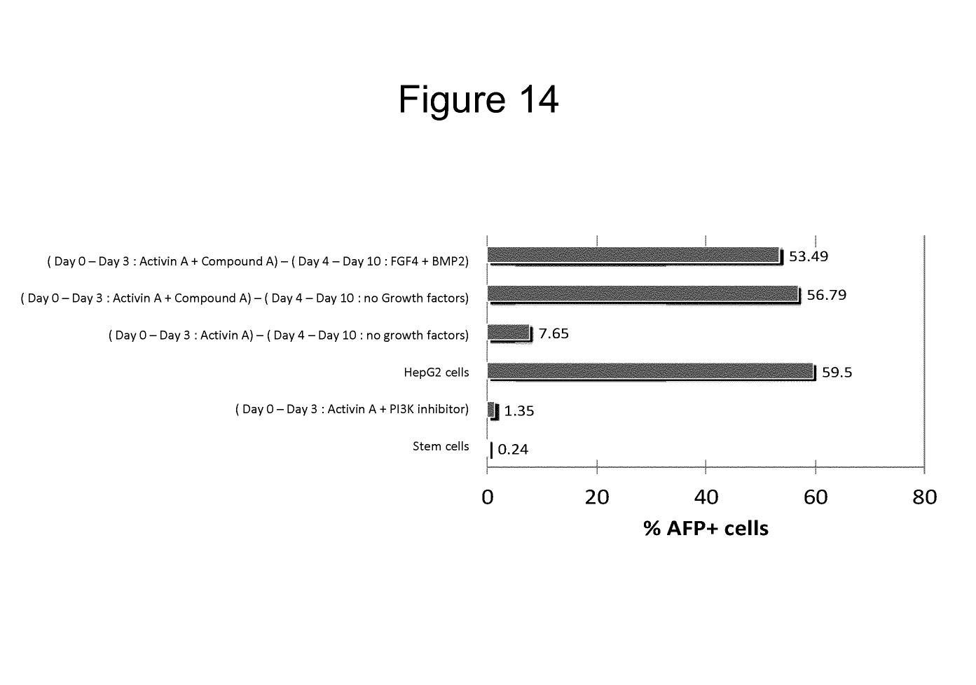

The results of this analysis are shown in FIG. 14. Endoderm cells obtained by treatment with Activin A alone that were subsequently cultured in hepatoblast medium in the absence of FGF4 and BMP2 exhibited low hepatocyte differentiation, with only 7.65% of the endoderm-derived cells expression AFP. In contrast, endoderm cells obtained by culture in the presence of Activin A and Compound A that were subsequently cultured in hepatoblast medium in the absence of FGF4 and BMP2 exhibited increased hepatocyte differentiation (with 56.79% of the cells expressing AFP). This indicates that the addition of the PI3K alpha inhibitor Compound A during endoderm differentiation greatly enhances hepatocyte conversion.

The hepatocyte cells derived from endoderm cells treated with Activin A and Compound A and differentiated without FGF4 and BMP2 treatment exhibited increased hepatocyte conversion with 56.79% (about 56%) of the cells expressing AFP as compared to hepatocyte cells derived from endoderm cells obtained by culture in the presence of Activin A and Compound A that were subsequently cultured in hepatoblast medium containing FGF4 and BMP2 (with 53.49% (about 53%) of cells expressing AFP). The level of AFP expression in hepatocyte cells derived from endoderm cells obtained by culture in the presence of Activin A and Compound A that were subsequently cultured without additional growth factors was comparable to the level of AFP expression in a population of HepG2 cells. These results indicate that endoderm obtained by treatment with Activin A and Compound A can differentiate into hepatocyte cells with high efficiency, even without the addition of growth factors.

### Example 15

### Characterization of Hepatocytes

A time course experiment was performed to determine the expression of AFP of hESC-derived hepatocyte cells over time. Briefly, hESC were differentiated into endoderm cells as described above. After three days in basal medium supplemented with Activin A and Compound A, the endoderm cells were cultured on matrigel in hepatoblast medium (DMEM/F12+Glutamax (Invitrogen, #10565) supplemented with B27 (Invitrogen, #17504-044). The medium was changed every other day. Two samples of cells were prepared. One sample of cells was harvested at day 10 and stained with anti-AFP, a in preparation for flow cytometry analysis, as described above. The second sample of cells was harvested and prepared for flow cytometry at day 20. As shown in FIG. 15, fewer cells in the population of stem-cell derived hepatocytes expressed AFP at day 20 (i.e., 30%) than at day 10 (i.e., 60%). These results are indicative of the maturation of hESC-derived hepatocyte cells.

FIG. 16 shows the results of an experiment measuring AFP levels. Day 0-Day 3: Activin A or Activin A+PI3K inhibitor. Day 4-Day 10−DMEM/F12+Glutamax+B27. At day 10 of differentiation, medium is changed. Twenty-four hours later, the medium is diluted by 1/500 (to be in the range) and analyzed by Alphalisa. When PI3K inhibitor is not used at the endoderm stage, AFP level is very low. When PI3K inhibitor is used at the endoderm stage, Fold is at almost 100 (for the 1/500 diluted sample). Expressing the data in fold allow for comparison of different samples/experiments. Fold=Signal medium contact with the cells/Signal raw medium without contact with cells.

FIG. 17 shows the results measuring albumin and HNF4a on stem cell derived hepatocytes at day 20. Stem cells derived hepatocytes population at Day 20: Day 0-Day 3: Activin A+PI3K inhibitors (Compound A). Day 3-Day 20: Basal medium (DMEM/F12+glutamax+B27).

In addition, the abilities of AA and AP cells to convert into hepatocyte progenitors, as depicted in the flowcharts below, were evaluated:

AA cells: Stem cells→(Activin A)→Endoderm→Hepatocytes

AP cells: Stem cells→(Activin A+Compound A)→Endoderm→Hepatocytes

Expression of AFP, a specific fetal liver marker (Roelandt, et al. (2010). “Human embryonic and rat adult stem cells with primitive endoderm-like phenotype can be fated to definitive endoderm, and finally hepatocyte-like cells.” PLoS One, 5(8): e12101) was used to identify hepatocyte progenitor cells. The expression levels of mature hepatocyte marker genes such as Albumin, A1AT as well as CK18 (Mild, T. (2011). Hepatic differentiation of human embryonic and induced pluripotent stem cells for regenerative medicine. In M. Kallos (Ed.), Embryonic Stem cells—Differentiation and pluripotent alternatives (pp. 303-320). InTech.) were also monitored to identify cells that had further differentiated. None of those markers were detected by immunofluorescence at Day 3 in AA or AP endoderm cells. At Day 13 of differentiation, AA and AP population exhibited different levels of markers expression. Analysis by flow cytometry showed that FoxA2 and AFP expression was not detected in AA cells whereas AP cells comprised 60% of the cells expressing FoxA2 and almost 50% of the cells expressing AFP (see Table 7 below).

TABLE 7

| | % AFP-expressing | % FoxA2-expressing |
|---|---|---|
| Conditions | cells | cells |
| AA Cells | 3% | 7% |
| AP Cells | 45% | 60% |

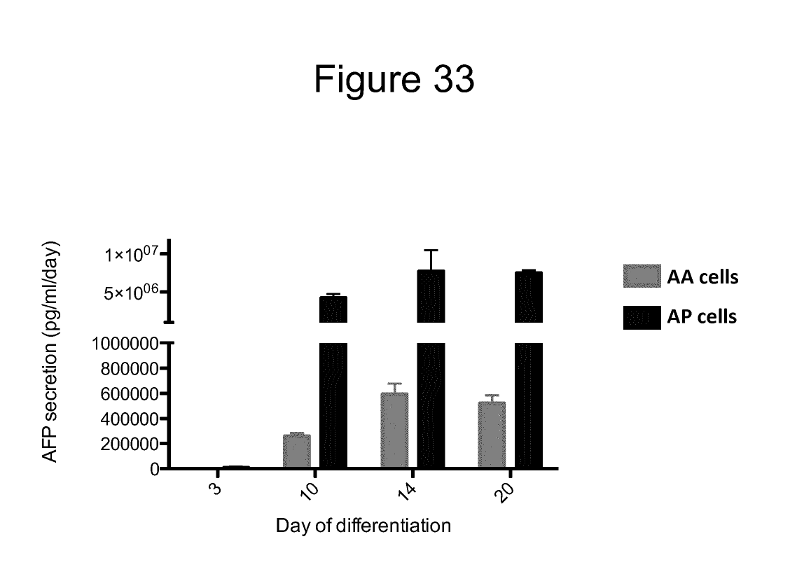

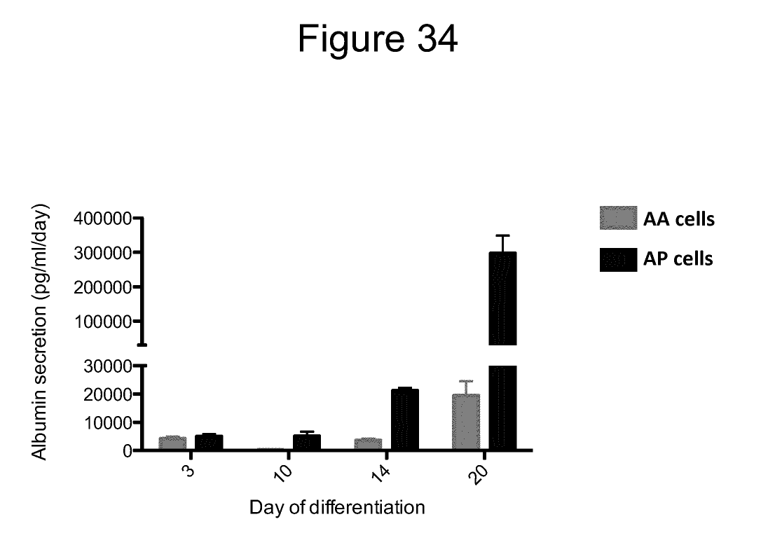

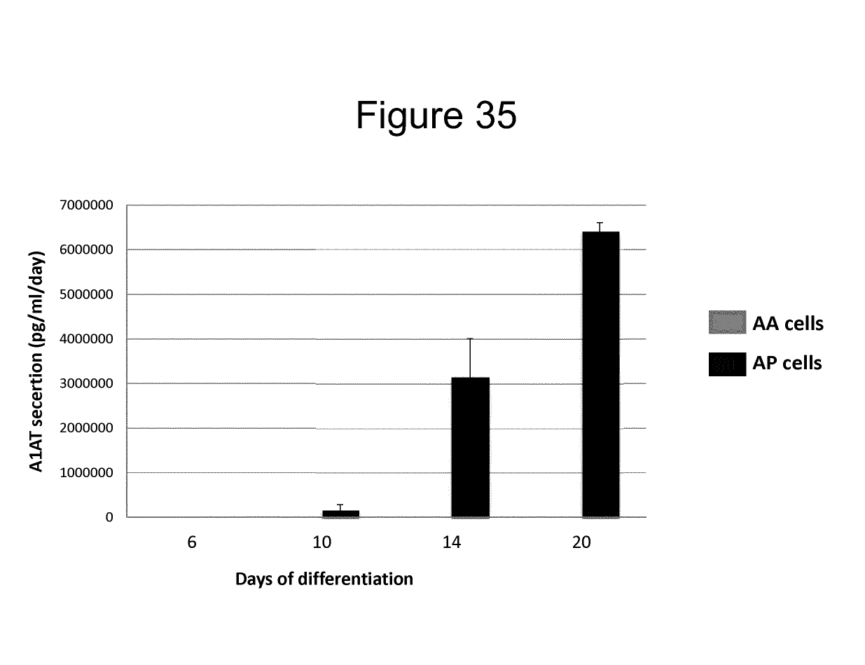

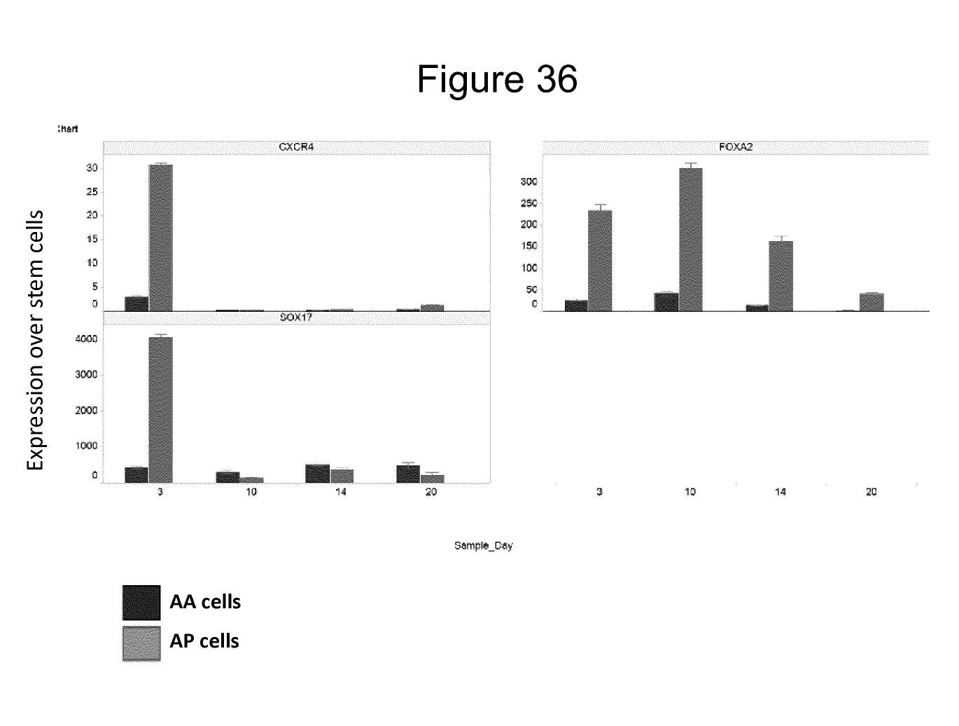

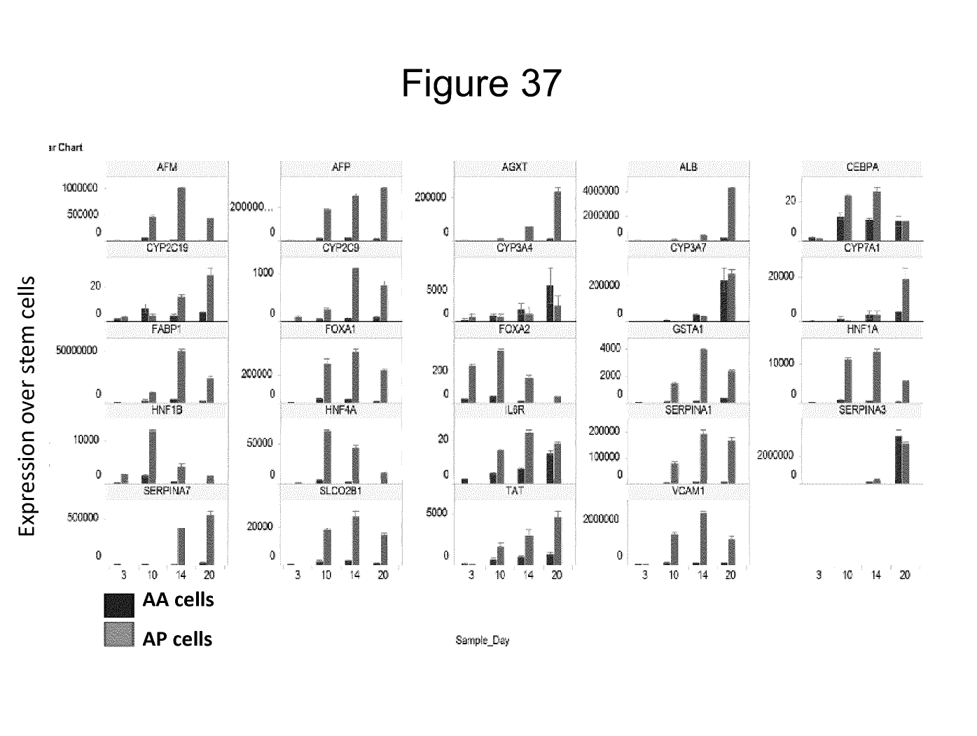

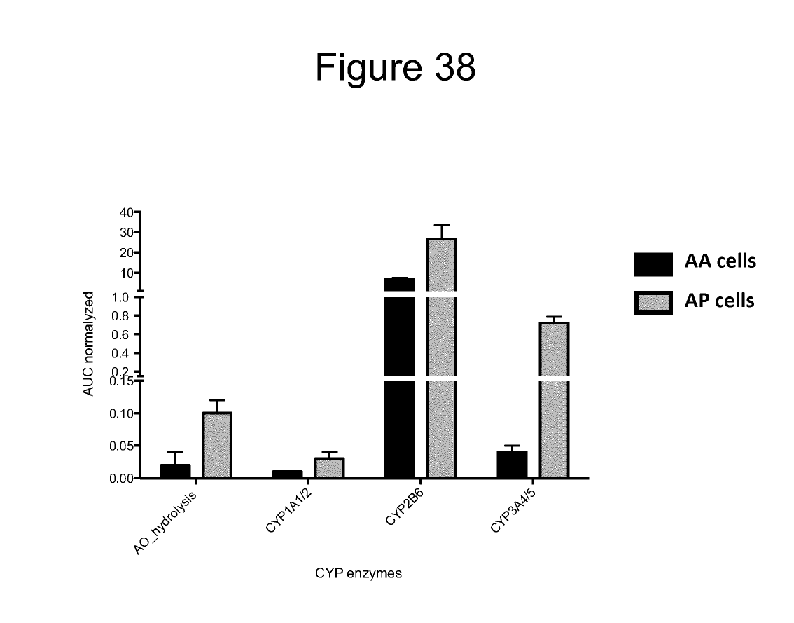

AFP and Albumin secretion in the medium were detected at different time points by Alphalisa assays (FIGS. 33 and 34). AFP secretion started increasing as soon as Day 10 for both AA and AP cells and reached a plateau at Day 14. AFP secretion for AP cells was almost 8,000 ng/ml/day at Day 14 and Day 20, which was 13 times higher than AA cells. Albumin, a marker for mature hepatocytes, was detected in the medium later in the differentiation. For AA cells, increasing albumin secretion was detected at Day 20. Albumin secretion by AP cells was detected as soon as Day 14, and secreted albumin levels at Day 20 were significantly increased as compared to AA cells. Albumin secretion reached almost 3,000 ng/ml for AP cells, which was 15 times higher than AA cells at the same time point. A similar time course was done for A1AT secretion via Alphalisa. For AA cells, levels of secreted A1AT were below the limit of detection at all time points tested. By contrast, A1AT secretion was detectable in AP cells as soon as Day 10 and reached 6000 ng/ml/day at Day 20 (FIG. 35). These significant differences between AA and AP cells at Day 20 were also seen via immunofluorescence. At Day 20, AA cells were expressing AFP but only a small portion of the cells expressed the rest of the markers. A large majority of the AP cells were expressing FoxA2, HNF4a, AFP, Albumin, A1AT and CK18 at Day 20. High expression of Albumin, A1AT and Ck18 shows that AP hepatocyte-like cells have a more mature phenotype. Gene expression analysis confirmed expression of AFP, Albumin and A1AT and their increased expression over time in AP cells (FIG. 38). Endoderm markers, SOX17 and CXCR4, were down regulated in AP cells from day 10 (FIG. 36). Gene expression analyses also showed expression of additional hepatic markers in AP—hepatocyte like cells: liver specific markers, AFM and AGTX, CYP enzymes including CYP2C19, CYP2C9, CYP3A4, CYP3A7, CYP7A1, phase II metabolism enzymes like GSTA1 secreted proteins like SERPINA1, SERPINA3, SERINA7, TAT, FABP1, transcription factors, HNF4a, HNF1B, C/EBPa, HNF1A, FOXA2, FOXA1, transporters like SLCO2B1 and surface proteins like IL6R and VCAM1(FIG. 37). Expression of hepatic markers was higher in AP-hepatocytes like cells than in AA differentiated cells (FIG. 37).

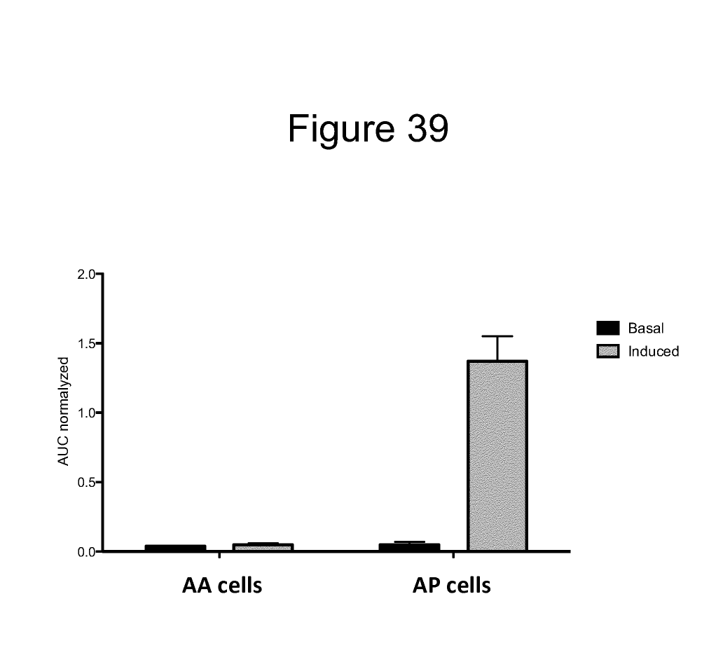

CYP activity was also analyzed via mass spectrometry. AP cells had higher CYP1A1/2, CYP2B6, CYP3A4/5 activity and aldehyde oxidase (AO) activity than AA cells at Day 24 (FIG. 38). Moreover, CYP1A1/2 activity was inducible in AP by 10 μM Rifampicin+1 mM Phenobarbital+1 μM 3-methylcholanthrene (3MC) (FIG. 39).

Thus, AP endoderm cells were multi potent and capable of differentiating into hepatocytes expressing lineage specific markers.

### Example 16

### Endoderm Cell Differentiation to Pancreatic Progenitor Cells and/or Pancreatic Cells

Multipotent endoderm cells are capable of differentiating into a variety of cell lineages, including, e.g., hepatocytes, lung cells, intestinal cells, pancreatic progenitor cells and pancreatic cells. Experiments were performed in which endoderm cells, produced as described above, were treated with different combinations of growth factors and tested for their capability to differentiate into pancreatic progenitor cells.

Stem cells were cultured as described above, in the presence of Activin A and Compound A. After 3 days of endoderm differentiation, cells were further differentiated into pancreatic cells. Endoderm cells were cultured for 3 days with 50 ng/ml FGF10 (Peprotech), 20 ng/ml FGF7 (Peprotech), 100 ng/ml Noggin (Peprotech) and a hedgehog inhibitor. The cells were cultured for four additional days in the same cocktail but with addition of 2 uM retinoic acid (Sigma). At this stage, pancreatic progenitors (Day 10) were cultured for 3 days with 1 uM Notch inhibitor DAPT (Sigma), 10 mM Nicotinamide (Sigma) and 50 ng/ml Exendin 4 (Tocris). For maturation, cells were cultured for seven additional days in 50 ng/ml Exendin 4, 50 ng/ml EGF (R&D) and 50 ng/ml IGF1 (R&D).

The abilities of AA and AP cells to convert into pancreatic progenitor cells, as depicted in the flowcharts below, were evaluated:

AA cells: Stem cells→(Activin A)→Endoderm→Pancreatic Progenitor Cells

AP cells: Stem cells→(Activin A+Compound A)→Endoderm→Pancreatic Progenitor Cells

At Day 12 of differentiation, significant differences in cell morphology were already noticeable between AA and AP cells. For example, at Day 12, both AA and AP cells were forming clusters. However, clusters derived from AP cells were in higher number and larger than clusters derived from AA cells.

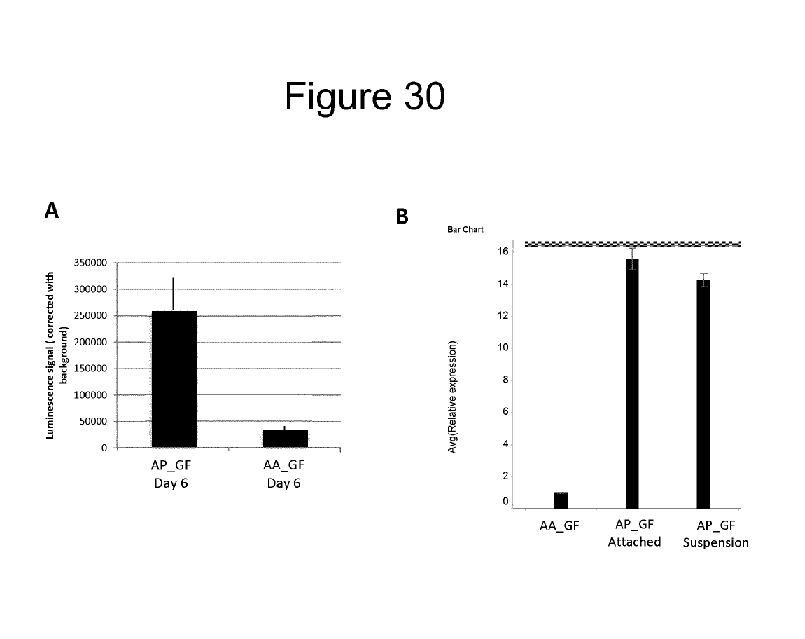

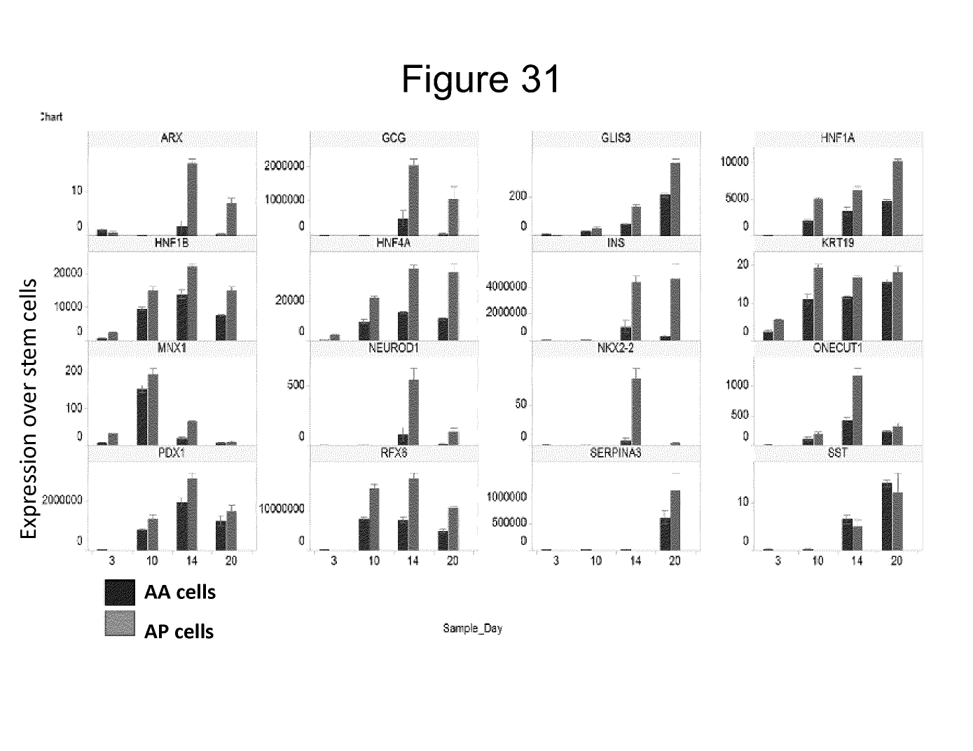

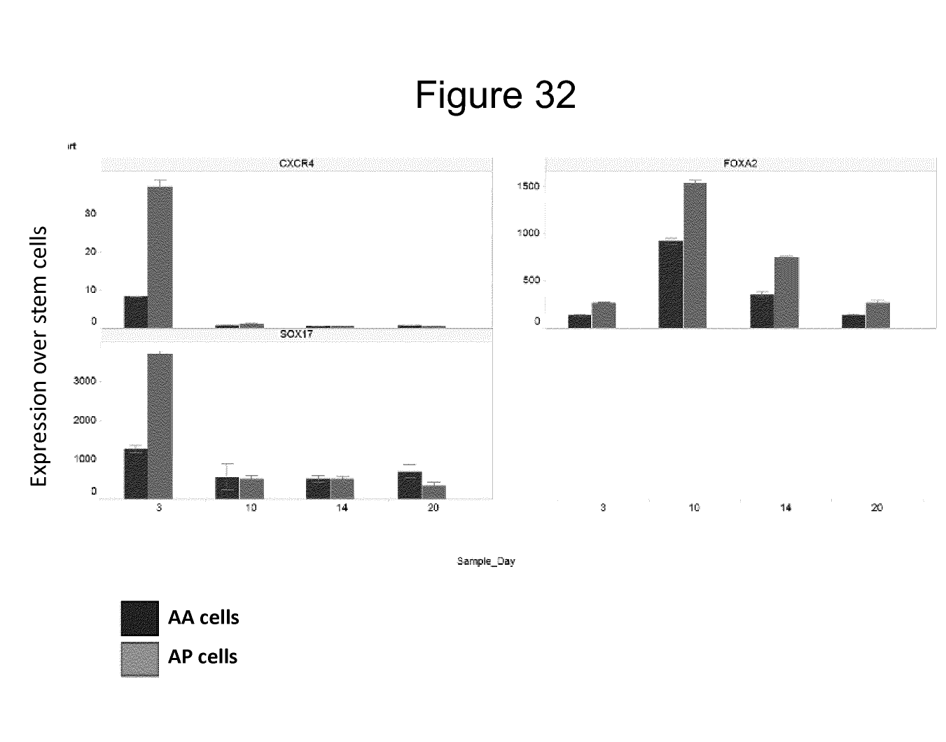

The expression of Pdx1, a specific pancreatic marker, was used to identify differentiated cells committed to the pancreatic lineage. The results of gene expression analyses showed a significant difference in Pdx1 expression levels between AP cells and AA cells at day 13: Pdx1 expression in AP cells was 15 times higher in AP cells compared to AA cells. (FIG. 30B) After further maturation (Day 20), insulin and glucagon expression were detected by immunofluorescence in multiple clusters of AP cells. By contrast, few AA-derived cells showed insulin or glucagon staining Clusters of pancreatic progenitor cells derived from the AP population were also positive for C peptide staining, indicating de novo insulin production. By contrast, only few cells in AA population expressed C peptide. Gene expression data (FIG. 31) confirmed that insulin and glucagon were more highly expressed in AP-derived pancreatic cells than in AA-derived pancreatic cells. The expression levels of additional pancreatic markers, including ARX, GLIS3, HNF1a, HNF1b, HNF4a, KRT19, MNX1, RFX6, SERPINA3, ONECUT1, NKX2-2, were also monitored in AP-derived and AA-derived pancreatic cells, and, as shown in FIG. 31, expression of these markers was higher in AP-derived cells than in AA-derived cells. Endoderm gene markers SOX17 and CXCR4 were down regulated from Day 10, and FoxA2 was maintained throughout the differentiation (FIG. 32). Gene markers for foregut development, HNF4a and HNF1b (Naujok, et al. (2011). “Insulin-producing Surrogate β-cells From Embryonic Stem Cells: Are We There Yet?” Molecular Therapy, 19(10), 1759-1768; Kroon et el. (2008). “Pancreatic endoderm derived from human embryonic stem cells generates glucose-responsive insulin-secreting cells in vivo.” Nat Biotechnol, 26(4), 443-452; and D'Amour et al. (2006). “Production of pancreatic hormone-expressing endocrine cells from human embryonic stem cells.” Nat Biotechnol, 24(11), 1392-1401) were expressed early in the differentiation. Posterior foregut marker, MNX1 (=HLXB9) (Naujok et al.; Kroon et al.; and D'Amour et al.) peaked on Day 10. Pancreatic endoderm markers, such as NKX2.2 (Naujok et al.; Kroon et al.; and D'Amour et al.) and ONECUT1 (=HNF6) reached their highest expression on Day 14. Finally, the expression of hormone cell markers, INS, GLC and SST, was detected starting on Day 14. The expression of specific pancreatic lineages markers confirmed that AP cells were capable of differentiation into pancreatic cells.

To explore the potential of three-dimensional culture, AA and AP endoderm cells were further differentiated in suspension. AA and AP endoderm cells behaved very differently after this transition. AA endoderm cells stayed as single cells in suspension whereas AP cells formed clusters as soon as Day 6. A cell viability assay (see FIG. 30A) showed that AA endoderm cells had poor viability in suspension at Day 6 of differentiation. AP endoderm cells, however, were viable within the clusters. The AP-derived clusters were positive for Pdx1 expression on Day 13, indicating that the cells were developmentally committed to the pancreatic lineage (FIG. 30B). Moreover, only the cells differentiating into pancreatic cells seem to stay viable in suspension by forming clusters

### Example 17

### Differentiation of Pancreatic Exocrine Cells and Pancreatic Ductal Cells from Pancreatic Progenitor Cells

Pancreatic progenitor cells are produced as described above. Growth factors, e.g., glucagon-like-peptide 1 (GLP1) as described in Shirasawa, S. et al. (2011). “A novel stepwise differentiation of functional pancreatic exocrine cells from embryonic stem cells.” Stem Cells Dev, 20(6): 1071-1078, compounds such as dexamethasone and dorsomorphin, as described in Delaspre, et al. (2013). “Directed pancreatic acinar differentiation of mouse embryonic stem cells via embryonic signaling molecules and exocrine transcription factors.” PLoS One, 8(1), e54243, and/or combinations thereof are added to the culture of pancreatic progenitor cells to form pancreatic exocrine cells.

To produce pancreatic ductal cells, pancreatic progenitor cells, produced as described above, are cultured with EGF, FGF10, PDGF-AA as described in Rhodes, J. A., Criscimanna, A., & Esni, F. (2012). “Induction of mouse pancreatic ductal differentiation, an in vitro assay.” In Vitro Cell Dev Biol Anim, 48(10), 641-649.

### Example 18

### Differentiation of Lung Progenitor Cells, Thyroid Progenitor Cells, and Airway Progenitor Cells from Endoderm

Endoderm cells are produced as described above. As described in Longmire, et al. (2012). “Efficient derivation of purified lung and thyroid progenitors from embryonic stem cells.” Cell Stem Cell, 10(4), 398-411, basal medium is supplemented with 100 ng/ml Noggin and 10 mM SB431542 (TGFbeta inhibitor). After 24 hours the media is replaced with Nkx2-1 induction media: cSFDM supplemented with 100 ng/ml mWnt3a, 10 ng/ml mKGF, 10 ng/ml hFGF10, 10 ng/ml mBMP4, 20 ng/ml hEGF, 500 ng/ml mFGF2 and 100 ng/ml Heparin Sodium Salt (Sigma). The cells are then cultured for 7 days in cSFDM supplemented with mFGF2 (500 ng/ml), hFGF10 (100 ng/ml), and 100 ng/ml Heparin Sodium Salt (Sigma). On day 22, cells are cultured in lung maturation media: Ham's F12 media+15 mM HEPES (pH 7.4)+0.8 mM CaCl2+0.25% BSA+5 mg/ml insulin+5 mg/ml transferrin+5 ng/ml Na selenite+50 nM Dexamethasone+0.1 mM 8-Br-cAMP+0.1 mM IBMX+10 ng/ml KGF.

Alternatively, endoderm cells are produced as described above and then treated as described in Mou, et al. (2012). “Generation of multipotent lung and airway progenitors from mouse ESCs and patient-specific cystic fibrosis iPSCs. Cell Stem Cell, 10(4), 385-397. Briefly, at Day 3 of endoderm differentiation, the cells exposed to 500 nM A-83-01 (TGF beta inhibitor) with or without 4 uM Dorsomorphin (BMP inhibitor) or 20 ng/ml BMP4 for 3 days. The cells are then exposed for 2-3 days to 10 ng/ml BMP4, 20 ng/ml FGF2+10 nM GSK3iXV. To obtain airway progenitor cells, the cells are cultured in 20 ng/ml BMP7, 20 ng/ml FGF7, 100 nM IWR-1 (WNT antagonist), and 1 mM PD98059 for 2 days.

### Example 19

### Differentiation of Intestinal Progenitor Cells from Endoderm

Endoderm cells are produced as described above. As described in Spence, et al. (2010). “Directed differentiation of human pluripotent stem cells into intestinal tissue in vitro.” Nature, 470(7332), 105-109, endoderm cells are treated with 500 ng/ml FGF4, 500 ng/ml WNt3a for up to 4 days. Cell colonies that are formed during this time are transferred to matrigel supplemented with 500 ng/ml R-spondin1, 100 ng/ml Noggin+50 ng/ml EGF.

Alternatively, endoderm cells are produced as described above and then treated as described in Cheng, et al. (2012). “Self-renewing endodermal progenitor lines generated from human pluripotent stem cells.” Cell Stem Cell, 10(4), 371-384. Briefly, at Day 3 of differentiation, endoderm cells are treated with BMP4 (500 ng/ml) and FGF4 (500 ng/ml) for 2 days to form colonies. The colonies are then harvested by digesting matrigel with collagenase B treatment at 37° C. for 1 hour. The colonies are then mixed with undiluted matrigel (BD) supplemented with FGF4 (50 ng/ml) Wnt3a (100 ng/ml), R-spondin1 (500 ng/ml), EGF (50 ng/ml) and Noggin (100 ng/ml).

1. An isolated population of endoderm cells wherein at least 83% of the cells express SOX17, at least 77% of the cells express FoxA2, or at least 76% of the cells express CXCR4.

2. The isolated population of endoderm cells of claim 1, wherein at least 83% of the cells express SOX17 and at least 77% of the cells express FoxA2.

3. The isolated population of endoderm cells of claim 1, wherein at least 77% of the cells express FoxA2 and at least 76% of the cells express CXCR4.

4. The isolated population of endoderm cells of claim 1, wherein at least 83% of the cells express SOX17 and at least 76% of the cells express CXCR4.

5. The isolated population of endoderm cells of claim 1, wherein at least 83% of the cells express SOX17, at least 77% of the cells express FoxA2, and at least 76% of the cells express CXCR4.

6. The isolated population of endoderm cells of claim 1, wherein the endoderm cells have the capability to become hepatocytes, pancreatic cells, pancreatic progenitor cells, liver cells, lung cells, airway progenitor cells, or lung epithelial cells.

7. A bank of stable endoderm cells comprising one or more populations of endoderm cells wherein at least 83% of the cells express SOX17, at least 77% of the cells express FoxA2, and/or at least 76% of the cells express CXCR4, wherein the population maintains this phenotype for at least 10 passages.

8. The bank of claim 7, wherein the endoderm cells have the capability to become hepatocytes, pancreatic cells, pancreatic progenitor cells, liver cells, lung cells, airway progenitor cells, or lung epithelial cells.

9. A method of obtaining a population of endoderm cells, the method comprising: contacting a population of stem cells with an effective amount of a selective inhibitor and an effective amount of an Activin A and culturing the cells under conditions sufficient to obtain the population of endoderm cells, the selective inhibitor is selected from the group consisting of an inhibitor of PI3K alpha, an inhibitor of mTOR, and a mixture thereof.

10. The method of claim 9, wherein the selective inhibitor is an inhibitor of PI3K alpha, at least 83% of the cells in the population of endoderm cells express SOX17, at least 77% of the cells in the population of endoderm cells express FoxA2, or at least 76% of the cells in the population of endoderm cells express CXCR4.

11. The method of claim 10, wherein at least 83% of the cells in the population of endoderm cells express SOX17 and at least 77% of the cells in the population of endoderm cells express FoxA2.

12. The method of claim 10, wherein at least 77% of the cells in the population of endoderm cells express FoxA2 and at least 76% of the cells in the population of endoderm cells express CXCR4.

13. The method of claim 10, wherein 83% of the cells in the population of endoderm cells express SOX17 and at least 76% of the cells in the population of endoderm cells express CXCR4

14. The method of claim 10, wherein at least 83% of the cells in the population of endoderm cells express SOX17, at least 77% of the cells in the population of endoderm cells express FoxA2 and at least 76% of the cells express CXCR4.

15. The method of claim 9, wherein the endoderm cells have the capability to become hepatocytes, pancreatic cells, pancreatic progenitor cells, liver cells, or lung epithelial cells.

16. The method of claim 9, wherein the selective inhibitor is an inhibitor of PI3K alpha, and the endoderm cells have greater viability and/or proliferation as compared to stem cells that have not been contacted with a selective inhibitor of PI3K alpha and Activin A.

17. The method of claim 9, wherein the stem cells are adult stem cells, embryonic stem cells, or induced pluripotent stem cells.

18. The method of claim 9, wherein the stem cells are cultured in qualified matrigel.

19. The method of claim 9, wherein the stem cells are cultured in suspension.

20. The method of claim 9, wherein the selective inhibitor is a selective inhibitor of PI3K alpha that is a compound which is a fused pyrimidine of formula (I): 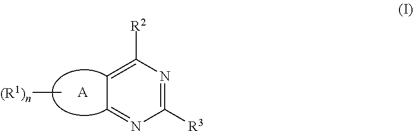
wherein
A represents a thiophene or furan ring;
n is 1 or 2;
R¹ is a group of formula: 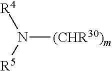
wherein
m is 0 or 1;
R³⁰ is H or C₁-C₆ alkyl;
R⁴ and R⁵ form, together with the N atom to which they are attached, a 5- or 6-membered saturated N-containing heterocyclic group which includes 0 or 1 additional heteroatoms selected from N, S and O, which may be fused to a benzene ring and which is unsubstituted or substituted; or one of R⁴ and R⁵ is alkyl and the other is a 5- or 6-membered saturated N-containing heterocyclic group as defined above or an alkyl group which is substituted by a 5- or 6-membered saturated N-containing heterocyclic group as defined above;
R² is selected from: 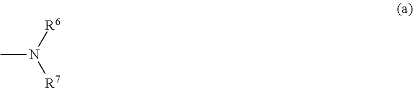
wherein R⁶ and R⁷ form, together with the nitrogen atom to which they are attached, a morpholine, thiomorpholine, piperidine, piperazine, oxazepane or thiazepane group which is unsubstituted or substituted; and 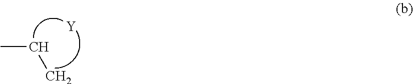
wherein Y is a C₂-C₄ alkylene chain which contains, between constituent carbon atoms of the chain and/or at one or both ends of the chain, 1 or 2 heteroatoms selected from O, N and S, and which is unsubstituted or substituted;
and R³ is an indazole group which is unsubstituted or substituted;
or a pharmaceutically acceptable salt thereof.

21. The method of claim 20 wherein the fused pyrimidine is of formula (Ia): 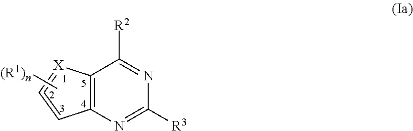
wherein X is S or O, and R¹, R², R³ and n are as defined in claim 20.

22. The method of claim 20, wherein the fused pyrimidine is of formula (Ib): 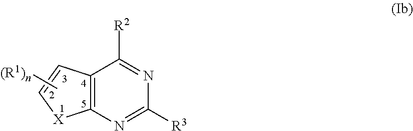
wherein X is S or 0, and R¹, R², R³ and n are as defined in claim 20.

23. The method of claim 20, wherein the compound is selected from:
2-(1H-Indazol-4-yl)-6-(4-methyl-piperazin-1-ylmethyl)-4-morpholin-4-yl-thieno[3,2-d]pyrimidine;
4-[2-(1H-Indazol-4-yl)-4-morpholin-4-yl-thieno[3,2-d]pyrimidin-6-ylmethyl]-piperazine-1-sulfonic acid dimethylamide;
{4-[2-(1H-Indazol-4-yl)-4-morpholin-4-yl-thieno[3,2-d]pyrimidin-6-ylmethyl]-piperazin-1-yl}-morpholin-4-yl-methanone;
4-[2-(1H-Indazol-4-yl)-4-morpholin-4-yl-thieno[3,2-d]pyrimidin-6-ylmethyl]-piperazine-1-carboxylic acid (2-methoxy-ethyl)-methyl-amide;
{4-[2-(1H-Indazol-4-yl)-4-morpholin-4-yl-thieno[3,2-d]pyrimidin-6-ylmethyl]-piperazin-1-yl}-N,N-dimethyl-acetamide;
4-[2-(1H-Indazol-4-yl)-4-morpholin-4-yl-thieno[3,2-d]pyrimidin-6-ylmethyl]-piperazine-1-carboxylic acid dimethylamide;
2-(1H-Indazol-4-yl)-4-morpholin-4-yl-6-[4-(3-morpholin-4-yl-propane-1-sulfonyl)-piperazin-1-ylmethyl]-thieno[3,2-d]pyrimidine;
{1-[2-(1H-Indazol-4-yl)-4-morpholin-4-yl-thieno[3,2-d]pyrimidin-6-ylmethyl]-piperidin-4-yl}-(2-methoxy-ethyl)-methyl-amine;
(3-{4-[2-(1H-Indazol-4-yl)-4-morpholin-4-yl-thieno[3,2-d]pyrimidin-6-ylmethyl]-piperazine-1-sulfonyl}-propyl)-dimethyl-amine;
2-{4-[2-(1H-Indazol-4-yl)-4-morpholin-4-yl-thieno[3,2-d]pyrimidin-6-ylmethyl]-piperazin-1-yl}-2-methyl-propan-1-ol;
1′-[2-(1H-Indazol-4-yl)-4-morpholin-4-yl-thieno[3,2-d]pyrimidin-6-ylmethyl]-[1,4′]bipiperidinyl;
2-(1H-Indazol-4-yl)-4-morpholin-4-yl-6-(4-morpholin-4-yl-piperidin-1-ylmethyl)-thieno[3,2-d]pyrimidine;
2-(1H-Indazol-4-yl)-4-morpholin-4-yl-6-(4-pyrimidin-2-yl-piperazin-1-ylmethyl)-thieno[3,2-d]pyrimidine;
1-(2-Hydroxy-ethyl)-4-[2-(1H-indazol-4-yl)-4-morpholin-4-yl-thieno[3,2-d]pyrimidin-6-ylmethyl]-piperazin-2-one;
6-(4-Cyclopropylmethyl-piperazin-1-ylmethyl)-2-(1H-indazol-4-yl)-4-morpholin-4-yl-thieno[3,2-d]pyrimidine;
2-(1H-Indazol-4-yl)-4-morpholin-4-yl-6-(4-pyridin-2-yl-piperazin-1-ylmethyl)-thieno[3,2-d]pyrimidine;
2-(1H-Indazol-4-yl)-4-morpholin-4-yl-6-[4-(2,2,2-trifluoro-ethyl)-piperazin-1-ylmethyl]-thieno[3,2-d]pyrimidine;
2-(1H-Indazol-4-yl)-4-morpholin-4-yl-6-(4-thiazol-2-yl-piperazin-1-ylmethyl)-thieno[3,2-d]pyrimidine;
2-(6-Fluoro-1H-indazol-4-yl)-6-(4-methyl-piperazin-1-ylmethyl)-4-morpholin-4-yl-thieno[3,2-d]pyrimidine;
2-(1H-Indazol-4-yl)-4-morpholin-4-yl-6-(4-pyridin-2-ylmethyl-piperazin-1-ylmethyl)-thieno[3,2-pyrimidine;
2-(1H-Indazol-4-yl)-4-morpholin-4-yl-6-(4-thiazol-2-ylmethyl-piperazin-1-ylmethyl)-thieno[3,2-d]pyrimidine;
2-(1H-Indazol-4-yl)-6-[4-(5-methyl-furan-2-ylmethyl)-piperazin-1-ylmethyl]-4-morpholin-4-yl-thieno[3,2-d]pyrimidine;
1-[2-(1H-Indazol-4-yl)-4-morpholin-4-yl-thieno[3,2-d]pyrimidin-6-ylmethyl]-piperidine-4-carboxylic acid amide;
2-(1H-Indazol-4-yl)-6-[4-(2-methoxy-1,1-dimethyl-ethyl)-piperazin-1-ylmethyl]-4-morpholin-4-yl-thieno[3,2-d]pyrimidine;
2-(1H-Indazol-4-yl)-6-[(3R,5S)-4-(2-methoxy-ethyl)-3,5-dimethyl-piperazin-1-ylmethyl]-4-morpholin-4-yl-thieno[3,2-d]pyrimidine;
1-[2-(1H-Indazol-4-yl)-4-morpholin-4-yl-thieno[3,2-d]pyrimidin-6-ylmethyl]-piperidine-4-carboxylic acid (2-methoxy-ethyl)-methyl-amide;
1-[2-(1H-Indazol-4-yl)-4-morpholin-4-yl-thieno[3,2-d]pyrimidin-6-ylmethyl]-piperidine-4-carboxylic acid dimethylamide;
2-(1H-Indazol-4-yl)-4-morpholin-4-yl-6-(4-pyridin-3-ylmethyl-piperazin-1-ylmethyl)-thieno[3,2-d]pyrimidine;
1-[2-(1H-Indazol-4-yl)-4-morpholin-4-yl-thieno[3,2-d]pyrimidin-6-ylmethyl]-piperidine-4-carboxylic acid methylamide;
2-{4-[2-(1H-Indazol-4-yl)-4-morpholin-4-yl-thieno[3,2-d]pyrimidin-6-ylmethyl]-piperazin-1-yl}-N-methyl-isobutyramide;
2-{4-[2-(1H-Indazol-4-yl)-4-morpholin-4-yl-thieno[3,2-d]pyrimidin-6-ylmethyl]-piperazin-1-yl}-2-methyl-1-pyrrolidin-1-yl-propan-1-one;
2-(1H-Indazol-4-yl)-6-[4-(1-methyl-1H-imidazol-2-ylmethyl)-piperazin-1-ylmethyl]-4-morpholin-4-yl-thieno[3,2-d]pyrimidine;
2-(1H-Indazol-4-yl)-6-[4-(5-methyl-isoxazol-3-ylmethyl)-piperazin-1-ylmethyl]-4-morpholin-4-yl-thieno[3,2-d]pyrimidine;
1-{4-[2-(1H-Indazol-4-yl)-4-morpholin-4-yl-thieno[3,2-d]pyrimidin-6-ylmethyl]-piperazin-1-yl}-2-methyl-propan-2-ol;
Cyclopropylmethyl-{1-[2-(1H-indazol-4-yl)-4-morpholin-4-yl-thieno[3,2-d]pyrimidin-6-ylmethyl]-piperidin-4-yl}-(2-methoxy-ethyl)-amine;
6-[4-(1-Ethyl-1-methoxymethyl-propyl)-piperazin-1-ylmethyl]-2-(1H-indazol-4-yl)-4-morpholin-4-yl-thieno[3,2-d]pyrimidine;
2-(1H-Indazol-4-yl)-6-[4-(1-methoxymethyl-cyclopropyl)-piperazin-1-ylmethyl]-4-morpholin-4-yl-thieno[3,2-d]pyrimidine;
{1-[2-(1H-Indazol-4-yl)-4-morpholin-4-yl-thieno[3,2-d]pyrimidin-6-ylmethyl]-piperidin-4-yl}-(2-methoxy-ethyl)-(2,2,2-trifluoro-ethyl)-amine;
2-(1H-Indazol-4-yl)-6-[4-(2-methoxy-ethyl)-piperazin-1-ylmethyl]-4-morpholin-4-yl-thieno[3,2-d]pyrimidine;
{1-[2-(1H-Indazol-4-yl)-4-morpholin-4-yl-thieno[3,2-d]pyrimidin-6-ylmethyl]-piperidin-4-yl}-methanol;
2-(1H-Indazol-4-yl)-4-morpholin-4-yl-6-(4-pyridin-4-ylmethyl-piperazin-1-ylmethyl)-thieno[3,2-d]pyrimidine;
2-(1H-Indazol-4-yl)-6-[4-(6-methyl-pyridin-2-ylmethyl)-piperazin-1-ylmethyl]-4-morpholin-4-yl-thieno[3,2-d]pyrimidine;
2-(1H-Indazol-4-yl)-6-[4-(4-methyl-thiazol-2-ylmethyl)-piperazin-1-ylmethyl]-4-morpholin-4-yl-thieno[3,2-d]pyrimidine;
{1-[2-(1H-Indazol-4-yl)-4-morpholin-4-yl-thieno[3,2-d]pyrimidin-6-ylmethyl]-piperidin-4-yl}-pyridin-2-yl-amine;
N-{1-[2-(1H-Indazol-4-yl)-4-morpholin-4-yl-thieno[3,2-d]pyrimidin-6-ylmethyl]-piperidin-4-yl}-2-methoxy-N-methyl-acetamide;
N-{1-[2-(1H-Indazol-4-yl)-4-morpholin-4-yl-thieno[3,2-d]pyrimidin-6-ylmethyl]-piperidin-4-yl}-N-methyl-methanesulfonamide;
{1-[2-(H-Indazol-4-yl)-4-morpholin-4-yl-thieno[3,2-d]pyrimidin-6-ylmethyl]-piperidin-4-yl}-(3-methoxy-propyl)-methyl-amine;
6-((3 S,5R)-3,5-Dimethyl-4-pyridin-2-ylmethyl-piperazin-1-ylmethyl)-2-(1H-indazol-4-yl)-4-morpholin-4-yl-thieno[3,2-d]pyrimidine;
2-(1H-Indazol-4-yl)-6-(4-methoxymethyl-piperidin-1-ylmethyl)-4-morpholin-4-yl-thieno[3,2-d]pyrimidine;
{1-[2-(1H-Indazol-4-yl)-4-morpholin-4-yl-thieno[3,2-d]pyrimidin-6-ylmethyl]-piperidin-4-yl}-(2-methoxy-ethyl)-thiazol-2-ylmethyl-amine;
1-[2-(1H-Indazol-4-yl)-4-morpholin-4-yl-thieno[3,2-d]pyrimidin-6-ylmethyl]-4-pyridin-2-ylmethyl-piperidin-4-ol;
{1-[2-(1H-Indazol-4-yl)-4-morpholin-4-yl-thieno[3,2-d]pyrimidin-6-ylmethyl]-piperidin-4-yl}-isopropyl-(2-methoxy-ethyl)-amine;
2-(H-Indazol-4-yl)-4-morpholin-4-yl-6-[4-(pyridin-2-yloxy)-piperidin-1-ylmethyl]-thieno[3,2-d]pyrimidine;
N-{1-[2-(1H-Indazol-4-yl)-4-morpholin-4-yl-thieno[3,2-d]pyrimidin-6-ylmethyl]-piperidin-4-yl}-N-(2-methoxy-ethyl)-methanesulfonamide;
2-{1-[2-(1H-Indazol-4-yl)-4-morpholin-4-yl-thieno[3,2-d]pyrimidin-6-ylmethyl]-piperidin-4-yl}-propan-2-ol;
2-(1H-Indazol-4-yl)-4-morpholin-4-yl-6-[4-(1-oxy-pyridin-3-ylmethyl)-piperazin-1-ylmethyl]-thieno[3,2-d]pyrimidine;
2-(1H-Indazol-4-yl)-4-morpholin-4-yl-6-(4-morpholin-4-ylmethyl-piperidin-1-ylmethyl)-thieno[3,2-d]pyrimidine;
{1-[2-(1H-Indazol-4-yl)-4-morpholin-4-yl-thieno[3,2-d]pyrimidin-6-ylmethyl]-piperidin-4-ylmethyl}-(2-methoxy-ethyl)-methyl-amine;
{1-2-(1H-Indazol-4-yl)-4-morpholin-4-yl-thieno[3,2-d]pyrimidin-6-ylmethyl]-piperidin-4-ylmethyl}-dimethyl-amine;
{1-[2-(1H-Indazol-4-yl)-4-morpholin-4-yl-thieno[3,2-d]pyrimidin-6-ylmethyl]-piperidin-3-yl}-(2-methoxy-ethyl)-methyl-amine;
1-[2-(1H-Indazol-4-yl)-4-morpholin-4-yl-thieno[3,2-d]pyrimidin-6-ylmethyl]-piperidine-3-carboxylic acid methylamide;
2-(1H-Indazol-4-yl)-6-(3-methoxymethyl-piperidin-1-ylmethyl)-4-morpholin-4-yl-thieno[3,2-d]pyrimidine;
2-(1H-Indazol-4-yl)-4-morpholin-4-yl-6-(4-pyridin-2-ylmethyl-piperidin-1-ylmethyl)-thieno[3,2-d]pyrimidine;
2-(1H-Indazol-4-yl)-6-[4-(2-methoxy-ethoxy)-piperidin-1-ylmethyl]-4-morpholin-4-yl-thieno[3,2-d]pyrimidine;
6((3R,5S)-3,5-Dimethyl-4-thiazol-2-ylmethyl-piperazin-1-ylmethyl)-2(1H-indazol-4-yl)-4-morpholin-4-yl-thieno[3,2-d]pyrimidine;
2-(1H-Indazol-4-yl)-4-morpholin-4-yl-6-[411-oxy-pyridin-2-ylmethyl)-piperazin-1-ylmethyl]-thieno[3,2-d]pyrimidine;
2-(1H-Indazol-4-yl)-6-[4-(2-methoxy-ethyl)-piperidin-1-ylmethyl]-4-morpholin-4-yl-thieno[3,2-d]pyrimidine;
2-(1H-Indazol-4-yl)-6-(4-methanesulfonyl-piperidin-1-ylmethyl)-4-morpholin-4-yl-thieno[3,2-d]pyrimidine;
{1-[2-(1H-Indazol-4-yl)-4-morpholin-4-yl-thieno[3,2-d]pyrimidin-6-ylmethyl]-piperidin-4-yl}-(3-methanesulfonyl-propyl)-methyl-amine;
2-(1H-Indazol-4-yl)-6-[4-(3-methoxy-propane-1-sulfonyl)-piperidin-1-ylmethyl]-4-morpholin-4-yl-thieno[3,2-d]pyrimidine;
(R)-1-[2-(1H-Indazol-4-yl)-4-morpholin-4-yl-thieno[3,2-d]pyrimidin-6-ylmethyl]-piperidine-3-carboxylic acid methylamide;
(S)-1-[2-(H-Indazol-4-yl)-4-morpholin-4-yl-thieno[3,2-d]pyrimidin-6-ylmethyl]-piperidine-3-carboxylic acid methylamide;
6-(4-Imidazol-1-ylmethyl-piperidin-1-ylmethyl)-2-(1H-indazol-4-yl)-4-morpholin-4-yl-thieno[3,2-d]pyrimidine;
2-(1H-Indazol-4-yl)-4-morpholin-4-yl-6-morpholin-4-ylmethyl-thieno[3,2-d]pyrimidine;
2-(1H-Indazol-4-yl)-6-(3-methyl-piperidin-1-ylmethyl)-4-morpholin-4-yl-thieno[3,2-d]pyrimidine;
{1-[2(1H-Indazol-4-yl)-4-morpholin-4-yl-thieno[3,2-d]pyrimidin-6-ylmethyl]-piperidin-3-yl}-methanol;
2-{1-[2-(1H-Indazol-4-yl)-4-morpholin-4-yl-thieno[3,2-d]pyrimidin-6-ylmethyl]-piperidin-4-yl}-ethanol;
1-[2-(1H-Indazol-4-yl)-4-morpholin-4-yl-thieno[3,2-d]pyrimidin-6-ylmethyl]-4-thiazol-2-yl-piperidin-4-ol;
2-(1-Methyl-1H-indazol-4-yl)-6-(4-methyl-piperazin-1-ylmethyl)-4-morpholin-4-yl-thieno[3,2-d]pyrimidine;
2-(2-Methyl-2H-indazol-4-yl)-6-(4-methyl-piperazin-1-ylmethyl)-4-morpholin-4-yl-thieno[3,2-d]pyrimidine;
2-(1H-Indazol-4-yl)-4-morpholin-4-yl-6-(4-thiazol-4-ylmethyl-piperazin-1-ylmethyl)-thieno[3,2-d]pyrimidine;
1-{4-[2-(1H-Indazol-4-yl)-4-morpholin-4-yl-thieno[3,2-d]pyrimidin-6-ylmethyl]-piperazin-1-yl}-3-phenoxy-propan-2-ol;
6-[4-(1H-Imidazol-2-ylmethyl)-piperazin-1-ylmethyl]-2-(1H-indazol-4-yl)-4-morpholin-4-yl-thieno[3,2-d]pyrimidine;
6-[4-(3H-Imidazol-4-ylmethyl)-piperazin-1-ylmethyl]-2-(1H-indazol-4-yl)-4-morpholin-4-yl-thieno[3,2-d]pyrimidine;
2-(1H-Indazol-4-yl)-4-morpholin-4-yl-6-((2S,6R)-2,4,6-trimethyl-piperazin-1-ylmethyl)-thieno[3,2-d]pyrimidine;
{4-[2-(1H-Indazol-4-yl)-4-morpholin-4-yl-thieno[3,2-d]pyrimidin-6-ylmethyl]-1-methanesulfonyl-piperazin-2-yl]-methanol; and
2-(1H-Indazol-4-yl)-6-(4-methanesulfonyl-3-methoxymethyl-piperazin-1-ylmethyl)-4-morpholin-4-yl-thieno[3,2-d]pyrimidine;
and the pharmaceutically acceptable salts of the above-mentioned free compounds.

24. The method of claim 20, wherein the selective inhibitor of PI3K alpha is selected from the following compounds: 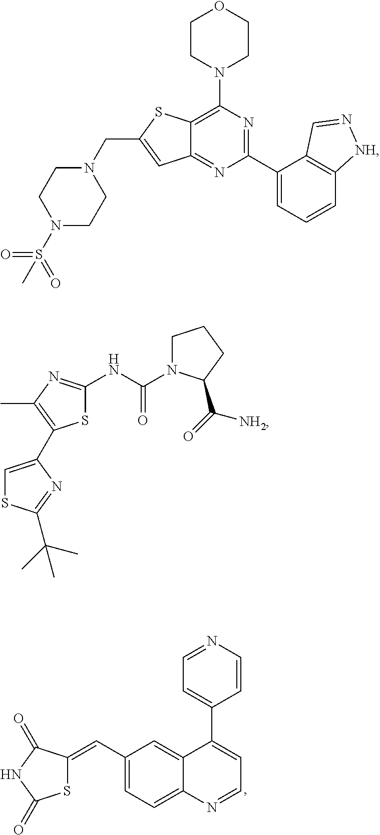 [SMILES: CS(=O)(=O)N1CCN(Cc2cc3nc(-c4cccc5[nH]ncc45)nc(N4CCOCC4)c3s2)CC1 ; Cc1nc(NC(=O)N2CCC[C@H]2C(N)=O)sc1-c1csc(C(C)(C)C)n1 ; O=C1NC(=O)/C(=C/c2ccc3nccc(-c4ccncc4)c3c2)S1]
INK1117, and BYL719.

25. The method of claim 20, wherein the selective inhibitor of PI3K alpha is selected from 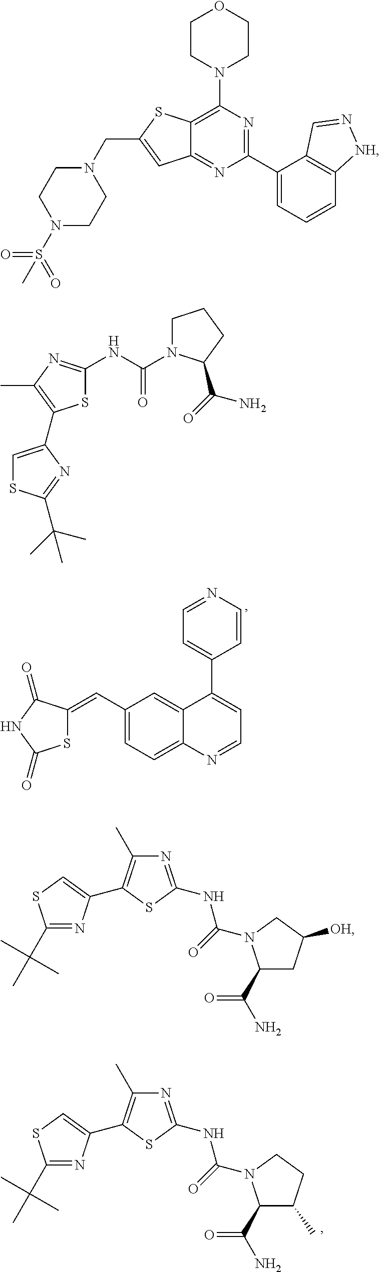 [SMILES: CS(=O)(=O)N1CCN(Cc2cc3nc(-c4cccc5[nH]ncc45)nc(N4CCOCC4)c3s2)CC1 ; Cc1nc(NC(=O)N2CCC[C@H]2C(N)=O)sc1-c1csc(C(C)(C)C)n1 ; O=C1NC(=O)/C(=C/c2ccc3nccc(-c4ccncc4)c3c2)S1 ; Cc1nc(NC(=O)N2C[C@@H](O)C[C@H]2C(N)=O)sc1-c1csc(C(C)(C)C)n1 ; Cc1nc(NC(=O)N2CC[C@H](C)[C@H]2C(N)=O)sc1-c1csc(C(C)(C)C)n1] 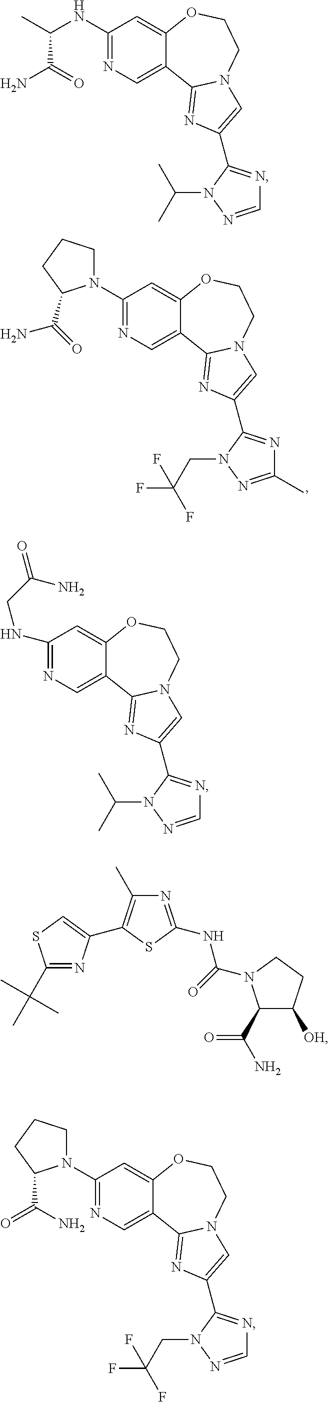 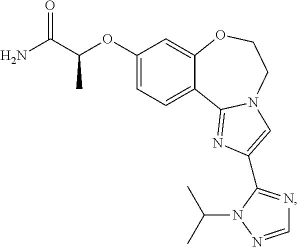 [SMILES: CC(C)n1ncnc1-c1cn2c(n1)-c1ccc(O[C@@H](C)C(N)=O)cc1OCC2]
INK1117, and BYL719.

26. The method of claim 20, wherein the selective inhibitor of P13K alpha is 4-[2(1H-indazol-4-yl)-6-[(4-methylsulfonylpiperazin-1-yl)methyl]thieno[3,2-d]pyrimidin-4-yl]morpholine.

27. The method of claim 9, wherein the selective inhibitor is an inhibitor of PI3K alpha that is also an inhibitor of PI3K delta.

28. The method of claim 9, wherein the selective inhibitor is an inhibitor of PI3K alpha, and the effective amount of the selective inhibitor of PI3K alpha is 750 nM.

29. The method of claim 9, wherein the effective amount of Activin A is 100 ng/ml of medium.

30. The method of claim 9, wherein culturing the cells under conditions sufficient to obtain the population of endoderm cells comprises culturing the cells in the absence of Wnt3a.

31. The method of claim 9, wherein the selective inhibitor comprises both an PI3K alpha inhibitor and an mTOR inhibitor.

32. The method of claim 9, wherein the method further comprises contacting the population of stem cells with a selective inhibitor of PI3K delta.

33. (canceled)

34. (canceled)

35. The method of claim 9, wherein the selective inhibitor is an inhibitor of mTOR, at least 61% of the cells in the population of endoderm cells express SOX17 or at least 40% of the cells in the population of endoderm cells express FoxA2.

36. The method of claim 35, wherein at least 61% of the cells in the population of endoderm cells express SOX17 and at least 40% of the cells in the population of endoderm cells express FoxA2.

37. (canceled)

38. The method of claim 9, wherein the selective inhibitor is an inhibitor of mTOR that is a siRNA or a small molecule.

39. The method of claim 38, wherein said small molecule is selected from the group consisting of: 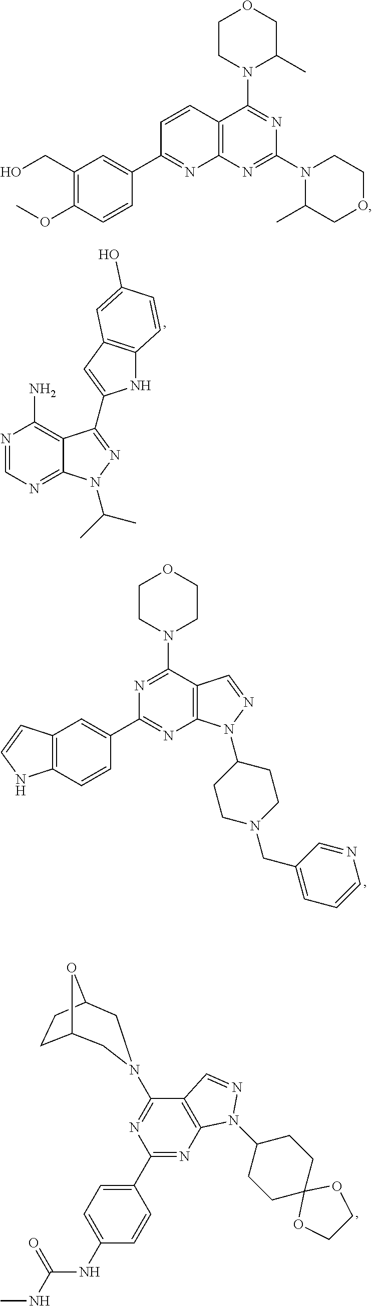 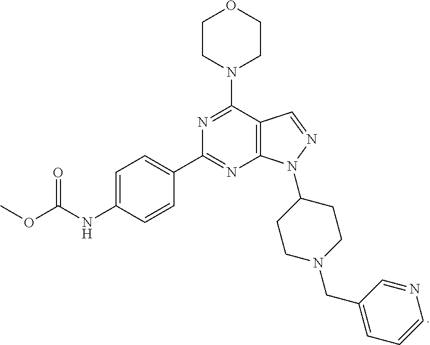 [SMILES: COC(=O)Nc1ccc(-c2nc(N3CCOCC3)c3cnn(C4CCN(Cc5cccnc5)CC4)c3n2)cc1]
AP23573, Torsel, INK128, AZD80555, AZD2012, CC-223, KU-0063794, OSI-027, sirolimus rapamycin, and everolimus.

40. The method of claim 38, wherein said small molecule is selected from the group consisting of:

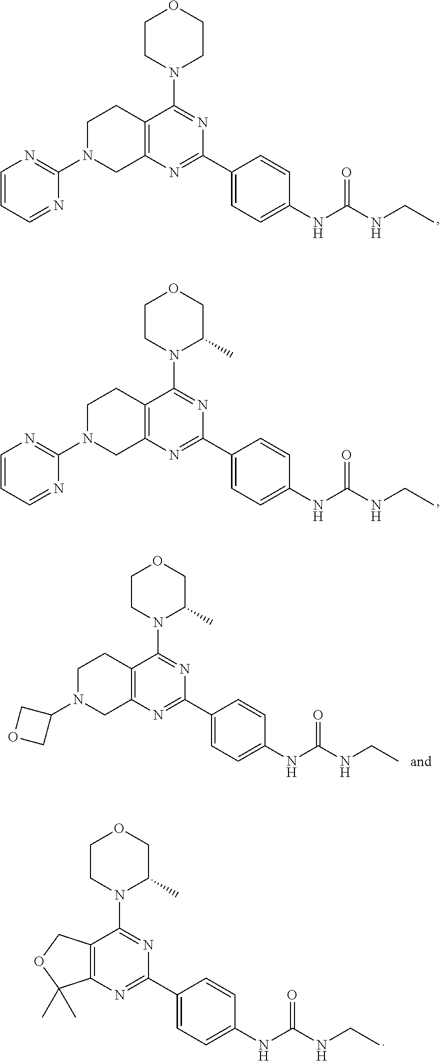 [SMILES: CCNC(=O)Nc1ccc(-c2nc3c(c(N4CCOCC4)n2)CCN(c2ncccn2)C3)cc1 ; CCNC(=O)Nc1ccc(-c2nc3c(c(N4CCOC[C@@H]4C)n2)CCN(c2ncccn2)C3)cc1 ; CCNC(=O)Nc1ccc(-c2nc3c(c(N4CCOC[C@@H]4C)n2)CCN(C2COC2)C3)cc1 ; "and" ; CCNC(=O)Nc1ccc(-c2nc(N3CCOC[C@@H]3C)c3c(n2)C(C)(C)OC3)cc1]

41. (canceled)

42. A method for identifying a factor that promotes the differentiation of endoderm cells into a cell type of interest, the method comprising: contacting a population of endoderm cells, wherein at least 83% of the cells in the population express SOX17, at least 77% of the cells in the population express FoxA2, or at least 76% of the cells in the population express CXCR4, with the factor, monitoring the population of endoderm cells for differentiation into the cell type of interest, thereby identifying the factor that promotes the differentiation of endoderm cells into a cell type of interest.

43. A method for identifying a factor that inhibits the differentiation of endoderm cells, the method comprising: contacting a population of endoderm cells, wherein at least 83% of the cells in the population express SOX17, at least 77% of the cells in the population express FoxA2, or at least 76% of the cells in the population express CXCR4, with the factor, monitoring the cells for differentiation, thereby identifying a factor that inhibits the differentiation of endoderm cells.

44. A method for screening a drug candidate for toxicity, the method comprising: contacting a population of endoderm cells, wherein at least 83% of the cells in the population express SOX17, at least 77% of the cells in the population express FoxA2, or at least 76% of the cells in the population express CXCR4, with the drug candidate and monitoring the cells for toxicity, thereby identifying whether the drug candidate is toxic.

45. A method of providing a cell-based therapy to a patient in need thereof, comprising administering to the patient a composition comprising calls selected from the group consisting of (i) a population of endoderm cells, wherein at least 83% of the cells in the population express SOX17, at least 77% of the cells in the population express FoxA2, or at least 76% of the cells in the population express CXCR4, (ii) a population of hepatocytes having one or more of the following properties: the hepatocytes secrete albumin, A1AT, or albumin and A1AT; CYP1A1/2 activity is inducible; or the hepatocytes express AFM, AFP, AGXT, ALB, CEBPA, CYP2C19, CYP2C9, CYP3A4, CYP3A7, CYP7A1, CABP1, FOXA1, FOXA2, GSTA1, HNF1A, HNF1B, HNF4a, IL6R, SERPINA1, SERPINA3, SERPINA7, SLCO2B1, TAT, VCAM1, or a combination thereof, (iii) a population of pancreatic progenitor cells wherein the pancreatic progenitor cells express one or more of the following markers: Pdx1, C-peptide, ARX, GLIS3, HNF1a, HNF1b, HNF4a, KRT19, MNX1, RFX6, SERPINA3, ONECUT1, NKX2-2, or any combination thereof, (iv) a population of differentiated pancreatic cells that form clusters in suspension and are viable in suspension, and (v) a mixture thereof.

46. The method of claim 45 wherein the patient is suffering from liver fibrosis, cirrhosis, liver failure, liver and pancreatic cancer, pancreatic failure, intestinal tissue replacement enzyme defects, Crohn's disease, inflammatory bowel syndrome, and intestinal cancer.

47. A method of obtaining a population of hepatocyte cells, the method comprising: culturing a population of endoderm cells, wherein at least 83% of the cells in the population express SOX17, at least 77% of the cells in the population express FoxA2, or at least 76% of the cells in the population express CXCR4, under conditions sufficient to obtain the population of hepatocyte cells.

48. The method of claim 47, wherein at least 56% of the hepatocyte cells in the population of hepatocyte cells express AFP.

49. The method of claim 47, wherein the endoderm cells are obtained by contacting a population of stem cells with an effective amount of a selective inhibitor of PI3K alpha and an effective amount of an Activin A and culturing the cells under conditions sufficient to obtain the population of hepatocyte cells.

50. A method of obtaining a population of hepatocyte cells, the method comprising: culturing a population of stem cells with an effective amount of a selective inhibitor of PI3K alpha and an effective amount of Activin A and culturing the cells under conditions sufficient to obtain the population of hepatocyte cells.

51. The method of claim 50, wherein the conditions sufficient to obtain the population of hepatocyte cells comprise culturing the endoderm cells in medium containing an effective amount of Activin A and lacking other growth factors.

52. The method of claim 50, wherein the other growth factors are selected from the group consisting of: FGF2, FGF4, BMP2, and BMP4.

53. (canceled)

54. An isolated population of hepatocytes wherein the hepatocytes have one or more of the following properties: the hepatocytes secrete albumin, A1AT, or albumin and A1AT; CYP1A1/2 activity is inducible; or the hepatocytes express AFM, AFP, AGXT, ALB, CEBPA, CYP2C19, CYP2C9, CYP3A4, CYP3A7, CYP7A1, CABP1, FOXA1, FOXA2, GSTA1, HNF1A, HNF1B, HNF4a, IL6R, SERPINA1, SERPINA3, SERPINA7, SLCO2B1, TAT, VCAM1, or a combination thereof.

55. (canceled)

56. A method of screening for a drug candidate for toxicity comprising contacting a population of hepatocytes obtained by the method of claim 47 with the drug candidate, and monitoring the hepatocytes for toxicity, thereby identifying whether the drug candidate is toxic.

57. A method for obtaining pancreatic progenitor cells, said method comprising:
A). culturing a population of stem cells with an effective amount of either (1) a mTOR inhibitor and an effective amount of Activin A or (2) a selective inhibitor of PI3K alpha and an effective amount of Activin A or (3) an mTOR inhibitor, a selective inhibitor of PI3K alpha, and effective amount of Activin A, and culturing the cells under conditions sufficient to obtain the population of endoderm cells; and
B). culturing the endoderm cells under conditions sufficient to promote the differentiation of endoderm cells to pancreatic progenitor cells.

58. A method for obtaining pancreatic progenitor cells, said method comprising: culturing a starting population of endoderm cells of claim 1 under conditions sufficient to promote the differentiation of endoderm cells to pancreatic progenitor cells.

59. The method of claim 58 wherein the pancreatic progenitor cells can differentiate into pancreatic endocrine cells, pancreatic exocrine cells and pancreatic ductal cells.

60. The method of claim 59 wherein the pancreatic endocrine cells are selected from the group consisting of alpha cells, beta cells, delta cells and gamma cells.

61. The method of claim 59 wherein the pancreatic endocrine cells are capable of producing one or more of: glucagon, insulin, somatostatin, and pancreatic polypeptide.

62. A method for obtaining differentiated pancreatic cells, said method comprising culturing pancreatic progenitor cells produced by the method of claim 58 under conditions sufficient to promote the differentiation of pancreatic progenitor cells to differentiated pancreatic cells.

63. The method of claim 62 wherein the differentiated pancreatic cells are selected from the group consisting of pancreatic endocrine cells, pancreatic exocrine cells and pancreatic ductal cells.

64. The method of claim 62 wherein the differentiated pancreatic cells are capable of producing one or more of: glucagon, insulin, somatostatin, and pancreatic polypeptide.

65. (canceled)

66. An isolated population of pancreatic progenitor cells wherein the pancreatic progenitor cells express one or more of the following markers: Pdx1, C-peptide, ARX, GLIS3, HNF1a, HNF1b, HNF4a, KRT19, MNX1, RFX6, SERPINA3, ONECUT1, NKX2-2, or any combination thereof.

67. (canceled)

68. An isolated population of differentiated pancreatic cells wherein the pancreatic cells form clusters in suspension and are viable in suspension.

69. (canceled)

70. (canceled)

71. A method of screening for a drug candidate for toxicity comprising contacting a population of pancreatic cells obtained by the method of claim 57 with a drug candidate, monitoring the pancreatic cells for toxicity, thereby identifying whether the drug candidate is toxic.
